(19) United States
(12) Patent Application Publication
(10) Pub. No.: US 2026/0083859 A1
(43) Pub. Date: Mar. 26, 2026

(54) SUSTAINED TRANSGENE EXPRESSION OF MODIFIED ERT2 PEPTIDE-SUICIDE PROTEIN FUSION POLYPEPTIDES

(71) Applicants: Senti Biosciences, Inc., South San Francisco, CA (US); Bluerock Therapeutics LP, Cambridge, MA (US)

(72) Inventors: Rebecca Tayler Cottman, San Francisco, CA (US); Michelle Elizabeth Hung, South San Francisco, CA (US); Russell Morrison Gordley, South San Francisco, CA (US); Timothy Kuan-Ta Lu, South San Francisco, CA (US); Stefan Irion, Cambridge, MA (US); Mark Tomishima, Cambridge, MA (US); Chew-Li Soh, Cambridge, MA (US); Conor McAuliffe, Cambridge, MA (US)

(21) Appl. No.: 19/287,644

(22) Filed: Jul. 31, 2025

Related U.S. Application Data
(63) Continuation of application No. PCT/US2024/014331, filed on Feb. 2, 2024.
(60) Provisional application No. 63/482,987, filed on Feb. 2, 2023.
(60) Provisional application No. 63/466,156, filed on May 12, 2023.

(51) Int. Cl. A61K 48/00; A61P 37/02; C07K 14/47; C07K 14/72; C12N 9/22; C12N 9/64; C12N 15/11; C12N 15/62; C12N 15/85

(52) U.S. Cl. CPC A61K 48/005; A61P 37/02; C07K 14/4702; C07K 14/721; C12N 9/222; C12N 9/6472; C12N 15/111; C12N 15/62; C12N 15/85; C07K 2319/715; C12N 2310/20; C12N 2840/203; C12Y 304/22062

(57) ABSTRACT

Provided herein are targeting constructs for sustained transgene expression of inducible cell death systems that include mutants of estrogen receptor alpha ligand binding domain (ER-LBD). Also provided are methods for use of the same, such as inducing cell death in a cell.

### CROSS-REFERENCE TO RELATED APPLICATIONS

This application is a continuation application of International Application No. PCT/US2024/014331 filed Feb. 2, 2024, now pending; which claims the benefit under 35 USC § 119(e) to U.S. Application Ser. No. 63/482,987 filed Feb. 2, 2023 and to U.S. Application Ser. No. 63/466,156 filed May 12, 2023, both now expired. The disclosure of each of the prior applications is considered part of and is incorporated by reference in the disclosure of this application.

### INCORPORATION BY REFERENCE OF SEQUENCE LISTING

The material in the accompanying sequence listing is hereby incorporated by reference into this application. The accompanying sequence listing xml filed, named SENTI1410-2_ST26.xml was created on Jul. 31, 2025 and is 107,627 bytes in size.

### BACKGROUND OF THE INVENTION

Currently available cell and gene therapy products can lack control of function, which can lead to safety concerns such as toxicity in subjects that receive the therapies. Thus, additional methods of controlling and regulating these therapies are needed.

### SUMMARY OF THE INVENTION

Estrogen receptor (ER) is a ligand-dependent transcription factor that binds endogenous hormone ligands such as estrogen and estradiol. Synthetic ligands that bind to ER have been developed for treating ER-positive cancers such as ER-positive breast cancer. For example, active metabolites of the drug tamoxifen induce nuclear translocation of ER and antagonize ER in a tissue-selective manner. Tamoxifen and its active metabolites are also utilized as a tool for controlling nuclear localization in the research setting. For example, it can be desirable to engineer cell and gene therapy products with sustained transgene expression of a “kill” switch, e.g., an inducible cell death system, as a safety mechanism to address adverse events related to administration of cell or gene therapy. In the context of cell therapies or gene therapies where the administered therapeutic cells or cells transfected with a gene therapy transform from one state to another (e.g., from iPSC to a differentiated cell), it is pertinent that a kill switch is functional even after transformation. Thus, sustained transgene expression of modified ERT2-based systems with improved sensitivity to and/or selectivity for synthetic ligands would be useful for suicide-switch mediated regulated cell killing in a clinical setting.

Provided herein is a targeting construct comprising:
(a) a first homology arm corresponding to a 5′ target sequence comprising a first region of homology to a target genomic locus;
(b) a nucleotide insert comprising a nucleotide sequence encoding a fusion polypeptide comprising:
   (i) a modified estrogen receptor ligand binding domain (ER-LBD); and
   (ii) a caspase 9 domain or derivative or functional fragment thereof;
(c) a second homology arm corresponding to a 3′ target sequence comprising second region of homology to the target genomic locus wherein the modified ER-LBD comprises an amino acid sequence corresponding to a hormone binding domain of a reference human estrogen receptor sequence (SEQ ID NO: 1), and wherein the modified ER-LBD comprises
a G400V amino acid substitution, an M543A amino acid substitution, an L544A amino acid substitution, and optionally a V595A amino acid substitution, with reference to SEQ ID NO: 1; and additional amino acid substitutions, wherein the additional amino acid substitutions comprise, with reference to SEQ ID NO: 1:
   (i) an L384M substitution, an L391V substitution, a N413D substitution, an M421L substitution, a S463P substitution, and a H524L substitution,
   (ii) an L391V substitution, a N413D substitution, a Q414E substitution, a S463P substitution, and a H524F substitution,
   (iii) an L354I substitution, a L391V substitution, a N413D substitution, a Q414E substitution, a M421L substitution, a M517A substitution, and a H524F substitution,
   (iv) an L354I substitution, a L391V substitution, a L409V substitution, a N413D substitution, a Q414E substitution, and a H524L substitution,
   (v) an L391V substitution, a Q414E substitution, an N413D substitution, an S463P substitution, an M421L substitution, an L354I substitution, an L384M substitution, and an H524L substitution,
   (vi) an L391V substitution, an N413D substitution, an S463P substitution, an M517A substitution, an M421L substitution, an L354I substitution, and an H524L substitution,
   (vii) an N413D substitution, an S463P substitution, an L354I substitution, an L384M substitution, and an H524L substitution, or
   (viii) an L391V substitution, an N413D substitution, an S463P substitution, an M517A substitution, an M421L substitution, an L354I substitution, and an H524L substitution,
wherein the targeting construct is configured such that upon its recombination with the target genomic locus, the fusion polypeptide coding sequence is integrated into a STEL gene and becomes operably linked to the STEL gene regulatory element.

Also provided herein, in various embodiments, is a targeting construct comprising: (a) a first homology arm corresponding to a 5′ target sequence comprising a first region of homology to a target genomic locus; (b) a nucleotide insert comprising a nucleotide sequence encoding a fusion polypeptide comprising: (i) a first modified estrogen receptor ligand binding domain (ER-LBD); (ii) a first caspase 9 domain or derivative or functional fragment thereof; (iii) a second modified estrogen receptor ligand binding domain (ER-LBD); and (iv) a second caspase 9 domain or derivative or functional fragment thereof; (c) a second homology arm corresponding to a 3′ target sequence comprising second region of homology to the target genomic locus, wherein the first modified ER-LBD and the second modified ER-LBD each comprise an amino acid sequence corresponding to a hormone binding domain of a reference human estrogen receptor sequence (SEQ ID NO: 1), and wherein the first modified ER-LBD and the second modified ER-LBD each independently comprise: a G400V amino acid substitution, an M543A amino acid substitution, an L544A amino acid substitution, and optionally a V595A amino acid substitution, with reference to SEQ ID NO: 1; and additional amino acid substitutions, wherein the additional amino acid substitutions comprise, with reference to SEQ ID NO: 1: (i) an L384M substitution, an L391V substitution, a N413D substitution, an M421L substitution, a S463P substitution, and a H524L substitution, (ii) an L391V substitution, a N413D substitution, a Q414E substitution, a S463P substitution, and a H524F substitution, (iii) an L354I substitution, a L391V substitution, a N413D substitution, a Q414E substitution, a M421L substitution, a M517A substitution, and a H524F substitution, (iv) an L354I substitution, a L391V substitution, a L409V substitution, a N413D substitution, a Q414E substitution, and a H524L substitution, (v) an L391V substitution, a Q414E substitution, an N413D substitution, an S463P substitution, an M421L substitution, an L354I substitution, an L384M substitution, and an H524L substitution, (vi) an L391V substitution, an N413D substitution, an S463P substitution, an M517A substitution, an M421L substitution, an L354I substitution, and an H524L substitution, (vii) an N413D substitution, an S463P substitution, an L354I substitution, an L384M substitution, an H524L substitution, or (viii) an L391V substitution, an N413D substitution, an S463P substitution, an M517A substitution, an M421L substitution, an L354I substitution, and an H524L substitution, wherein the targeting construct is configured such that upon its recombination with the target genomic locus, the fusion polypeptide coding sequence is integrated into a STEL gene and becomes operably linked to the STEL gene regulatory element.

In some embodiments, the additional amino acid substitutions comprise: an L384M substitution, an L391V substitution, a N413D substitution, an M421L substitution, a S463P substitution, and a H524L substitution, and wherein the modified ER-LBD comprises an amino acid sequence having at least 80%, at least 85%, at least 90%, or at least 95% sequence identity to the amino acid sequence of (SEQ ID NO:2). In some embodiments, the modified ER-LBD comprises an amino acid sequence having at least 98% sequence identity to the amino acid sequence of SEQ ID NO:2. In some embodiments, the modified ER-LBD comprises an amino acid sequence having at least 99% sequence identity to the amino acid sequence of SEQ ID NO:2. In some embodiments, the modified ER-LBD comprises or consists of an amino acid sequence having 100% sequence identity to the amino acid sequence of SEQ ID NO:2.

In some embodiments, the additional amino acid substitutions comprise: an L391V substitution, a N413D substitution, a Q414E substitution, a S463P substitution, and a H524F substitution, and wherein the modified ER-LBD comprises an amino acid sequence having at least 80%, at least 85%, at least 90%, or at least 95% sequence identity to the amino acid sequence of SEQ ID NO:3. In some embodiments, the modified ER-LBD comprises an amino acid sequence having at least 98% sequence identity to the amino acid sequence of SEQ ID NO:3. In some embodiments, the modified ER-LBD comprises an amino acid sequence having at least 99% sequence identity to the amino acid sequence of SEQ ID NO:3. In some embodiments, the modified ER-LBD comprises or consists of an amino acid sequence having 100% sequence identity to the amino acid sequence of SEQ ID NO:3.

In some embodiments, the additional amino acid substitutions comprise: an L354I substitution, a L391V substitution, a N413D substitution, a Q414E substitution, a M421L substitution, a M517A substitution, and a H524F substitution, and wherein the modified ER-LBD comprises an amino acid sequence having at least 80%, at least 85%, at least 90%, or at least 95% sequence identity to the amino acid sequence of SEQ ID NO:4. In some embodiments, the modified ER-LBD comprises an amino acid sequence having at least 98% sequence identity to the amino acid sequence of SEQ ID NO:4. In some embodiments, the modified ER-LBD comprises an amino acid sequence having at least 99% sequence identity to the amino acid sequence of SEQ ID NO:4. In some embodiments, the modified ER-LBD comprises or consists of an amino acid sequence having 100% sequence identity to the amino acid sequence of SEQ ID NO:4. In some embodiments, the additional amino acid substitutions comprise: an L354I substitution, a L391V substitution, a L409V substitution, a N413D substitution, a Q414E substitution, and a H524L substitution, and wherein the modified ER-LBD comprises an amino acid sequence having at least 80%, at least 85%, at least 90%, or at least 95% sequence identity to the amino acid sequence of SEQ ID NO:5. In some embodiments, the modified ER-LBD comprises an amino acid sequence having at least 98% sequence identity to the amino acid sequence of SEQ ID NO:5. In some embodiments, the modified ER-LBD comprises an amino acid sequence having at least 99% sequence identity to the amino acid sequence of SEQ ID NO:5. In some embodiments, the modified ER-LBD comprises or consists of an amino acid sequence having 100% sequence identity to the amino acid sequence of SEQ ID NO:5.

In some embodiments, the additional amino acid substitutions comprise: an L391V substitution, a Q414E substitution, an N413D substitution, an S463P substitution, an M421L substitution, an L354I substitution, an L384M substitution, and an H524L substitution, and the modified ER-LBD comprises an amino acid sequence having at least 80%, at least 85%, at least 90%, or at least 95% sequence identity to the amino acid sequence of SEQ ID NO:40. In some embodiments, the modified ER-LBD comprises an amino acid sequence having at least 98% sequence identity to the amino acid sequence of SEQ ID NO:40. In some embodiments, the modified ER-LBD comprises an amino acid sequence having at least 99% sequence identity to the amino acid sequence of SEQ ID NO:40. In some embodiments, the modified ER-LBD comprises or consists of an amino acid sequence having 100% sequence identity to the amino acid sequence of SEQ ID NO:40.

In some embodiments, the additional amino acid substitutions comprise: an L391V substitution, an N413D substitution, an S463P substitution, an M517A substitution, an M421L substitution, an L354I substitution, and an H524L substitution, and the modified ER-LBD comprises an amino acid sequence having at least 80%, at least 85%, at least 90%, or at least 95% sequence identity to the amino acid sequence of SEQ ID NO:41. In some embodiments, the modified ER-LBD comprises an amino acid sequence having at least 98% sequence identity to the amino acid sequence of SEQ ID NO:41. In some embodiments, the modified ER-LBD comprises an amino acid sequence having at least 99% sequence identity to the amino acid sequence of SEQ ID NO:41. In some embodiments, the modified ER-LBD comprises or consists of an amino acid sequence having 100% sequence identity to the amino acid sequence of SEQ ID NO:41.

In some embodiments, the additional amino acid substitutions comprise: an N413D substitution, an S463P substitution, an L354I substitution, an L384M substitution, and an H524L substitution, and the modified ER-LBD comprises an amino acid sequence having at least 80%, at least 85%, at least 90%, or at least 95% sequence identity to the amino acid sequence of SEQ ID NO:42. In some embodiments, the modified ER-LBD comprises an amino acid sequence having at least 98% sequence identity to the amino acid sequence of SEQ ID NO:42. In some embodiments, the modified ER-LBD comprises an amino acid sequence having at least 99% sequence identity to the amino acid sequence of SEQ ID NO:42. In some embodiments, the modified ER-LBD comprises or consists of an amino acid sequence having 100% sequence identity to the amino acid sequence of SEQ ID NO:42.

In some embodiments, the additional amino acid substitutions comprise: an L391V substitution, an N413D substitution, an S463P substitution, an M517A substitution, an M421L substitution, an L354I substitution, and an H524L substitution, and the modified ER-LBD comprises an amino acid sequence having at least 80%, at least 85%, at least 90%, or at least 95% sequence identity to the amino acid sequence of SEQ ID NO:43. In some embodiments, the modified ER-LBD comprises an amino acid sequence having at least 98% sequence identity to the amino acid sequence of SEQ ID NO:43. In some embodiments, the modified ER-LBD comprises an amino acid sequence having at least 99% sequence identity to the amino acid sequence of SEQ ID NO:43. In some embodiments, the modified ER-LBD comprises or consists of an amino acid sequence having 100% sequence identity to the amino acid sequence of SEQ ID NO:43.

In some embodiments, the modified ERT2 peptide further comprises an N-terminal serine (S) residue.

In some embodiments, the modified ER-LBD comprises the V595A amino acid substitution.

In some embodiments, the caspase 9 domain or derivative or functional fragment thereof does not comprise a Caspase Activation and Recruitment Domain (CARD) domain sequence. In some embodiments, the caspase 9 domain or derivative or functional fragment thereof comprises (SEQ ID NO:6).

In some embodiments, the modified estrogen receptor ligand binding domain (ER-LBD) is N-terminal to the caspase 9 domain or derivative or functional fragment thereof. In some embodiments, the modified estrogen receptor ligand binding domain (ER-LBD) is C-terminal to the caspase 9 domain or derivative or functional fragment thereof.

In some embodiments, the fusion polypeptide comprises a linker between the modified estrogen receptor ligand binding domain (ER-LBD) and the caspase 9 domain or derivative or functional fragment thereof.

In some embodiments, the targeting construct is configured such that upon its recombination with the target genomic locus, the STEL gene is modified such to incorporate the fusion polypeptide coding sequence 3′ to the STEL protein coding sequence. In some embodiments, the targeting construct is configured such that upon its recombination with the target genomic locus, the STEL gene is modified such to incorporate the fusion polypeptide coding sequence 5′ to the STEL protein coding sequence.

In some embodiments, the nucleotide insert further comprises a nucleotide sequence encoding a separator sequence. In some embodiments, the targeting construct is configured such that upon recombination of the targeting construct with the target genomic locus, the separator sequence coding sequence is positioned between the coding sequence of the STEL protein and the fusion polypeptide coding sequence. In some embodiments, the separator sequence is an internal ribosome entry site (“IRES”). In some embodiments, the separator sequence is a self-cleaving peptide. In some embodiments, the self-cleaving peptide is a 2A peptide. In some embodiments, the self-cleaving peptide is T2A, P2A, E2A, F2A, PQR, Opt2A, or Opt2A_2.0.

In some embodiments, the STEL gene encodes a polypeptide involved in one or more of: glycolysis, ribonucleopolypeptide complex formation, focal adhesion, cell-substrate adherens junction, cell-substrate junction, cell anchoring, extracellular exosome, extracellular vesicle, intracellular organelle, anchoring junction, RNA binding, nucleic acid binding (e.g., rRNA or mRNA binding), and polypeptide binding.

In some embodiments, the STEL gene encodes a ribosomal polypeptide. In some embodiments, the STEL gene is RPL13A, RPLP0, RPL10, RPL13, RPSJ8, RPL3, RPLP1, RPL15, RPL41, RPL11, RPL32, RPL18A, RPL19, RPL28, RPL29, RPL9, RPL8, RPL6, RPL18, RPL7, RPL7A, RPL21, RPL37A, RPL 12, RPL5, RPL34, RPL35A, RPL30, RPL24, RPL39, RPL37, RPL14, RPL27A, RPLP2, RPL23A, RPL26, RPL36, RPL35, RPL23, RPL4, or RPL22.

In some embodiments, the STEL gene encodes a ribosomal polypeptide small subunit (RPS). In some embodiments, the STEL gene is RPS2, RPS19, RPS14, RPS3A, RPS12, RPS3, RPS6, RPS23, RPS27A, RPS8, RPS4X, RPS7, RPS24, RPS27, RPS15A, RPS9, RPS28, RPS13, RPSA, RPS5, RPS 16, RPS25, RPS15, RPS20, or RPS11.

In some embodiments, the STEL gene encodes a mitochondrial polypeptide. In some embodiments, the STEL gene is MT-CO1, MT-C02, MT-ND4, MT-ND1, or MT-ND2.

In some embodiments, the STEL gene encodes an actin polypeptide. In some embodiments, the STEL gene is ACTG1 or ACTB.

In some embodiments, STEL gene encodes a eukaryotic translation factor. In some embodiments, the STEL gene is EEF1A1, EEF2, or EIF1.

In some embodiments, the STEL gene encodes a histone. In some embodiments, the STEL gene is H3F3A or H3F3B.

In some embodiments, the STEL gene is FTL, FTH1, TPT1, IMSB10, GAPDH, PTMA, GNB2L1, NACA, YBX1, NPM1, FAU, UBA52, HSP90AB1, MYL6, SERF2, or SRP14. In some embodiments, the STEL gene is GAPDH.

In some embodiments, the STEL gene is RPL13A. In some embodiments, the STEL gene is RPL7. In some embodiments, the STEL gene is RPLP0.

In some embodiments, the nucleotide insert further comprises a transgene. In some embodiments, the transgene is linked to the fusion polypeptide coding sequence. In some embodiments, the fusion polypeptide coding sequence and transgene are connected via a nucleotide sequence encoding a separator sequence. In some embodiments, the separator sequence is an internal ribosome entry site (“IRES”). In some embodiments, the separator sequence is a nucleotide sequence encoding a self-cleaving peptide (the “self-cleaving peptide coding sequence”). In some embodiments, the self-cleaving peptide is a 2A peptide. In some embodiments, the self-cleaving peptide is T2A, P2A, E2A, F2A, PQR, Opt2A, or Opt2A_2.0. In some embodiments, the transgene encodes a therapeutic polypeptide.

Provided herein is a system comprising:
(a) the targeting construct disclosed herein;
(b) a CRISPR-associated endonuclease (“Cas polypeptide”) or a nucleic acid encoding a Cas polypeptide; and
(c) a guide RNA (“gRNA”) comprising a scaffold for binding the Cas polypeptide and a spacer sequence corresponding to the STEL gene, or a nucleic acid encoding the gRNA.

In some embodiments, the guide RNA is a single guide RNA (“sgRNA”). In some embodiments, the system comprises the Cas polypeptide and gRNA. In some embodiments, the system is in the form of a ribonucleoprotein particle (“RNP”).

Provided herein is a method of producing a gene-edited target cell, comprising: (a) introducing the system of disclosed herein into a target cell; and (b) culturing the target cell under conditions in which gene editing occurs, thereby producing gene-edited target cell.

In some embodiments, the target cell is a stem cell or a cell differentiated from a stem cell. In some embodiments, the target cell is a stem cell. In some embodiments, the stem cell is a human embryonic stem cell, an induced pluripotent stem cell (“iPSC”) or a cell differentiated therefrom. In some embodiments, the target cell is: (a) a regulatory T cell, a myeloid cell, a dendritic cell, and a macrophage (e.g., an immunosuppressive macrophage), or a precursor or progenitor cell thereof; a cell in the human nervous system, optionally selected from dopaminergic neuron, a microglial cell, an oligodendrocyte, an astrocyte, a cortical neuron, a spinal or oculomotor neuron, an enteric neuron, a Placode-derived cell, a Schwann cell, and a trigeminal or sensory neuron, or a precursor or progenitor cell thereof; a cell in the human cardiovascular system, optionally selected from a cardiomyocyte, an endothelial cell, and a nodal cell, or a precursor or progenitor cell thereof; a cell in the human metabolic system, optionally selected from a hepatocyte, a cholangiocyte, and a pancreatic beta cell, or a precursor or progenitor cell thereof, or (b) a cell in the human ocular system, optionally selected from a retinal pigment epithelial cell, a photoreceptor cone cell, a photoreceptor rod cell, a bipolar cell, a ganglion cell, or a precursor or progenitor cell thereof.

In some embodiments, the gene-edited target cell is of ectoderm lineage, optionally wherein the gene-edited target cell is a neuron. In some embodiments, the gene-edited target cell is of mesoderm lineage, optionally wherein the gene-edited target cell is a cardiomyocyte.

Provided herein is a gene-edited target cell obtained or obtainable by any one of the above methods.

Provided herein is a gene-edited target cell comprising a STEL gene that comprises a nucleic acid encoding a fusion polypeptide under the transcriptional control of a STEL gene regulatory element, the fusion polypeptide comprising: (a) a modified estrogen receptor ligand binding domain (ER-LBD); and (b) a caspase 9 domain or derivative or functional fragment thereof,
wherein the modified ER-LBD comprises an amino acid sequence corresponding to a hormone binding domain of a reference human estrogen receptor sequence (SEQ ID NO: 1), and wherein the modified ER-LBD comprises
a G400V amino acid substitution, an M543A amino acid substitution, an L544A amino acid substitution, and optionally a V595A amino acid substitution, with reference to SEQ ID NO: 1; and
additional amino acid substitutions, wherein the additional amino acid substitutions comprise, with reference to SEQ ID NO: 1:
(i) an L384M substitution, an L391V substitution, a N413D substitution, an M421L substitution, a S463P substitution, and a H524L substitution,
(ii) an L391V substitution, a N413D substitution, a Q414E substitution, a S463P substitution, and a H524F substitution,
(iii) an L354I substitution, a L39TV substitution, a N413D substitution, a Q414E substitution, a M421L substitution, a M517A substitution, and a H524F substitution,
(iv) an L354I substitution, a L391V substitution, a L409V substitution, a N413D substitution, a Q414E substitution, and a H524L substitution,
(v) an L391V substitution, a Q414E substitution, an N413D substitution, an S463P substitution, an M421L substitution, an L354I substitution, an L384M substitution, and an H524L substitution,
(vi) an L391V substitution, an N413D substitution, an S463P substitution, an M517A substitution, an M421L substitution, an L354I substitution, and an H524L substitution,
(vii) an N413D substitution, an S463P substitution, an L354I substitution, an L384M substitution, and an H524L substitution, or
(viii) an L391V substitution, an N413D substitution, an S463P substitution, an M517A substitution, an M421L substitution, an L354I substitution, and an H524L substitution.

In some embodiments, the modified ER-LBD is as defined as provided above. In some embodiments, the caspase 9 domain or derivative or functional fragment thereof is as defined above. In some embodiments, the fusion polypeptide is as defined above. In some embodiments, the STEL gene is configured as defined above. In some embodiments, the gene-edited target cell further comprises a transgene in the STEL gene. In some embodiments, the transgene is as defined above. In some embodiments, the target cell is as defined above.

Provided herein is a pharmaceutical composition comprising the gene-edited target cell as provided herein and a pharmaceutically acceptable carrier.

Provided herein is a method of treating a patient in need thereof, comprising administering to the patient the gene-edited target cell as provided herein or a pharmaceutical composition comprising the gene-edited target cell and a pharmaceutically acceptable carrier. In some embodiments, the method further comprises controlling the gene-edited target cell population in the patient by: (a) monitoring, optionally, the gene-edited target cell population in the patient; and (b) administering an inducer of the modified ER-LBD if the patient experiences adverse events related to the gene-edited target cell population.

Provided herein is a method of mitigating adverse events or a safety risk associated with cell therapy in the form of the gene-edited target cells as defined above or the pharmaceutical composition of as defined above, the method comprising administering to a patient who received the gene-edited target cells or pharmaceutical composition an inducer of a modified ER-LBD if the patient experiences adverse events or a safety risk related to the gene-edited target cells or pharmaceutical composition.

In some embodiments of the foregoing administration methods, a single inducer of the modified ER-LBD is administered. In other embodiments, a combination of two or more inducers of the modified ER-LBD are administered. In some embodiments, the inducer(s) of the modified ER-LBD comprise tamoxifen and/or a tamoxifen metabolite. In some embodiments, the inducer(s) of the modified ER-LBD comprise tamoxifen. In some embodiments, the inducer(s) of the modified ER-LBD comprise a tamoxifen metabolite. In some embodiments, the tamoxifen metabolite is 4-hydroxytamoxifen, N-desmethyltamoxifen, tamoxifen-N-oxide, or endoxifen.

In some embodiments, the targeting construct comprises an amino acid sequence at least 80%, at least 85%, at least 90%, at least 91%, at least 92%, at least 93%, at least 94%, at least 95%, at least 96%, at least 97%, at least 98%, at least 99%, at least 99.5%, or at least 99.9% identical to an amino acid sequence set forth in SEQ ID NO: 13, 15, 17, 19, 21, 23, 25, 27, 44, 46, 48, or 50. In some embodiments, the targeting construct comprises an amino acid sequence set forth in SEQ ID NO: 13, 15, 17, 19, 21, 23, 25, 27, 44, 46, 48, or 50.

### BRIEF DESCRIPTION OF THE DRAWINGS

FIGS. 1A-1B are schematic diagrams of exemplary constructs of the disclosure, comprising a nucleic acid sequence encoding a modified ERT2 peptide-suicide protein fusion polypeptide disclosed herein flanked by sustained transcription expression locus (STEL) homology arms. FIG. 1A is an exemplary construct, which comprises from 5′ to 3′, a STEL left homology arm (LHA), a separator sequence (SS), a coding sequence for at least one modified estrogen receptor ligand binding domain (modified ER-LBD, also referred to interchangeably herein as modified ERT2 peptide), a suicide protein coding sequence for a caspase 9 domain or derivative or functional fragment thereof, and a STEL right homology arm (RHA). FIG. 1B is another exemplary construct, which comprises from 5′ to 3′, a STEL left homology arm, a separator sequence, a modified ERT2 peptide encoding sequence disclosed herein, a suicide protein coding sequence for a caspase 9 domain or derivative or functional fragment thereof (“Casp9”), an optional separator sequence, an optional transgene, and a STEL right homology arm. Modified ERT2 peptides and Caspase-9 sequences in the constructs depicted in FIGS. 1A and 1B are described in Sections 0 and 0, respectively. Although in FIGS. 1A-1B the modified ERT2 peptide coding sequence is shown 5′ to the suicide protein coding sequence and encoding a fusion polypeptide with a modified ERT2 peptide N-terminal to the suicide protein, the fusion polypeptides of the disclosure may additionally or alternatively include a modified ERT2 peptide C-terminal to the suicide protein. The modified ERT2 peptide and suicide protein sequences may be separated by a linker.

FIGS. 2A-2B are schematic diagrams of exemplary constructs of the disclosure, comprising a sequence encoding a modified ERT2 peptide-suicide protein fusion polypeptide, flanked by GAPDH homology arms, wherein the sequence encoding a modified ERT2 peptide-suicide protein fusion polypeptide comprises a modified ERT2 peptide encoding sequence disclosed herein and a suicide protein coding sequence for a caspase 9 domain or derivative or functional fragment thereof (“Casp9”). FIG. 2A is an exemplary construct, which comprises from 5′ to 3′, a GAPDH left homology arm (LHA), a T2A sequence, an ERT2 peptide sequence, a Casp9 sequence, and a GAPDH right homology arm (RHA). FIG. 2B is an exemplary construct, which comprises from 5′ to 3′, a GAPDH left homology arm, a T2A sequence, an ERT2 peptide sequence, a Casp9 sequence, and IRES, an RFP marker, and a GAPDH right homology arm. Although in FIGS. 2A-2B the ERT2 peptide coding sequence is shown 5′ to the suicide protein coding sequence and encoding a fusion polypeptide with an ERT2 peptide N-terminal to the suicide protein, the fusion polypeptides of the disclosure may additionally or alternatively include an ERT2 peptide C-terminal to the suicide protein. The ERT2 peptide and suicide protein sequences may be separated by a linker.

FIG. 3 is an illustration depicting the mechanism of apoptosis in cells transfected with a construct of the disclosure. In the absence of an ERT2 inducer such as tamoxifen, a modified ERT2 peptide-suicide protein fusion polypeptide of the disclosure comprising a caspase-9 peptide and ERT2, do not dimerize and remain inactive. Addition of a suitable hormone or hormone analog (e.g., tamoxifen) that binds to the modified ERT2 peptide allows dimerization of the modified ERT2 peptide-suicide protein fusion polypeptide. This dimerization activates Caspase-9, which leads to apoptosis.

FIGS. 4A-4B are graphs demonstrating targeted integration of modified ERT2 peptide-suicide protein fusion polypeptides Construct 1, Construct 2, Construct 3, and Construct 4 at GAPDH loci in the presence and absence of 4-OHT. FIG. 4A is a bar graph depicting junction GAPT2A ddPCR results in a clonal human iPSC line with a mono-allelic integration of a T2A containing construct at GAPDH or depicting pooled junction GAPT2A ddPCR results in one of the modified ERT2 peptide-suicide protein fusion polypeptide constructs, Construct 1 or Construct 2. FIG. 4B is a bar graph depicting junction GAPT2A ddPCR results in a clonal human iPSC line with a mono-allelic integration of a T2A containing construct at GAPDH or depicting pooled junction GAPT2A ddPCR results in one of the modified ERT2 peptide-suicide protein polypeptide constructs, Construct 3 or Construct 4.

FIGS. 5A-5B are agarose gel photographs showing targeted integration of modified ERT2 peptide-suicide protein fusion polypeptides Construct 1, Construct 2, Construct 3, and Construct 4 at GAPDH loci in a pooled human iPSC population in the presence and absence of 4-OHT. FIG. 5A shows the results of a PCR analysis of a human iPSC line edited with mono-allelic integration of a T2A containing construct at GAPDH or PCR analysis of pools of human iPSCs transfected with one of the modified ERT2 peptide-suicide protein fusion polypeptide constructs, Construct 1 or Construct 2, in the absence or presence of 4-OHT. FIG. 5B shows the results of a PCR analysis of a human iPSC line edited with mono-allelic integration of a T2A containing construct at GAPDH or PCR analysis of pools of human iPSCs transfected with one of the modified ERT2 peptide-suicide protein fusion polypeptide constructs, Construct 3 or Construct 4, in the absence or presence of 4-OHT. “Parental wt” represents data for unedited parental cells. “NTC” is a no template PCR control.

FIG. 6 is a bar graph depicting zygosity assessment of several clones via junction GAPT2A ddPCR.

FIGS. 7A-7E are images of iPSC cultures at one or two days of treatment with 1 nM 4-OHT or were left untreated for the same duration. FIG. 7A panels show the images of unedited cells. FIG. 7B panels show the images of cells transfected with the modified ERT2 peptide-suicide protein fusion polypeptide construct, Construct 1. FIG. 7C panels show the images of cells transfected with the modified ERT2 peptide-suicide protein fusion polypeptide construct, Construct 2. FIG. 7D panels show the images of cells transfected with the modified ERT2 peptide-suicide protein fusion polypeptide construct, Construct 3. FIG. 7E panels show the images of cells transfected with the modified ERT2 peptide-suicide protein fusion polypeptide construct, Construct 4.

FIG. 8 shows cytometric dot plots demonstrating apoptotic activity in human iPSCs transfected with the modified ERT2 peptide-suicide protein fusion polypeptide construct, Construct 1, following a two-day treatment with 1 nM 4-OHT.

FIG. 9 shows cytometric dot plots demonstrating lack of apoptotic activity in human iPSCs transfected with the modified ERT2 peptide-suicide protein fusion polypeptide construct, Construct 2, following a two-day treatment with 1 nM 4-OHT.

FIGS. 10A-10B are graphs demonstrating apoptotic efficacy of 4-OHT treatment of Construct 1, Construct 2, Construct 3, and Construct 4 clones. FIG. 10A shows that most clones treated with 5 nM 4-OHT display apoptotic efficacy. FIG. 10B shows that the same set of clones display differential sensitivity to 1 nM 4-OHT treatment. Among these clones, Construct 1 clone displayed the highest apoptotic efficacy.

FIGS. 11A-11B are graphs demonstrating apoptotic efficacy of Endoxifen treatment of Construct 1, Construct 2, Construct 3, and Construct 4 clones. FIG. 11A compares apoptotic efficacy in clones treated with 10 nM Endoxifen or 400 μg/mL hygromycin (as a positive apoptotic control). FIG. 11B shows the differential sensitivity of clones to 1 nM Endoxifen treatment.

FIGS. 12A-12B are graphs depicting the apoptotic efficacy of treatment of Construct 1 clone with combinations of 4-OHT and Endoxifen. FIG. 12A depicts the apoptotic efficacy of treatment of Construct 1 clone with 1.25 nM Endoxifen and 0.125 nM to 1 nM 4-OHT. FIG. 12B depicts the apoptotic efficacy of treatment of Construct 1 clone with 0.625 nM or 1.25 nM Endoxifen and 0.125 nM 4-OHT.

FIGS. 13A-13E show the design and results of an experiment assessing the apoptotic efficacy in PSC-derived myeloid progenitor cells, following engineering of PSCs with TamCasp9 Construct 1, differentiation of the engineered PSCs into myeloid progenitor cells, and endoxifen treatment. FIG. 13A shows a schematic of an experimental workflow performed to validate conservation of kill switch function after differentiation of PSCs to myeloid progenitor cells. FIG. 13B shows flow cytometry evaluation of the differentiated cells, indicating successful differentiation into myeloid progenitor cells. FIG. 13C shows brightfield images with fluorescent overlay. Cells were stained with AO/PI, with dead cells appearing darker after uptake of Propidium Iodide. FIG. 13D shows quantitation of the kill switch activity in myeloid progenitor cells following differentiation of unedited PSCs at the indicated endoxifen treatment conditions. FIG. 13E shows quantitation of the kill switch activity in myeloid progenitor cells following differentiation of TamCasp9-edited PSCs, at the indicated endoxifen treatment conditions.

FIG. 14 is a graph depicting quantitation of kill switch activity in myeloid progenitor cells following differentiation of PSCs to myeloid progenitor cells with or without TamCasp9 editing at the indicated endoxifen and 4-OHT treatment conditions.

FIG. 15 is a schematic depicting a construct including a kill switch fusion polypeptide encoding sequences inserted between left and right homology arms targeting the GAPDH STEL, with a T2A peptide sequence linking a modified ERT2 peptide-suicide protein fusion polypeptide encoding sequence to the left homology arm. The construct also includes a linked puromycin-resistance (PuroR) selection cassette.

FIG. 16 is a schematic depicting a construct including a kill switch fusion polypeptide encoding sequences inserted between left and right homology arms targeting the GAPDH STEL with a T2A peptide sequence linking a tandem modified ERT2 peptide-suicide protein fusion polypeptide encoding sequence to the left homology arm. The construct also includes a linked puromycin-resistance (PuroR) selection cassette.

FIGS. 17A-17D are graphs demonstrating iPSC death (based on percentage confluency) following combination 4-OHT and endoxifen treatment of pools of puromycin-selected edited iPSCs containing Constructs 9-12. FIG. 17A depicts results for Construct 9. FIG. 17B depicts results for Construct 10. FIG. 17C depicts results for Construct 11. FIG. 17D depicts results for Construct 12.

### DETAILED DESCRIPTION OF THE INVENTION

### Definitions

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cell and tissue culture, molecular biology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art.

Gene-Edited Target Cell: As used herein, the term “gene-edited target cell” refers to a cell engineered to express a modified ERT2 peptide-suicide protein fusion polypeptide of the disclosure from a STEL locus, e.g., via introduction of a targeting construct of the disclosure, or its descendants and progeny. A gene-edited target cell need not be of the same cell type as the cell into which the targeting construct was initially introduced. For example, the targeting construct may be introduced into a stem cell, such as an iPSC or a hESC, upon which the nucleotide sequence flanked by the homology arms of the targeting construct is integrated into the STEL locus of the stem cell. The stem cell can then be differentiated to produce a differentiated cell type, for example any of the cell types disclosed in Section 0. Both the stem cell and the differentiated cell are referred to herein as a “gene-edited target cell”. In addition to encoding a modified ERT2 peptide-suicide protein fusion polypeptide of the disclosure, the gene-edited target cell may include a transgene. In some embodiments, the transgene is introduced via the same targeting construct as the fusion polypeptide coding sequence and both the transgene and the fusion polypeptide are expressed from the same allele of the STEL gene (whether on a heterozygous or homozygous basis). In other embodiments, the fusion protein coding sequence and the transgene are positioned in different alleles of the STEL gene. In yet other embodiments, the fusion protein coding sequence is in a STEL gene and the transgene is in a different locus, whether a different STEL locus or a non-STEL locus.

iPSC: The term “induced pluripotent stem cell” or “iPSC” refers to a type of pluripotent stem cell artificially prepared from a non-pluripotent cell, such as an adult somatic cell, partially differentiated cell or terminally differentiated cell, such as a fibroblast, a cell of hematopoietic lineage, a myocyte, a neuron, an epidermal cell, or the like, by introducing or contacting the cell with one or more reprogramming factors. iPSCs can be derived from multiple different cell types, including terminally differentiated cells. iPSCs have an embryonic stem (ES) cell-like morphology, growing as flat colonies with large nucleo-cytoplasmic ratios, defined borders and prominent nuclei. In addition, iPSCs express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, Fox03, GDF3, Cyp26al, TERT, and zfp42.

Examples of methods of generating and characterizing iPSCs may be found in, for example, US Patent Publication Nos. US20090047263, US20090068742, US20090191159, US20090227032, US20090246875, and US20090304646, and PCT patent publications WO2013177133 and WO2022204567, the disclosures of which are incorporated herein by reference. Generally, to generate iPSCs, somatic cells are provided with reprogramming factors (e.g., Oct4, SOX2, KLF4, MYC, Nanog, Lin28, etc.) known in the art to reprogram the somatic cells to become pluripotent stem cells.

Kill Switch: In the context of the present disclosure, the term “kill switch” refers to an inducible suicide gene or protein, for example a drug-inducible suicide gene or protein. Exemplary drug-inducible suicide proteins include the modified ERT2 peptide-suicide protein fusion polypeptides of the disclosure. The addition of appropriate drugs (e.g., estrogen antagonists such as tamoxifen in the context of modified ERT2 peptide-suicide protein fusion polypeptides disclosed herein) triggers the suicide proteins and kills the cells that express them. These features allow clinicians to kill off exogenously administered cell therapies, for example if cytokine release syndrome (CRS) develops or the cells become tumorigenic.

Linker or Linker Sequence: The terms “linker” or “linker sequence” as used in reference to a fusion polypeptide, refers to a part that connects two or more domains, parts, or entities. In some embodiments, the linker may comprise an amino acid or a peptide. Generally, linkers have no specific biological activity other than to join or to preserve some minimum distance or other spatial relationship between the parts.

Nucleic Acid: The terms “nucleic acid” or “oligonucleotide” or “polynucleotide” as used herein means at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

Operably linked: The term “operably linked” refers to a functional relationship between two or more peptide or polypeptide domains or nucleic acid (e.g., DNA) segments. In the context of transcriptional regulation, the term refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system.

Polypeptide, peptide, and protein: The terms “polypeptide,” “peptide” and “protein” are used interchangeably herein to refer to polymers of amino acids of any length. The polymer may be linear or branched, it may comprise modified amino acids, and it may be interrupted by non-amino acids.

Pluripotent: As used herein, the term “pluripotent” or “pluripotency” refers to the capacity of a cell to self-renew and to differentiate into cells of any of the three germ layers: endoderm, mesoderm, or ectoderm. “Pluripotent stem cells” or “PSCs” include, for example, embryonic stem cells derived from the inner cell mass of a blastocyst or derived by somatic cell nuclear transfer, and iPSCs derived from non-pluripotent cells.

STEL: The terms “sustained transgene expression locus” or “STEL” refer to a locus in the genome of a cell that enables persistent and stable expression of a transgene in that cell, e.g., through differentiation of the cell from one state or type to another state or type. STEL of the present disclosure include, without limitation, loci of robustly expressed endogenous genes, for instance certain housekeeping genes that are active in multiple cell types such as those involved in gene expression (e.g., transcription factors and histones), cellular metabolism (e.g., GAPDH and NADH dehydrogenase), or cellular structures (e.g., actin), or those that encode ribosomal proteins (e.g., large or small ribosomal subunits, such as RPL13A, RPLP0 and RPL7). Additional examples of STEL include those that form ribonucleoprotein complex, focal adhesion, cell-substrate adherens junction, cell-substrate junction, cell anchoring, extracellular exosome, extracellular vesicle, intracellular organelle, or anchoring junction. Some of the proteins are involved in RNA binding, nucleic acid binding (e.g., rRNA or mRNA binding), or protein binding.

STEL Protein, STEL Polypeptide: The terms “STEL protein” and “STEL polypeptide” are used interchangeably herein to refer to a polypeptide encoded by a STEL gene, or a polypeptide having at least 85% (e.g., at least 90%, at least 95%, at least 96%, at least 97%, at least 98% or at least 99%) sequence identity thereto.

Target Cell: The term “target cell” refers to a host cell into which is introduced a targeting construct that following integration into the host cell genome results in the production of a recombinant nucleic acid encoding a modified ERT2 peptide-suicide protein fusion polypeptide disclosed herein. It should be understood that such terms are intended to refer not only to the particular subject cell but to the progeny of such a cell. Such progeny need not be identical to the parent cell into which the expression vector or targeting construct was initially introduced but include counterparts and progeny of the cell which carry the expression cassette or into which the targeting construct has integrated, as well as cells differentiated therefrom. Such counterparts and progeny are still included within the scope of the term “target cell” as used herein.

Targeting Construct: The term “targeting construct” refers to a recombinant nucleic acid molecule that can specifically interact with a STEL locus and which may further comprise a nucleotide sequence encoding a modified ERT2 peptide-suicide protein fusion polypeptide disclosed herein. Recombination of the targeting construct and the STEL locus leads to the modification of the STEL locus, e.g., to introduce a modified ERT2 peptide-suicide protein fusion polypeptide disclosed herein into the STEL locus. Typically, a targeting construct comprises homology arms that allow integration of the targeting construct into a particular STEL locus.

Transfection: The term “transfection” refers to the introduction of nucleic acid molecules, such as targeting constructs, into cells, e.g., into eukaryotic cells. In the context of the present disclosure, the term “transfection” encompasses any method known to the skilled person for introducing nucleic acid molecules into cells, e.g., into eukaryotic cells, such as into mammalian cells. Such methods encompass, for example, electroporation, nucleofection, lipofection, e.g., based on cationic lipids and/or liposomes, calcium phosphate precipitation, nanoparticle-based transfection, virus-based transfection, or transfection based on cationic polymers, such as DEAE-dextran or polyethylenimine.

### Targeting Constructs

The present disclosure provides targeting constructs comprising nucleotide sequences encoding modified ERT2 peptide-suicide protein fusion polypeptides disclosed herein (“fusion polypeptide coding sequences”) flanked by homology arms that direct the integration of the fusion polypeptide coding sequences into a STEL locus in a target cell genome.

The targeting constructs may be in the form of vectors. The term “vector” as used herein refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome.

A targeting construct in the form of a vector may be linearized or released from a circular vector prior to its introduction into a target cell.

Alternatively, a targeting construct may be synthesized in vitro, e.g., using a DNA polymerase such as T7 prior to its introduction into a target cell.

Exemplary STEL loci are disclosed in Section 0.

Suitable homology arms are disclosed in Section 0.

The targeting constructs may further comprise nucleotide sequences encoding separator sequences, as described in Section 0, that allow the fusion polypeptide coding sequences to be positioned under common transcriptional regulatory control with a STEL protein (e.g., a protein encoded by the endogenous STEL locus) but allows the separate translation of the fusion polypeptide and the STEL protein or the post-translational cleavage of the fusion polypeptide and the STEL protein. Targeting constructs may further comprise one or more ribonucleoprotein (RNP) cut sites that flank the outer sides of the homology arms. These RNP cut sites allow for the endonuclease to cleave the targeting constructs, e.g., to linearize a circular targeting construct. In some embodiments, the targeting construct comprises no RNP cut sites, one RNP cut site, or two RNP cut sites.

Target cells into which the targeting constructs can be introduced are disclosed in Section 0.

In some embodiments, genome editing of a cell with any one of the targeting constructs disclosed herein results in insertion of more than one copies (e.g., 1, 2, 3, 4, or 5 or more copies) of any one of the nucleotide inserts disclosed here that comprise a nucleotide sequence encoding the fusion polypeptides disclosed herein.

### STEL Loci

Sustained Transgene Expression Loci (“STEL loci”) are genetic loci endogenous to a target cell that are expressed robustly and consistently at a sustained level across and between different cell types (for example, as a cell differentiates, such as from a PSC to PSC derived cell). For example, the expression level of the endogenous gene does not change (e.g., decrease) by more than 50%, more than 40%, more than 35%, more than 30%, more than 25%, more than 20%, more than 15%, more than 10%, or more than 5% over five or more, ten or more, or 15 or more passages or as the cell state changes (e.g., state of pluripotency and/or differentiation).

In some embodiments, a STEL locus is a gene that is associated with cellular metabolism, such as GAPDH, NADH dehydrogenase, and phosphoglycerate kinase 1 (PGK1).

In some embodiments, a STEL locus is a ribosomal protein gene or ribosomal protein gene locus, such as an RPL or RPS gene locus. Examples of RPL genes are RPL10, RPL13, RPS18, RPL3, RPLP1, RPL13A, RPL15, RPL41, RPL11, RPL32, RPL18A, RPL19, RPL28, RPL29, RPL9, RPL8, RPL6, RPL18, RPL7, RPL7A, RPL21, RPL37A, RPL12, RPL5, RPL34, RPL35A, RPL30, RPL24, RPL39, RPL37, RPL14, RPL27A, RPLP2, RPLP0, RPL23A, RPL26, RPL36, RPL35, RPL23, RPL4, and RPL22. Examples of RPS genes are RPS2, RPS19, RPS14, RPS3A, RPS12, RPS3, RPS6, RPS23, RPS27A, RPS8, RPS4X, RPS7, RPS24, RPS27, RPS15A, RPS9, RPS28, RPS13, RPSA, RPS5, RPS16, RPS25, RPS15, RPS20, and RPSIH

In some embodiments, a STEL gene encodes a mitochondrial protein. Examples of such genes are MT-C01, MT-C02, MT-ND4, MT-ND1, and MT-ND2.

In some embodiments, a STEL gene encodes a cytoskeletal protein, such as actin. Examples of actin genes are ACTG1 and ACTB.

In some embodiments, a STEL gene encodes a eukaryotic translation elongation factor, such as EEF1A1 and EEF2, or a eukaryotic translation initiation factor, such as EIFL.

In some embodiments, a STEL gene encodes a histone, such as H3F3A or H3F3B.

In other embodiments, a STEL gene is selected from FTL, FTH1, TPT1, IMSB10, PTMA, GNB2L1, NACA, YBX1, NPM1, FAU, UBA52, HSP90AB1, MYL6, SERF2, and SRP14.

In some embodiments, a modified ERT2 peptide-suicide protein fusion polypeptide sequence disclosed herein is introduced into a STEL gene, so that the STEL gene is modified to include the modified ERT2 peptide-suicide protein fusion polypeptide, optionally separated from the coding sequence of the STEL protein via a separator sequence. In some embodiments, the STEL locus is the GAPDH gene. In some embodiments, a modified ERT2 peptide-suicide protein fusion polypeptide sequence disclosed herein in introduced into a STEL gene, by generating a break in an exon of a STEL site that is required for cell survival, such that if proper integration of the modified ERT2 peptide-suicide protein fusion polypeptide sequence does not occur, cells having such improper integration do not survive. Such screening of improper integration events may be performed in accordance with methods described in WO 2021/226151, which is incorporated herein by reference.

### Homology Arms

Targeting constructs that are intended for integration into a STEL locus of a target cell genome typically comprise a heterologous sequence that is not present in the target cell genome, e.g., a nucleotide sequence encoding a modified ERT2 peptide-suicide protein fusion polypeptide disclosed herein.

The targeting constructs typically include one or more regions that are homologous to regions of DNA within or near (e.g., flanking or adjoining) a STEL locus sequence. These homologous regions are referred to here as “homology arms.” For ease of reference, the homology arms are referred to herein as first and second (i.e., 5′ and 3′, upstream and downstream, or left and right) homology arms. This terminology relates to the relative position of the homology arms to the nucleic acid insert within the targeting construct. The first and second homology arms correspond to regions within or near (e.g., flanking or adjoining) a STEL locus sequence, which are referred to herein as “first region of homology” and “second region of homology,” respectively. The regions within or near (e.g., flanking or adjoining) a STEL locus sequence are sometimes referred to herein as “target” sequences.

The current disclosure provides a targeting construct comprising a first homology arm that corresponds to a first region of homology, a nucleic acid insert, and a second homology arm that corresponds to a second region of homology to a STEL locus.

A homology arm and a target sequence “correspond” or are “corresponding” to one another when the two regions share a sufficient level of sequence identity to one another to act as substrates for a homologous recombination reaction, whereby the homology arms are suitable for directing recombination of a nucleic acid insert with a desired target sequence to facilitate genomic integration and/or replacement of endogenous sequence.

The term “homology” includes DNA sequences that are either identical or share sequence identity to a corresponding sequence. The sequence identity between a given target sequence and the corresponding homology arm found in the exogenous donor nucleic acid can be any degree of sequence identity that allows for homologous recombination to occur. For example, the amount of sequence identity shared by the homology arm of the exogenous donor nucleic acid (or a fragment thereof) and the target sequence (or a fragment thereof) can be at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination. Moreover, a corresponding region of homology between the homology arm and the corresponding target sequence can be of any length that is sufficient to promote homologous recombination. In some targeting vectors, the intended mutation in the target genomic locus is included in an insert nucleic acid flanked by the homology arms.

In some embodiments, the first homology arm is between 50 to 250 nucleotides in length. In some embodiments, the first homology arm is between 50-2000 nucleotides in length. In some embodiments, the first homology arm is between 50-1500 nucleotides in length. In some embodiments, the first homology arm is between 50-1000 nucleotides in length. In some embodiments, the first homology arm is between 50-500 nucleotides in length. In some embodiments, the first homology arm is between 150 to 250 nucleotides in length. In some embodiments, the first homology arm is 2000 nucleotides or less in length. In some embodiments, the first homology arm is 1500 nucleotides or less in length. In some embodiments, the first homology arm is 1000 nucleotides or less in length. In some embodiments, the first homology arm is 700 nucleotides or less in length. In some embodiments, the first homology arm is 650 nucleotides or less in length. In some embodiments, the first homology arm is 600 nucleotides or less in length. In some embodiments, the first homology arm is 550 nucleotides or less in length. In some embodiments, the first homology arm is 500 nucleotides or less in length. In some embodiments, the first homology arm is 400 nucleotides or less in length. In some embodiments, the first homology arm is 300 nucleotides or less in length. In some embodiments, the first homology arm is 250 nucleotides or less in length. In some embodiments, the first homology arm is 200 nucleotides or less in length. In some embodiments, the first homology arm is 150 nucleotides or less in length. In some embodiments, the first homology arm is less than 100 nucleotides in length. In some embodiments, the first homology arm is 50 nucleotides in length or less. In some embodiments, the first homology arm is 250, 200, 190, 180, 170, 160, 150, 140, 130, 120, 110, 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 nucleotides in length. In some embodiments, the first homology arm is at least 20 nucleotides in length. In some embodiments, the first homology arm is at least 40 nucleotides in length. In some embodiments, the first homology arm is at least 50 nucleotides in length. In some embodiments, the first homology arm is at least 70 nucleotides in length. In some embodiments, the first homology arm is at least 100 nucleotides in length. In some embodiments, the first homology arm is at least 200 nucleotides in length. In some embodiments, the first homology arm is at least 300 nucleotides in length. In some embodiments, the first homology arm is at least 400 nucleotides in length. In some embodiments, the first homology arm is at least 500 nucleotides in length. In some embodiments, the first homology arm is at least 600 nucleotides in length. In some embodiments, the first homology arm is at least 700 nucleotides in length. In some embodiments, the first homology arm is at least 1000 nucleotides in length. In some embodiments, the first homology arm is at least 1500 nucleotides in length. In some embodiments, the first homology arm is at least 2000 nucleotides in length. In some embodiments, the first homology arm is about 20 nucleotides in length. In some embodiments, the first homology arm is about 40 nucleotides in length. In some embodiments, the first homology arm is 250 nucleotides in length or less. In some embodiments, the first homology arm is about 100 nucleotides in length. In some embodiments, the first homology arm is about 200 nucleotides in length.

In some embodiments, the second homology arm is between 50 to 250 nucleotides in length. In some embodiments, the second homology arm is between 50-2000 nucleotides in length. In some embodiments, the second homology arm is between 50-1500 nucleotides in length. In some embodiments, the second homology arm is between 50-1000 nucleotides in length. In some embodiments, the second homology arm is between 50-500 nucleotides in length. In some embodiments, the second homology arm is between 150 to 250 nucleotides in length. In some embodiments, the second homology arm is 2000 nucleotides or less in length. In some embodiments, the second homology arm is 1500 nucleotides or less in length. In some embodiments, the second homology arm is 1000 nucleotides or less in length. In some embodiments, the second homology arm is 700 nucleotides or less in length. In some embodiments, the second homology arm is 650 nucleotides or less in length. In some embodiments, the second homology arm is 600 nucleotides or less in length. In some embodiments, the second homology arm is 550 nucleotides or less in length. In some embodiments, the second homology arm is 500 nucleotides or less in length. In some embodiments, the second homology arm is 400 nucleotides or less in length. In some embodiments, the second homology arm is 300 nucleotides or less in length. In some embodiments, the second homology arm is 200 nucleotides in length or less. In some embodiments, the second homology arm is 150 nucleotides in length or less. In some embodiments, the second homology arm is 100 nucleotides in length or less. In some embodiments, the second homology arm is 50 nucleotides in length or less. In some embodiments, the second homology arm is 250, 200, 190, 180, 170, 160, 150, 140, 130, 120, 110, 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 nucleotides in length. In some embodiments, the second homology arm is at least 20 nucleotides in length. In some embodiments, the second homology arm is at least 40 nucleotides in length. In some embodiments, the second homology arm is at least 50 nucleotides in length. In some embodiments, the second homology arm is at least 70 nucleotides in length. In some embodiments, the second homology arm is at least 100 nucleotides in length. In some embodiments, the second homology arm is at least 200 nucleotides in length. In some embodiments, the second homology arm is at least 300 nucleotides in length. In some embodiments, the second homology arm is at least 400 nucleotides in length. In some embodiments, the second homology arm is at least 500 nucleotides in length. In some embodiments, the second homology arm is at least 600 nucleotides in length. In some embodiments, the second homology arm is at least 700 nucleotides in length. In some embodiments, the second homology arm is at least 1000 nucleotides in length. In some embodiments, the second homology arm is at least 1500 nucleotides in length. In some embodiments, the second homology arm is at least 2000 nucleotides in length. In some embodiments, the second homology arm is about 20 nucleotides in length. In some embodiments, the second homology arm is about 40 nucleotides in length. In some embodiments, the second homology arm is 250 nucleotides in length or less. In some embodiments, the second homology arm is about 100 nucleotides in length. In some embodiments, the second homology arm is about 200 nucleotides in length.

The first and second homology arms can be of the same length or can differ in length. In some embodiments, the first and second homology arms are amplified to allow for the quantitative assessment of gene editing events, such as targeted integration, at a target nucleic acid. In some embodiments, the assessment of the gene editing events may rely on the amplification of both the 5′ junction and 3′ junction at the site of targeted integration by amplifying the whole or a part of the homology arm using a single pair of PCR primers in a single amplification reaction. Accordingly, although the length of the first and second homology arms may differ, the length of each homology arm should be capable of amplification (e.g., using PCR), as desired.

In some embodiments, the length of the first and second homology arms does not differ by more than 75 nucleotides. Thus, in some embodiments, when the first and second homology arms differ in length, the length difference between the homology arms is less than 70, 60, 50, 40, 30, 20, 10, 9, 8, 7, 6, 5, 4, 3, 2, 1 nucleotides or base pairs. In some embodiments, the first and second homology arms differ in length by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 nucleotides. In some embodiments, the length difference between the first and second homology arms is less than 70, 60, 50, 40, 30, 20, 10, 9, 8, 7, 6, 5, 4, 3, 2, 1 base pairs. In some embodiments, the first and second homology arms differ in length by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 base pairs.

Homology arms are capable of directing recombination of a nucleic acid insert within or near a desired target STEL gene to facilitate genomic integration and/or replacement of endogenous sequence, e.g., integrate a modified ERT2 peptide-suicide protein fusion polypeptide sequence disclosed herein into a STEL gene. Regardless of the format used, a donor template can be designed to avoid undesirable sequences. In certain embodiments, one or both homology arms can be shortened to avoid overlap with certain sequence repeat elements, e.g., Alu repeats, LINE elements, etc.

In some embodiments, the homology arms are designed such that, following integration of the targeting construct, the modified ERT2 peptide-suicide protein fusion polypeptide coding sequence is positioned 3′ to the STEL polypeptide coding sequence. In other embodiments, the homology arms are designed such that, following integration of the targeting construct, the modified ERT2 peptide-suicide protein fusion polypeptide coding sequence is positioned 5′ to the STEL polypeptide coding sequence. Typically, the STEL polypeptide coding sequence and the modified ERT2 peptide-suicide protein fusion polypeptide coding sequence are separated by a separator sequence, such as an IRES or a 2A peptide coding sequence.

### Separator Sequences

The targeting constructs and recombinant target cell genomes described herein can also comprise a separator sequence between STEL polypeptide coding sequence and the modified ERT2 peptide-suicide protein fusion polypeptide coding sequence. Such separator sequences can allow separate expression of the STEL polypeptide and the modified ERT2 peptide-suicide protein fusion polypeptide under common control of STEL gene regulatory elements.

In some embodiments, the separator sequence is an internal ribosome entry site (IRES), which allows the STEL polypeptide to be translated separately from the modified ERT2 peptide-suicide protein fusion polypeptide.

In some embodiments, the separator sequence is a self-cleaving peptide, associated with ribosomal skipping during translation, in which the ribosomes skip the peptide bond between a C-terminal Gly and Pro, resulting in the production of two separate polypeptides, i.e., the STEL polypeptide and the modified ERT2 peptide-suicide protein fusion polypeptide. A self-cleaving peptide causes ribosomal skipping during translation. Examples of self-cleaving peptides are 2A peptides, which are viral derived peptides with a typical length of 18-22 amino acids. 2A peptides include T2A, P2A, E2A, F2A, PQR (Lo et al., 2015, Cell Reports 13:2634-2644), Opt2A, and Opt2A_2.0. By way of example, P2A is a peptide of 19 amino acids; after the cleavage, a few amino acid residues from the P2A are left on the upstream polypeptide and a proline is left at the beginning of the second polypeptide. 2A residues left on the STEL polypeptide and the modified ERT2 peptide-suicide protein fusion polypeptide do not affect their functionality.

In some embodiments, a 2A peptide a P2A peptide. In some embodiments, a P2A peptide comprises an amino acid sequence as set forth in SEQ ID NO: 195. In some embodiments, a P2A peptide comprises an amino acid sequence as set forth in SEQ ID NO: 11. In some embodiments, a P2A peptide is encoded by a nucleic acid sequence as set forth in SEQ ID NO: 52.

P2A amino acid sequence:
(SEQ ID NO: 11)
ATNFSLLKQAGDVEENPGP
P2A nucleic acid sequence:
(SEQ ID NO: 52)
GCGACGAATTTTAGTCTACTGAAACAAGCGGGAGACGTGGAGGAAAACCC
TGGACCT

In some embodiments, a 2A peptide comprises a T2A peptide. In some embodiments, a T2A peptide comprises an amino acid sequence as set forth in SEQ ID NO: 53. In some embodiments, a T2A peptide is encoded by a nucleic acid sequence as set forth in SEQ ID NO: 54.

T2A amino acid sequence:
(SEQ ID NO: 53)
EGRGSLLTCGDVEENPGP
T2A nucleic acid sequence:
(SEQ ID NO: 54)
GAAGGGCGCGGGTCTCTCCTCACTTGTGGAGATGTTGAGGAAAATCCAGG
ACCA

In some embodiments, a 2A peptide comprises an E2A G4S T2A (Opt2A) peptide. In some embodiments, an E2A G4S T2A (Opt2A) peptide comprises an amino acid sequence as set forth in SEQ ID NO: 55. In some embodiments, an Opt2A peptide is encoded by a nucleic acid sequence as set forth in SEQ ID NO: 56.

Opt2A amino acid sequence:
(SEQ ID NO: 55)
QCTNYALLKLAGDVESNPGPGSGEGRGSLLTCGDVEENPGP
Opt2A nucleic acid sequence:
(SEQ ID NO: 56)
CAGTGCACAAATTATGCACTGCTGAAGCTCGCCGGGGATGTCGAGAGTAA
CCCAGGACCTGGAAGCGGAGAAGGTCGTGGTAGTCTACTAACGTGTGGTG
ATGTAGAAGAAAATCCTGGACCT

In some embodiments, a 2A peptide comprises a P2A 3-T2A 2 (Opt2A_2.0) peptide. In some embodiments, a P2A 3-T2A 2 (Opt2A_2.0) peptide comprises an amino acid sequence as set forth in SEQ ID NO: 12. In some embodiments, an Opt2A_2.0 peptide is encoded by a nucleic acid sequence as set forth in SEQ ID NO: 57.

Opt2A_2.0 amino acid sequence:
(SEQ ID NO: 12)
ATNFSLLKQAGDVEENPGPGSGEGRGSLLTCGDVEENPGP
Opt2A 2.0 nucleic acid sequence:
(SEQ ID NO: 57)
GCGACGAATTTTAGTCTACTGAAACAAGCGGGAGACGTGGAGGAAAACCC
TGGACCTGGAAGCGGAGAAGGTCGTGGTAGTCTACTAACGTGTGGTGATG
TAGAAGAAAATCCTGGACCT

### Modified ERT2 Peptide-Suicide Protein Fusion Polypeptides

The modified ERT2 peptide-suicide protein fusion polypeptides disclosed herein allow for temporal control of activity of a suicide protein that is activated upon dimerization. The modified ERT2 peptide can allow reversible control over their activity by administrating or removing an inducing agent such as tamoxifen or a metabolite thereof, for example, 4-hydroxytamoxifen (4-OHT), N-desmethyltamoxifen, tamoxifen-N-oxide, and endoxifen. For example, without being bound by theory, it is thought that in the absence of an inducing agent, the modified ERT2 peptide domains, together with their suicide protein fusion partner, remain largely in monomeric form, such that they cannot induce apoptosis. In the presence of tamoxifen or a metabolite thereof, however, it is believed that the modified ERT2 peptide can then dimerize, allowing the suicide protein components to also dimerize and initiate apoptosis.

The modified ERT2 peptide-suicide protein fusion polypeptides disclosed herein comprise (a) a modified ERT2 peptide disclosed herein, (b) a suicide protein disclosed herein, and (c) an optional linker separating the modified ERT2 peptide and suicide protein. In some embodiments, the modified ERT2 peptide-suicide protein fusion polypeptide comprises a modified ERT2 peptide N-terminal to the suicide protein. In some embodiments, the modified ERT2 peptide-suicide protein fusion polypeptide comprises a modified ERT2 peptide C-terminal the suicide protein. In yet further embodiments, a modified ERT2 peptide-suicide protein fusion polypeptide comprises a modified ERT2 peptide both N-terminal and C-terminal to the suicide protein. The N- and C-terminal modified ERT2 peptides can be the same or different. In each case, the suicide protein and the modified ERT2 peptide sequence may be separated by a linker sequence.

Modified ERT2 peptides are set forth in Section 0 below.

Suicide proteins are set forth in Section 0 below.

Exemplary linker sequences are set forth in Section 0 below.

### The Modified ERT2 Peptide

Estrogen receptor (ER) is a ligand-dependent transcription factor that binds endogenous hormone ligands such as estrogen and estradiol. Synthetic ligands that bind to ER, such as tamoxifen and its active metabolites, e.g., 4-OHT, N-desmethyltamoxifen, tamoxifen-N-oxide, and endoxifen, have been developed for treating ER-positive cancers.

Suicide protein activity can be regulated by fusing the suicide protein to a modified ER-LBD (also referred to herein as modified ERT2 peptide). The modified ERT2 peptide comprises an amino acid sequence corresponding to a hormone binding domain of a reference human estrogen receptor sequence (SEQ ID NO: 1), and comprises
a G400V amino acid substitution, an M543A amino acid substitution, an L544A amino acid substitution, and optionally a V595A amino acid substitution, with reference to SEQ ID NO: 1; and additional amino acid substitutions, wherein the additional amino acid substitutions comprise, with reference to SEQ ID NO: 1:
(i) an L384M substitution, an L391V substitution, a N413D substitution, an M421L substitution, a S463P substitution, and a H524L substitution,
(ii) an L391V substitution, a N413D substitution, a Q414E substitution, a S463P substitution, and a H524F substitution,
(iii) an L354I substitution, a L39TV substitution, a N413D substitution, a Q414E substitution, a M421L substitution, a M517A substitution, and a H524F substitution,
(iv) an L354I substitution, a L391V substitution, a L409V substitution, a N413D substitution, a Q414E substitution, and a H524L substitution,
(v) an L391V substitution, a Q414E substitution, an N413D substitution, an S463P substitution, an M421L substitution, an L354I substitution, an L384M substitution, and an H524L substitution,
(vi) an L391V substitution, an N413D substitution, an S463P substitution, an M517A substitution, an M421L substitution, an L354I substitution, and an H524L substitution,
(vii) an N413D substitution, an S463P substitution, an L354I substitution, an L384M substitution, and an H524L substitution, or
(viii) an L391V substitution, an N413D substitution, an S463P substitution, an M517A substitution, an M421L substitution, an L354I substitution, and an H524L substitution.

Fusing a suicide protein to a modified ERT2 peptide is believed to maintain the suicide protein in monomeric form in the absence of a modified ERT2 peptide inducer, such that the suicide protein cannot induce apoptosis. In the presence of estrogen receptor antagonists (e.g., tamoxifen), the modified ERT2 peptide, and consequently the suicide protein, can dimerize, activating the apoptosis-inducing capability of the suicide protein. This dimerization can be achieved by administering one or more estrogen receptor antagonists. Exemplary estrogen receptor antagonists include, but are not limited to, tamoxifen and its metabolites, e.g., 4-OHT, N-desmethyltamoxifen, tamoxifen-N-oxide, and endoxifen.

It is to be understood that the additional amino acid substitutions to the modified ERT2 peptide disclosed herein are with reference to SEQ ID NO: 1.

In some embodiments, the hormone binding domain of a reference human estrogen receptor sequence corresponds to positions 282-595 of human estrogen receptor (SEQ ID NO: 1). It is to be understood that the hormone binding domain does not necessarily require all of amino acid residues 282-595 of SEQ ID NO: 1. By way of example only, it is to be understood that positions 283-594 of SEQ ID NO: 1, or other functional truncations or fragments thereof, may function as the hormone binding domain.

In some embodiments, the additional amino acid substitutions comprise: an L384M substitution, an L391V substitution, a N413D substitution, an M421L substitution, a S463P substitution, and a H524L substitution, and the modified ER-LBD comprises an amino acid sequence having at least 80%, at least 85%, at least 90%, at least 95%, at least 98%, at least 99%, or 100% sequence identity to the amino acid sequence of SEQ ID NO:2.

In some embodiments, the additional amino acid substitutions comprise: an L391V substitution, a N413D substitution, a Q414E substitution, a S463P substitution, and a H524F substitution, and the modified ER-LBD comprises an amino acid sequence having at least 80%, at least 85%, at least 90%, or at least 95%, at least 98%, at least 99%, or 100% sequence identity to the amino acid sequence of SEQ ID NO:3.

In some embodiments, the additional amino acid substitutions comprise: an L354I substitution, a L391V substitution, a N413D substitution, a Q414E substitution, a M421L substitution, a M517A substitution, and a H524F substitution, and the modified ER-LBD comprises an amino acid sequence having at least 80%, at least 85%, at least 90%, or at least 95% sequence identity to the amino acid sequence of SEQ ID NO:4.

In some embodiments, the additional amino acid substitutions comprise: an L354I substitution, a L391V substitution, a L409V substitution, a N413D substitution, a Q414E substitution, and a H524L substitution, and the modified ER-LBD comprises an amino acid sequence having at least 80%, at least 85%, at least 90%, or at least 95% sequence identity to the amino acid sequence of SEQ ID NO:5.

In some embodiments, the additional amino acid substitutions comprise: an L391V substitution, a Q414E substitution, an N413D substitution, an S463P substitution, an M421L substitution, an L354I substitution, an L384M substitution, and an H524L substitution, and the modified ER-LBD comprises an amino acid sequence having at least 80%, at least 85%, at least 90%, or at least 95% sequence identity to the amino acid sequence of SEQ ID NO:40.

In some embodiments, the additional amino acid substitutions comprise: an L391V substitution, an N413D substitution, an S463P substitution, an M517A substitution, an M421L substitution, an L354I substitution, and an H524L substitution, and the modified ER-LBD comprises an amino acid sequence having at least 80%, at least 85%, at least 90%, or at least 95% sequence identity to the amino acid sequence of SEQ ID NO:41.

In some embodiments, the additional amino acid substitutions comprise: an N413D substitution, an S463P substitution, an L354I substitution, an L384M substitution, and an H524L substitution, and the modified ER-LBD comprises an amino acid sequence having at least 80%, at least 85%, at least 90%, or at least 95% sequence identity to the amino acid sequence of SEQ ID NO:42.

In some embodiments, the additional amino acid substitutions comprise: an L391V substitution, an N413D substitution, an S463P substitution, an M517A substitution, an M421L substitution, an L354I substitution, and an H524L substitution, and the modified ER-LBD comprises an amino acid sequence having at least 80%, at least 85%, at least 90%, or at least 95% sequence identity to the amino acid sequence of SEQ ID NO:43.

TABLE 1

| | | SEQ |
|---|---|---|
| | | ID |
| Name | Sequence | NO: |
| Estrogen | MTMTLHTKASGMALLHQIQGNELEPLNRPQLKIP | 1 |
| Receptor | LERPLGEVYLDSSKPAVYNYPEGAAYEFNAAAAA | |
| (Reference | NAQVYGQTGLPYGPGSEAAAFGSNGLGGFPPLNS | |
| Amino Acid | VSPSPLMLLHPPPQLSPFLQPHGQQVPYYLENEP | |
| Sequence) | SGYTVREAGPPAFYRPNSDNRRQGGRERLASTND | |
| Italics = | KGSMAMESAKETRYCAVCNDYASGYHYGVWSCEG | |
| amino acid | CKAFFKRSIQGHNDYMCPATNQCTIDKNRRKSCQ | |
| positions | ACRLRKCYEVGMMKGGIRKDRRGGRMLKHKRQRD | |
| 282-595 | DGEGRGEVGSAGDMRAANLWPSPLMIKRSKKNSL | |
| | ALSLTADQMVSALLDAEPPILYSEYDPTRPFSEA | |
| | SMMGLLTNLADRELVHMINWAKRVPGFVDLTLHD | |
| | QVHLLECAWLEILMIGLVWRSMEHPGKLLFAPNL | |
| | LLDRNQGKCVEGMVEIFDMLLATSSRFRMMNLQG | |
| | EEFVCLKSIILLNSGVYTFLSSTLKSLEEKDHIH | |
| | RVLDKITDTLIHLMAKAGLTLQQQHQRLAQLLLI | |
| | LSHIRHMSNKGMEHLYSMKCKNVVPLYDLLLEML | |
| | DAHRLHAPTSRGGASVEETDQSHLATAGSTSSHS | |
| | LQKYYITGEAEGFPATV | |
| ERT2*mut81 | AGDMRAANLWPSPLMIKRSKKNSLALSLTADQMV | 2 |
| | SALLDAEPPILYSEYDPTRPFSEASMMGLLTNLA | |
| | DRELVHMINWAKRVPGFVDLTLHDQVHLLECAWM | |
| | EILMIGVVWRSMEHPVKLLFAPNLLLDRDQGKCV | |
| | EGLVEIFDMLLATSSRFRMMNLQGEEFVCLKSII | |
| | LLNSGVYTFLPSTLKSLEEKDHIHRVLDKITDTL | |
| | IHLMAKAGLTLQQQHQRLAQLLLILSHIRHMSNK | |
| | GMELLYSMKCKNVVPLYDLLLEAADAHRLHAPTS | |
| | RGGASVEETDQSHLATAGSTSSHSLQKYYITGEA | |
| | EGFPAT | |
| ERT2*mut88 | AGDMRAANLWPSPLMIKRSKKNSLALSLTADQMV | 3 |
| | SALLDAEPPILYSEYDPTRPFSEASMMGLLTNLA | |
| | DRELVHMINWAKRVPGFVDLTLHDQVHLLECAWL | |
| | EILMIGVVWRSMEHPVKLLFAPNLLLDRDEGKCV | |
| | EGMVEIFDMLLATSSRFRMMNLQGEEFVCLKSII | |
| | LLNSGVYTFLPSTLKSLEEKDHIHRVLDKITDTL | |
| | IHLMAKAGLTLQQQHQRLAQLLLILSHIRHMSNK | |
| | GMEFLYSMKCKNVVPLYDLLLEAADAHRLHAPTS | |
| | RGGASVEETDQSHLATAGSTSSHSLQKYYITGEA | |
| | EGFPAT | |
| ERT2*mut63 | AGDMRAANLWPSPLMIKRSKKNSLALSLTADQMV | 4 |
| | SALLDAEPPILYSEYDPTRPFSEASMMGLLTNLA | |
| | DREIVHMINWAKRVPGFVDLTLHDQVHLLECAWL | |
| | EILMIGVVWRSMEHPVKLLFAPNLLLDRDEGKCV | |
| | EGLVEIFDMLLATSSRFRMMNLQGEEFVCLKSII | |
| | LLNSGVYTFLSSTLKSLEEKDHIHRVLDKITDTL | |
| | IHLMAKAGLTLQQQHQRLAQLLLILSHIRHASNK | |
| | GMEFLYSMKCKNVVPLYDLLLEAADAHRLHAPTS | |
| | RGGASVEETDQSHLATAGSTSSHSLQKYYITGEA | |
| | EGFPAT | |
| ERT2*mut41 | AGDMRAANLWPSPLMIKRSKKNSLALSLTADQMV | 5 |
| | SALLDAEPPILYSEYDPTRPFSEASMMGLLTNLA | |
| | DREIVHMINWAKRVPGFVDLTLHDQVHLLECAWL | |
| | EILMIGVVWRSMEHPVKLLFAPNLVLDRDEGKCV | |
| | EGMVEIFDMLLATSSRFRMMNLQGEEFVCLKSII | |
| | LLNSGVYTFLSSTLKSLEEKDHIHRVLDKITDTL | |
| | IHLMAKAGLTLQQQHQRLAQLLLILSHIRHMSNK | |
| | GMELLYSMKCKNVVPLYDLLLEAADAHRLHAPTS | |
| | RGGASVEETDQSHLATAGSTSSHSLQKYYITGEA | |
| | EGFPAT | |
| ERT2*mut51 | AGDMRAANLWPSPLMIKRSKKNSLALSLTADQMV | 40 |
| | SALLDAEPPILYSEYDPTRPFSEASMMGLLTNLA | |
| | DREIVHMINWAKRVPGFVDLTLHDQVHLLECAWM | |
| | EILMIGVVWRSMEHPVKLLFAPNLLLDRDEGKCV | |
| | EGLVEIFDMLLATSSRFRMMNLQGEEFVCLKSII | |
| | LLNSGVYTFLPSTLKSLEEKDHIHRVLDKITDTL | |
| | IHLMAKAGLTLQQQHQRLAQLLLILSHIRHMSNK | |
| | GMELLYSMKCKNVVPLYDLLLEAADAHRLHAPTS | |
| | RGGASVEETDQSHLATAGSTSSHSLQKYYITGEA | |
| | EGFPAT | |
| ERT2*mut37 | AGDMRAANLWPSPLMIKRSKKNSLALSLTADQMV | 41 |
| | SALLDAEPPILYSEYDPTRPFSEASMMGLLTNLA | |
| | DREIVHMINWAKRVPGFVDLTLHDQVHLLECAWL | |
| | EILMIGVVWRSMEHPVKLLFAPNLLLDRDQGKCV | |
| | EGLVEIFDMLLATSSRFRMMNLQGEEFVCLKSII | |
| | LLNSGVYTFLPSTLKSLEEKDHIHRVLDKITDTL | |
| | IHLMAKAGLTLQQQHQRLAQLLLILSHIRHASNK | |
| | GMELLYSMKCKNVVPLYDLLLEAADAHRLHAPTS | |
| | RGGASVEETDQSHLATAGSTSSHSLQKYYITGEA | |
| | EGFPAT | |
| ERT2*mut94 | AGDMRAANLWPSPLMIKRSKKNSLALSLTADQMV | 42 |
| | SALLDAEPPILYSEYDPTRPFSEASMMGLLTNLA | |
| | DREIVHMINWAKRVPGFVDLTLHDQVHLLECAWM | |
| | EILMIGLVWRSMEHPVKLLFAPNLLLDRDQGKCV | |
| | EGMVEIFDMLLATSSRFRMMNLQGEEFVCLKSII | |
| | LLNSGVYTFLPSTLKSLEEKDHIHRVLDKITDTL | |
| | IHLMAKAGLTLQQQHQRLAQLLLILSHIRHMSNK | |
| | GMELLYSMKCKNVVPLYDLLLEAADAHRLHAPTS | |
| | RGGASVEETDQSHLATAGSTSSHSLQKYYITGEA | |
| | EGFPAT | |
| ERT2*mut41 | AGDMRAANLWPSPLMIKRSKKNSLALSLTADQMV | 43 |
| v2 | SALLDAEPPILYSEYDPTRPFSEASMMGLLTNLA | |
| | DREIVHMINWAKRVPGFVDLTLHDQVHLLECAWL | |
| | EILMIGVVWRSMEHPVKLLFAPNLVLDRDQGKCV | |
| | EGLVEIFDMLLATSSRFRMMNLQGEEFVCLKSII | |
| | LLNSGVYTFLPSTLKSLEEKDHIHRVLDKITDTL | |
| | IHLMAKAGLTLQQQHQRLAQLLLILSHIRHASNK | |
| | GMELLYSMKCKNVVPLYDLLLEAADAHRLHAPTS | |
| | RGGASVEETDQSHLATAGSTSSHSLQKYYITGEA | |
| | EGFPAT | |

In some embodiments, the modified ERT2 peptide further comprises an N-terminal serine (S) residue.

In further embodiments, the modified ERT2 peptide may also include a V595A amino acid substitution.

### The Suicide Protein

The modified ERT2 peptide-suicide protein fusion polypeptides comprises a caspase 9 domain or derivative or functional fragment thereof (“suicide protein”).

The activity of the suicide protein is inducible by dimerization. Thus, in some embodiments, the suicide protein is inactive or has low/reduced activity in monomeric form, but has suicide protein activity in dimeric form.

In some embodiments, the caspase 9 sequence is a truncated caspase 9 (“truncCasp9”) with its Caspase Activation and Recruitment Domain (CARD) motif removed. In the presence of an estrogen antagonist such as tamoxifen or its metabolites, the ERT2 domain dimerizes, which is believed to subsequently cause homodimerization of truncCasp9 and induction of apoptosis.

The dimerization of modified ERT2 peptide-tuncCasp9 fusion polypeptides can be achieved by administering one or more estrogen antagonists. Exemplary estrogen antagonists include tamoxifen and/or its metabolites, such as 4-OHT, N-desmethyltamoxifen, tamoxifen-N-oxide, and endoxifen. Without being bound by theory, it is believed that administering a combination of two or more estrogen antagonists (such as a combination of tamoxifen and one or more of its metabolites or a combination of two or more tamoxifen metabolites) can result in synergism, thereby lowering the minimally effective dosage of each agent included in the combination. Consequently, apoptosis can be achieved by administration of pharmacologically relevant concentrations of each agent in a combination.

In some embodiments, ERT2 peptide-truncCasp9 fusion polypeptide dimerization is achieved by administering only one estrogen antagonist, such as tamoxifen, 4-OHT, N-desmethyltamoxifen, tamoxifen-N-oxide, or endoxifen. In other embodiments, ERT2 peptide-truncCasp9 fusion polypeptide homodimerization is achieved by administering any combination of two or more estrogen antagonists, such as tamoxifen and/or its metabolites, including but not limited to 4-OHT, N-desmethyltamoxifen, tamoxifen-N-oxide, and endoxifen, in any combination. In some embodiments, a combination comprising 4-OHT and endoxifen is administered. In some embodiments, the dose of each agent administered in combination is less than the dose that would be administered to achieve dimerization using a single agent.

An exemplary truncCasp9 sequence is set forth below as SEQ ID NO:6:
The derivative of the caspase 9 domain may comprise at least 95% identity, at least 96% identity, at least 97% identity, at least 98% identity, at least 99% identity, or 100% identity to SEQ ID NO: 6.

MDVGALESLRGNADLAYILSMEPCGHCLIINNVNFCRESGLRTRT
GSNIDCEKLRRRFSSLHFMVEVKGDLTAKKMVLALLELARQDHGA
LDCCVVVILSHGCQASHLQFPGAVYGTDGCPVSVEKIVNIFNGTS
CPSLGGKPKLFFIQACGGEQKDHGFEVASTSPEDESPGSNPEPDA
TPFQEGLRTFDQLDAISSLPTPSDIFVSYSTFPGFVSWRDPKSGS
WYVETLDDIFEQWAHSEDLQSLLLRVANAVSVKGIYKQMPGCFNF
LRKKLFFKTS

### The Linker

The modified ERT2 peptide-suicide protein fusion polypeptide of the disclosure may comprise an optional linker sequence between the modified ERT2 peptide and the suicide protein sequence.

Suitable linkers for use in the fusion polypeptide of the present disclosure are well known to those of skill in the art and include peptide linkers. In particular embodiments, the linker is used to separate the modified ERT2 peptide and the suicide protein sequence by a distance sufficient to ensure that the suicide protein retains its function. Preferred peptide linker sequences adopt a flexible extended conformation and do not exhibit a propensity for developing an ordered secondary structure.

Typical amino acids in flexible peptide linkers include Gly, Asn and Ser. Accordingly, in particular embodiments, the linker comprises a combination of one or more of Gly, Asn and Ser amino acids. Other near neutral amino acids, such as Thr and Ala, also may be used in the linker sequence. Exemplary linkers are disclosed in Maratea et al., 1985, Gene 40: 39-46; Murphy et al., 1986, Proc. Nat'l. Acad. Sci. USA 83: 8258-62; U.S. Pat. Nos. 4,935,233; and 4,751,180, the contents of which are incorporated herein in their entireties.

Peptide linkers can be one amino acid sequence or repeats of one or more amino acid sequences. In some embodiments, a sequence can be used in repeats of 2. In some embodiments, a sequence can be used in repeats of 3. In some embodiments, a sequence can be used in repeats of 4. In some embodiments, a sequence can be used in repeats of 5 or more.

In some embodiments, the peptide linker is between 1 and 30 amino acids in length. In various aspects, the peptide linker is between 1 and 3 amino acids in length, between 3 and 8 amino acids in length, between 3 and 10 amino acids in length, between 5 and 15 amino acids in length, between 11 and 20 amino acids in length, between 15 and 25 amino acids in length, between 21 and 30 amino acids in length, or is a length range bounded by any pair of the forgoing values (e.g., between 3 and 15 amino acids in length, between 8 and 20 amino acids in length, between 25 and 30 amino acids in length, and so on and so forth).

Non-limiting examples of linker sequences are set forth in Table 2 below.

TABLE 2

| Linker Amino Acid | SEQ ID |
|---|---|
| Sequence | NO: |
| GGS | 29 |
| GSGSGSGSGG | 30 |
| GGGS | 31 |
| GSG | 32 |
| GGSG | 33 |
| GGGS | 34 |
| GGGGS | 35 |
| GGSGGSGGS | 36 |
| GGGGSGGGGS | 37 |
| GGGGSGGGGSGGGGS | 38 |
| GGGGSGGGGSGGGGSVDGF | 39 |

### Construct Sequences

In some embodiments, a targeting construct comprises one or more modified ERT2 peptide sequences and one or more suicide proteins. In some embodiments, a targeting construct comprises 1 or more, 2 or more, 3 or more, 4 or more, 5 or more, 6 or more, 7 or more, or 8 or more distinct modified ERT2 peptide sequences. In some embodiments, a targeting construct comprises 1 or more, 2 or more, 3 or more, 4 or more, or 5 or more copies of a single modified ERT2 peptide sequence. In some embodiments, a targeting construct comprises 1 or more, 2 or more, 3 or more, 4 or more, 5 or more copies of distinct modified ERT2-Caspase9 constructs. In some embodiments, a targeting construct comprises 1 or more, 2 or more, 3 or more, 4 or more, 5 or more copies of distinct modified ERT2-truncCaspase9 constructs.

In some embodiments, a targeting construct comprises an ERT2*mut81 sequence and an ERT2*mut88 sequence. In some embodiments, a targeting construct comprises an ERT2*mut81 sequence and an ERT2*mut63 sequence. In some embodiments, a targeting construct comprises an ERT2*mut81 sequence and an ERT2*mut41 sequence. In some embodiments, a targeting construct comprises an ERT2*mut81 sequence and an ERT2*mut51 sequence. In some embodiments, a targeting construct comprises an ERT2*mut81 sequence and an ERT2*mut37 sequence. In some embodiments, a targeting construct comprises an ERT2*mut81 sequence and an ERT2*mut94 sequence. In some embodiments, a targeting construct comprises an ERT2*mut81 sequence and an ERT2*mut41v2 sequence. In some embodiments, a targeting construct comprises an ERT2*mut88 sequence and an ERT2*mut63 sequence. In some embodiments, a targeting construct comprises an ERT2*mut88 sequence and an ERT2*mut41 sequence. In some embodiments, a targeting construct comprises an ERT2*mut88 sequence and an ERT2*mut51 sequence. In some embodiments, a targeting construct comprises an ERT2*mut88 sequence and an ERT2*mut37 sequence. In some embodiments, a targeting construct comprises an ERT2*mut88 sequence and an ERT2*mut94 sequence. In some embodiments, a targeting construct comprises an ERT2*mut88 sequence and an ERT2*mut41v2 sequence. In some embodiments, a targeting construct comprises an ERT2*mut63 sequence and an ERT2*mut41 sequence. In some embodiments, a targeting construct comprises an ERT2*mut63 sequence and an ERT2*mut51 sequence. In some embodiments, a targeting construct comprises an ERT2*mut63 sequence and an ERT2*mut37 sequence. In some embodiments, a targeting construct comprises an ERT2*mut63 sequence and an ERT2*mut94 sequence. In some embodiments, a targeting construct comprises an ERT2*mut63 sequence and an ERT2*mut41v2 sequence. In some embodiments, a targeting construct comprises an ERT2*mut41 sequence and an ERT2*mut51 sequence. In some embodiments, a targeting construct comprises an ERT2*mut41 sequence and an ERT2*mut37 sequence. In some embodiments, a targeting construct comprises an ERT2*mut41 sequence and an ERT2*mut94 sequence. In some embodiments, a targeting construct comprises an ERT2*mut41 sequence and an ERT2*mut41v2 sequence. In some embodiments, a targeting construct comprises an ERT2*mut51 sequence and an ERT2*mut37 sequence. In some embodiments, a targeting construct comprises an ERT2*mut51 sequence and an ERT2*mut94 sequence. In some embodiments, a targeting construct comprises an ERT2*mut51 sequence and an ERT2*mut41v2 sequence. In some embodiments, a targeting construct comprises an ERT2*mut37 sequence and an ERT2*mut94 sequence. In some embodiments, a targeting construct comprises an ERT2*mut37 sequence and an ERT2*mut41v2 sequence. In some embodiments, a targeting construct comprises an ERT2*mut94 sequence and an ERT2*mut41v2 sequence.

In some embodiments, a targeting construct comprises an amino acid sequence at least 80%, at least 85%, at least 90%, at least 91%, at least 92%, at least 93%, at least 94%, at least 95%, at least 96%, at least 97%, at least 98%, at least 99%, at least 99.5%, or at least 99.9% identical to an amino acid sequence set forth in SEQ ID NO: 13, 15, 17, 19, 21, 23, 25, 27, 44, 46, 48, or 50.

In some embodiments, a targeting construct comprises an amino acid sequence at least 80%, at least 85%, at least 90%, at least 91%, at least 92%, at least 93%, at least 94%, at least 95%, at least 96%, at least 97%, at least 98%, at least 99%, at least 99.5%, or at least 99.9% identical to the amino acid sequence set forth in SEQ ID NO: 13. In some embodiments, a targeting construct comprises an amino acid sequence at least 80% identical to the amino acid sequence set forth in SEQ ID NO: 13. In some embodiments, a targeting construct comprises an amino acid sequence at least 85% identical to the amino acid sequence set forth in SEQ ID NO: 13. In some embodiments, a targeting construct comprises an amino acid sequence at least 90% identical to the amino acid sequence set forth in SEQ ID NO: 13. In some embodiments, a targeting construct comprises an amino acid sequence at least 91% identical to the amino acid sequence set forth in SEQ ID NO: 13. In some embodiments, a targeting construct comprises an amino acid sequence at least 92% identical to the amino acid sequence set forth in SEQ ID NO: 13. In some embodiments, a targeting construct comprises an amino acid sequence at least 93% identical to the amino acid sequence set forth in SEQ ID NO: 13. In some embodiments, a targeting construct comprises an amino acid sequence at least 94% identical to the amino acid sequence set forth in SEQ ID NO: 13. In some embodiments, a targeting construct comprises an amino acid sequence at least 95% identical to the amino acid sequence set forth in SEQ ID NO: 13. In some embodiments, a targeting construct comprises an amino acid sequence at least 96% identical to the amino acid sequence set forth in SEQ ID NO: 13. In some embodiments, a targeting construct comprises an amino acid sequence at least 97% identical to the amino acid sequence set forth in SEQ ID NO: 13. In some embodiments, a targeting construct comprises an amino acid sequence at least 98% identical to the amino acid sequence set forth in SEQ ID NO: 13. In some embodiments, a targeting construct comprises an amino acid sequence at least 99% identical to the amino acid sequence set forth in SEQ ID NO: 13. In some embodiments, a targeting construct comprises an amino acid sequence at least 99.5% identical to the amino acid sequence set forth in SEQ ID NO: 13. In some embodiments, a targeting construct comprises an amino acid sequence at least 99.9% identical to the amino acid sequence set forth in SEQ ID NO: 13. In some embodiments, a targeting construct comprises the amino acid sequence set forth in SEQ ID NO: 13.

In some embodiments, a targeting construct comprises an amino acid sequence at least 80%, at least 85%, at least 90%, at least 91%, at least 92%, at least 93%, at least 94%, at least 95%, at least 96%, at least 97%, at least 98%, at least 99%, at least 99.5%, or at least 99.9% identical to the amino acid sequence set forth in SEQ ID NO: 15. In some embodiments, a targeting construct comprises an amino acid sequence at least 80% identical to the amino acid sequence set forth in SEQ ID NO: 15. In some embodiments, a targeting construct comprises an amino acid sequence at least 85% identical to the amino acid sequence set forth in SEQ ID NO: 15. In some embodiments, a targeting construct comprises an amino acid sequence at least 90% identical to the amino acid sequence set forth in SEQ ID NO: 15. In some embodiments, a targeting construct comprises an amino acid sequence at least 91% identical to the amino acid sequence set forth in SEQ ID NO: 15. In some embodiments, a targeting construct comprises an amino acid sequence at least 92% identical to the amino acid sequence set forth in SEQ ID NO: 15. In some embodiments, a targeting construct comprises an amino acid sequence at least 93% identical to the amino acid sequence set forth in SEQ ID NO: 15. In some embodiments, a targeting construct comprises an amino acid sequence at least 94% identical to the amino acid sequence set forth in SEQ ID NO: 15. In some embodiments, a targeting construct comprises an amino acid sequence at least 95% identical to the amino acid sequence set forth in SEQ ID NO: 15. In some embodiments, a targeting construct comprises an amino acid sequence at least 96% identical to the amino acid sequence set forth in SEQ ID NO: 15. In some embodiments, a targeting construct comprises an amino acid sequence at least 97% identical to the amino acid sequence set forth in SEQ ID NO: 15. In some embodiments, a targeting construct comprises an amino acid sequence at least 98% identical to the amino acid sequence set forth in SEQ ID NO: 15. In some embodiments, a targeting construct comprises an amino acid sequence at least 99% identical to the amino acid sequence set forth in SEQ ID NO: 15. In some embodiments, a targeting construct comprises an amino acid sequence at least 99.5% identical to the amino acid sequence set forth in SEQ ID NO: 15. In some embodiments, a targeting construct comprises an amino acid sequence at least 99.9% identical to the amino acid sequence set forth in SEQ ID NO: 15. In some embodiments, a targeting construct comprises the amino acid sequence set forth in SEQ ID NO: 15.

In some embodiments, a targeting construct comprises an amino acid sequence at least 80%, at least 85%, at least 90%, at least 91%, at least 92%, at least 93%, at least 94%, at least 95%, at least 96%, at least 97%, at least 98%, at least 99%, at least 99.5%, or at least 99.9% identical to the amino acid sequence set forth in SEQ ID NO: 17. In some embodiments, a targeting construct comprises an amino acid sequence at least 80% identical to the amino acid sequence set forth in SEQ ID NO: 17. In some embodiments, a targeting construct comprises an amino acid sequence at least 85% identical to the amino acid sequence set forth in SEQ ID NO: 17. In some embodiments, a targeting construct comprises an amino acid sequence at least 90% identical to the amino acid sequence set forth in SEQ ID NO: 17. In some embodiments, a targeting construct comprises an amino acid sequence at least 91% identical to the amino acid sequence set forth in SEQ ID NO: 17. In some embodiments, a targeting construct comprises an amino acid sequence at least 92% identical to the amino acid sequence set forth in SEQ ID NO: 17. In some embodiments, a targeting construct comprises an amino acid sequence at least 93% identical to the amino acid sequence set forth in SEQ ID NO: 17. In some embodiments, a targeting construct comprises an amino acid sequence at least 94% identical to the amino acid sequence set forth in SEQ ID NO: 17. In some embodiments, a targeting construct comprises an amino acid sequence at least 95% identical to the amino acid sequence set forth in SEQ ID NO: 17. In some embodiments, a targeting construct comprises an amino acid sequence at least 96% identical to the amino acid sequence set forth in SEQ ID NO: 17. In some embodiments, a targeting construct comprises an amino acid sequence at least 97% identical to the amino acid sequence set forth in SEQ ID NO: 17. In some embodiments, a targeting construct comprises an amino acid sequence at least 98% identical to the amino acid sequence set forth in SEQ ID NO: 17. In some embodiments, a targeting construct comprises an amino acid sequence at least 99% identical to the amino acid sequence set forth in SEQ ID NO: 17. In some embodiments, a targeting construct comprises an amino acid sequence at least 99.5% identical to the amino acid sequence set forth in SEQ ID NO: 17. In some embodiments, a targeting construct comprises an amino acid sequence at least 99.9% identical to the amino acid sequence set forth in SEQ ID NO: 17. In some embodiments, a targeting construct comprises the amino acid sequence set forth in SEQ ID NO: 17.

In some embodiments, a targeting construct comprises an amino acid sequence at least 80%, at least 85%, at least 90%, at least 91%, at least 92%, at least 93%, at least 94%, at least 95%, at least 96%, at least 97%, at least 98%, at least 99%, at least 99.5%, or at least 99.9% identical to the amino acid sequence set forth in SEQ ID NO: 19. In some embodiments, a targeting construct comprises an amino acid sequence at least 80% identical to the amino acid sequence set forth in SEQ ID NO: 19. In some embodiments, a targeting construct comprises an amino acid sequence at least 85% identical to the amino acid sequence set forth in SEQ ID NO: 19. In some embodiments, a targeting construct comprises an amino acid sequence at least 90% identical to the amino acid sequence set forth in SEQ ID NO: 19. In some embodiments, a targeting construct comprises an amino acid sequence at least 91% identical to the amino acid sequence set forth in SEQ ID NO: 19. In some embodiments, a targeting construct comprises an amino acid sequence at least 92% identical to the amino acid sequence set forth in SEQ ID NO: 19. In some embodiments, a targeting construct comprises an amino acid sequence at least 93% identical to the amino acid sequence set forth in SEQ ID NO: 19. In some embodiments, a targeting construct comprises an amino acid sequence at least 94% identical to the amino acid sequence set forth in SEQ ID NO: 19. In some embodiments, a targeting construct comprises an amino acid sequence at least 95% identical to the amino acid sequence set forth in SEQ ID NO: 19. In some embodiments, a targeting construct comprises an amino acid sequence at least 96% identical to the amino acid sequence set forth in SEQ ID NO: 19. In some embodiments, a targeting construct comprises an amino acid sequence at least 97% identical to the amino acid sequence set forth in SEQ ID NO: 19. In some embodiments, a targeting construct comprises an amino acid sequence at least 98% identical to the amino acid sequence set forth in SEQ ID NO: 19. In some embodiments, a targeting construct comprises an amino acid sequence at least 99% identical to the amino acid sequence set forth in SEQ ID NO: 19. In some embodiments, a targeting construct comprises an amino acid sequence at least 99.5% identical to the amino acid sequence set forth in SEQ ID NO: 19. In some embodiments, a targeting construct comprises an amino acid sequence at least 99.9% identical to the amino acid sequence set forth in SEQ ID NO: 19. In some embodiments, a targeting construct comprises the amino acid sequence set forth in SEQ ID NO: 19.

In some embodiments, a targeting construct comprises an amino acid sequence at least 80%, at least 85%, at least 90%, at least 91%, at least 92%, at least 93%, at least 94%, at least 95%, at least 96%, at least 97%, at least 98%, at least 99%, at least 99.5%, or at least 99.9% identical to the amino acid sequence set forth in SEQ ID NO: 21. In some embodiments, a targeting construct comprises an amino acid sequence at least 80% identical to the amino acid sequence set forth in SEQ ID NO: 21. In some embodiments, a targeting construct comprises an amino acid sequence at least 85% identical to the amino acid sequence set forth in SEQ ID NO: 21. In some embodiments, a targeting construct comprises an amino acid sequence at least 90% identical to the amino acid sequence set forth in SEQ ID NO: 21. In some embodiments, a targeting construct comprises an amino acid sequence at least 91% identical to the amino acid sequence set forth in SEQ ID NO: 21. In some embodiments, a targeting construct comprises an amino acid sequence at least 92% identical to the amino acid sequence set forth in SEQ ID NO: 21. In some embodiments, a targeting construct comprises an amino acid sequence at least 93% identical to the amino acid sequence set forth in SEQ ID NO: 21. In some embodiments, a targeting construct comprises an amino acid sequence at least 94% identical to the amino acid sequence set forth in SEQ ID NO: 21. In some embodiments, a targeting construct comprises an amino acid sequence at least 95% identical to the amino acid sequence set forth in SEQ ID NO: 21. In some embodiments, a targeting construct comprises an amino acid sequence at least 96% identical to the amino acid sequence set forth in SEQ ID NO: 21. In some embodiments, a targeting construct comprises an amino acid sequence at least 97% identical to the amino acid sequence set forth in SEQ ID NO: 21. In some embodiments, a targeting construct comprises an amino acid sequence at least 98% identical to the amino acid sequence set forth in SEQ ID NO: 21. In some embodiments, a targeting construct comprises an amino acid sequence at least 99% identical to the amino acid sequence set forth in SEQ ID NO: 21. In some embodiments, a targeting construct comprises an amino acid sequence at least 99.5% identical to the amino acid sequence set forth in SEQ ID NO: 21. In some embodiments, a targeting construct comprises an amino acid sequence at least 99.9% identical to the amino acid sequence set forth in SEQ ID NO: 21. In some embodiments, a targeting construct comprises the amino acid sequence set forth in SEQ ID NO: 21.

In some embodiments, a targeting construct comprises an amino acid sequence at least 80%, at least 85%, at least 90%, at least 91%, at least 92%, at least 93%, at least 94%, at least 95%, at least 96%, at least 97%, at least 98%, at least 99%, at least 99.5%, or at least 99.9% identical to the amino acid sequence set forth in SEQ ID NO: 23. In some embodiments, a targeting construct comprises an amino acid sequence at least 80% identical to the amino acid sequence set forth in SEQ ID NO: 23. In some embodiments, a targeting construct comprises an amino acid sequence at least 85% identical to the amino acid sequence set forth in SEQ ID NO: 23. In some embodiments, a targeting construct comprises an amino acid sequence at least 90% identical to the amino acid sequence set forth in SEQ ID NO: 23. In some embodiments, a targeting construct comprises an amino acid sequence at least 91% identical to the amino acid sequence set forth in SEQ ID NO: 23. In some embodiments, a targeting construct comprises an amino acid sequence at least 92% identical to the amino acid sequence set forth in SEQ ID NO: 23. In some embodiments, a targeting construct comprises an amino acid sequence at least 93% identical to the amino acid sequence set forth in SEQ ID NO: 23. In some embodiments, a targeting construct comprises an amino acid sequence at least 94% identical to the amino acid sequence set forth in SEQ ID NO: 23. In some embodiments, a targeting construct comprises an amino acid sequence at least 95% identical to the amino acid sequence set forth in SEQ ID NO: 23. In some embodiments, a targeting construct comprises an amino acid sequence at least 96% identical to the amino acid sequence set forth in SEQ ID NO: 23. In some embodiments, a targeting construct comprises an amino acid sequence at least 97% identical to the amino acid sequence set forth in SEQ ID NO: 23. In some embodiments, a targeting construct comprises an amino acid sequence at least 98% identical to the amino acid sequence set forth in SEQ ID NO: 23. In some embodiments, a targeting construct comprises an amino acid sequence at least 99% identical to the amino acid sequence set forth in SEQ ID NO: 23. In some embodiments, a targeting construct comprises an amino acid sequence at least 99.5% identical to the amino acid sequence set forth in SEQ ID NO: 23. In some embodiments, a targeting construct comprises an amino acid sequence at least 99.9% identical to the amino acid sequence set forth in SEQ ID NO: 23. In some embodiments, a targeting construct comprises the amino acid sequence set forth in SEQ ID NO: 23.

In some embodiments, a targeting construct comprises an amino acid sequence at least 80%, at least 85%, at least 90%, at least 91%, at least 92%, at least 93%, at least 94%, at least 95%, at least 96%, at least 97%, at least 98%, at least 99%, at least 99.5%, or at least 99.9% identical to the amino acid sequence set forth in SEQ ID NO: 25. In some embodiments, a targeting construct comprises an amino acid sequence at least 80% identical to the amino acid sequence set forth in SEQ ID NO: 25. In some embodiments, a targeting construct comprises an amino acid sequence at least 85% identical to the amino acid sequence set forth in SEQ ID NO: 25. In some embodiments, a targeting construct comprises an amino acid sequence at least 90% identical to the amino acid sequence set forth in SEQ ID NO: 25. In some embodiments, a targeting construct comprises an amino acid sequence at least 91% identical to the amino acid sequence set forth in SEQ ID NO: 25. In some embodiments, a targeting construct comprises an amino acid sequence at least 92% identical to the amino acid sequence set forth in SEQ ID NO: 25. In some embodiments, a targeting construct comprises an amino acid sequence at least 93% identical to the amino acid sequence set forth in SEQ ID NO: 25. In some embodiments, a targeting construct comprises an amino acid sequence at least 94% identical to the amino acid sequence set forth in SEQ ID NO: 25. In some embodiments, a targeting construct comprises an amino acid sequence at least 95% identical to the amino acid sequence set forth in SEQ ID NO: 25. In some embodiments, a targeting construct comprises an amino acid sequence at least 96% identical to the amino acid sequence set forth in SEQ ID NO: 25. In some embodiments, a targeting construct comprises an amino acid sequence at least 97% identical to the amino acid sequence set forth in SEQ ID NO: 25. In some embodiments, a targeting construct comprises an amino acid sequence at least 98% identical to the amino acid sequence set forth in SEQ ID NO: 25. In some embodiments, a targeting construct comprises an amino acid sequence at least 99% identical to the amino acid sequence set forth in SEQ ID NO: 25. In some embodiments, a targeting construct comprises an amino acid sequence at least 99.5% identical to the amino acid sequence set forth in SEQ ID NO: 25. In some embodiments, a targeting construct comprises an amino acid sequence at least 99.9% identical to the amino acid sequence set forth in SEQ ID NO: 25. In some embodiments, a targeting construct comprises the amino acid sequence set forth in SEQ ID NO: 25.

In some embodiments, a targeting construct comprises an amino acid sequence at least 80%, at least 85%, at least 90%, at least 91%, at least 92%, at least 93%, at least 94%, at least 95%, at least 96%, at least 97%, at least 98%, at least 99%, at least 99.5%, or at least 99.9% identical to the amino acid sequence set forth in SEQ ID NO: 27. In some embodiments, a targeting construct comprises an amino acid sequence at least 80% identical to the amino acid sequence set forth in SEQ ID NO: 27. In some embodiments, a targeting construct comprises an amino acid sequence at least 85% identical to the amino acid sequence set forth in SEQ ID NO: 27. In some embodiments, a targeting construct comprises an amino acid sequence at least 90% identical to the amino acid sequence set forth in SEQ ID NO: 27. In some embodiments, a targeting construct comprises an amino acid sequence at least 91% identical to the amino acid sequence set forth in SEQ ID NO: 27. In some embodiments, a targeting construct comprises an amino acid sequence at least 92% identical to the amino acid sequence set forth in SEQ ID NO: 27. In some embodiments, a targeting construct comprises an amino acid sequence at least 93% identical to the amino acid sequence set forth in SEQ ID NO: 27. In some embodiments, a targeting construct comprises an amino acid sequence at least 94% identical to the amino acid sequence set forth in SEQ ID NO: 27. In some embodiments, a targeting construct comprises an amino acid sequence at least 95% identical to the amino acid sequence set forth in SEQ ID NO: 27. In some embodiments, a targeting construct comprises an amino acid sequence at least 96% identical to the amino acid sequence set forth in SEQ ID NO: 27. In some embodiments, a targeting construct comprises an amino acid sequence at least 97% identical to the amino acid sequence set forth in SEQ ID NO: 27. In some embodiments, a targeting construct comprises an amino acid sequence at least 98% identical to the amino acid sequence set forth in SEQ ID NO: 27. In some embodiments, a targeting construct comprises an amino acid sequence at least 99% identical to the amino acid sequence set forth in SEQ ID NO: 27. In some embodiments, a targeting construct comprises an amino acid sequence at least 99.5% identical to the amino acid sequence set forth in SEQ ID NO: 27. In some embodiments, a targeting construct comprises an amino acid sequence at least 99.9% identical to the amino acid sequence set forth in SEQ ID NO: 27. In some embodiments, a targeting construct comprises the amino acid sequence set forth in SEQ ID NO: 27.

In some embodiments, a targeting construct comprises an amino acid sequence at least 80%, at least 85%, at least 90%, at least 91%, at least 92%, at least 93%, at least 94%, at least 95%, at least 96%, at least 97%, at least 98%, at least 99%, at least 99.5%, or at least 99.9% identical to the amino acid sequence set forth in SEQ ID NO: 44. In some embodiments, a targeting construct comprises an amino acid sequence at least 80% identical to the amino acid sequence set forth in SEQ ID NO: 44. In some embodiments, a targeting construct comprises an amino acid sequence at least 85% identical to the amino acid sequence set forth in SEQ ID NO: 44. In some embodiments, a targeting construct comprises an amino acid sequence at least 90% identical to the amino acid sequence set forth in SEQ ID NO: 44. In some embodiments, a targeting construct comprises an amino acid sequence at least 91% identical to the amino acid sequence set forth in SEQ ID NO: 44. In some embodiments, a targeting construct comprises an amino acid sequence at least 92% identical to the amino acid sequence set forth in SEQ ID NO: 44. In some embodiments, a targeting construct comprises an amino acid sequence at least 93% identical to the amino acid sequence set forth in SEQ ID NO: 44. In some embodiments, a targeting construct comprises an amino acid sequence at least 94% identical to the amino acid sequence set forth in SEQ ID NO: 44. In some embodiments, a targeting construct comprises an amino acid sequence at least 95% identical to the amino acid sequence set forth in SEQ ID NO: 44. In some embodiments, a targeting construct comprises an amino acid sequence at least 96% identical to the amino acid sequence set forth in SEQ ID NO: 44. In some embodiments, a targeting construct comprises an amino acid sequence at least 97% identical to the amino acid sequence set forth in SEQ ID NO: 44. In some embodiments, a targeting construct comprises an amino acid sequence at least 98% identical to the amino acid sequence set forth in SEQ ID NO: 44. In some embodiments, a targeting construct comprises an amino acid sequence at least 99% identical to the amino acid sequence set forth in SEQ ID NO: 44. In some embodiments, a targeting construct comprises an amino acid sequence at least 99.5% identical to the amino acid sequence set forth in SEQ ID NO: 44. In some embodiments, a targeting construct comprises an amino acid sequence at least 99.9% identical to the amino acid sequence set forth in SEQ ID NO: 44. In some embodiments, a targeting construct comprises the amino acid sequence set forth in SEQ ID NO: 44.

In some embodiments, a targeting construct comprises an amino acid sequence at least 80%, at least 85%, at least 90%, at least 91%, at least 92%, at least 93%, at least 94%, at least 95%, at least 96%, at least 97%, at least 98%, at least 99%, at least 99.5%, or at least 99.9% identical to the amino acid sequence set forth in SEQ ID NO: 46. In some embodiments, a targeting construct comprises an amino acid sequence at least 80% identical to the amino acid sequence set forth in SEQ ID NO: 46. In some embodiments, a targeting construct comprises an amino acid sequence at least 85% identical to the amino acid sequence set forth in SEQ ID NO: 46. In some embodiments, a targeting construct comprises an amino acid sequence at least 90% identical to the amino acid sequence set forth in SEQ ID NO: 46. In some embodiments, a targeting construct comprises an amino acid sequence at least 91% identical to the amino acid sequence set forth in SEQ ID NO: 46. In some embodiments, a targeting construct comprises an amino acid sequence at least 92% identical to the amino acid sequence set forth in SEQ ID NO: 46. In some embodiments, a targeting construct comprises an amino acid sequence at least 93% identical to the amino acid sequence set forth in SEQ ID NO: 46. In some embodiments, a targeting construct comprises an amino acid sequence at least 94% identical to the amino acid sequence set forth in SEQ ID NO: 46. In some embodiments, a targeting construct comprises an amino acid sequence at least 95% identical to the amino acid sequence set forth in SEQ ID NO: 46. In some embodiments, a targeting construct comprises an amino acid sequence at least 96% identical to the amino acid sequence set forth in SEQ ID NO: 46. In some embodiments, a targeting construct comprises an amino acid sequence at least 97% identical to the amino acid sequence set forth in SEQ ID NO: 46. In some embodiments, a targeting construct comprises an amino acid sequence at least 98% identical to the amino acid sequence set forth in SEQ ID NO: 46. In some embodiments, a targeting construct comprises an amino acid sequence at least 99% identical to the amino acid sequence set forth in SEQ ID NO: 46. In some embodiments, a targeting construct comprises an amino acid sequence at least 99.5% identical to the amino acid sequence set forth in SEQ ID NO: 46. In some embodiments, a targeting construct comprises an amino acid sequence at least 99.9% identical to the amino acid sequence set forth in SEQ ID NO: 46. In some embodiments, a targeting construct comprises the amino acid sequence set forth in SEQ ID NO: 46.

In some embodiments, a targeting construct comprises an amino acid sequence at least 80%, at least 85%, at least 90%, at least 91%, at least 92%, at least 93%, at least 94%, at least 95%, at least 96%, at least 97%, at least 98%, at least 99%, at least 99.5%, or at least 99.9% identical to the amino acid sequence set forth in SEQ ID NO: 48. In some embodiments, a targeting construct comprises an amino acid sequence at least 80% identical to the amino acid sequence set forth in SEQ ID NO: 48. In some embodiments, a targeting construct comprises an amino acid sequence at least 85% identical to the amino acid sequence set forth in SEQ ID NO: 48. In some embodiments, a targeting construct comprises an amino acid sequence at least 90% identical to the amino acid sequence set forth in SEQ ID NO: 48. In some embodiments, a targeting construct comprises an amino acid sequence at least 91% identical to the amino acid sequence set forth in SEQ ID NO: 48. In some embodiments, a targeting construct comprises an amino acid sequence at least 92% identical to the amino acid sequence set forth in SEQ ID NO: 48. In some embodiments, a targeting construct comprises an amino acid sequence at least 93% identical to the amino acid sequence set forth in SEQ ID NO: 48. In some embodiments, a targeting construct comprises an amino acid sequence at least 94% identical to the amino acid sequence set forth in SEQ ID NO: 48. In some embodiments, a targeting construct comprises an amino acid sequence at least 95% identical to the amino acid sequence set forth in SEQ ID NO: 48. In some embodiments, a targeting construct comprises an amino acid sequence at least 96% identical to the amino acid sequence set forth in SEQ ID NO: 48. In some embodiments, a targeting construct comprises an amino acid sequence at least 97% identical to the amino acid sequence set forth in SEQ ID NO: 48. In some embodiments, a targeting construct comprises an amino acid sequence at least 98% identical to the amino acid sequence set forth in SEQ ID NO: 48. In some embodiments, a targeting construct comprises an amino acid sequence at least 99% identical to the amino acid sequence set forth in SEQ ID NO: 48. In some embodiments, a targeting construct comprises an amino acid sequence at least 99.5% identical to the amino acid sequence set forth in SEQ ID NO: 48. In some embodiments, a targeting construct comprises an amino acid sequence at least 99.9% identical to the amino acid sequence set forth in SEQ ID NO: 48. In some embodiments, a targeting construct comprises the amino acid sequence set forth in SEQ ID NO: 48.

In some embodiments, a targeting construct comprises an amino acid sequence at least 80%, at least 85%, at least 90%, at least 91%, at least 92%, at least 93%, at least 94%, at least 95%, at least 96%, at least 97%, at least 98%, at least 99%, at least 99.5%, or at least 99.9% identical to the amino acid sequence set forth in SEQ ID NO: 50. In some embodiments, a targeting construct comprises an amino acid sequence at least 80% identical to the amino acid sequence set forth in SEQ ID NO: 50. In some embodiments, a targeting construct comprises an amino acid sequence at least 85% identical to the amino acid sequence set forth in SEQ ID NO: 50. In some embodiments, a targeting construct comprises an amino acid sequence at least 90% identical to the amino acid sequence set forth in SEQ ID NO: 50. In some embodiments, a targeting construct comprises an amino acid sequence at least 91% identical to the amino acid sequence set forth in SEQ ID NO: 50. In some embodiments, a targeting construct comprises an amino acid sequence at least 92% identical to the amino acid sequence set forth in SEQ ID NO: 50. In some embodiments, a targeting construct comprises an amino acid sequence at least 93% identical to the amino acid sequence set forth in SEQ ID NO: 50. In some embodiments, a targeting construct comprises an amino acid sequence at least 94% identical to the amino acid sequence set forth in SEQ ID NO: 50. In some embodiments, a targeting construct comprises an amino acid sequence at least 95% identical to the amino acid sequence set forth in SEQ ID NO: 50. In some embodiments, a targeting construct comprises an amino acid sequence at least 96% identical to the amino acid sequence set forth in SEQ ID NO: 50. In some embodiments, a targeting construct comprises an amino acid sequence at least 97% identical to the amino acid sequence set forth in SEQ ID NO: 50. In some embodiments, a targeting construct comprises an amino acid sequence at least 98% identical to the amino acid sequence set forth in SEQ ID NO: 50. In some embodiments, a targeting construct comprises an amino acid sequence at least 99% identical to the amino acid sequence set forth in SEQ ID NO: 50. In some embodiments, a targeting construct comprises an amino acid sequence at least 99.5% identical to the amino acid sequence set forth in SEQ ID NO: 50. In some embodiments, a targeting construct comprises an amino acid sequence at least 99.9% identical to the amino acid sequence set forth in SEQ ID NO: 50. In some embodiments, a targeting construct comprises the amino acid sequence set forth in SEQ ID NO: 50.

### Target Cells

In some embodiments, a targeting construct is introduced into target cells or populations of target cells in order to produce engineered target cells in which a modified ERT2 peptide-suicide protein fusion polypeptide coding sequence is integrated into and expressed from a STEL locus.

In some embodiments, the methods of the disclosure may be employed to express a modified ERT2 peptide-suicide protein fusion polypeptide in mitotic or post-mitotic target cells in vivo and/or ex vivo and/or in vitro (e.g., to produce engineered target cells that can be reintroduced into an individual).

Any type of cell that may be of interest may be engineered to incorporate a modified ERT2 peptide-suicide protein fusion polypeptide coding sequence into a STEL locus. In various embodiments, the target cell is a stem cell, e.g., a human embryonic stem cell (hESC), an induced pluripotent stem cell (iPSC), a germ cell; a somatic cell, e.g., a fibroblast, a hematopoietic cell, a neuron, a muscle cell, a bone cell, a hepatocyte, a pancreatic cell; an in vitro or in vivo embryonic cell of an embryo at any stage, e.g., a 1-cell, 2-cell, 4-cell, 8-cell, etc. stage zebrafish embryo; etc.). Cells may be from established cell lines, or they may be primary cells, where “primary cells”, “primary cell lines”, and “primary cultures” are used interchangeably herein to refer to cells and cells cultures that have been derived from a subject and allowed to grow in vitro for a limited number of passages, e.g., splittings, of the culture. For example, primary cultures include cultures that may have been passaged 0 times, 1 time, 2 times, 4 times, 5 times, 10 times, or 15 times, but not enough times to go through the crisis stage. Primary cell lines can be maintained for fewer than 10 passages in vitro. Target cells are, in some embodiments, unicellular organisms, or are grown in culture. Preferably, the target cells are of human origin.

In some embodiments, a cell engineered to incorporate a modified ERT2 peptide-suicide protein fusion polypeptide coding sequence is a cell therapy modality. In some embodiments, the cell therapy modality is an autologous cell therapy modality. In some embodiments, the cell therapy modality is an allogeneic cell therapy modality.

If the cells are primary cells, such cells may be harvested from an individual by any suitable method. For example, leukocytes may be suitably harvested by apheresis, leukocytapheresis, density gradient separation, etc., while cells from tissues such as skin, muscle, bone marrow, spleen, liver, pancreas, lung, intestine, stomach, etc. are most suitably harvested by biopsy. An appropriate solution may be used for dispersion or suspension of the harvested cells. Such solution will generally be a balanced salt solution, e.g., normal saline, phosphate-buffered saline (PBS), Hank's balanced salt solution, etc., suitably supplemented with fetal calf serum or other naturally occurring factors, in conjunction with an acceptable buffer at low concentration, e.g., from 5-25 mM. Suitable buffers include HEPES, phosphate buffers, lactate buffers, etc. The cells may be used immediately, or they may be stored, frozen, for long periods of time, being thawed and capable of being reused. In such cases, the cells will generally be frozen in 10% dimethyl sulfoxide (DMSO), 50% serum, 40% buffered medium, or some other such solution as is commonly used in the art to preserve cells at such freezing temperatures and thawed in a manner as commonly known in the art for thawing frozen cultured cells.

In some embodiments, a targeting construct is introduced into a target cell via nucleofection, as part of a gene editing system (e.g., a CRISPR/Cas-based gene editing system) design to cleave target sequences within or near (e.g., flanking or adjoining) a STEL locus to facilitate homologous recombination of the targeting constructs of the target sequences. In some embodiments, the gene editing system comprises an endonuclease (e.g., a Cas nuclease), a guide RNA (e.g., single guide RNA or sgRNA), as well as the targeting construct. In some embodiments, the gene editing system is in the form of a composition known as a ribonucleoprotein or RNP complex. An RNP complex is assembled by combining an endonuclease with a ribonucleic acid.

### Stem Cells

In some embodiments, the target cells that are engineered to express a modified ERT2 peptide-suicide protein fusion polypeptide from a STEL locus are stem cells, particularly pluripotent stem cells (PSCs) such as induced pluripotent stem cells (iPSCs) or human embryonic stem cells (hESCs), which are the starting point for the potential generation of large numbers of a specific cell type that can be delivered for regenerative medicine in patients with many different diseases. In some embodiments, the stem cells are human stem cells.

Following engineering to a PSC to express a modified ERT2 peptide-suicide protein fusion polypeptide the PSC can be differentiated into a cell type of interest for cell therapy.

The PSCs, e.g., ESCs or recombinant PSCs can be differentiated into cells suitable for therapy, including the cells in the endoderm (e.g., lung, thyroid, or pancreatic cells, or progenitors thereof), ectoderm (e.g., skin, neuronal, or pigment cells, or progenitors thereof), and mesoderm (e.g., cardiac cells, skeletal muscle cells, red blood cells, smooth muscle cells, or progenitors thereof) lineages.

In some embodiments, the PSCs, e.g., ESCs or recombinant PSCs are differentiated into cardiovascular cells (cardiovascular cells (e.g., cardiomyocytes, pacemaker cell, cardiac fibroblasts, epicardial cells, endocardial cells, valvular interstitial cells, cardiac and vascular smooth muscle cells, cardiac and vascular endothelial cells, Purkinje fibers, or His-bundle cells)

In some embodiments, the PSCs, e.g., ESCs or recombinant PSCs are differentiated into cells in the endoderm (e.g., lung, thyroid, or pancreatic cells, or progenitors thereof), ectoderm (e.g., skin, neuronal, or pigment cells, or progenitors thereof) or mesoderm (e.g., cardiac cells, skeletal muscle cells, red blood cells, smooth muscle cells, or progenitors thereof) lineages.

In some embodiments, a recombinant PSC of the disclosure is differentiated into a cardiac cell, or a precursor or progenitor cell thereof. In various embodiments, the cardiac cell is a cardiac progenitor cell or a mature or immature (atrial or ventricular) cardiomyocyte. In other embodiments, the cardiac cell is a cardiac endothelial cell or a nodal cell.

In some embodiments, a recombinant PSC of the disclosure is differentiated into a regulatory T cell, a myeloid cell, a dendritic cell, a macrophage (e.g., an immunosuppressive macrophage), a myeloid progenitor cell, or a precursor or progenitor cell thereof. Details on differentiation of PSCs into myeloid progenitor cells can be found in International (PCT) Publication Numbers WO 2023/150089 A1 and WO 2017/152081 A1.

In some embodiments, a recombinant PSC of the disclosure is a neural cell or a precursor or progenitor cell thereof. In some embodiments, a neural cell is a microglia, a macroglia (e.g., oligodendrocytes, an astrocytes, a Schwann cells, or an enteric glia) and neurons, or neural stem and precursor cells or progenitor cells of any of the foregoing cells. In some embodiments, a recombinant PSC of the disclosure is differentiated into an oligodendrocyte progenitor cell or an oligodendrocyte.

In some embodiments, a recombinant PSC of the disclosure is differentiated into a neural lineage cell, for example a neural crest cells, an astrocyte, a dopaminergic neuron progenitor cell, a dopaminergic neuron cells, a midbrain dopaminergic neuron progenitor cell, a midbrain dopaminergic neuron, an authentic midbrain dopamine (DA) neuron, a dopaminergic neuron precursor cell, a floor plate midbrain progenitor cell, a floor plate midbrain DA neuron.

In some embodiments, a recombinant PSC of the disclosure is differentiated into a cell of the ocular system, such as a photoreceptor cell, a photoreceptor precursor cell, a retinal pigmented epithelium cell, a neural retinal cell, a neural retinal progenitor cell, or a precursor or progenitor cell thereof.

In further embodiments, a recombinant PSC of the disclosure is differentiated into a microglial cell or a microglial progenitor cell.

In further embodiments, a recombinant PSC of the disclosure is differentiated into a cell in the human metabolic system, optionally selected from a hepatocyte, a cholangiocyte, and a pancreatic beta cell, or a precursor or progenitor cell thereof.

In further embodiments, a recombinant PSC of the disclosure is differentiated into an enteric progenitor cell or an enteric cell.

### Differentiated Cells

In various embodiments, a cell at any stage of differentiation is engineered to express a modified ERT2 peptide-suicide protein fusion polypeptide. The differentiated cell may be derived from an engineered iPSC disclosed herein.

Exemplary differentiated cell types that can be engineered to express a modified ERT2 peptide-suicide protein fusion polypeptide include the cells in the endoderm (e.g., lung, thyroid, or pancreatic cells, or progenitors thereof), ectoderm (e.g., skin, neuronal, or pigment cells, or progenitors thereof) and mesoderm (e.g., cardiac cells, skeletal muscle cells, red blood cells, smooth muscle cells, or progenitors thereof) lineages. Alternatively, PSCs can be differentiated into cells in these lineages and then engineered with a targeting construct of the disclosure.

In some embodiments, a cardiac cell is engineered to express a modified ERT2 peptide-suicide protein fusion polypeptide. In some embodiments, the cardiac cell is a cardiac progenitor cell or a mature or immature (atrial or ventricular) cardiomyocyte. In other embodiments, the cardiac cell is a cardiac endothelial cell or a nodal cell.

Differentiated cell types can include cardiovascular cells (e.g., cardiomyocytes, pacemaker cell, cardiac fibroblasts, epicardial cells, endocardial cells, valvular interstitial cells, cardiac and vascular smooth muscle cells, cardiac and vascular endothelial cells, Purkinje fibers, or His-bundle cells).

In some embodiments, a human immune cell selected from a regulatory T cell, a myeloid cell, a dendritic cell, and/or a macrophage (e.g., an immunosuppressive macrophage), or a precursor or progenitor cell thereof is engineered to express a modified ERT2 peptide-suicide protein fusion polypeptide.

In some embodiments, a neural cell or a precursor or progenitor cell thereof, is engineered to express a modified ERT2 peptide-suicide protein fusion polypeptide. In some embodiments, a neural cell is a microglia, a macroglia (e.g., oligodendrocytes, an astrocytes, a Schwann cells, or an enteric glia) and neurons, or neural stem and precursor cells of any of the foregoing cells. In some embodiments, an oligodendrocyte progenitor cell or an oligodendrocyte is engineered to express a modified ERT2 peptide-suicide protein fusion polypeptide.

In some embodiments, a neural lineage cell is engineered to express a modified ERT2 peptide-suicide protein fusion polypeptide. In various embodiments, the neural lineage cell is a neural crest cell, an astrocyte, a dopaminergic neuron progenitor cell, a dopaminergic neuron cells, a midbrain dopaminergic neuron progenitor cell, a midbrain dopaminergic neuron, an authentic midbrain dopamine (DA) neuron, a dopaminergic neuron precursor cell, a floor plate midbrain progenitor cell, a floor plate midbrain DA neuron.

In some embodiments, a cell of the ocular system or a precursor or progenitor cell thereof is engineered to express a modified ERT2 peptide-suicide protein fusion polypeptide. In various embodiments, the cell of the ocular system is a photoreceptor cell, a photoreceptor precursor cell, a retinal pigmented epithelium cell, a neural retinal cell, or a neural retinal progenitor cell.

In further embodiments, a microglial cell or a microglial progenitor cell is engineered to express a modified ERT2 peptide-suicide protein fusion polypeptide.

In further embodiments, a cell in the human metabolic system is engineered to express a modified ERT2 peptide-suicide protein fusion polypeptide. In various embodiments, the cell in the human metabolic system is optionally selected from a hepatocyte, a cholangiocyte, and a pancreatic beta cell or a precursor or progenitor cell thereof.

In further embodiments, an enteric progenitor cell or an enteric cell is engineered to express a modified ERT2 peptide-suicide protein fusion polypeptide.

In some embodiments of any of the aforementioned cells, the cell is a human cell.

Any of the foregoing differentiated cell types can be differentiated from PSCs prior to engineering them to express a modified ERT2 peptide-suicide protein fusion polypeptide.

### Methods of Administration

The present disclosure provides methods of using the therapeutic cells disclosed herein for treating a patient in need of cell therapy. The methods comprise administering to the patient a gene-edited target cell engineered to express a modified ERT2 peptide-suicide protein fusion polypeptide from a STEL locus, for example a gene-edited target cell as disclosed in Section 0 or any subsection thereof. In some embodiments, the gene-edited target cell is comprised in a pharmaceutical composition comprising a pharmaceutically acceptable carrier.

The methods can further comprise controlling the gene-edited target cell population in the patient by (a) optionally monitoring the gene-edited target cell population in the patient; and/or (b) administering an inducer of the modified ER-LBD (e.g., an estrogen antagonist) if the patient experiences adverse events related to the gene-edited target cell population.

The present disclosure further provides methods of mitigating adverse events or a safety risk associated with cell therapy in the form of gene-edited target cells engineered to express a modified ERT2 peptide-suicide protein fusion polypeptide from a STEL locus, e.g., gene-edited target cell as disclosed in Section 0 or any subsection thereof, comprising administering to a patient who received the gene-edited target cells an inducer of a modified ER-LBD (e.g., an estrogen antagonist) if the patient experiences adverse events or a safety risk related to the gene-edited target cells or pharmaceutical composition.

In some embodiments, the inducer of the modified ER-LBD is an estrogen agonist such as tamoxifen or a tamoxifen metabolite.

In some aspects, the methods comprise administering a single inducer of the modified ER-LBD. In some embodiments, the single inducer of the modified ER-LBD is tamoxifen, 4-OHT, N-desmethyltamoxifen, tamoxifen-N-oxide, or endoxifen.

In some aspects, the methods comprise administering a combination of two or more inducers of the modified ER-LBD. In some embodiments, the combination of inducers of the modified ER-LBD comprises one or more of, or optionally two or more of, tamoxifen, 4-OHT, N-desmethyltamoxifen, tamoxifen-N-oxide, and endoxifen, in any combination (e.g., a combination comprising 4-OHT and endoxifen). Without being bound by theory, it is believed that administering a combination of two or more inducers of the modified ER-LBD can result in synergistic activity, thereby lowering the dosages required to achieve apoptosis as compared to administration of a single agent.

### Additional Embodiments

1. A targeting construct comprising:
   (a) a first homology arm corresponding to a 5′ target sequence comprising a first region of homology to a target genomic locus;
   (b) a nucleotide insert comprising a nucleotide sequence encoding a fusion polypeptide comprising:
      (i) a modified estrogen receptor ligand binding domain (ER-LBD); and
      (ii) a caspase 9 domain or derivative or functional fragment thereof;
   (c) a second homology arm corresponding to a 3′ target sequence comprising second region of homology to the target genomic locus
wherein the modified ER-LBD comprises an amino acid sequence corresponding to a hormone binding domain of a reference human estrogen receptor sequence (SEQ ID NO: 1), and wherein the modified ER-LBD comprises
   a G400V amino acid substitution, an M543A amino acid substitution, an L544A amino acid substitution, and optionally a V595A amino acid substitution, with reference to SEQ ID NO: 1; and
   additional amino acid substitutions, wherein the additional amino acid substitutions comprise, with reference to SEQ ID NO: 1:
   (i) an L384M substitution, an L391V substitution, a N413D substitution, an M421L substitution, a S463P substitution, and a H524L substitution,
   (ii) an L391V substitution, a N413D substitution, a Q414E substitution, a S463P substitution, and a H524F substitution,
   (iii) an L354I substitution, a L391V substitution, a N413D substitution, a Q414E substitution, a M421L substitution, a M517A substitution, and a H524F substitution,
   (iv) an L354I substitution, a L391V substitution, a L409V substitution, a N413D substitution, a Q414E substitution, and a H524L substitution,
   (v) an L391V substitution, a Q414E substitution, an N413D substitution, an S463P substitution, an M421L substitution, an L354I substitution, an L384M substitution, and an H524L substitution,
   (vi) an L391V substitution, an N413D substitution, an S463P substitution, an M517A substitution, an M421L substitution, an L354I substitution, and an H524L substitution,
   (vii) an N413D substitution, an S463P substitution, an L354I substitution, an L384M substitution, an H524L substitution, or
   (viii) an L391V substitution, an N413D substitution, an S463P substitution, an M517A substitution, an M421L substitution, an L354I substitution, and an H524L substitution,
wherein the targeting construct is configured such that upon its recombination with the target genomic locus, the fusion polypeptide coding sequence is integrated into a STEL gene and becomes operably linked to the STEL gene regulatory element.
2. The targeting construct of embodiment 1 or 108, wherein the additional amino acid substitutions comprise: an L384M substitution, an L391V substitution, a N413D substitution, an M421L substitution, a S463P substitution, and a H524L substitution, and wherein the modified ER-LBD comprises an amino acid sequence having at least 80%, at least 85%, at least 90%, or at least 95% sequence identity to the amino acid sequence of (SEQ ID NO:2).
3. The targeting construct of embodiment 2, wherein the modified ER-LBD comprises an amino acid sequence having at least 98% sequence identity to the amino acid sequence of SEQ ID NO:2.
4. The targeting construct of embodiment 2, wherein the modified ER-LBD comprises an amino acid sequence having at least 99% sequence identity to the amino acid sequence of SEQ ID NO:2.
5. The targeting construct of embodiment 1 or 108, wherein the modified ER-LBD comprises or consists of an amino acid sequence having 100% sequence identity to the amino acid sequence of SEQ ID NO:2.
6. The targeting construct of embodiment 1 or 108, wherein the additional amino acid substitutions comprise: an L391V substitution, a N413D substitution, a Q414E substitution, a S463P substitution, and a H524F substitution, and wherein the modified ER-LBD comprises an amino acid sequence having at least 80%, at least 85%, at least 90%, or at least 95% sequence identity to the amino acid sequence of SEQ ID NO:3.
7. The targeting construct of embodiment 6, wherein the modified ER-LBD comprises an amino acid sequence having at least 98% sequence identity to the amino acid sequence of SEQ ID NO:3.
8. The targeting construct of embodiment 6, wherein the modified ER-LBD comprises an amino acid sequence having at least 99% sequence identity to the amino acid sequence of SEQ ID NO:3.
9. The targeting construct of embodiment 1 or 108, wherein the modified ER-LBD comprises or consists of an amino acid sequence having 100% sequence identity to the amino acid sequence of SEQ ID NO:3.
10. The targeting construct of embodiment 1 or 108, wherein the additional amino acid substitutions comprise: an L354I substitution, a L391V substitution, a N413D substitution, a Q414E substitution, a M421L substitution, a M517A substitution, and a H524F substitution, and wherein the modified ER-LBD comprises an amino acid sequence having at least 80%, at least 85%, at least 90%, or at least 95% sequence identity to the amino acid sequence of SEQ ID NO:4.
11. The targeting construct of embodiment 10, wherein the modified ER-LBD comprises an amino acid sequence having at least 98% sequence identity to the amino acid sequence of SEQ ID NO:4.
12. The targeting construct of embodiment 10, wherein the modified ER-LBD comprises an amino acid sequence having at least 99% sequence identity to the amino acid sequence of SEQ ID NO:4.
13. The targeting construct of embodiment 1 or 108, wherein the modified ER-LBD comprises or consists of an amino acid sequence having 100% sequence identity to the amino acid sequence of SEQ ID NO:4.
14. The targeting construct of embodiment 1 or 108, wherein the additional amino acid substitutions comprise: an L354I substitution, a L391V substitution, a L409V substitution, a N413D substitution, a Q414E substitution, and a H524L substitution, and wherein the modified ER-LBD comprises an amino acid sequence having at least 80%, at least 85%, at least 90%, or at least 95% sequence identity to the amino acid sequence of SEQ ID NO:5.
15. The targeting construct of embodiment 14, wherein the modified ER-LBD comprises an amino acid sequence having at least 98% sequence identity to the amino acid sequence of SEQ ID NO:5.
16. The targeting construct of embodiment 14, wherein the modified ER-LBD comprises an amino acid sequence having at least 99% sequence identity to the amino acid sequence of SEQ ID NO:5.
17. The targeting construct of embodiment 1 or 108, wherein the modified ER-LBD comprises or consists of an amino acid sequence having 100% sequence identity to the amino acid sequence of SEQ ID NO:5.
18. The targeting construct of embodiment 1 or 108, wherein the additional amino acid substitutions comprise: an L391V substitution, a Q414E substitution, an N413D substitution, an S463P substitution, an M421L substitution, an L354I substitution, an L384M substitution, and an H524L substitution, and the modified ER-LBD comprises an amino acid sequence having at least 80%, at least 85%, at least 90%, or at least 95% sequence identity to the amino acid sequence of SEQ ID NO:40.
19. The targeting construct of embodiment 18, wherein the modified ER-LBD comprises an amino acid sequence having at least 98% sequence identity to the amino acid sequence of SEQ ID NO:40.
20. The targeting construct of embodiment 18, wherein the modified ER-LBD comprises an amino acid sequence having at least 99% sequence identity to the amino acid sequence of SEQ ID NO:40.
21. The targeting construct of embodiment 1 or 108, wherein the modified ER-LBD comprises or consists of an amino acid sequence having 100% sequence identity to the amino acid sequence of SEQ ID NO:40.
22. The targeting construct of embodiment 1 or 108, wherein the additional amino acid substitutions comprise: an L391V substitution, an N413D substitution, an S463P substitution, an M517A substitution, an M421L substitution, an L354I substitution, and an H524L substitution, and the modified ER-LBD comprises an amino acid sequence having at least 80%, at least 85%, at least 90%, or at least 95% sequence identity to the amino acid sequence of SEQ ID NO:41.
23. The targeting construct of embodiment 22, wherein the modified ER-LBD comprises an amino acid sequence having at least 98% sequence identity to the amino acid sequence of SEQ ID NO:41.
24. The targeting construct of embodiment 22, wherein the modified ER-LBD comprises an amino acid sequence having at least 99% sequence identity to the amino acid sequence of SEQ ID NO:41.
25. The targeting construct of embodiment 1 or 108, wherein the modified ER-LBD comprises or consists of an amino acid sequence having 100% sequence identity to the amino acid sequence of SEQ ID NO:41.
26. The targeting construct of embodiment 1 or 108, wherein the additional amino acid substitutions comprise: an N413D substitution, an S463P substitution, an L354I substitution, an L384M substitution, an H524L substitution, and the modified ER-LBD comprises an amino acid sequence having at least 80%, at least 85%, at least 90%, or at least 95% sequence identity to the amino acid sequence of SEQ ID NO:42.
27. The targeting construct of embodiment 26, wherein the modified ER-LBD comprises an amino acid sequence having at least 98% sequence identity to the amino acid sequence of SEQ ID NO:42.
28. The targeting construct of embodiment 26, wherein the modified ER-LBD comprises an amino acid sequence having at least 99% sequence identity to the amino acid sequence of SEQ ID NO:42.
29. The targeting construct of embodiment 1 or 108, wherein the modified ER-LBD comprises or consists of an amino acid sequence having 100% sequence identity to the amino acid sequence of SEQ ID NO:42.
30. The targeting construct of embodiment 1 or 108, wherein the additional amino acid substitutions comprise: an L391V substitution, an N413D substitution, an S463P substitution, an M517A substitution, an M421L substitution, an L354I substitution, and an H524L substitution, and the modified ER-LBD comprises an amino acid sequence having at least 80%, at least 85%, at least 90%, or at least 95% sequence identity to the amino acid sequence of SEQ ID NO:43.
31. The targeting construct of embodiment 30, wherein the modified ER-LBD comprises an amino acid sequence having at least 98% sequence identity to the amino acid sequence of SEQ ID NO:43.
32. The targeting construct of embodiment 30, wherein the modified ER-LBD comprises an amino acid sequence having at least 99% sequence identity to the amino acid sequence of SEQ ID NO:43.
33. The targeting construct of embodiment 1 or 108, wherein the modified ER-LBD comprises or consists of an amino acid sequence having 100% sequence identity to the amino acid sequence of SEQ ID NO:43.
34. The targeting construct of any one of embodiments 1 to 33 or 108, wherein the modified ERT2 peptide further comprises an N-terminal serine (S) residue.
35. The targeting construct of any one of embodiments 1 to 34 or 108, wherein the modified ER-LBD comprises the V595A amino acid substitution.
36. The targeting construct of any one of embodiments 1 to 35 or 108, wherein the caspase 9 domain or derivative or functional fragment thereof does not comprise a Caspase Activation and Recruitment Domain (CARD) domain sequence.
37. The targeting construct of any one of embodiments 1 to 36 or 108, wherein the caspase 9 domain or derivative or functional fragment thereof comprises (SEQ ID NO:6).
38. The targeting construct of any one of embodiments 1 to 37 or 108, wherein the modified estrogen receptor ligand binding domain (ER-LBD) is N-terminal to the caspase 9 domain or derivative or functional fragment thereof.
39. The targeting construct of any one of embodiments 1 to 38 or 108, wherein the modified estrogen receptor ligand binding domain (ER-LBD) is C-terminal to the caspase 9 domain or derivative or functional fragment thereof.
40. The targeting construct of any one of embodiments 1 to 39 or 108, wherein the fusion polypeptide comprises a linker between the modified estrogen receptor ligand binding domain (ER-LBD) and the caspase 9 domain or derivative or functional fragment thereof.
41. The targeting construct of any one of embodiments 1 to 40 or 108, which is configured such that upon its recombination with the target genomic locus, the STEL gene is modified such to incorporate the fusion polypeptide coding sequence 3′ to the STEL protein coding sequence.
42. The targeting construct of any one of embodiments 1 to 40 or 108, which is configured such that upon its recombination with the target genomic locus, the STEL gene is modified such to incorporate the fusion polypeptide coding sequence 5′ to the STEL protein coding sequence.
43. The targeting construct of any one of embodiments 1 to 41 or 108, wherein the nucleotide insert further comprises a nucleotide sequence encoding a separator sequence.
44. The targeting construct of embodiment 43, which is configured such that upon recombination of the targeting construct with the target genomic locus, the separator sequence coding sequence is positioned between the coding sequence of the STEL protein and the fusion polypeptide coding sequence.
45. The targeting construct of embodiment 43 or embodiment 44, wherein the separator sequence is an internal ribosome entry site (“IRES”).
46. The targeting construct of embodiment 43 or embodiment 44, wherein the separator sequence is a self-cleaving peptide.
47. The targeting construct of embodiment 46, wherein the self-cleaving peptide is a 2A peptide.
48. The targeting construct of embodiment 46 or embodiment 47, wherein the self-cleaving peptide is T2A, P2A, E2A, F2A, PQR, Opt2A, or Opt2A_2.0.
49. The targeting construct of any one of embodiments 1 to 48 or 108, wherein the STEL gene encodes a polypeptide involved in one or more of: glycolysis, ribonucleopolypeptide complex formation, focal adhesion, cell-substrate adherens junction, cell-substrate junction, cell anchoring, extracellular exosome, extracellular vesicle, intracellular organelle, anchoring junction, RNA binding, nucleic acid binding (e.g., rRNA or mRNA binding), and polypeptide binding.
50. The targeting construct of any one of embodiments 1 to 49 or 108, wherein the STEL gene encodes a ribosomal polypeptide.
51. The targeting construct of embodiment 50, wherein the STEL gene is RPL13A, RPLP0, RPL10, RPL13, RPSJ8, RPL3, RPLP1, RPL15, RPL41, RPL11, RPL32, RPL18 A, RPL19, RPL28, RPL29, RPL9, RPL8, RPL6, RPL18, RPL7, RPL7A, RPL21, RPL37A, RPL 12, RPL5, RPL34, RPL35A, RPL30, RPL24, RPL39, RPL37, RPL14, RPL27A, RPLP2, RPL23A, RPL26, RPL36, RPL35, RPL23, RPL4, or RPL22.
52. The targeting construct of any one of embodiments 1 to 50 or 108, wherein the STEL gene encodes a ribosomal polypeptide small subunit (RPS).
53. The targeting construct of embodiment 52, wherein the STEL gene is RPS2, RPS19, RPS14, RPS3A, RPS12, RPS3, RPS6, RPS23, RPS27A, RPS8, RPS4X, RPS7, RPS24, RPS27, RPS15A, RPS9, RPS28, RPS13, RPSA, RPS5, RPS 16, RPS25, RPS15, RPS20, or RPS11.
54. The targeting construct of any one of embodiments 1 to 49 or 108, wherein the STEL gene encodes a mitochondrial polypeptide.
55. The targeting construct of embodiment 54, wherein the STEL gene is MT-CO1, MT-C02, MT-ND4, MT-ND1, or MT-ND2.
56. The targeting construct of any one of embodiments 1 to 49 or 108, wherein the STEL gene encodes an actin polypeptide.
57. The targeting construct of embodiment 56, wherein the STEL gene is ACTG1 or ACTB.
58. The targeting construct of any one of embodiments 1 to 49 or 108, wherein the STEL gene encodes a eukaryotic translation factor.
59. The targeting construct of embodiment 58, wherein the STEL gene is EEF1A1, EEF2, or EIF1.
60. The targeting construct of any one of embodiments 1 to 49 or 108, wherein the STEL gene encodes a histone.
61. The targeting construct of embodiment 60, wherein the STEL gene is H3F3A or H3F3B.
62. The targeting construct of any one of embodiments 1 to 49 or 108, wherein the STEL gene is FTL, FTH1, TPT1, IMSB10, GAPDH, PTMA, GNB2L1, NACA, YBX1, NPM1, FAU, UBA52, HSP90AB1, MYL6, SERF2, or SRP14.
63. The targeting construct of embodiment 62, wherein the STEL gene is GAPDH.
64. The targeting construct of embodiment 62, wherein the STEL gene is RPL13A.
65. The targeting construct of embodiment 62, wherein the STEL gene is RPL7.
66. The targeting construct of embodiment 62, wherein the STEL gene is RPLP0.
67. The targeting construct of any one of embodiments 1 to 66 or 108, wherein the nucleotide insert further comprises a transgene.
68. The targeting construct of embodiment 67, wherein the transgene is linked to the fusion polypeptide coding sequence.
69. The targeting construct of embodiment 68, wherein the fusion polypeptide coding sequence and transgene are connected via a nucleotide sequence encoding a separator sequence.
70. The targeting construct of embodiment 69, wherein the separator sequence is an internal ribosome entry site (“IRES”).
71. The targeting construct of embodiment 69, wherein the separator sequence is a nucleotide sequence encoding a self-cleaving peptide (the “self-cleaving peptide coding sequence”).
72. The targeting construct of embodiment 71, wherein the self-cleaving peptide is a 2A peptide.
73. The targeting construct of embodiment 71 or embodiment 72, wherein the self-cleaving peptide is T2A, P2A, E2A, F2A, PQR, Opt2A, or Opt2A_2.0.
74. The targeting construct of any one of embodiments 67 to 73, wherein the transgene encodes a therapeutic polypeptide.
75. A system comprising:
   (a) the targeting construct of any one of embodiments 1 to 74, or 106-108;
   (b) a CRISPR-associated endonuclease (“Cas polypeptide”) or a nucleic acid encoding a Cas polypeptide; and
   (c) a guide RNA (“gRNA”) comprising a scaffold for binding the Cas polypeptide and a spacer sequence corresponding to the STEL gene, or a nucleic acid encoding the gRNA.
76. The system of embodiment 75, wherein the guide RNA is a single guide RNA (“sgRNA”).
77. The system of embodiment 75 or embodiment 76, which comprises the Cas polypeptide and gRNA.
78. The system of any one of embodiments 75 to 77, which is in the form of a ribonucleoprotein particle (“RNP”).
79. A method of producing a gene-edited target cell, comprising:
   (a) introducing the system of any one of embodiments 75 to 78 into a target cell; and
   (b) culturing the target cell under conditions in which gene editing occurs, thereby producing gene-edited target cell.
80. The method of embodiment 79, wherein the target cell is a stem cell or a cell differentiated from a stem cell.
81. The method of embodiment 79 or embodiment 80, wherein the target cell is a stem cell.
82. The method of embodiment 81, wherein the stem cell is a human embryonic stem cell, an induced pluripotent stem cell (“iPSC”) or a cell differentiated therefrom.
83. The method of any one of embodiments 79 to 81, wherein the target cell is:
   (a) a regulatory T cell, a myeloid cell, a dendritic cell, a macrophage (e.g., an immunosuppressive macrophage), a myeloid progenitor cell, or a precursor or progenitor cell thereof;
   (b) a cell in the human nervous system, optionally selected from dopaminergic neuron, a microglial cell, an oligodendrocyte, an astrocyte, a cortical neuron, a spinal or oculomotor neuron, an enteric neuron, a Placode-derived cell, a Schwann cell, and a trigeminal or sensory neuron, or a precursor or progenitor cell thereof;
   (c) a cell in the human cardiovascular system, optionally selected from a cardiomyocyte, an endothelial cell, and a nodal cell, or a precursor or progenitor cell thereof;
   (d) a cell in the human metabolic system, optionally selected from a hepatocyte, a cholangiocyte, and a pancreatic beta cell, or a precursor or progenitor cell thereof, or
   (e) a cell in the human ocular system, optionally selected from a retinal pigment epithelial cell, a photoreceptor cone cell, a photoreceptor rod cell, a bipolar cell, a ganglion cell, or a precursor or progenitor cell thereof.
84. The method of any one of embodiments 79 to 83, wherein the gene-edited target cell is of ectoderm lineage, optionally wherein the gene-edited target cell is a neuron.
85. The method of any one of embodiments 79 to 83, wherein the gene-edited target cell is of mesoderm lineage, optionally wherein the gene-edited target cell is a cardiomyocyte.
86. A gene-edited target cell obtained or obtainable by the method of any one of embodiments 79 to 85.
87. A gene-edited target cell comprising a STEL gene that comprises a nucleic acid encoding a fusion polypeptide under the transcriptional control of a STEL gene regulatory element, the fusion polypeptide comprising:
   (a) a modified estrogen receptor ligand binding domain (ER-LBD); and
   (b) a caspase 9 domain or derivative or functional fragment thereof,
wherein the modified ER-LBD comprises an amino acid sequence corresponding to a hormone binding domain of a reference human estrogen receptor sequence (SEQ ID NO: 1), and wherein the modified ER-LBD comprises
a G400V amino acid substitution, an M543A amino acid substitution, an L544A amino acid substitution, and optionally a V595A amino acid substitution, with reference to SEQ ID NO: 1; and
additional amino acid substitutions, wherein the additional amino acid substitutions comprise, with reference to SEQ ID NO: 1:
(i) an L384M substitution, an L391V substitution, a N413D substitution, an M421L substitution, a S463P substitution, and aH524L substitution,
(ii) an L391V substitution, a N413D substitution, a Q414E substitution, a S463P substitution, and a H524F substitution,
(iii) an L354I substitution, a L391V substitution, a N413D substitution, a Q414E substitution, a M421L substitution, a M517A substitution, and a H524F substitution,
(iv) an L354I substitution, a L39TV substitution, a L409V substitution, a N413D substitution, a Q414E substitution, and a H524L substitution,
(v) an L391V substitution, a Q414E substitution, an N413D substitution, an S463P substitution, an M421L substitution, an L354I substitution, an L384M substitution, and an H524L substitution,
(vi) an L391V substitution, an N413D substitution, an S463P substitution, an M517A substitution, an M421L substitution, an L354I substitution, and an H524L substitution,
(vii) an N413D substitution, an S463P substitution, an L354I substitution, an L384M substitution, an H524L substitution, or
(viii) an L391V substitution, an N413D substitution, an S463P substitution, an M517A substitution, an M421L substitution, an L354I substitution, and an H524L substitution.
88. The gene-edited target cell of embodiment 87, wherein the modified ER-LBD is as defined in any one of embodiments 2 to 35.
89. The gene-edited target cell of embodiment 87 or embodiment 88, wherein the caspase 9 domain or derivative or functional fragment thereof is as defined in any one of embodiments 36 to 37.
90. The gene-edited target cell of any one of embodiments 87 to 89, wherein the fusion polypeptide is as defined in any one of embodiments 38 to 40.
91. The gene-edited target cell of any one of embodiments 87 to 90, wherein the STEL gene is configured as defined in any one of embodiments 41 to 66.
92. The gene-edited target cell of any one of embodiments 87 to 91, which further comprises a transgene in the STEL gene.
93. The gene-edited target cell of embodiment 92, wherein the transgene is as defined in any one of embodiments 67 to 74.
94. The gene-edited target cell of any one of embodiments 87 to 93, wherein the target cell is as defined in any one of embodiments 80 to 86.
95. A pharmaceutical composition comprising the gene-edited target cell of any one of embodiments 86 to 94 and a pharmaceutically acceptable carrier.
96. A method of treating a patient in need thereof, comprising administering to the patient the gene-edited target cell of any one of embodiments 86 to 94 or a pharmaceutical composition the gene-edited target cell of any one of embodiments 86 to 94 and a pharmaceutically acceptable carrier.
97. The method of embodiment 96, which further comprises controlling the gene-edited target cell population in the patient by:
   (a) Monitoring, optionally, the gene-edited target cell population in the patient; and/or
   (b) administering an inducer of the modified ER-LBD if the patient experiences adverse events related to the gene-edited target cell population.
98. A method of mitigating adverse events or a safety risk associated with cell therapy in the form of the gene-edited target cells any one of embodiments 86 to 94 or the pharmaceutical composition of embodiment 95, the method comprising administering to a patient who received the gene-edited target cells or pharmaceutical composition an inducer of a modified ER-LBD if the patient experiences adverse events or a safety risk related to the gene-edited target cells or pharmaceutical composition.
99. The method of embodiment 97 or embodiment 98, wherein the inducer of the modified ER-LBD is tamoxifen or a tamoxifen metabolite.
100. The method of embodiment 99, wherein the inducer of the modified ER-LBD is tamoxifen.
101. The method of embodiment 99, wherein the inducer of the modified ER-LBD is a tamoxifen metabolite.
102. The method of embodiment 101, wherein the inducer of the modified ER-LBD is 4-hydroxytamoxifen, N-desmethyltamoxifen, tamoxifen-N-oxide, or endoxifen.
103. The method of any one of embodiments 81 to 85, wherein a combination of two or more inducers of the modified ER-LBD is administered.
104. The method of embodiment 103, wherein the combination comprises 4-OHT and endoxifen.
105. The method of embodiment 103 or embodiment 104, wherein the combination has a synergistic effect and/or utilizes reduced dosing (e.g., reduced dosing amount and/or frequency) than would be required a single inducer of the modified ER-LBD.
106. The targeting construct of any one of embodiments 1-74 or 108, wherein the targeting construct comprises an amino acid sequence at least 80%, at least 85%, at least 90%, at least 91%, at least 92%, at least 93%, at least 94%, at least 95%, at least 96%, at least 97%, at least 98%, at least 99%, at least 99.5%, or at least 99.9% identical to an amino acid sequence set forth in SEQ ID NO: 13, 15, 17, 19, 21, 23, 25, 27, 44, 46, 48, or 50.
107. The targeting construct of embodiment 107, wherein the targeting construct comprises an amino acid sequence set forth in SEQ ID NO: 13, 15, 17, 19, 21, 23, 25, 27, 44, 46, 48, or 50.
108. A targeting construct comprising:
   (a) a first homology arm corresponding to a 5′ target sequence comprising a first region of homology to a target genomic locus;
   (b) a nucleotide insert comprising a nucleotide sequence encoding a fusion polypeptide comprising:
      (i) a first modified estrogen receptor ligand binding domain (ER-LBD);
      (ii) a first caspase 9 domain or derivative or functional fragment thereof;
      (iii) a second modified estrogen receptor ligand binding domain (ER-LBD); and
      (iv) a second caspase 9 domain or derivative or functional fragment thereof;
   (c) a second homology arm corresponding to a 3′ target sequence comprising second region of homology to the target genomic locus,
wherein the first modified ER-LBD and the second modified ER-LBD each comprise an amino acid sequence corresponding to a hormone binding domain of a reference human estrogen receptor sequence (SEQ ID NO: 1), and wherein the first modified ER-LBD and the second modified ER-LBD each independently comprise:
   a G400V amino acid substitution, an M543A amino acid substitution, an L544A amino acid substitution, and optionally a V595A amino acid substitution, with reference to SEQ ID NO: 1; and
   additional amino acid substitutions, wherein the additional amino acid substitutions comprise, with reference to SEQ ID NO: 1:
   (i) an L384M substitution, an L391V substitution, a N413D substitution, an M421L substitution, a S463P substitution, and a H524L substitution,
   (ii) an L391V substitution, a N413D substitution, a Q414E substitution, a S463P substitution, and a H524F substitution,
   (iii) an L354I substitution, a L391V substitution, a N413D substitution, a Q414E substitution, a M421L substitution, a M517A substitution, and a H524F substitution,
   (iv) an L354I substitution, a L391V substitution, a L409V substitution, a N413D substitution, a Q414E substitution, and a H524L substitution,
   (v) an L391V substitution, a Q414E substitution, an N413D substitution, an S463P substitution, an M421L substitution, an L354I substitution, an L384M substitution, and an H524L substitution,
   (vi) an L391V substitution, an N413D substitution, an S463P substitution, an M517A substitution, an M421L substitution, an L354I substitution, and an H524L substitution,
   (vii) an N413D substitution, an S463P substitution, an L354I substitution, an L384M substitution, an H524L substitution, or
   (viii) an L391V substitution, an N413D substitution, an S463P substitution, an M517A substitution, an M421L substitution, an L354I substitution, and an H524L substitution,
wherein the targeting construct is configured such that upon its recombination with the target genomic locus, the fusion polypeptide coding sequence is integrated into a STEL gene and becomes operably linked to the STEL gene regulatory element.

### EXAMPLES

### Example 1

### Design and Generation of Modified ERT2 Peptide-Suicide Protein Constructs

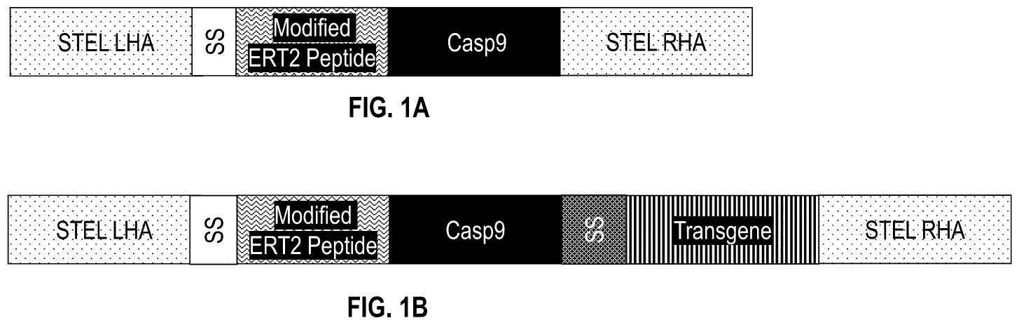

Kill switch constructs of the disclosure were designed to trigger apoptosis in response to tamoxifen and/or its metabolites, which are small drugs (e.g., that may already be in routine clinical use) and may be able to penetrate through the blood-brain barrier. However, implementation of such kill switches in pluripotent stem cell (PSC)-derived cell products can be difficult because expression of transgenes can be lost after differentiating a PSC to another cell type (e.g., an immune cell such as a T cell or macrophage, a CNS cell such as a neuron, microglia, macroglia, or precursor thereof, or a cardiovascular cell). Thus, to implement such kill switches in pluripotent stem cell (PSC)-derived cell products, constructs were designed such that a modified ERT2 peptide-suicide protein fusion polypeptide sequence was flanked by homology arms complementary to genomic sequences of a sustained transcription expression locus (STEL) (FIG. 1A). See WO2021072329A1, which is incorporated herein by reference in its entirety.

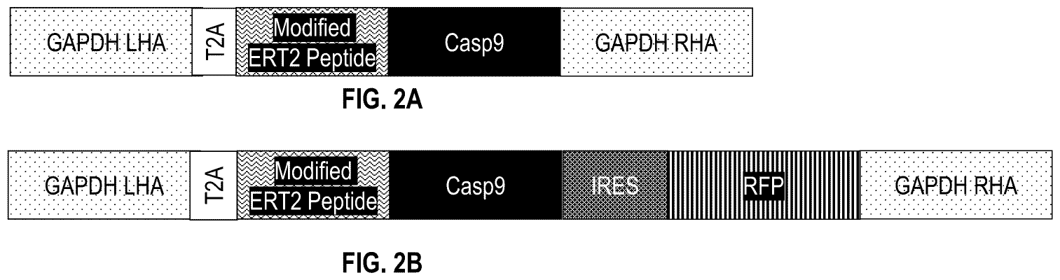

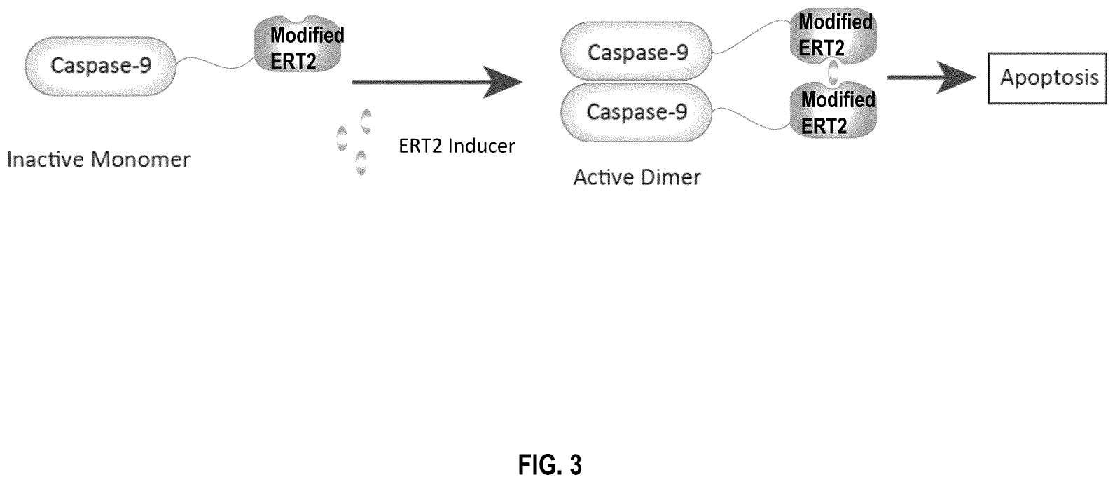

Four modified ERT2 peptides (ERT2 mutants) were selected to be fused to Caspase-9 (Table 3). All four engineered ERT2-Casp9 candidates showed greater than 95% killing efficiency after the addition of 1 μM 4-OHT, a tamoxifen metabolite in HEK293T cells within 48 hours of induction (data not shown). The resulting four kill-switch fusion polypeptide encoding sequences were inserted between the left and right homology arms targeting the GAPDH STEL with a T2A peptide sequence linking a modified ERT2 peptide-suicide protein fusion polypeptide encoding sequence to the left homology arm. (FIG. 2A).

TABLE 3

| | modified ERT2 | |
|---|---|---|
| | peptide-suicide | |
| | protein fusion | |
| Plasmid No. | polypeptide | Sequence |
| Construct 1 | ERT2*mut81-Casp9 | AGDMRAANLWPSPLMIKRSKKNSLALSLTADQMVSALLDAEPPIL |
| | | YSEYDPTRPFSEASMMGLLTNLADRELVHMINWAKRVPGFVDLTL |
| | | HDQVHLLECAWMEILMIGVVWRSMEHPVKLLFAPNLLLDRDQGKC |
| | | VEGLVEIFDMLLATSSRFRMMNLQGEEFVCLKSIILLNSGVYTFL |
| | | PSTLKSLEEKDHIHRVLDKITDTLIHLMAKAGLTLQQQHQRLAQL |
| | | LLILSHIRHMSNKGMELLYSMKCKNVVPLYDLLLEAADAHRLHAP |
| | | TSRGGASVEETDQSHLATAGSTSSHSLQKYYITGEAEGFPATGGG |
| | | GSGGGGSGGGGSVDGFMDVGALESLRGNADLAYILSMEPCGHCLI |
| | | INNVNFCRESGLRTRTGSNIDCEKLRRRFSSLHFMVEVKGDLTAK |
| | | KMVLALLELARQDHGALDCCVVVILSHGCQASHLQFPGAVYGTDG |
| | | CPVSVEKIVNIFNGTSCPSLGGKPKLFFIQACGGEQKDHGFEVAS |
| | | TSPEDESPGSNPEPDATPFQEGLRTFDQLDAISSLPTPSDIFVSY |
| | | STFPGFVSWRDPKSGSWYVETLDDIFEQWAHSEDLQSLLLRVANA |
| | | VSVKGIYKQMPGCFNFLRKKLFFKTS |
| | | (SEQ ID NO: 44) |
| | | Bold = ERT2*mut81 |
| | | Italics = linker |
| | | Plain = Casp9 |
| | | Nucleotide sequence: ATGGCTGGAGACATGAGAGCTGCCA |
| | | ACCTTTGGCCAAGCCCGCTCATGATCAAACGCTCTAAGAAGAACA |
| | | GCCTGGCCTTGTCCCTGACGGCCGACCAGATGGTCAGTGCCTTGT |
| | | TGGATGCTGAGCCCCCCATACTCTATTCCGAGTATGATCCTACCA |
| | | GACCCTTCAGTGAAGCTTCGATGATGGGCTTACTGACCAACCTGG |
| | | CAGACAGGGAGCTGGTTCACATGATCAACTGGGCGAAGAGGGTGC |
| | | CAGGCTTTGTGGATTTGACCCTCCATGATCAGGTCCACCTTCTAG |
| | | AATGTGCCTGGATGGAGATCCTGATGATTGGTGTGGTCTGGCGCT |
| | | CCATGGAGCACCCAGTGAAGCTACTGTTTGCTCCTAACTTGCTCT |
| | | TGGACAGGGACCAGGGAAAATGTGTAGAGGGCCTGGTGGAGATCT |
| | | TCGACATGCTGCTGGCTACATCATCTCGGTTCCGCATGATGAATC |
| | | TGCAGGGAGAGGAGTTTGTGTGCCTCAAATCTATTATTTTGCTTA |
| | | ATTCTGGAGTGTACACATTTCTGCCCAGCACCCTGAAGTCTCTGG |
| | | AAGAGAAGGACCATATCCACCGAGTCCTGGACAAGATCACAGACA |
| | | CTTTGATCCACCTGATGGCCAAGGCAGGCCTGACCCTGCAGCAGC |
| | | AGCACCAGCGGCTGGCCCAGCTCCTCCTCATCCTCTCCCACATCA |
| | | GGCACATGAGTAACAAAGGCATGGAGCTGCTGTACAGCATGAAGT |
| | | GCAAGAACGTGGTGCCCCTCTATGACCTGCTGCTGGAGGCGGCGG |
| | | ACGCCCACCGCCTACATGCGCCCACTAGCCGTGGAGGGGCATCCG |
| | | TGGAGGAGACGGACCAAAGCCACTTGGCCACTGCGGGCTCTACTT |
| | | CATCGCATTCCTTGCAAAAGTATTACATCACGGGGGAGGCAGAGG |
| | | GTTTCCCTGCCACAGGGGGTGGAGGTTCAGGGGGTGGAGGTTCAG |
| | | GTGGTGGCGGTAGTGTCGATGGCTTCGATGTCGGTGCTCTTGAGA |
| | | GTTTGAGGGGAAATGCAGATTTGGCTTACATCCTGAGCATGGAGC |
| | | CCTGTGGCCACTGCCTCATTATCAACAATGTGAACTTCTGCCGTG |
| | | AGTCCGGGCTCCGCACCCGCACTGGCTCCAACATCGACTGTGAGA |
| | | AGTTGCGGCGTCGCTTCTCCTCGCTGCATTTCATGGTGGAGGTGA |
| | | AGGGCGACCTGACTGCCAAGAAAATGGTGCTGGCTTTGCTGGAGC |
| | | TGGCGCGGCAGGACCACGGTGCTCTGGACTGCTGCGTGGTGGTCA |
| | | TTCTCTCTCACGGCTGTCAGGCCAGCCACCTGCAGTTCCCAGGGG |
| | | CTGTCTACGGCACAGATGGATGCCCTGTGTCGGTCGAGAAGATTG |
| | | TGAACATCTTCAATGGGACCAGCTGCCCCAGCCTGGGAGGGAAGC |
| | | CCAAGCTCTTTTTCATCCAGGCCTGTGGTGGGGAGCAGAAAGACC |
| | | ATGGGTTTGAGGTGGCCTCCACTTCCCCTGAAGACGAGTCCCCTG |
| | | GCAGTAACCCCGAGCCAGATGCCACCCCGTTCCAGGAAGGTTTGA |
| | | GGACCTTCGACCAGCTGGACGCCATATCTAGTTTGCCCACACCCA |
| | | GTGACATCTTTGTGTCCTACTCTACTTTCCCAGGTTTTGTTTCCT |
| | | GGAGGGACCCCAAGAGTGGCTCCTGGTACGTTGAGACCCTGGACG |
| | | ACATCTTTGAGCAGTGGGCTCACTCTGAAGACCTGCAGTCCCTCC |
| | | TGCTTAGGGTCGCTAATGCTGTTTCGGTGAAAGGGATTTATAAAC |
| | | AGATGCCTGGTTGCTTTAATTTCCTCCGGAAAAAACTTTTCTTTA |
| | | AAACATCA |
| | | (SEQ ID NO: 45) |
| 2 | ERT2*mut88-Casp9 | AGDMRAANLWPSPLMIKRSKKNSLALSLTADQMVSALLDAEPPIL |
| | | YSEYDPTRPFSEASMMGLLTNLADRELVHMINWAKRVPGFVDLTL |
| | | HDQVHLLECAWLEILMIGVVWRSMEHPVKLLFAPNLLLDRDEGKC |
| | | VEGMVEIFDMLLATSSRFRMMNLQGEEFVCLKSIILLNSGVYTFL |
| | | PSTLKSLEEKDHIHRVLDKITDTLIHLMAKAGLTLQQQHQRLAQL |
| | | LLILSHIRHMSNKGMEFLYSMKCKNVVPLYDLLLEAADAHRLHAP |
| | | TSRGGASVEETDQSHLATAGSTSSHSLQKYYITGEAEGFPATGGG |
| | | GSGGGGSGGGGSVDGFMDVGALESLRGNADLAYILSMEPCGHCLI |
| | | INNVNFCRESGLRTRTGSNIDCEKLRRRFSSLHFMVEVKGDLTAK |
| | | KMVLALLELARQDHGALDCCVVVILSHGCQASHLQFPGAVYGTDG |
| | | CPVSVEKIVNIFNGTSCPSLGGKPKLFFIQACGGEQKDHGFEVAS |
| | | TSPEDESPGSNPEPDATPFQEGLRTFDQLDAISSLPTPSDIFVSY |
| | | STFPGFVSWRDPKSGSWYVETLDDIFEQWAHSEDLQSLLLRVANA |
| | | VSVKGIYKQMPGCFNFLRKKLFFKTS |
| | | (SEQ ID NO: 46) |
| | | Bold = ERT2*mut88 |
| | | Italics = linker |
| | | Plain = Casp9 |
| | | Nucleotide sequence: |
| | | ATGgctggagacatgagagctgccaacctttggccaagcccgctc |
| | | atgatcaaacgctctaagaagaacagcctggccttgtccctgacg |
| | | gccgaccagatggtcagtgccttgttggatgctgagccccccata |
| | | ctctattccgagtatgatcctaccagacccttcagtgaagcttcg |
| | | atgatgggcttactgaccaacctggcagacagggagCTGGTTCAC |
| | | ATGATCAACTGGGCGAAGAGGGTGCCAGGCTTTGTGGATTTGACC |
| | | CTCCATGATCAGGTCCACCTTCTAGAATGTGCCTGGCTAGAGATC |
| | | CTGATGATTGGTGTGGTCTGGCGCTCCATGGAGCACCCAGTGAAG |
| | | CTACTGTTTGCTCCTAACTTGCTCTTGGACAGGGACGAGGGAAAA |
| | | TGTGTAGAGGGCATGGTGGAGATCTTCGACATGCTGCTGGCTACA |
| | | TCATCTCGGTTCCGCATGATGAATCTGCAGGGAGAGGAGTTTGTG |
| | | TGCCTCAAATCTATTATTTTGCTTAATTCTGGAGTGTACACATTT |
| | | CTGCCCAGCACCCTGAAGTCTCTGGAAGAGAAGGACCATATCCAC |
| | | CGAGTCCTGGACAAGATCACAGACACTTTGATCCACCTGATGGCC |
| | | AAGGCAGGCCTGACCCTGCAGCAGCAGCACCAGCGGCTGGCCCAG |
| | | CTCCTCCTCATCCTCTCCCACATCAGGCACATGAGTAACAAAGGC |
| | | ATGGAGTTTctgtacagcatgaagtgcaagaacgtggtgcccctc |
| | | tatGaCctgctgctggaggcggcggacgcccaccgcctacatgcg |
| | | cccactagccgtggaggggcatccgtggaggagacggaccaaagc |
| | | cacttggccactgcgggctctacttcatcgcattccttgcaaaag |
| | | tattacatcacgggggaggcagagggtttccctgccacagggggt |
| | | ggaggttcagggggtggaggttcaggtggtggcggtagtgtcgat |
| | | ggcttcgatgtcggtgctcttgagagtttgaggggaaatgcagat |
| | | ttggcttacatcctgagcatggagccctgtggccactgcctcatt |
| | | atcaacaatgtgaacttctgccgtgagtccgggctccgcacccgc |
| | | actggctccaacatcgactgtgagaagttgcggcgtcgcttctcc |
| | | tcgctgcatttcatggtggaggtgaagggcgacctgactgccaag |
| | | aaaatggtgctggctttgctggagctggcgcggcaggaccacggt |
| | | gctctggactgctgcgtggtggtcattctctctcacggctgtcag |
| | | gccagccacctgcagttcccaggggctgtctacggcacagatgga |
| | | tgccctgtgtcggtcgagaagattgtgaacatcttcaatgggacc |
| | | agctgccccagcctgggagggaagcccaagctctttttcatccag |
| | | gcctgtggtggggagcagaaagaccatgggtttgaggtggcctcc |
| | | acttcccctgaagacgagtcccctggcagtaaccccgagccagat |
| | | gccaccccgttccaggaaggtttgaggaccttcgaccagctggac |
| | | gccatatctagtttgcccacacccagtgacatctttgtgtcctac |
| | | tctactttcccaggttttgtttcctggagggaccccaagagtggc |
| | | tcctggtacgttgagaccctggacgacatctttgagcagtgggct |
| | | cactctgaagacctgcagtccctcctgcttagggtcgctaatgct |
| | | gtttcggtgaaagggatttataaacagatgcctggttgctttaat |
| | | ttcctccggaaaaaacttttctttaaaacatca |
| | | (SEQ ID NO: 47) |
| 3 | ERT2*mut63-Casp9 | AGDMRAANLWPSPLMIKRSKKNSLALSLTADQMVSALLDAEPPIL |
| | | YSEYDPTRPFSEASMMGLLTNLADREIVHMINWAKRVPGFVDLTL |
| | | HDQVHLLECAWLEILMIGVVWRSMEHPVKLLFAPNLLLDRDEGKC |
| | | VEGLVEIFDMLLATSSRFRMMNLQGEEFVCLKSIILLNSGVYTFL |
| | | SSTLKSLEEKDHIHRVLDKITDTLIHLMAKAGLTLQQQHQRLAQL |
| | | LLILSHIRHASNKGMEFLYSMKCKNVVPLYDLLLEAADAHRLHAP |
| | | TSRGGASVEETDQSHLATAGSTSSHSLQKYYITGEAEGFPATGGG |
| | | GSGGGGSGGGGSVDGFMDVGALESLRGNADLAYILSMEPCGHCLI |
| | | INNVNFCRESGLRTRTGSNIDCEKLRRRFSSLHFMVEVKGDLTAK |
| | | KMVLALLELARQDHGALDCCVVVILSHGCQASHLQFPGAVYGTDG |
| | | CPVSVEKIVNIFNGTSCPSLGGKPKLFFIQACGGEQKDHGFEVAS |
| | | TSPEDESPGSNPEPDATPFQEGLRTFDQLDAISSLPTPSDIFVSY |
| | | STFPGFVSWRDPKSGSWYVETLDDIFEQWAHSEDLQSLLLRVANA |
| | | VSVKGIYKQMPGCFNFLRKKLFFKTS |
| | | (SEQ ID NO: 48) |
| | | Bold = ERT2*mut63 |
| | | Italics = linker |
| | | Plain = Casp9 |
| | | Nucleotide sequence: |
| | | ATGgctggagacatgagagctgccaacctttggccaagcccgctc |
| | | atgatcaaacgctctaagaagaacagcctggccttgtccctgacg |
| | | gccgaccagatggtcagtgccttgttggatgctgagccccccata |
| | | ctctattccgagtatgatcctaccagacccttcagtgaagcttcg |
| | | atgatgggcttactgaccaacctggcagacagggagATCGTTCAC |
| | | ATGATCAACTGGGCGAAGAGGGTGCCAGGCTTTGTGGATTTGACC |
| | | CTCCATGATCAGGTCCACCTTCTAGAATGTGCCTGGCTAGAGATC |
| | | CTGATGATTGGTGTGGTCTGGCGCTCCATGGAGCACCCAGTGAAG |
| | | CTACTGTTTGCTCCTAACTTGCTCTTGGACAGGGACGAGGGAAAA |
| | | TGTGTAGAGGGCCTGGTGGAGATCTTCGACATGCTGCTGGCTACA |
| | | TCATCTCGGTTCCGCATGATGAATCTGCAGGGAGAGGAGTTTGTG |
| | | TGCCTCAAATCTATTATTTTGCTTAATTCTGGAGTGTACACATTT |
| | | CTGTCCAGCACCCTGAAGTCTCTGGAAGAGAAGGACCATATCCAC |
| | | CGAGTCCTGGACAAGATCACAGACACTTTGATCCACCTGATGGCC |
| | | AAGGCAGGCCTGACCCTGCAGCAGCAGCACCAGCGGCTGGCCCAG |
| | | CTCCTCCTCATCCTCTCCCACATCAGGCACGCCAGTAACAAAGGC |
| | | ATGGAGTTTctgtacagcatgaagtgcaagaacgtggtgcccctc |
| | | tatGaCctgctgctggaggcggcggacgcccaccgcctacatgcg |
| | | cccactagccgtggaggggcatccgtggaggagacggaccaaagc |
| | | cacttggccactgcgggctctacttcatcgcattccttgcaaaag |
| | | tattacatcacgggggaggcagagggtttccctgccacagggggt |
| | | ggaggttcagggggtggaggttcaggtggtggcggtagtgtcgat |
| | | ggcttcgatgtcggtgctcttgagagtttgaggggaaatgcagat |
| | | ttggcttacatcctgagcatggagccctgtggccactgcctcatt |
| | | atcaacaatgtgaacttctgccgtgagtccgggctccgcacccgc |
| | | actggctccaacatcgactgtgagaagttgcggcgtcgcttctcc |
| | | tcgctgcatttcatggtggaggtgaagggcgacctgactgccaag |
| | | aaaatggtgctggctttgctggagctggcgcggcaggaccacggt |
| | | gctctggactgctgcgtggtggtcattctctctcacggctgtcag |
| | | gccagccacctgcagttcccaggggctgtctacggcacagatgga |
| | | tgccctgtgtcggtcgagaagattgtgaacatcttcaatgggacc |
| | | agctgccccagcctgggagggaagcccaagctctttttcatccag |
| | | gcctgtggggggagcagaaagaccatgggtttgaggtggcctcca |
| | | cttcccctgaagacgagtcccctggcagtaaccccgagccagatg |
| | | ccaccccgttccaggaaggtttgaggaccttcgaccagctggacg |
| | | ccatatctagtttgcccacacccagtgacatctttgtgtcctact |
| | | ctactttcccaggttttgtttcctggagggaccccaagagtggct |
| | | cctggtacgttgagaccctggacgacatctttgagcagtgggctc |
| | | actctgaagacctgcagtccctcctgcttagggtcgctaatgctg |
| | | tttcggtgaaagggatttataaacagatgcctggttgctttaatt |
| | | tcctccggaaaaaacttttctttaaaacatca |
| | | (SEQ ID NO: 49) |
| 4 | ERT2*mut41-Casp9 | AGDMRAANLWPSPLMIKRSKKNSLALSLTADQMVSALLDAEPPIL |
| | | YSEYDPTRPFSEASMMGLLTNLADREIVHMINWAKRVPGFVDLTL |
| | | HDQVHLLECAWLEILMIGVVWRSMEHPVKLLFAPNLVLDRDEGKC |
| | | VEGMVEIFDMLLATSSRFRMMNLQGEEFVCLKSIILLNSGVYTFL |
| | | SSTLKSLEEKDHIHRVLDKITDTLIHLMAKAGLTLQQQHQRLAQL |
| | | LLILSHIRHMSNKGMELLYSMKCKNVVPLYDLLLEAADAHRLHAP |
| | | TSRGGASVEETDQSHLATAGSTSSHSLQKYYITGEAEGFPATGGG |
| | | GSGGGGSGGGGSVDGFMDVGALESLRGNADLAYILSMEPCGHCLI |
| | | INNVNFCRESGLRTRTGSNIDCEKLRRRFSSLHFMVEVKGDLTAK |
| | | KMVLALLELARQDHGALDCCVVVILSHGCQASHLQFPGAVYGTDG |
| | | CPVSVEKIVNIFNGTSCPSLGGKPKLFFIQACGGEQKDHGFEVAS |
| | | TSPEDESPGSNPEPDATPFQEGLRTFDQLDAISSLPTPSDIFVSY |
| | | STFPGFVSWRDPKSGSWYVETLDDIFEQWAHSEDLQSLLLRVANA |
| | | VSVKGIYKQMPGCFNFLRKKLFFKTS |
| | | (SEQ ID NO: 50) |
| | | Bold = ERT2*mut41 |
| | | Italics = linker |
| | | Plain = Casp9 |
| | | Nucleotide sequence: |
| | | ATGgctggagacatgagagctgccaacctttggccaagcccgctc |
| | | atgatcaaacgctctaagaagaacagcctggccttgtccctgacg |
| | | gccgaccagatggtcagtgccttgttggatgctgagccccccata |
| | | ctctattccgagtatgatcctaccagacccttcagtgaagcttcg |
| | | atgatgggcttactgaccaacctggcagacagggagATCGTTCAC |
| | | ATGATCAACTGGGCGAAGAGGGTGCCAGGCTTTGTGGATTTGACC |
| | | CTCCATGATCAGGTCCACCTTCTAGAATGTGCCTGGCTAGAGATC |
| | | CTGATGATTGGTGTGGTCTGGCGCTCCATGGAGCACCCAGTGAAG |
| | | CTACTGTTTGCTCCTAACTTGGTGTTGGACAGGGACGAGGGAAAA |
| | | TGTGTAGAGGGCATGGTGGAGATCTTCGACATGCTGCTGGCTACA |
| | | TCATCTCGGTTCCGCATGATGAATCTGCAGGGAGAGGAGTTTGTG |
| | | TGCCTCAAATCTATTATTTTGCTTAATTCTGGAGTGTACACATTT |
| | | CTGTCCAGCACCCTGAAGTCTCTGGAAGAGAAGGACCATATCCAC |
| | | CGAGTCCTGGACAAGATCACAGACACTTTGATCCACCTGATGGCC |
| | | AAGGCAGGCCTGACCCTGCAGCAGCAGCACCAGCGGCTGGCCCAG |
| | | CTCCTCCTCATCCTCTCCCACATCAGGCACATGAGTAACAAAGGC |
| | | ATGGAGCTGctgtacagcatgaagtgcaagaacgtggtgcccctc |
| | | tatGaCctgctgctggaggcggcggacgcccaccgcctacatgcg |
| | | cccactagccgtggaggggcatccgtggaggagacggaccaaagc |
| | | cacttggccactgcgggctctacttcatcgcattccttgcaaaag |
| | | tattacatcacgggggaggcagagggtttccctgccacagggggt |
| | | ggaggttcagggggtggaggttcaggtggtggcggtagtgtcgat |
| | | ggcttcgatgtcggtgctcttgagagtttgaggggaaatgcagat |
| | | ttggcttacatcctgagcatggagccctgtggccactgcctcatt |
| | | atcaacaatgtgaacttctgccgtgagtccgggctccgcacccgc |
| | | actggctccaacatcgactgtgagaagttgcggcgtcgcttctcc |
| | | tcgctgcatttcatggtggaggtgaagggcgacctgactgccaag |
| | | aaaatggtgctggctttgctggagctggcgcggcaggaccacggt |
| | | gctctggactgctgcgtggtggtcattctctctcacggctgtcag |
| | | gccagccacctgcagttcccaggggctgtctacggcacagatgga |
| | | tgccctgtgtcggtcgagaagattgtgaacatcttcaatgggacc |
| | | agctgccccagcctgggagggaagcccaagctctttttcatccag |
| | | gcctgtggggggagcagaaagaccatgggtttgaggtggcctcca |
| | | cttcccctgaagacgagtcccctggcagtaaccccgagccagatg |
| | | ccaccccgttccaggaaggtttgaggaccttcgaccagctggacg |
| | | ccatatctagtttgcccacacccagtgacatctttgtgtcctact |
| | | ctactttcccaggttttgtttcctggagggaccccaagagtggct |
| | | cctggtacgttgagaccctggacgacatctttgagcagtgggctc |
| | | actctgaagacctgcagtccctcctgcttagggtcgctaatgctg |
| | | tttcggtgaaagggatttataaacagatgcctggttgctttaatt |
| | | tcctccggaaaaaacttttctttaaaacatca |
| | | (SEQ ID NO: 51) |

Another set of constructs were designed to include a transgene sequence after a separator sequence downstream to the modified ERT2 peptide-suicide protein fusion polypeptide (FIG. 1B), wherein the transgene might be a reporter gene or a therapeutic gene. Hence, four more constructs were generated using the modified ERT2 peptide-suicide protein fusion polypeptides presented in Table 3 by the insertion of an IRES sequence and an RFP sequence between the kill switch polypeptide and the right homology arm targeting the GAPDH locus (FIG. 2B).

The sequences of the left and right homology arms for targeting the GAPDH locus are shown below as SEQ ID NOs:7 and 8, respectively.

(SEQ ID NO: 7)
TTGGTATCGTGGAAGGACTCATGGTATGAGAGCTGGGGAATGGGA
CTGAGGCTCCCACCTTTCTCATCCAAGACTGGCTCCTCCCTGCCG
GGGCTGCGTGCAACCCTGGGGTTGGGGGTTCTGGGGACTGGCTTT
CCCATAATTTCCTTTCAAGGTGGGGAGGGAGGTAGAGGGGTGATG
TGGGGAGTACGCTGCAGGGCCTCACTCCTTTTGCAGACCACAGTC
CATGCCATCACTGCCACCCAGAAGACTGTGGATGGCCCCTCCGGG
AAACTGTGGCGTGATGGCCGCGGGGCTCTCCAGAACATCATCCCT
GCCTCTACTGGCGCTGCCAAGGCTGTGGGCAAGGTCATCCCTGAG
CTGAACGGGAAGCTCACTGGCATGGCCTTCCGTGTCCCCACTGCC
AACGTGTCAGTGGTGGACCTGACCTGCCGTCTAGAAAAACCTGCC
AAATATGATGACATCAAGAAGGTGGTGAAGCAGGCGTCGGAGGGC
CCCCTCAAGGGCATCCTGGGCTACACTGAGCACCAGGTGGTCTCC
TCTGACTTCAACAGCGACACCCACTCCTCCACCTTTGACGCTGGG
GCTGGCATTGCCCTCAACGACCACTTTGTCAAGCTCATTTCCTGG
TATGTGGCTGGGGCCAGAGACTGGCTCTTAAAAAGTGCAGGGTCT
GGCGCCCTCTGGTGGCTGGCTCAGAAAAAGGGCCCTGACAACTCT
TTACATCTTCTAGGTATGACAACGAATTTGGCTACAGCAACAGGG
TGGTGGACCTCATGGCCCACATGGCCTCCAAGGAG
(SEQ ID NO: 8)
GACCCCTGGACCACCAGCCAAAGCAAGAGCACAAGAGGAAGAGAG
AGACCCTCACTGCTGGGGAGTCCCTGCCACACTCAGTCCCCCACC
ACACTGAATCTCCCCTCCTCACAGTTGCCATGTAGACCCCTTGAA
GAGGGGAGGGGCCTAGGGAGCCGCACCTTGTCATGTACCATCAAT
AAAGTACCCTGTGCTCAACCAGTTACTTGTCCTGTCTTATTCTAG
GGTCTGGGGCAGAGGGGAGGGAAGCTGGGCTTGTGTCAAGGTGAG
ACATTCTTGCTGGGGAGGGACCTGGTATGTTCTCCTCAGACTGAG
GGTAGGGCCTCCAAACAGCCTTGCTTGCTTCGAGAACCATTTGCT
TCCCGCTCAGACGTCTTGAGTGCTACAGGAAGCTGGCACCACTAC
TTCAGAGAACAAGGCCTTTTCCTCTCCTCGCTCCAGTCCTAGGCT
ATCTGCTGTTGGCCAAACATGGAAGAAGCTATTCTGTGGGCAGCC
CCAGGGAGGCTGACAGGTGGAGGAAGTCAGGGCTCGCACTGGGCT
CTGACGCTGACTGGTTAGTGGAGCTCAGCCTGGAGCTGAGCTGCA
GCGGGCAATTCCAGCTTGGCCTCCGCAGCTGTGAGGTCTTGAGCA
CGTGCTCTATTGCTTTCTGTGCCCTCGTGTCTTATCTGAGGACAT
CGTGGCCAGCCCCTAAGGTCTTCAAGCAGGATTCATCTAGGTAAA
CCAAGTACCTAAAACCATGCCCAAGGCGGTAAGGACTATATAATG
TTTAAAAATCGGTAAAAATGCCCACCTCGCATAGT

The Sequences 7 and 8 are positioned 3′ or 5′ of GAPDH RNP flanking or cut sites TTTCATCTTCTAGGTATGACAACGATTACA (SEQ ID NO: 58) and TGTAATCGTTGTCATACCTAGAAGATGAAA (SEQ ID NO: 59), respectively, which allow the nucleic acid to be cut by the endonuclease at the sequence flanking the LHA towards its 5′ end and the sequence flanking the RHA towards its 3′ end. That is, SEQ ID NO: 7 is positioned 3′ of the 5′ GAPDH RNP flanking or cut site TTTCATCTTCTAGGTATGACAACGATTACA (SEQ ID NO: 58), and SEQ ID NO: 8 is positioned 5′ of the 3′ GAPDH RNP flanking or cut site TGTAATCGTTGTCATACCTAGAAGATGAAA (SEQ ID NO: 59). The cut site is designed to be within exon 9 of GAPDH. Having a cut site in an exon of GAPDH allows for enrichment and selection of cells with proper targeted integration, because cells with improper integration results in cell death (see WO2021226151).

The IRES-RFP nucleotide sequence is as follows:

(SEQ ID NO: 9)
ttggatagttgtggaaagagtcaaatggctctcctcaagcgtatt
caacaaggggctgaaggatgcccagaaggtaccccattgtatggg
atctgatctggggcctcggtgcacatgctttacatgtgtttagtc
gaggttaaaaaacgtctaggccccccgaaccacggggacgtggtt
ttcctttgaaaaacacgatgataatggccacagaattcatggtga
gcaagggcgaggagctgatcaaggagaacatgcacagcaagctgt
acctggaaggcagcgtgaacggccaccagttcaagtgcacccacg
aaggggagggcaagccctacgagggcacccagaccaacaggatca
aggtggtggagggaggccccctgccgttcgcattcgacatcctgg
ccaccatgtttatgtacgggagcaaggccttcatcaagtacccca
agggcctccccgattattttaagcagtccttccctgagggcttca
catgggagagagtcatggtgttcgaagacgggggcgtgctgaccg
ccacccaggacaccagcctccaggacggctgcctcatctacaacg
tgaagctgagaggggtgaacttcccagccaacggccccgtgatga
agcagacaacactgggctgggagcccagcaccgagaccctgtacc
ccgctgacggcgccctggaaggcagatgcgacatggccctgaagc
tcgtggggggggccacctgcactgcaacttcaagaccacatacaa
atccaagaaacccgctacaaacctcaagatgcccggcgtccacta
cgtggaccgcagactggaaagaatcaaggaggccgacaacgagac
ctacgtcgagcagcacgaggtggctgtggccagatactgcgacct
ccctagcaaactggggcacaaacttaatggCATGGACGAGCTGTA
CAAGtga

### Example 2

### Pooled Screening for Targeted Integration of Modified ERT2 Peptide-Suicide Protein Fusion Polypeptide Sequences at the GAPHD Locus

Modified ERT2 peptide-suicide protein fusion polypeptide encoding constructs were delivered to human iPSCs via Nucleofection with a GAPDH targeting ribonucleoprotein (RNP) complex comprising of Cpf1 endonuclease and a single guide RNA (sgRNA) targeting the coding sequence of the GAPDH gene. The sgRNA comprised a sequence 5′ ATCTTCTAGGTATGACAACGA 3′ (SEQ ID NO: 60). Briefly, iPSCs were cultured in Essential 8 (E8) medium and maintained at 37° C. at 5% CO₂ level between passages. Passages were done at 75 to 80% confluency. On the day of Nucleofection, iPSCs were thawed and immediately Nucleofected in Lonza P3 Primary Cell Nucleofection buffer. 7.5 μg of each plasmid construct (BRC00058-61) were Nucleofected into iPSCs using GAPDH Cpf1 RNP with LONZA 4D Nucleofector. The Nucleofected cells were then plated at 50,000 cells/cm² per well of a 6-well plate and grown in E8 medium (plus Rock inhibitor Y-27632 from Tocris) for 72 to 96 hours. Knock-in pools were screened for integration 7d post-Nucleofection using ddPCR, as well as conventional PCR.

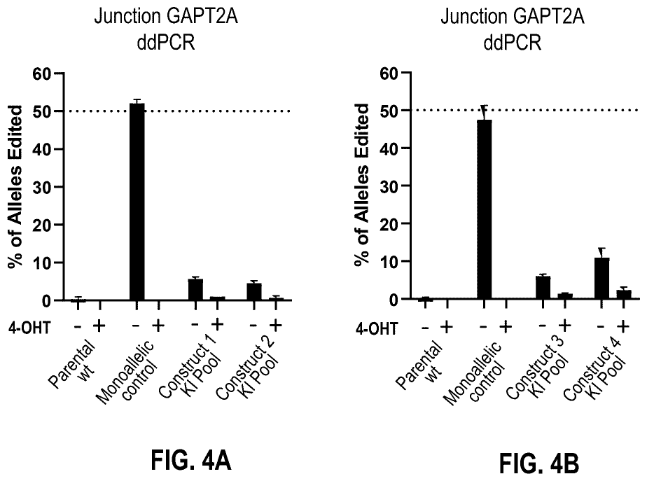

Junction GAPT2A ddPCR analysis revealed that in the absence of ERT2-activating drug 4-OHT, a tamoxifen derivative, fusion polypeptide sequence integration was between 4.5% and 10.9% (FIGS. 4A and 4B) in pools of iPSCs transfected with one of the four constructs. Whereas the integration dropped from 5.7% to 1% in Construct 1-transfected cell pools and from 4.5% to 0.7% in Construct 2-transfected cell pools when treated with 1 nM 4-OHT (FIG. 4A). Similarly, 1 nM 4-OHT treatment reduced integration from 6% to 1.4% and from 10.9% to 2.3% in Construct 3- and Construct 4-transfected cell pools, respectively (FIG. 4B).

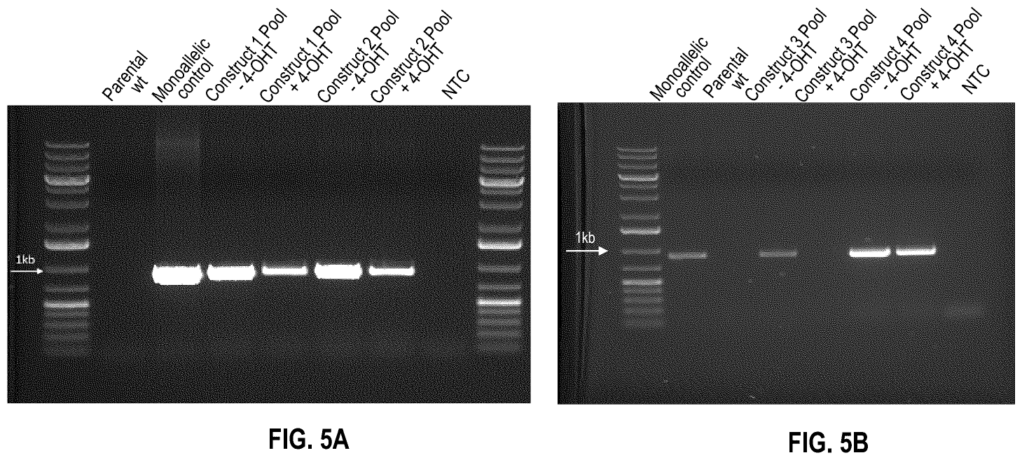

Agarose gel photographs displaying the results of a conventional PCR assessment showed reduced intensity in bands for the iPSC pools treated with 1 nM 4-OHT (FIGS. 5A and 5B). These results confirmed that 4-OHT treatment reduced the amount of cells that carried integration of the kill-switch fusion sequences at the GAPDH locus.

### Example 3

### Screening of Clones for Targeted Integration of Modified ERT2 Peptide-Suicide Protein Fusion Polypeptide Sequences at the Gapdh Locus

Individual clones successfully transfected with each of the four constructs in Table 3, Construct 1, Construct 2, Construct 3, or Construct 4, were screened using PCR, assessed for integration at the 5′ junction, 3′ junction, as well as zygosity of the integrated sequence.

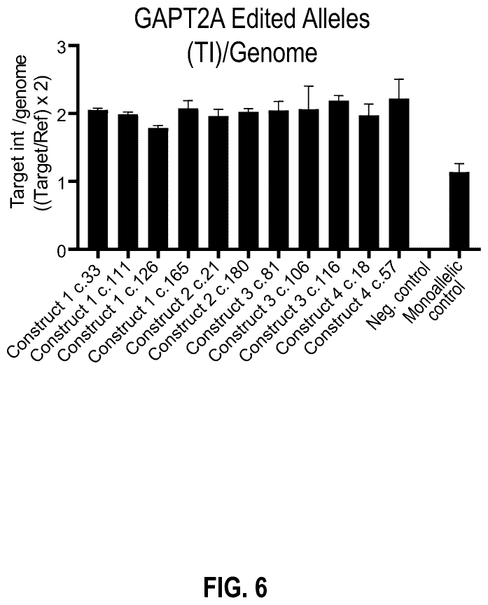

Select clones were further evaluated using ddPCR. Junction GAPT2A ddPCR was used to determine zygosity. All clones selected for testing with this assay displayed approximately two GAPDH-T2A junctions/genome indicating that all clones contain bi-allelic integrations of constructs Construct 1, Construct 2, Construct 3, or Construct 4 (FIG. 6).

### Example 4

### Apoptosis Induction Via 4-OHT Treatment in IPCSs Transfected with Modified ERT2 Peptide-Suicide Protein Constructs

To determine whether modified ERT2 peptide-suicide protein fusion polypeptides of the disclosure were able to induce apoptosis, iPSCs transfected with constructs in Table 3 of the disclosure were treated with 1 nM 4-OHT for up to two days and monitored by microscopy and Incucyte tracking.

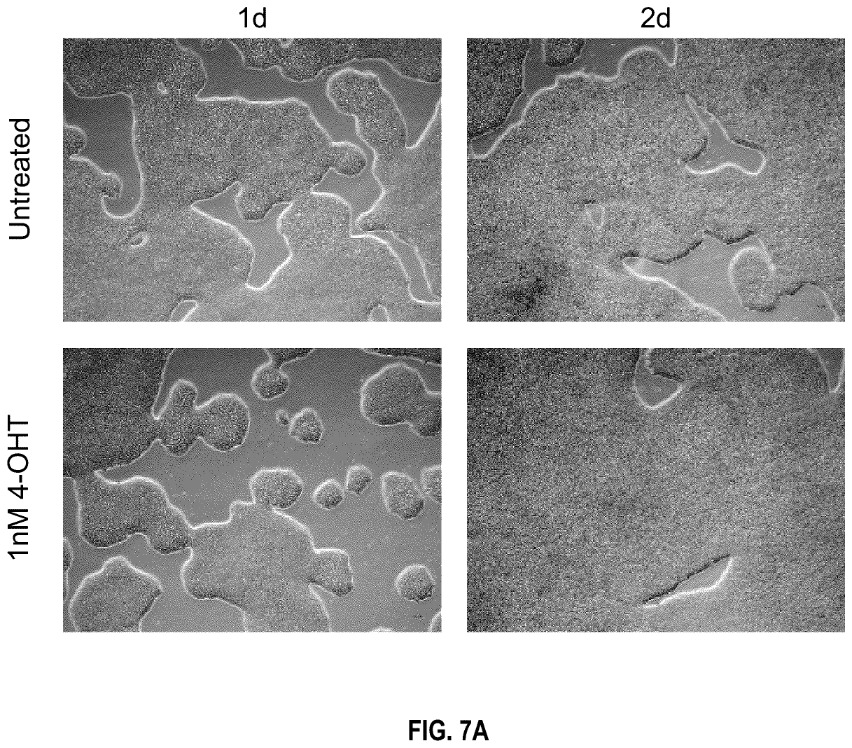

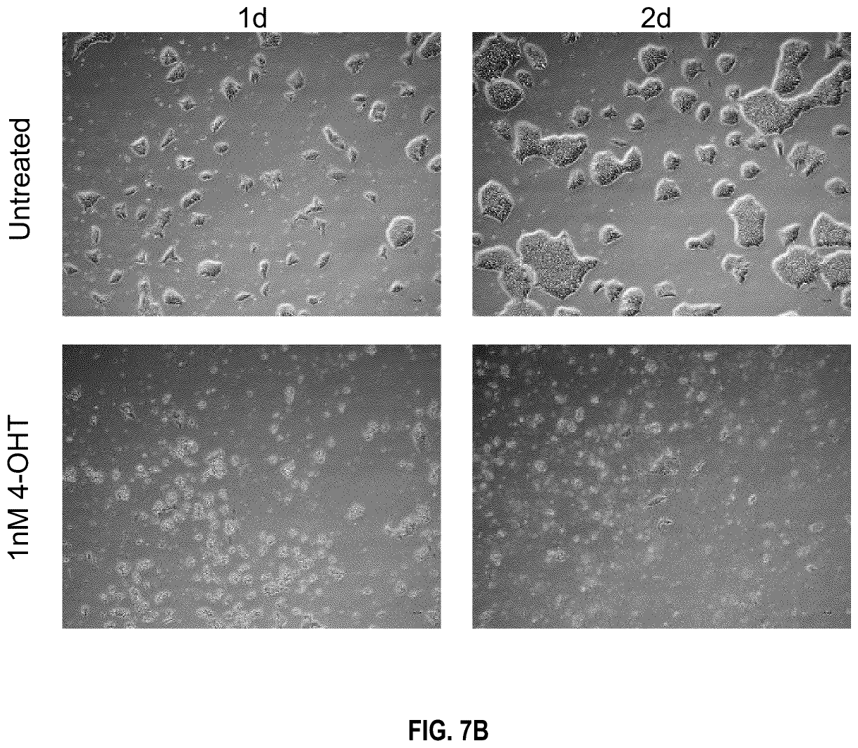

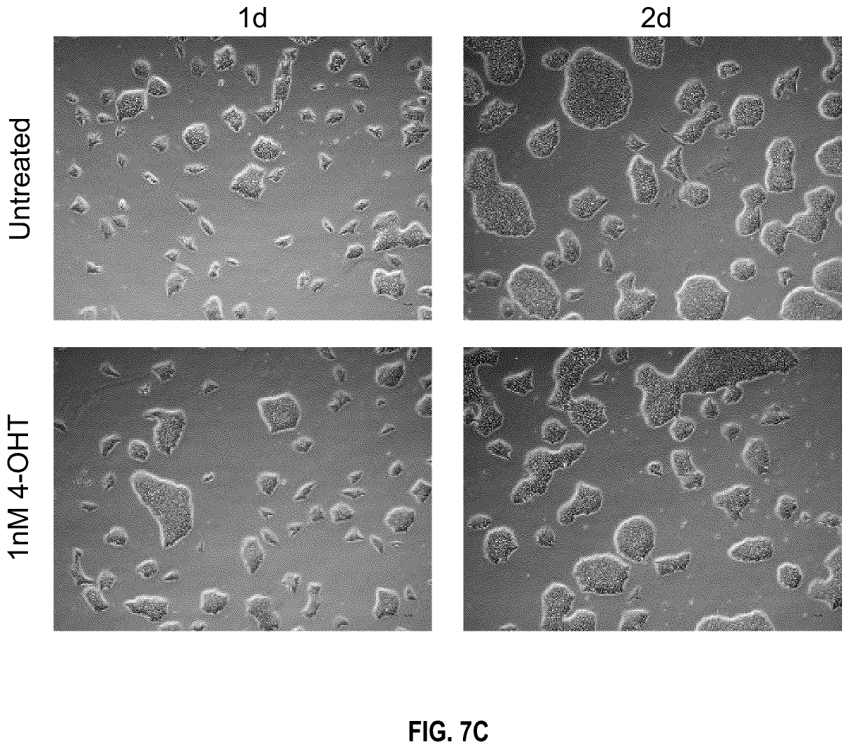

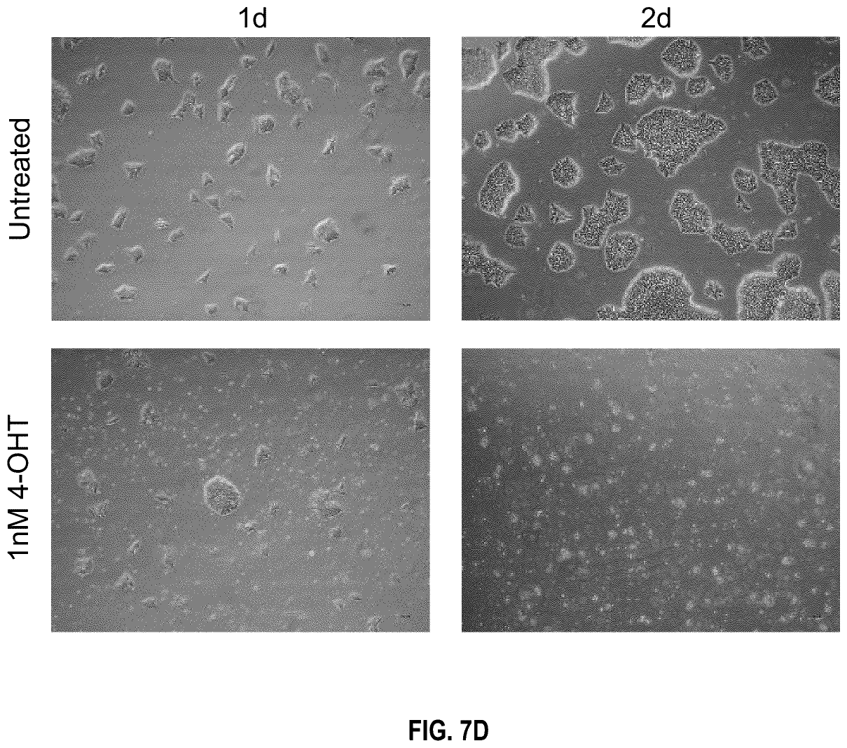

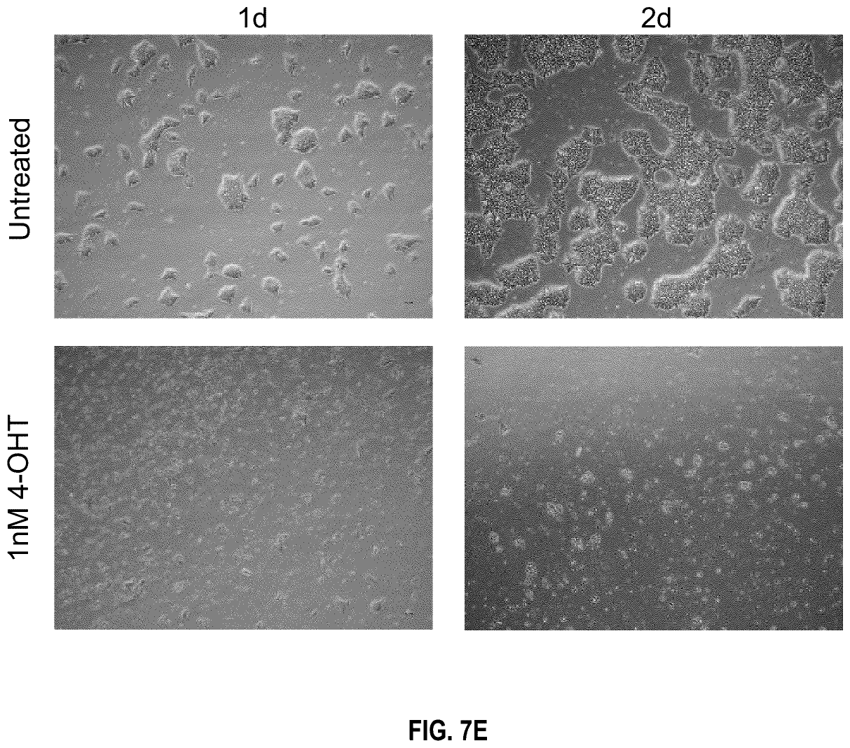

In unedited control cells, there were no differences in cell growth and density between untreated and 4-OHT-treated cells at any time point (FIG. 7A), indicating that 4-OHT at this concentration has no detectable effect on cell health and growth. In cells transfected with Construct 1, Construct 3, or Construct 4, treatment with 1 nM 4-OHT lead to apoptosis, so that cells were completely killed by day 2 (FIGS. 7B, 7D, and 7E). However, 1 nM 4-OHT treatment did not induce apoptosis in cells transfected with Construct 2 (FIG. 7C).

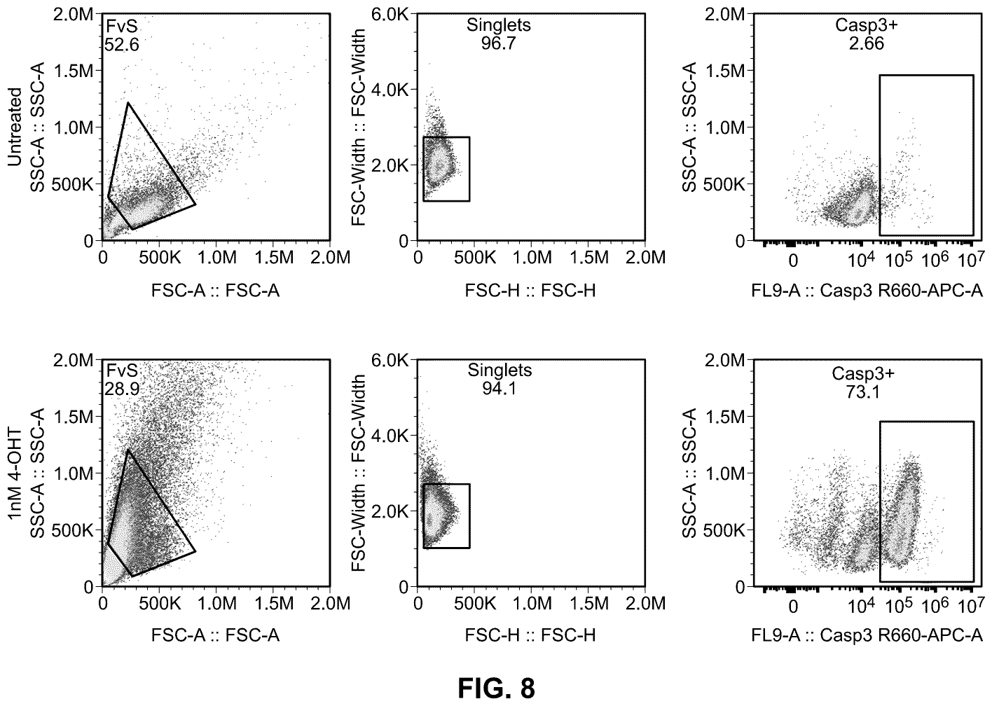

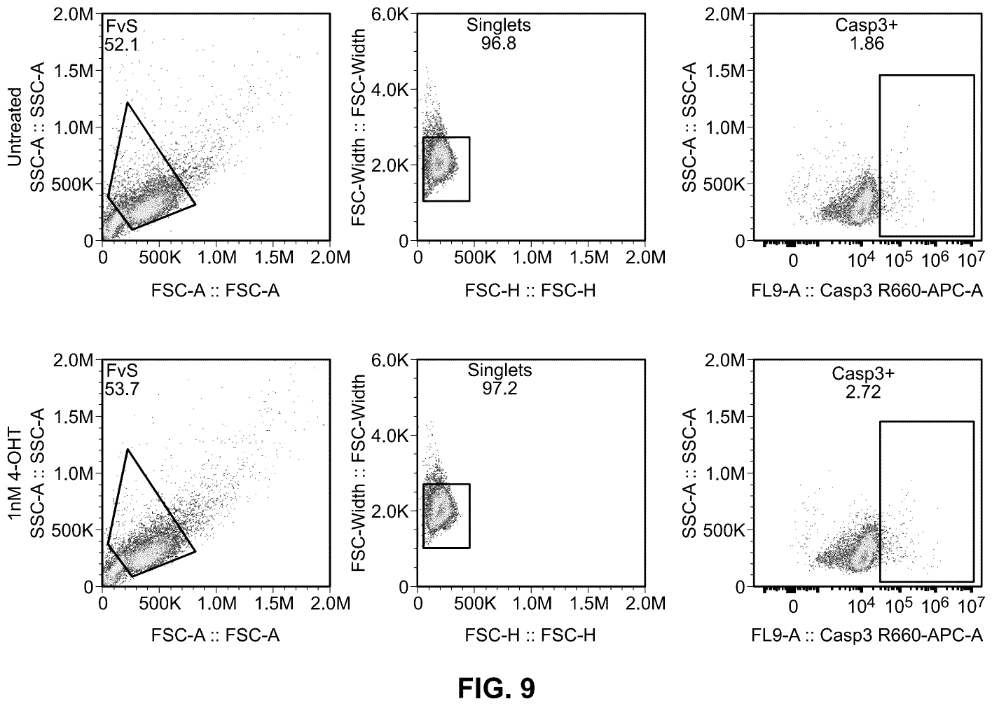

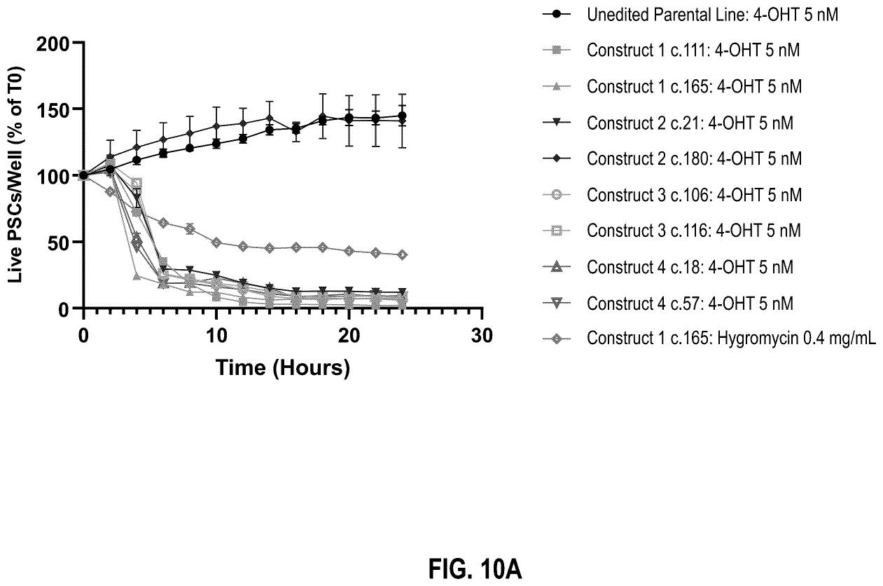

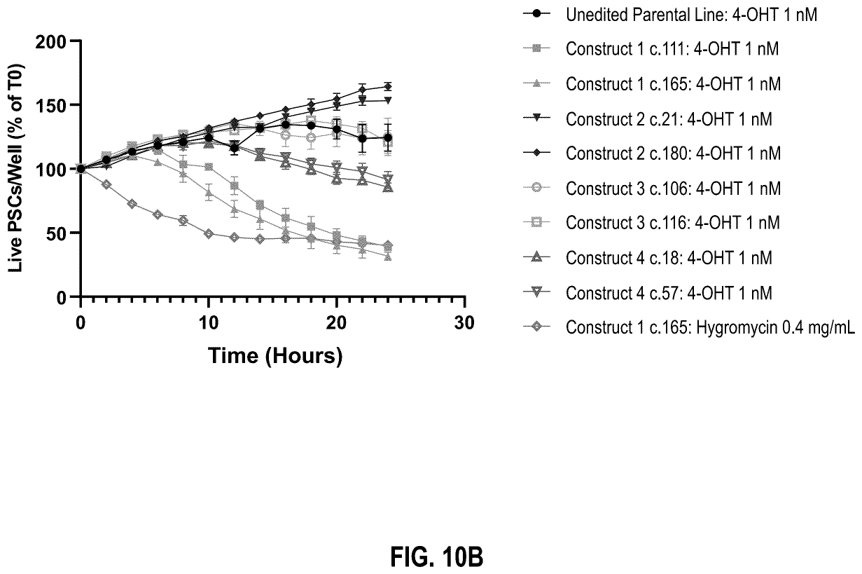

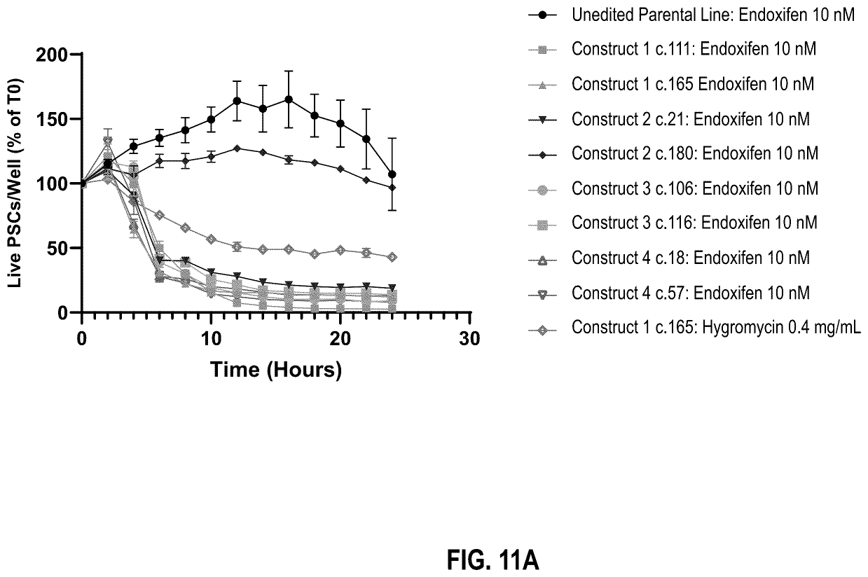

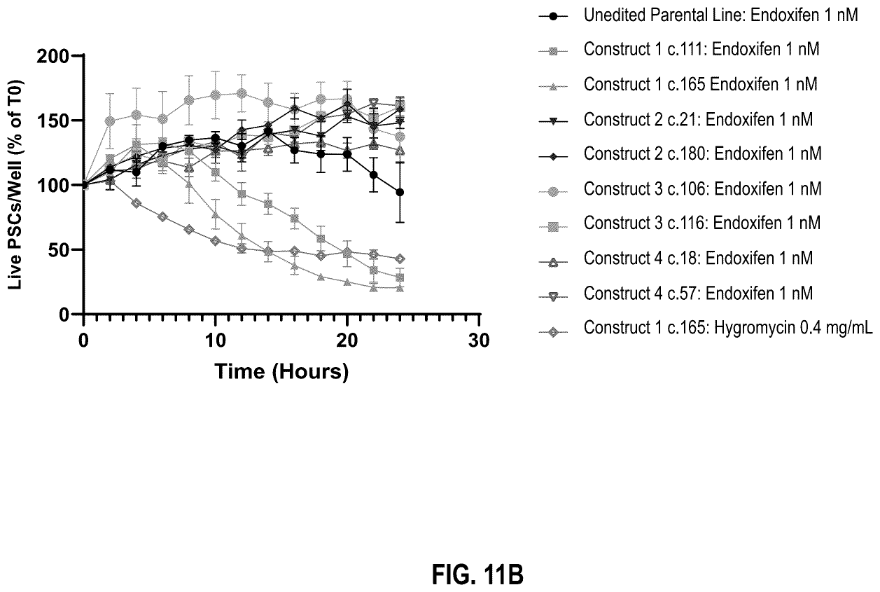

In another set of assessments, Construct 1 or Construct 2 transfected iPSCs were harvested for flow cytometric analysis. The primary antibody used was an anti-human/mouse cleaved Caspase-3. The primary antibody detects human and mouse Caspase 3 cleaved at Asp175. The results confirmed that the Construct 1 fusion polypeptides were able to induce apoptosis in the majority of the cells when treated with 1 nM 4-OHT (FIG. 8) but Construct 2 fusion polypeptides failed to do so at this concentration of 4-OHT (FIG. 9). Later assessments showed that Construct 2 fusion polypeptides were able to induce apoptosis at 5 nM 4-OHT (FIG. 10A) and 10 nM Endoxifen (FIG. 11A), at least for one of the Construct 2-derived clones (c. 21).

### Example 5

### Comparison of 4-OHT-Induced Apoptotic Efficacy in Individual Construct Clones

Next, individual clones that were determined to be suitable for further assessments were evaluated for 24 hours in the presence of 4-OHT at two different concentrations. When treated with 5 nM 4-OHT, all clones except one Construct 2 clone, displayed killing efficacy (FIG. 10A). However, when treated with 1 nM 4-OHT, Construct 1 clones displayed higher efficacy than Construct 2-4 clones (FIG. 10B).

### Example 6

### Comparison of Endoxifen-Induced Apoptotic Efficacy in Individual Construct Clones

Individual Construct 1-4 clones were also assessed for their responses to another tamoxifen metabolite, Endoxifen. Clones were evaluated for 24 hours in the presence of Endoxifen at three different concentrations. When treated with 10 nM Endoxifen, all clones except Construct 2 clone displayed killing compared to a hygromycin kill control (FIG. 11A). Yet, when treated with 1 nM Endoxifen, most clones failed to induce apoptosis, except Construct 1 clones, which approached the level of apoptosis by hygromycin control at 24 hours (FIG. 11B).

### Example 7

### Evaluation of Apoptotic Efficacy in Construct 1 C.165 Treated with Combinations of 4-OHT and Endoxifen

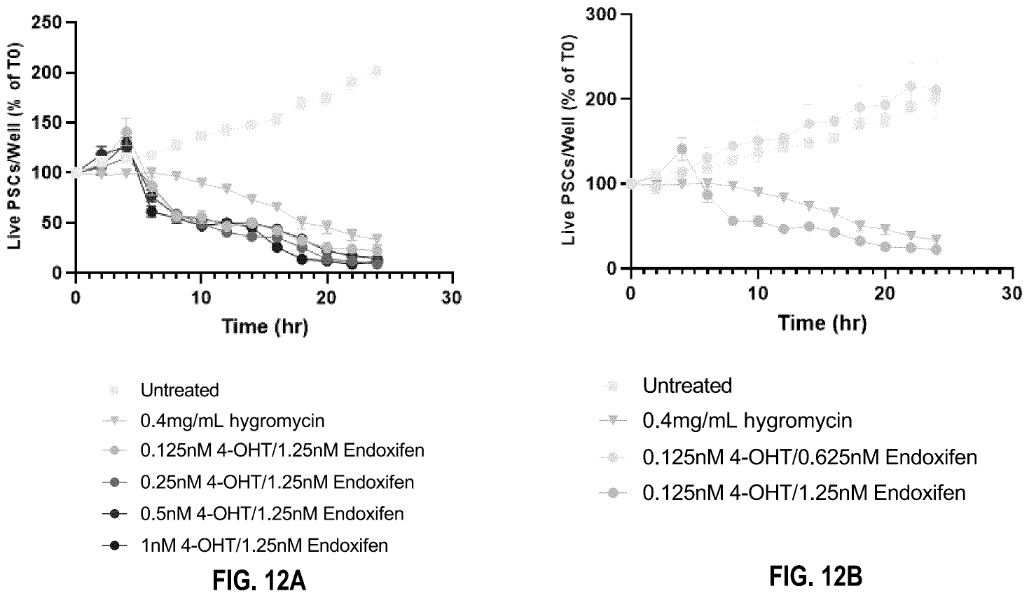

Tamoxifen is a prodrug that is broken down into its active metabolites in vivo. To model predicted drug concentrations in a patient, the clone Construct 1 c.165 was treated with various combinations of 4-OHT and Endoxifen, wherein 1.25 nM Endoxifen was combined with a concentration of 4-OHT ranging between 0.125 nM to 1 nM (FIG. 12A). All tested combinations resulted in comparable apoptotic efficacy (FIG. 12A). In the next evaluation, 0.125 nM 4-OHT was combined with either 0.625 or 1.25 nM Endoxifen (FIG. 12B). The combination with 1.25 nM Endoxifen was associated with apoptotic efficacy comparable to that observed in FIG. 12A. However, the reduction of Endoxifen concentration to 0.625 nM diminished the apoptotic efficacy of the 4-OHT-Endoxifen combination (FIG. 12B), indicating that the lowest effective concentrations for 4-OHT-Endoxifen combination were 0.125 nM and 1.25 nM, respectively.

### Example 8

### Evaluation of Apoptotic Efficacy in PSC-Derived Microglia Precursor Cells or Myeloid Progenitor Cells

To validate conservation of kill switch function after PSCs are differentiated to precursor cells or myeloid progenitor cells, PSCs engineered to incorporate the modified ERT2 peptide-suicide protein fusion polypeptide constructs outlined in Table 3 were differentiated into microglial precursor cells or myeloid progenitor cells. Thereafter PSC-derived microglial precursor cells or myeloid progenitor cells are treated with Endoxifen, 4-OHT or media alone and tested for incorporation of the modified ERT2 peptide-suicide protein fusion polypeptide in the STEL site.

To test function of the modified ERT2 peptide-suicide protein fusion polypeptide constructs in PSC-derived microglial precursor cells or myeloid progenitor cells, cells are cultured, then treated with either lnM, 5 nM, 50 nM, or 100 nM 4-OHT, or 5 nM, 10 nM, 50 nM, or 100 nM Endoxifen, before assessment of apoptosis. It is found that treatment of PSC-derived microglial precursor cells or myeloid progenitor cells with 4-OHT or Endoxifen resulted in apoptosis.

### Example 9

### Evaluation of Apoptotic Efficacy in PSC-Derived Dopaminergic Neurons

To validate conservation of kill switch function after PSCs are differentiated to dopaminergic neurons, PSCs engineered to incorporate the modified ERT2 peptide-suicide protein fusion constructs outlined in Table 3 are differentiated into dopaminergic neurons. Thereafter PSC-derived dopaminergic neurons are treated with Endoxifen, 4-OHT or media alone and tested for incorporation of the modified ERT2 peptide-suicide protein fusion polypeptide construct in the STEL site.

To test function of the modified ERT2 peptide-suicide protein fusion polypeptides in PSC-derived dopaminergic neurons, dopaminergic neurons are cultured, then treated with either lnM, 5 nM, 50 nM, or 100 nM 4-OHT; or 5 nM, 10 nM, 50 nM, or 100 nM Endoxifen, before assessment of apoptosis. It is found that treatment of PSC-dopaminergic neurons with 4-OHT or endoxifen resulted in apoptosis.

### Example 10

### Evaluation of Apoptotic Efficacy in PSC-Derived Myeloid Progenitor Cells

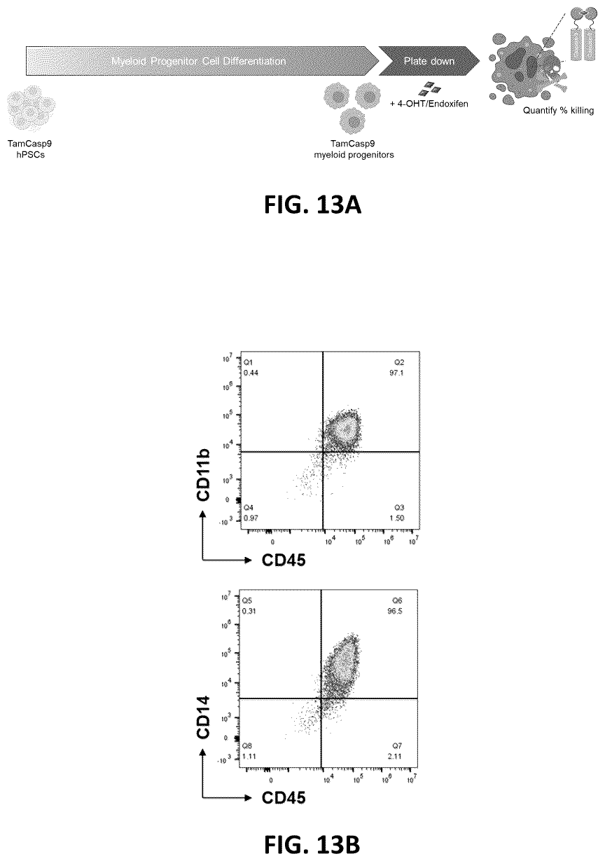

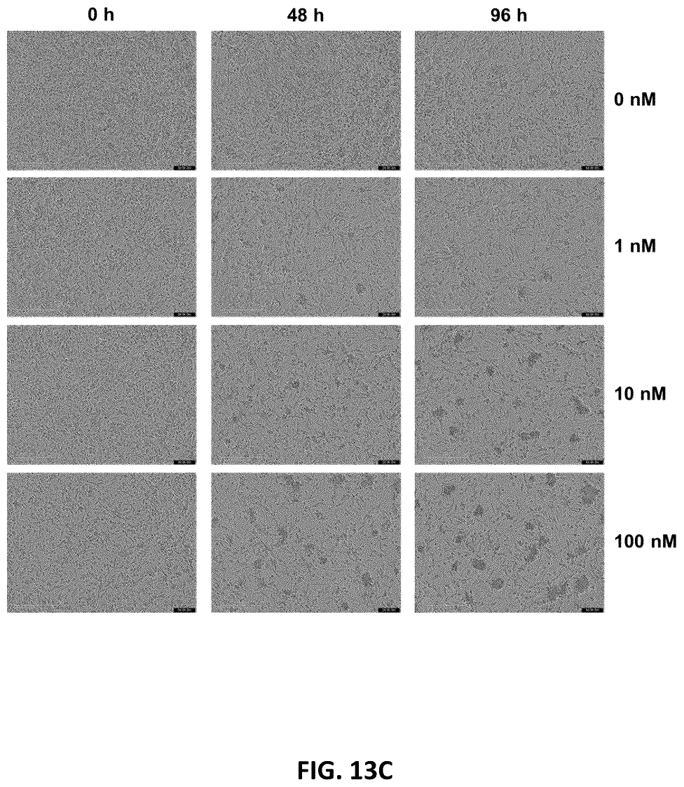

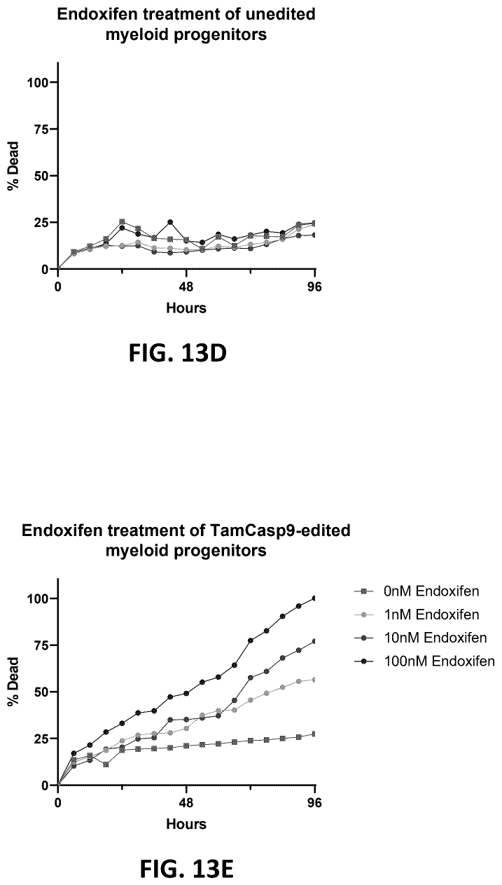

FIG. 13A shows a schematic of an experimental workflow performed to validate conservation of kill switch function after differentiation of PSCs to myeloid progenitor cells.

A hPSC line engineered with TamCasp9 Construct 1 and an unedited parental control hPSC line were differentiated into myeloid progenitor cells through the addition of various growth factors and small molecules. Differentiated cells were plated at 1×10⁵ cells/cm² in an ultra-low attachment cell culture plate for further myeloid cell maturation. After 7 days in maturation medium, cells were collected and frozen down.

FIG. 13B shows flow cytometry evaluation of the differentiated cells, indicating successful differentiation into myeloid progenitor cells.

For testing of TamCasp9 Safety Switch activation, myeloid cells were plated at 1.5×10⁵ cells/cm² in a 96-well tissue culture plate with a 1:750 dilution of ViaStain AOPI Staining Solution (Nexcelom) in X-VIVO 15 Medium with 10 ng/mL GM-CSF. The kill switch was induced with 0, 1, 10 or 100 uM of Endoxifen over 3 days and images were collected every 6 hours on the Incucyte SX5 (Sartorius).

FIG. 13C shows brightfield images with fluorescence overlay. Cells were stained with AO/PI, with dead cells appearing darker after uptake of Propidium Iodide.

FIG. 13D shows quantitation of the kill switch activity in myeloid progenitor cells following differentiation of unedited PSCs at the indicated endoxifen treatment conditions.

FIG. 13E shows quantitation of the kill switch activity in myeloid progenitor cells following differentiation of TamCasp9-edited PSCs, at the indicated endoxifen treatment conditions.

Taken together, the results indicate conservation of the safety switch function post-differentiation, as hPSCs engineered with Construct 1 were differentiated into myeloid progenitor cells and shown to have induced apoptosis following endoxifen treatment.

### Example 11

### Evaluation of Apoptotic Efficacy in PSC-Derived Microglia Precursor Cells or Myeloid Progenitor Cells Containing Construct 1 C.165 Treated with Combinations of 4-OHT and Endoxifen

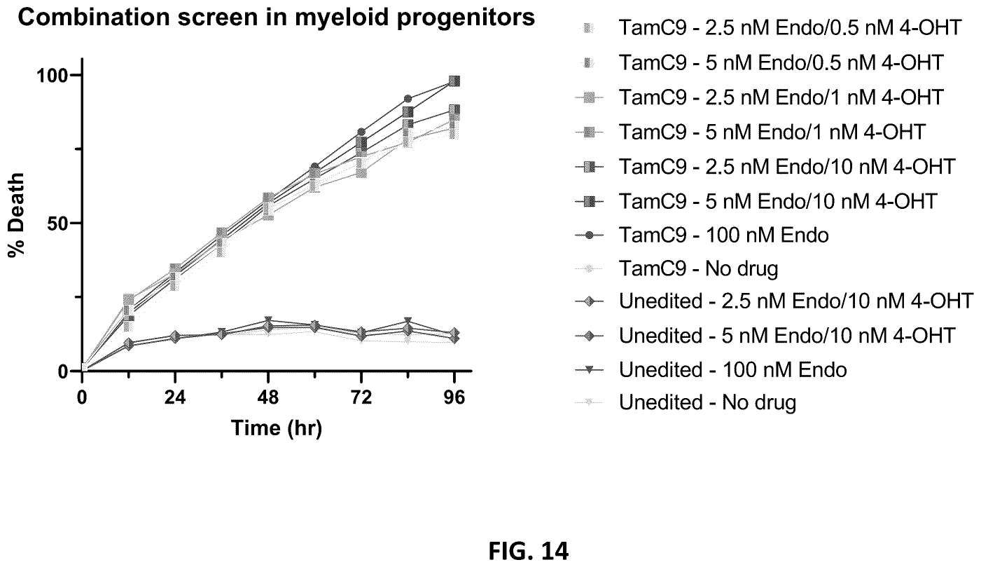

To better model predicted drug concentrations in a patient by testing activation of the kill switch against a range of tamoxifen metabolites, the PSC clone Construct 1 c.165, engineered to incorporate an exemplary modified ERT2 peptide-suicide protein fusion polypeptide construct (Table 3), was differentiated to myeloid progenitor cells as depicted in FIG. 13A. Differentiated cells were plated at 1×10⁵ cells/cm² in an ultra-low attachment cell culture plate for further myeloid cell maturation. After 7 days in maturation medium, cells were collected and frozen down. For testing of TamCasp9 Safety Switch activation, myeloid cells were plated at 1.0×10⁵ cells/cm² in a 96-well tissue culture plate with a 1:750 dilution of ViaStain AOPI Staining Solution (Nexcelom) in X-VIVO 15 Medium with 10 ng/mL GM-CSF. The kill switch was induced with varying concentrations of combination 4-OHT and Endoxifen treatment over 4 days and images were collected every 4 hours on the Incucyte SX5, with 12 hour timepoints plotted on a graph (Sartorius) (FIG. 14). Either 2.5 nM or 5 nM of Endoxifen was tested in combination with either 0.5 nM, 1 nM or 10 nM of 4-OHT. 100 nM Endoxifen was added as positive killing control as per FIG. 13E. All tested combinations resulted in greater than 80% apoptotic efficacy in differentiated myeloid progenitor cells, with the lowest tested combination being 2.5 nM Endoxifen and 0.5 nM 4-OHT. Treatment with 5 nM Endoxifen and 10n M 4-OHT killed all cells by 4 days, comparable to the 100 nM Endoxifen positive killing control. Treatment of unedited differentiated myeloid progenitors did not induce apoptosis. These data demonstrate that synergistic activation of TamCasp9 using a combination of 4-OHT and Endoxifen can lower the minimally effective dosage of both metabolites in PSC-derived Microglia Precursor Cells or Myeloid Progenitor Cells.

### Example 12

### Design and Generation of Improved Modified ERT2 Peptide-Suicide Protein Constructs, and Evaluation of Apoptotic Efficacy in PSCs and in PSC-Derived Myeloid Progenitor Cells

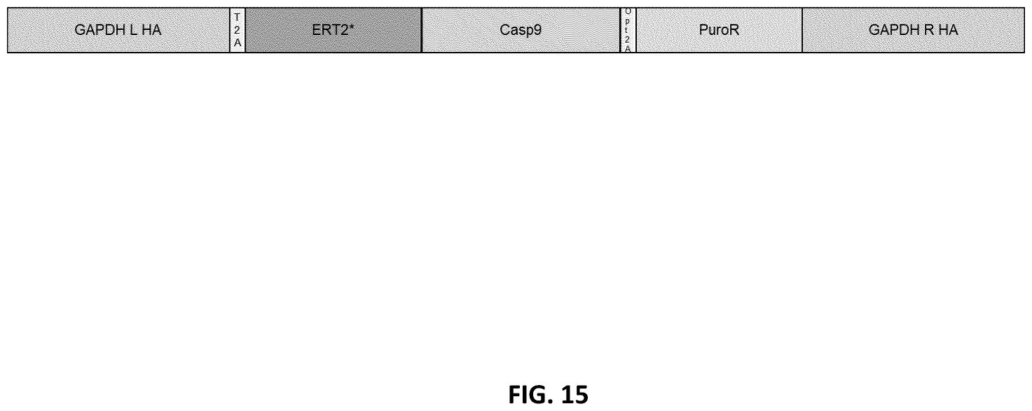

An additional four modified ERT2 peptides (ERT2 mutants; Constructs 5-8) are selected to be fused to Caspase-9. All four additional engineered ERT2-Casp9 candidates show killing efficiency after the addition of 2.5 nM Endoxifen, in HEK293T cells within 48 hours of induction (data not shown). In this context, Constructs 5-8 are designed to include a separator sequence, an optimized 2A linker (Opt2a), followed by a puromycin resistance (PuroR) marker SEQ ID NO: 10 to allow for puromycin selection after transfection of constructs into iPSCs, downstream to the modified ERT2 peptide-suicide protein fusion polypeptide (Table 4). The resulting four kill switch fusion polypeptide encoding sequences are inserted between the left and right homology arms targeting the GAPDH STEL with a T2A peptide sequence linking a modified ERT2 peptide-suicide protein fusion polypeptide encoding sequence to the left homology arm (FIG. 15).

Opt2a- linker amino acid sequence
(SEQ ID NO: 61):
GSGQCTNYALLKLAGDVESNPGPGSGEGRGSLLTCGDVEENPGP
PuroR amino acid sequence
(SEQ ID NO: 62)
MTEYKPTVRLATRDDVPRAVRTLAAAFADYPATRHTVDPDRHIER
VTELQELFLTRVGLDIGKVWVADDGAAVAVWTTPESVEAGAVFAE
IGPRMAELSGSRLAAQQQMEGLLAPHRPKEPAWFLATVGVSPDHQ
GKGLGSAVVLPGVEAAERAGVPAFLETSAPRNLPFYERLGFTVTA
DVEVPEGPRTWCMTRKPGA

TABLE 4

| | modified ERT2 | |
|---|---|---|
| | peptide- | |
| | suicide protein | |
| Plasmid | fusion | |
| No. | polypeptide | Sequence |
| Construct 5 | ERT2*mut41v2-Casp9- | SAGDMRAANLWPSPLMIKRSKKNSLALSLTADQMVSALLDAEPPI |
| | Opt2A-PuroR | LYSEYDPTRPFSEASMMGLLTNLADREIVHMINWAKRVPGFVDLT |
| | | LHDQVHLLECAWLEILMIGVVWRSMEHPVKLLFAPNLVLDRDQGK |
| | | CVEGLVEIFDMLLATSSRFRMMNLQGEEFVCLKSIILLNSGVYTF |
| | | LPSTLKSLEEKDHIHRVLDKITDTLIHLMAKAGLTLQQQHQRLAQ |
| | | LLLILSHIRHASNKGMELLYSMKCKNVVPLYDLLLEAADAHRLHA |
| | | PTSRGGASVEETDQSHLATAGSTSSHSLQKYYITGEAEGFPATAG |
| | | GGGSGGGGSGGGGSVDGFDVGALESLRGNADLAYILSMEPCGHCL |
| | | IINNVNFCRESGLRTRTGSNIDCEKLRRRFSSLHFMVEVKGDLTA |
| | | KKMVLALLELARQDHGALDCCVVVILSHGCQASHLQFPGAVYGTD |
| | | GCPVSVEKIVNIFNGTSCPSLGGKPKLFFIQACGGEQKDHGFEVA |
| | | STSPEDESPGSNPEPDATPFQEGLRTFDQLDAISSLPTPSDIFVS |
| | | YSTFPGFVSWRDPKSGSWYVETLDDIFEQWAHSEDLQSLLLRVAN |
| | | AVSVKGIYKQMPGCFNFLRKKLFFKTSGSGQCTNYALLKLAGDVE |
| | | SNPGPGSGEGRGSLLTCGDVEENPGPMTEYKPTVRLATRDDVPRA |
| | | VRTLAAAFADYPATRHTVDPDRHIERVTELQELFLTRVGLDIGKV |
| | | WVADDGAAVAVWTTPESVEAGAVFAEIGPRMAELSGSRLAAQQQM |
| | | EGLLAPHRPKEPAWFLATVGVSPDHQGKGLGSAVVLPGVEAAERA |
| | | GVPAFLETSAPRNLPFYERLGFTVTADVEVPEGPRTWCMTRKPGA |
| | | (SEQ ID NO: 13) |
| | | Bold = ERT2*mut41v2 |
| | | Italics = linker |
| | | Plain = Casp9 |
| | | GSGQCTNYALLKLAGDVESNPGPGSGEGRGSLLTCGDVEENPGP |
| | | (SEQ ID NO: 61) = Opt2A linker |
| | | MTEYKPTVRLATRDDVPRAVRTLAAAFADYPATRHTVDPDRHIER |
| | | VTELQELFLTRVGLDIGKVWVADDGAAVAVWTTPESVEAGAVFAE |
| | | IGPRMAELSGSRLAAQQQMEGLLAPHRPKEPAWFLATVGVSPDHQ |
| | | GKGLGSAVVLPGVEAAERAGVPAFLETSAPRNLPFYERLGFTVTA |
| | | DVEVPEGPRTWCMTRKPGA |
| | | (SEQ ID NO:62) = PuroR |
| | | Nucleotide sequence: |
| | | ATGtctgctggagacatgagagctgccaacctttggccaagcccg |
| | | ctcatgatcaaacgctctaagaagaacagcctggccttgtccctg |
| | | acggccgaccagatggtcagtgccttgttggatgctgagcccccc |
| | | atactctattccgagtatgatcctaccagacccttcagtgaagct |
| | | tcgatgatgggcttactgaccaacctggcagacagggagATCgtt |
| | | cacatgatcaactgggcgaagagggtgccaggctttgtggatttg |
| | | accctccatgatcaggtccaccttctagaatgtgcctggCTAgag |
| | | atcctgatgattggtGTGgtctggcgctccatggagcacccagtg |
| | | aagctactgtttgctcctaacttgGTGttggacagggacCAGgga |
| | | aaatgtgtagagggcCTGGTGGAGATCTTCGACATGCTGCTGGCT |
| | | ACATCATCTCGGTTCCGCATGATGAATCTGCAGGGAGAGGAGTTT |
| | | GTGTGCCTCAAATCTATTATTTTGCTTAATTCTGGAGTGTACACA |
| | | TTTCTGCCCAGCACCCTGAAGTCTCTGGAAGAGAAGGACCATATC |
| | | CACCGAGTCCTGGACAAGATCACAGACACTTTGATCCACCTGATG |
| | | GCCAAGGCAGGCCTGACCCTGCAGCAGCAGCACCAGCGGCTGGCC |
| | | CAGCTCCTCCTCATCCTCTCCCACATCAGGCACGCCagtaacaaa |
| | | ggcatggagctgctgtacagcatgaagtgcaagaacgtggtgccc |
| | | ctctatGaCctgctgctggaggcggcggacgcccaccgcctacat |
| | | gcgcccactagccgtggaggggcatccgtggaggagacggaccaa |
| | | agccacttggccactgcgggctctacttcatcgcattccttgcaa |
| | | aagtattacatcacgggggaggcagagggtttccctgccacagct |
| | | gggggtggaggttcagggggtggaggttcaggtggtggcggtagt |
| | | gtcgatggcttcgatgtcggtgctcttgagagtttgaggggaaat |
| | | gcagatttggcttacatcctgagcatggagccctgtggccactgc |
| | | ctcattatcaacaatgtgaacttctgccgtgagtccgggctccgc |
| | | acccgcactggctccaacatcgactgtgagaagttgcggcgtcgc |
| | | ttctcctcgctgcatttcatggtggaggtgaagggcgacctgact |
| | | gccaagaaaatggtgctggctttgctggagctggcgcggcaggac |
| | | cacggtgctctggactgctgcgtggtggtcattctctctcacggc |
| | | tgtcaggccagccacctgcagttcccaggggctgtctacggcaca |
| | | gatggatgccctgtgtcggtcgagaagattgtgaacatcttcaat |
| | | gggaccagctgccccagcctgggagggaagcccaagctctttttc |
| | | atccaggcctgtggtggggagcagaaagaccatgggtttgaggtg |
| | | gcctccacttcccctgaagacgagtcccctggcagtaaccccgag |
| | | ccagatgccaccccgttccaggaaggtttgaggaccttcgaccag |
| | | ctggacgccatatctagtttgcccacacccagtgacatctttgtg |
| | | tcctactctactttcccaggttttgtttcctggagggaccccaag |
| | | agtggctcctggtacgttgagaccctggacgacatctttgagcag |
| | | tgggctcactctgaagacctgcagtccctcctgcttagggtcgct |
| | | aatgctgtttcggtgaaagggatttataaacagatgcctggttgc |
| | | tttaatttcctccggaaaaaacttttctttaaaacatcaGGCTCC |
| | | GGCCAGTGCACAAATTATGCACTGCTGAAGCTCGCCGGGGATGTC |
| | | GAGAGTAACCCAGGACCTGGAAGCGGAGAAGGTCGTGGTAGTCTA |
| | | CTAACGTGTGGTGATGTAGAAGAAAATCCTGGACCTatgaccgag |
| | | tacaagcccacggtgcgcctcgccacccgcgacgacgtccccagg |
| | | gccgtacgcaccctcgccgccgcgttcgccgactaccccgccacg |
| | | cgccacaccgtcgatccggaccgccacatcgagcgggtcaccgag |
| | | ctgcaagaactcttcctcacgcgcgtcgggctcgacatcggcaag |
| | | gtgtgggtcgcggacgacggcgccgcggtggcggtctggaccacg |
| | | ccggagagcgtcgaagcgggggcggtgttcgccgagatcggcccg |
| | | cgcatggccgagttgagcggttcccggctggccgcgcagcaacag |
| | | atggaaggcctcctggcgccgcaccggcccaaggagcccgcgtgg |
| | | ttcctggccaccgtcggcgtctcgcccgaccaccagggcaagggt |
| | | ctgggcagcgccgtcgtgctccccggagtggaggcggccgagcgc |
| | | gccggggtgcccgccttcctggagacctccgcgccccgcaacctc |
| | | cccttctacgagcggctcggcttcaccgtcaccgccgacgtcgag |
| | | gtgcccgaaggaccgcgcacctggtgcatgacccgcaagcccggt |
| | | gcc |
| | | (SEQ ID NO: 14) |
| Construct 6 | ERT2*mut51-Casp9- | SAGDMRAANLWPSPLMIKRSKKNSLALSLTADQMVSALLDAEPPI |
| | Opt2A-PuroR | LYSEYDPTRPFSEASMMGLLTNLADREIVHMINWAKRVPGFVDLT |
| | | LHDQVHLLECAWMEILMIGVVWRSMEHPVKLLFAPNLLLDRDEGK |
| | | CVEGLVEIFDMLLATSSRFRMMNLQGEEFVCLKSIILLNSGVYTF |
| | | LPSTLKSLEEKDHIHRVLDKITDTLIHLMAKAGLTLQQQHQRLAQ |
| | | LLLILSHIRHMSNKGMELLYSMKCKNVVPLYDLLLEAADAHRLHA |
| | | PTSRGGASVEETDQSHLATAGSTSSHSLQKYYITGEAEGFPATAG |
| | | GGGSGGGGSGGGGSVDGFDVGALESLRGNADLAYILSMEPCGHCL |
| | | IINNVNFCRESGLRTRTGSNIDCEKLRRRFSSLHFMVEVKGDLTA |
| | | KKMVLALLELARQDHGALDCCVVVILSHGCQASHLQFPGAVYGTD |
| | | GCPVSVEKIVNIFNGTSCPSLGGKPKLFFIQACGGEQKDHGFEVA |
| | | STSPEDESPGSNPEPDATPFQEGLRTFDQLDAISSLPTPSDIFVS |
| | | YSTFPGFVSWRDPKSGSWYVETLDDIFEQWAHSEDLQSLLLRVAN |
| | | AVSVKGIYKQMPGCFNFLRKKLFFKTSGSGQCTNYALLKLAGDVE |
| | | SNPGPGSGEGRGSLLTCGDVEENPGPMTEYKPTVRLATRDDVPRA |
| | | VRTLAAAFADYPATRHTVDPDRHIERVTELQELFLTRVGLDIGKV |
| | | WVADDGAAVAVWTTPESVEAGAVFAEIGPRMAELSGSRLAAQQQM |
| | | EGLLAPHRPKEPAWFLATVGVSPDHQGKGLGSAVVLPGVEAAERA |
| | | GVPAFLETSAPRNLPFYERLGFTVTADVEVPEGPRTWCMTRKPGA |
| | | (SEQ ID NO: 15) |
| | | Bold = ERT2*mut51 |
| | | Italics = linker |
| | | Plain = Casp9 |
| | | GSGQCTNYALLKLAGDVESNPGPGSGEGRGSLLTCGDVEENPGP |
| | | (SEQ ID NO: 61) = Opt2A linker |
| | | MTEYKPTVRLATRDDVPRAVRTLAAAFADYPATRHTVDPDRHIER |
| | | VTELQELFLTRVGLDIGKVWVADDGAAVAVWTTPESVEAGAVFAE |
| | | IGPRMAELSGSRLAAQQQMEGLLAPHRPKEPAWFLATVGVSPDHQ |
| | | GKGLGSAVVLPGVEAAERAGVPAFLETSAPRNLPFYERLGFTVTA |
| | | DVEVPEGPRTWCMTRKPGA |
| | | (SEQ ID NO: 62) = PuroR |
| | | Nucleotide sequence: |
| | | ATGtctgctggagacatgagagctgccaacctttggccaagcccg |
| | | ctcatgatcaaacgctctaagaagaacagcctggccttgtccctg |
| | | acggccgaccagatggtcagtgccttgttggatgctgagcccccc |
| | | atactctattccgagtatgatcctaccagacccttcagtgaagct |
| | | tcgatgatgggcttactgaccaacctggcagacagggagATCgtt |
| | | cacatgatcaactgggcgaagagggtgccaggctttgtggatttg |
| | | accctccatgatcaggtccaccttctagaatgtgcctggATGgag |
| | | atcctgatgattggtGTGgtctggcgctccatggagcacccagtg |
| | | aagctactgtttgctcctaacttgCTCttggacagggacGAGgga |
| | | aaatgtgtagagggcCTGGTGGAGATCTTCGACATGCTGCTGGCT |
| | | ACATCATCTCGGTTCCGCATGATGAATCTGCAGGGAGAGGAGTTT |
| | | GTGTGCCTCAAATCTATTATTTTGCTTAATTCTGGAGTGTACACA |
| | | TTTCTGCCCAGCACCCTGAAGTCTCTGGAAGAGAAGGACCATATC |
| | | CACCGAGTCCTGGACAAGATCACAGACACTTTGATCCACCTGATG |
| | | GCCAAGGCAGGCCTGACCCTGCAGCAGCAGCACCAGCGGCTGGCC |
| | | CAGCTCCTCCTCATCCTCTCCCACATCAGGCACATGagtaacaaa |
| | | ggcatggagctgctgtacagcatgaagtgcaagaacgtggtgccc |
| | | ctctatGaCctgctgctggaggcggcggacgcccaccgcctacat |
| | | gcgcccactagccgtggaggggcatccgtggaggagacggaccaa |
| | | agccacttggccactgcgggctctacttcatcgcattccttgcaa |
| | | aagtattacatcacgggggaggcagagggtttccctgccacagct |
| | | gggggtggaggttcagggggtggaggttcaggtggtggcggtagt |
| | | gtcgatggcttcgatgtcggtgctcttgagagtttgaggggaaat |
| | | gcagatttggcttacatcctgagcatggagccctgtggccactgc |
| | | ctcattatcaacaatgtgaacttctgccgtgagtccgggctccgc |
| | | acccgcactggctccaacatcgactgtgagaagttgcggcgtcgc |
| | | ttctcctcgctgcatttcatggtggaggtgaagggcgacctgact |
| | | gccaagaaaatggtgctggctttgctggagctggcgcggcaggac |
| | | cacggtgctctggactgctgcgtggtggtcattctctctcacggc |
| | | tgtcaggccagccacctgcagttcccaggggctgtctacggcaca |
| | | gatggatgccctgtgtcggtcgagaagattgtgaacatcttcaat |
| | | gggaccagctgccccagcctgggagggaagcccaagctctttttc |
| | | atccaggcctgtggtggggagcagaaagaccatgggtttgaggtg |
| | | gcctccacttcccctgaagacgagtcccctggcagtaaccccgag |
| | | ccagatgccaccccgttccaggaaggtttgaggaccttcgaccag |
| | | ctggacgccatatctagtttgcccacacccagtgacatctttgtg |
| | | tcctactctactttc\|ccaggttttgtttcctggagggaccccaa |
| | | gagtggctcctggtacgttgagaccctggacgacatctttgagca |
| | | gtgggctcactctgaagacctgcagtccctcctgcttagggtcgc |
| | | taatgctgtttcggtgaaagggatttataaacagatgcctggttg |
| | | ctttaatttcctccggaaaaaacttttctttaaaacatcaGGCTC |
| | | CGGCCAGTGCACAAATTATGCACTGCTGAAGCTCGCCGGGGATGT |
| | | CGAGAGTAACCCAGGACCTGGAAGCGGAGAAGGTCGTGGTAGTCT |
| | | ACTAACGTGTGGTGATGTAGAAGAAAATCCTGGACCTatgaccga |
| | | gtacaagcccacggtgcgcctcgccacccgcgacgacgtccccag |
| | | ggccgtacgcaccctcgccgccgcgttcgccgactaccccgccac |
| | | gcgccacaccgtcgatccggaccgccacatcgagcgggtcaccga |
| | | gctgcaagaactcttcctcacgcgcgtcgggctcgacatcggcaa |
| | | ggtgtgggtcgcggacgacggcgccgcggtggcggtctggaccac |
| | | gccggagagcgtcgaagcgggggcggtgttcgccgagatcggccc |
| | | gcgcatggccgagttgagcggttcccggctggccgcgcagcaaca |
| | | gatggaaggcctcctggcgccgcaccggcccaaggagcccgcgtg |
| | | gttcctggccaccgtcggcgtctcgcccgaccaccagggcaaggg |
| | | tctgggcagcgccgtcgtgctccccggagtggaggcggccgagcg |
| | | cgccggggtgcccgccttcctggagacctccgcgccccgcaacct |
| | | ccccttctacgagcggctcggcttcaccgtcaccgccgacgtcga |
| | | ggtgcccgaaggaccgcgcacctggtgcatgacccgcaagcccgg |
| | | tgcc |
| | | (SEQ ID NO: 16) |
| Construct 7 | ERT2*mut37-Casp9- | SAGDMRAANLWPSPLMIKRSKKNSLALSLTADQMVSALLDAEPPI |
| | Opt2A-PuroR | LYSEYDPTRPFSEASMMGLLTNLADREIVHMINWAKRVPGFVDLT |
| | | LHDQVHLLECAWMEILMIGLVWRSMEHPVKLLFAPNLLLDRDQGK |
| | | CVEGMVEIFDMLLATSSRFRMMNLQGEEFVCLKSIILLNSGVYTF |
| | | LPSTLKSLEEKDHIHRVLDKITDTLIHLMAKAGLTLQQQHQRLAQ |
| | | LLLILSHIRHMSNKGMELLYSMKCKNVVPLYDLLLEAADAHRLHA |
| | | PTSRGGASVEETDQSHLATAGSTSSHSLQKYYITGEAEGFPATAG |
| | | GGGSGGGGSGGGGSVDGFDVGALESLRGNADLAYILSMEPCGHCL |
| | | IINNVNFCRESGLRTRTGSNIDCEKLRRRFSSLHFMVEVKGDLTA |
| | | KKMVLALLELARQDHGALDCCVVVILSHGCQASHLQFPGAVYGTD |
| | | GCPVSVEKIVNIFNGTSCPSLGGKPKLFFIQACGGEQKDHGFEVA |
| | | STSPEDESPGSNPEPDATPFQEGLRTFDQLDAISSLPTPSDIFVS |
| | | YSTFPGFVSWRDPKSGSWYVETLDDIFEQWAHSEDLQSLLLRVAN |
| | | AVSVKGIYKQMPGCFNFLRKKLFFKTSGSGQCTNYALLKLAGDVE |
| | | SNPGPGSGEGRGSLLTCGDVEENPGPMTEYKPTVRLATRDDVPRA |
| | | VRTLAAAFADYPATRHTVDPDRHIERVTELQELFLTRVGLDIGKV |
| | | WVADDGAAVAVWTTPESVEAGAVFAEIGPRMAELSGSRLAAQQQM |
| | | EGLLAPHRPKEPAWFLATVGVSPDHQGKGLGSAVVLPGVEAAERA |
| | | GVPAFLETSAPRNLPFYERLGFTVTADVEVPEGPRTWCMTRKPGA |
| | | (SEQ ID NO: 17) |
| | | Bold = ERT2*mut37 |
| | | Italics = linker |
| | | Plain = Casp9 |
| | | GSGQCTNYALLKLAGDVESNPGPGSGEGRGSLLTCGDVEENPGP |
| | | (SEQ ID NO: 61) = Opt2A linker |
| | | MTEYKPTVRLATRDDVPRAVRTLAAAFADYPATRHTVDPDRHIER |
| | | VTELQELFLTRVGLDIGKVWVADDGAAVAVWTTPESVEAGAVFAE |
| | | IGPRMAELSGSRLAAQQQMEGLLAPHRPKEPAWFLATVGVSPDHQ |
| | | GKGLGSAVVLPGVEAAERAGVPAFLETSAPRNLPFYERLGFTVTA |
| | | DVEVPEGPRTWCMTRKPGA |
| | | (SEQ ID NO:62) = PuroR |
| | | Nucleotide sequence: |
| | | ATGtctgctggagacatgagagctgccaacctttggccaagcccg |
| | | ctcatgatcaaacgctctaagaagaacagcctggccttgtccctg |
| | | acggccgaccagatggtcagtgccttgttggatgctgagcccccc |
| | | atactctattccgagtatgatcctaccagacccttcagtgaagct |
| | | tcgatgatgggcttactgaccaacctggcagacagggagATCgtt |
| | | cacatgatcaactgggcgaagagggtgccaggctttgtggatttg |
| | | accctccatgatcaggtccaccttctagaatgtgcctggATGgag |
| | | atcctgatgattggtCTCgtctggcgctccatggagcacccagtg |
| | | aagctactgtttgctcctaacttgCTCttggacagggacCAGgga |
| | | aaatgtgtagagggcATGGTGGAGATCTTCGACATGCTGCTGGCT |
| | | ACATCATCTCGGTTCCGCATGATGAATCTGCAGGGAGAGGAGTTT |
| | | GTGTGCCTCAAATCTATTATTTTGCTTAATTCTGGAGTGTACACA |
| | | TTTCTGCCCAGCACCCTGAAGTCTCTGGAAGAGAAGGACCATATC |
| | | CACCGAGTCCTGGACAAGATCACAGACACTTTGATCCACCTGATG |
| | | GCCAAGGCAGGCCTGACCCTGCAGCAGCAGCACCAGCGGCTGGCC |
| | | CAGCTCCTCCTCATCCTCTCCCACATCAGGCACATGagtaacaaa |
| | | ggcatggagctgctgtacagcatgaagtgcaagaacgtggtgccc |
| | | ctctatGaCctgctgctggaggcggcggacgcccaccgcctacat |
| | | gcgcccactagccgtggaggggcatccgtggaggagacggaccaa |
| | | agccacttggccactgcgggctctacttcatcgcattccttgcaa |
| | | aagtattacatcacgggggaggcagagggtttccctgccacagct |
| | | gggggtggaggttcagggggtggaggttcaggtggtggcggtagt |
| | | gtcgatggcttcgatgtcggtgctcttgagagtttgaggggaaat |
| | | gcagatttggcttacatcctgagcatggagccctgtggccactgc |
| | | ctcattatcaacaatgtgaacttctgccgtgagtccgggctccgc |
| | | acccgcactggctccaacatcgactgtgagaagttgcggcgtcgc |
| | | ttctcctcgctgcatttcatggtggaggtgaagggcgacctgact |
| | | gccaagaaaatggtgctggctttgctggagctggcgcggcaggac |
| | | cacggtgctctggactgctgcgtggtggtcattctctctcacggc |
| | | tgtcaggccagccacctgcagttcccaggggctgtctacggcaca |
| | | gatggatgccctgtgtcggtcgagaagattgtgaacatcttcaat |
| | | gggaccagctgccccagcctgggagggaagcccaagctctttttc |
| | | atccaggcctgtggtggggagcagaaagaccatgggtttgaggtg |
| | | gcctccacttcccctgaagacgagtcccctggcagtaaccccgag |
| | | ccagatgccaccccgttccaggaaggtttgaggaccttcgaccag |
| | | ctggacgccatatctagtttgcccacacccagtgacatctttgtg |
| | | tcctactctactttcccaggttttgtttcctggagggaccccaag |
| | | agtggctcctggtacgttgagaccctggacgacatctttgagcag |
| | | tgggctcactctgaagacctgcagtccctcctgcttagggtcgct |
| | | aatgctgtttcggtgaaagggatttataaacagatgcctggttgc |
| | | tttaatttcctccggaaaaaacttttctttaaaacatcaGGCTCC |
| | | GGCCAGTGCACAAATTATGCACTGCTGAAGCTCGCCGGGGATGTC |
| | | GAGAGTAACCCAGGACCTGGAAGCGGAGAAGGTCGTGGTAGTCTA |
| | | CTAACGTGTGGTGATGTAGAAGAAAATCCTGGACCTatgaccgag |
| | | tacaagcccacggtgcgcctcgccacccgcgacgacgtccccagg |
| | | gccgtacgcaccctcgccgccgcgttcgccgactaccccgccacg |
| | | cgccacaccgtcgatccggaccgccacatcgagcgggtcaccgag |
| | | ctgcaagaactcttcctcacgcgcgtcgggctcgacatcggcaag |
| | | gtgtgggtcgcggacgacggcgccgcggtggcggtctggaccacg |
| | | ccggagagcgtcgaagcgggggcggtgttcgccgagatcggcccg |
| | | cgcatggccgagttgagcggttcccggctggccgcgcagcaacag |
| | | atggaaggcctcctggcgccgcaccggcccaaggagcccgcgtgg |
| | | ttcctggccaccgtcggcgtctcgcccgaccaccagggcaagggt |
| | | ctgggcagcgccgtcgtgctccccggagtggaggcggccgagcgc |
| | | gccggggtgcccgccttcctggagacctccgcgccccgcaacctc |
| | | cccttctacgagcggctcggcttcaccgtcaccgccgacgtcgag |
| | | gtgcccgaaggaccgcgcacctggtgcatgacccgcaagcccggt |
| | | gcc |
| | | (SEQ ID NO: 18) |
| Construct 8 | ERT2*mut94-Casp9- | SAGDMRAANLWPSPLMIKRSKKNSLALSLTADQMVSALLDAEPPI |
| | Opt2A-PuroR | LYSEYDPTRPFSEASMMGLLTNLADREIVHMINWAKRVPGFVDLT |
| | | LHDQVHLLECAWLEILMIGVVWRSMEHPVKLLFAPNLLLDRDQGK |
| | | CVEGLVEIFDMLLATSSRFRMMNLQGEEFVCLKSIILLNSGVYTF |
| | | LPSTLKSLEEKDHIHRVLDKITDTLIHLMAKAGLTLQQQHQRLAQ |
| | | LLLILSHIRHASNKGMELLYSMKCKNVVPLYDLLLEAADAHRLHA |
| | | PTSRGGASVEETDQSHLATAGSTSSHSLQKYYITGEAEGFPATAG |
| | | GGGSGGGGSGGGGSVDGFDVGALESLRGNADLAYILSMEPCGHCL |
| | | IINNVNFCRESGLRTRTGSNIDCEKLRRRFSSLHFMVEVKGDLTA |
| | | KKMVLALLELARQDHGALDCCVVVILSHGCQASHLQFPGAVYGTD |
| | | GCPVSVEKIVNIFNGTSCPSLGGKPKLFFIQACGGEQKDHGFEVA |
| | | STSPEDESPGSNPEPDATPFQEGLRTFDQLDAISSLPTPSDIFVS |
| | | YSTFPGFVSWRDPKSGSWYVETLDDIFEQWAHSEDLQSLLLRVAN |
| | | AVSVKGIYKQMPGCFNFLRKKLFFKTSGSGQCTNYALLKLAGDVE |
| | | SNPGPGSGEGRGSLLTCGDVEENPGPMTEYKPTVRLATRDDVPRA |
| | | VRTLAAAFADYPATRHTVDPDRHIERVTELQELFLTRVGLDIGKV |
| | | WVADDGAAVAVWTTPESVEAGAVFAEIGPRMAELSGSRLAAQQQM |
| | | EGLLAPHRPKEPAWFLATVGVSPDHQGKGLGSAVVLPGVEAAERA |
| | | GVPAFLETSAPRNLPFYERLGFTVTADVEVPEGPRTWCMTRKPGA |
| | | (SEQ ID NO: 19) |
| | | Bold = ERT2*mut94 |
| | | Italics = linker |
| | | Plain = Casp9 |
| | | GSGQCTNYALLKLAGDVESNPGPGSGEGRGSLLTCGDVEENPGP |
| | | (SEQ ID NO: 61) = Opt2A linker |
| | | MTEYKPTVRLATRDDVPRAVRTLAAAFADYPATRHTVDPDRHIER |
| | | VTELQELFLTRVGLDIGKVWVADDGAAVAVWTTPESVEAGAVFAE |
| | | IGPRMAELSGSRLAAQQQMEGLLAPHRPKEPAWFLATVGVSPDHQ |
| | | GKGLGSAVVLPGVEAAERAGVPAFLETSAPRNLPFYERLGFTVTA |
| | | DVEVPEGPRTWCMTRKPGA |
| | | (SEQ ID NO:62) = PuroR |
| | | Nucleotide sequence: |
| | | ATGtctgctggagacatgagagctgccaacctttggccaagcccg |
| | | ctcatgatcaaacgctctaagaagaacagcctggccttgtccctg |
| | | acggccgaccagatggtcagtgccttgttggatgctgagcccccc |
| | | atactctattccgagtatgatcctaccagacccttcagtgaagct |
| | | tcgatgatgggcttactgaccaacctggcagacagggagATCgtt |
| | | cacatgatcaactgggcgaagagggtgccaggctttgtggatttg |
| | | accctccatgatcaggtccaccttctagaatgtgcctggCTAgag |
| | | atcctgatgattggtGTGgtctggcgctccatggagcacccagtg |
| | | aagctactgtttgctcctaacttgCTCttggacagggacCAGgga |
| | | aaatgtgtagagggcCTGGTGGAGATCTTCGACATGCTGCTGGCT |
| | | ACATCATCTCGGTTCCGCATGATGAATCTGCAGGGAGAGGAGTTT |
| | | GTGTGCCTCAAATCTATTATTTTGCTTAATTCTGGAGTGTACACA |
| | | TTTCTGCCCAGCACCCTGAAGTCTCTGGAAGAGAAGGACCATATC |
| | | CACCGAGTCCTGGACAAGATCACAGACACTTTGATCCACCTGATG |
| | | GCCAAGGCAGGCCTGACCCTGCAGCAGCAGCACCAGCGGCTGGCC |
| | | CAGCTCCTCCTCATCCTCTCCCACATCAGGCACGCCagtaacaaa |
| | | ggcatggagctgctgtacagcatgaagtgcaagaacgtggtgccc |
| | | ctctatGaCctgctgctggaggcggcggacgcccaccgcctacat |
| | | gcgcccactagccgtggaggggcatccgtggaggagacggaccaa |
| | | agccacttggccactgcgggctctacttcatcgcattccttgcaa |
| | | aagtattacatcacgggggaggcagagggtttccctgccacagct |
| | | gggggtggaggttcagggggtggaggttcaggtggtggcggtagt |
| | | gtcgatggcttcgatgtcggtgctcttgagagtttgaggggaaat |
| | | gcagatttggcttacatcctgagcatggagccctgtggccactgc |
| | | ctcattatcaacaatgtgaacttctgccgtgagtccgggctccgc |
| | | acccgcactggctccaacatcgactgtgagaagttgcggcgtcgc |
| | | ttctcctcgctgcatttcatggtggaggtgaagggcgacctgact |
| | | gccaagaaaatggtgctggctttgctggagctggcgcggcaggac |
| | | cacggtgctctggactgctgcgtggtggtcattctctctcacggc |
| | | tgtcaggccagccacctgcagttcccaggggctgtctacggcaca |
| | | gatggatgccctgtgtcggtcgagaagattgtgaacatcttcaat |
| | | gggaccagctgccccagcctgggagggaagcccaagctctttttc |
| | | atccaggcctgtggtggggagcagaaagaccatgggtttgaggtg |
| | | gcctccacttcccctgaagacgagtcccctggcagtaaccccgag |
| | | ccagatgccaccccgttccaggaaggtttgaggaccttcgaccag |
| | | ctggacgccatatctagtttgcccacacccagtgacatctttgtg |
| | | tcctactctactttcccaggttttgtttcctggagggaccccaag |
| | | agtggctcctggtacgttgagaccctggacgacatctttgagcag |
| | | tgggctcactctgaagacctgcagtccctcctgcttagggtcgct |
| | | aatgctgtttcggtgaaagggatttataaacagatgcctggttgc |
| | | tttaatttcctccggaaaaaacttttctttaaaacatcaGGCTCC |
| | | GGCCAGTGCACAAATTATGCACTGCTGAAGCTCGCCGGGGATGTC |
| | | GAGAGTAACCCAGGACCTGGAAGCGGAGAAGGTCGTGGTAGTCTA |
| | | CTAACGTGTGGTGATGTAGAAGAAAATCCTGGACCTatgaccgag |
| | | tacaagcccacggtgcgcctcgccacccgcgacgacgtccccagg |
| | | gccgtacgcaccctcgccgccgcgttcgccgactaccccgccacg |
| | | cgccacaccgtcgatccggaccgccacatcgagcgggtcaccgag |
| | | ctgcaagaactcttcctcacgcgcgtcgggctcgacatcggcaag |
| | | gtgtgggtcgcggacgacggcgccgcggtggcggtctggaccacg |
| | | ccggagagcgtcgaagcgggggcggtgttcgccgagatcggcccg |
| | | cgcatggccgagttgagcggttcccggctggccgcgcagcaacag |
| | | atggaaggcctcctggcgccgcaccggcccaaggagcccgcgtgg |
| | | ttcctggccaccgtcggcgtctcgcccgaccaccagggcaagggt |
| | | ctgggcagcgccgtcgtgctccccggagtggaggcggccgagcgc |
| | | gccggggtgcccgccttcctggagacctccgcgccccgcaacctc |
| | | cccttctacgagcggctcggcttcaccgtcaccgccgacgtcgag |
| | | gtgcccgaaggaccgcgcacctggtgcatgacccgcaagcccggt |
| | | gcc |
| | | (SEQ ID NO: 20) |

The modified ERT2 peptide-suicide protein fusion polypeptide encoding constructs (Constructs 5-8) are delivered to human iPSCs via Nucleofection with a GAPDH targeting ribonucleoprotein (RNP) complex comprising of Cpf1 endonuclease and a single guide RNA (sgRNA) targeting the coding sequence of the GAPDH gene, as per Example 2. Individual clones are derived from the transfected pools via puromycin selection and apoptotic efficacy is determined in individual clones edited with Constructs 5-8 by screening clones in the presence of a range of 4-OHT and Endoxifen in PSCs and PSC-derived Myeloid Progenitor cells, as per Example 11.

### Example 13

### Design and Generation of Tandem Modified ERT2 Peptide-Suicide Protein Constructs, and Evaluation for Improved Apoptotic Efficacy in PSCs and in PSC-Derived Myeloid Progenitor Cells

To determine if apoptotic efficacy could be improved by the presence of tandem copies of the modified ERT2 peptides (ERT2 mutants) fused to Caspase-9, two exemplary constructs, Construct 1 (ERT2*mut81-Casp9) and Construct 5 (ERT2*mut41v2-Casp9-Opt2A-PuroR) (Table 3) are designed as tandem copies linked by a separator sequence (SS), either a P2A sequence (SEQ ID NO: 11) or an optimized 2A linker designated Opt2A_2.0 (SEQ ID NO: 12) (Table 5). The sequences of the separator sequences are shown below:

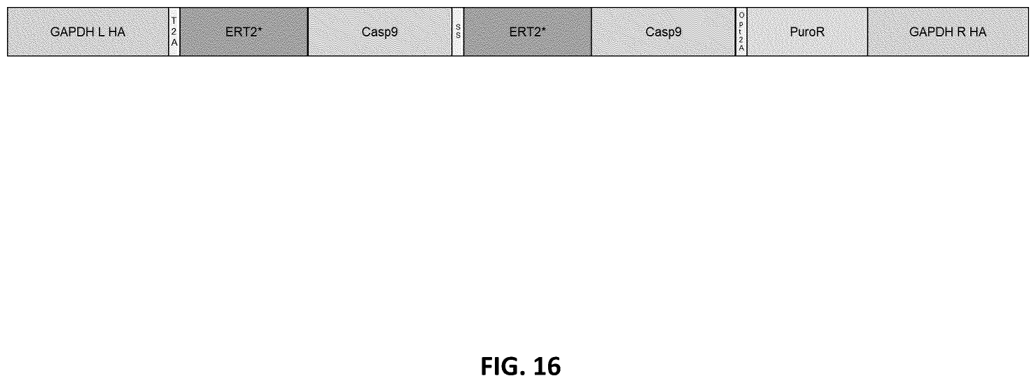

All four designed constructs (Constructs 9-12) are also designed to include a separator sequence, an optimized 2A linker (Opt2a), followed by a puromycin resistance (PuroR) marker SEQ ID NO: 10 to allow for puromycin selection after transfection of constructs into iPSCs, downstream to the modified ERT2 peptide-suicide protein fusion polypeptide (Table 5). The resulting four kill switch fusion polypeptide encoding sequences are inserted between the left and right homology arms targeting the GAPDH STEL with a T2A peptide sequence linking the tandem modified ERT2 peptide-suicide protein fusion polypeptide encoding sequence to the left homology arm (FIG. 16).

TABLE 5

| | modified ERT2 peptide- | |
|---|---|---|
| | suicide protein | |
| Plasmid No. | fusion polypeptide | Sequence |
| Construct 9 | ERT2*mut81-Casp9-Opt2A- | SAGDMRAANLWPSPLMIKRSKKNSLALSLDQMVSALLDAEPPILYST |
| | ERT2*mut81-Casp9-Opt2A- | AEYDPTRPFSEASMMGLLTNLADRELVHMINWAKRVPGFVDLTLHDQ |
| | PuroR | VHLLECAWMEILMIGVVWRSMEHPVKLLFAPNLLLDRDQGKCVEGLV |
| | | EIFDMLLATSSRFRMMNLQGEEFVCLKSIILLNSGVYTFLPSTLKSL |
| | | EEKDHIHRVLDKITDTLIHLMAKAGLTLQQQHQRLAQLLLILSHIRH |
| | | MSNKGMELLYSMKCKNVVPLYDLLLEAADAHRLHAPTSRGGASVEET |
| | | DQSHLATAGSTSSHSLQKYYITGEAEGFPATAGGGGSGGGGSGGGGS |
| | | VDGFDVGALESLRGNADLAYILSMEPCGHCLIINNVNFCRESGLRTR |
| | | TGSNIDCEKLRRRFSSLHFMVEVKGDLTAKKMVLALLELARQDHGAL |
| | | DCCVVVILSHGCQASHLQFPGAVYGTDGCPVSVEKIVNIFNGTSCPS |
| | | LGGKPKLFFIQACGGEQKDHGFEVASTSPEDESPGSNPEPDATPFQE |
| | | GLRTFDQLDAISSLPTPSDIFVSYSTFPGFVSWRDPKSGSWYVETLD |
| | | DIFEQWAHSEDLQSLLLRVANAVSVKGIYKQMPGCFNFLRKKLFFKT |
| | | SGSGQCTNYALLKLAGDVESNPGPGSGEGRGSLLTCGDVEENPGPMS |
| | | AGDMRAANLWPSPLMIKRSKKNSLALSLTADQMVSALLDAEPPILYS |
| | | EYDPTRPFSEASMMGLLTNLADRELVHMINWAKRVPGFVDLTLHDQV |
| | | HLLECAWMEILMIGVVWRSMEHPVKLLFAPNLLLDRDQGKCVEGLVE |
| | | IFDMLLATSSRFRMMNLQGEEFVCLKSIILLNSGVYTFLPSTLKSLE |
| | | EKDHIHRVLDKITDTLIHLMAKAGLTLQQQHQRLAQLLLILSHIRHM |
| | | SNKGMELLYSMKCKNVVPLYDLLLEAADAHRLHAPTSRGGASVEETD |
| | | QSHLATAGSTSSHSLQKYYITGEAEGFPATAGGGGSGGGGSGGGGSV |
| | | DGFDVGALESLRGNADLAYILSMEPCGHCLIINNVNFCRESGLRTRT |
| | | GSNIDCEKLRRRFSSLHFMVEVKGDLTAKKMVLALLELARQDHGALD |
| | | CCVVVILSHGCQASHLQFPGAVYGTDGCPVSVEKIVNIFNGTSCPSL |
| | | GGKPKLFFIQACGGEQKDHGFEVASTSPEDESPGSNPEPDATPFQEG |
| | | LRTFDQLDAISSLPTPSDIFVSYSTFPGFVSWRDPKSGSWYVETLDD |
| | | IFEQWAHSEDLQSLLLRVANAVSVKGIYKQMPGCFNFLRKKLFFKTS |
| | | GSGQCTNYALLKLAGDVESNPGPGSGEGRGSLLTCGDVEENPGPMTE |
| | | YKPTVRLATRDDVPRAVRTLAAAFADYPATRHTVDPDRHIERVTELQ |
| | | ELFLTRVGLDIGKVWVADDGAAVAVWTTPESVEAGAVFAEIGPRMAE |
| | | LSGSRLAAQQQMEGLLAPHRPKEPAWFLATVGVSPDHQGKGLGSAVV |
| | | LPGVEAAERAGVPAFLETSAPRNLPFYERLGFTVTADVEVPEGPRTW |
| | | CMTRKPGA |
| | | (SEQ ID NO: 21) |
| | | Bold = ERT2*mut81 |
| | | Italics = linker |
| | | Plain = Casp9 |
| | | GSGQCTNYALLKLAGDVESNPGPGSGEGRGSLLTCGDVEENPGP |
| | | (SEQ ID NO: 61) = Opt2A linker |
| | | MTEYKPTVRLATRDDVPRAVRTLAAAFADYPATRHTVDPDRHIERVT |
| | | ELQELFLTRVGLDIGKVWVADDGAAVAVWTTPESVEAGAVFAEIGPR |
| | | MAELSGSRLAAQQQMEGLLAPHRPKEPAWFLATVGVSPDHQGKGLGS |
| | | AVVLPGVEAAERAGVPAFLETSAPRNLPFYERLGFTVTADVEVPEGP |
| | | RTWCMTRKPGA |
| | | (SEQ ID NO: 62) = PuroR |
| | | Nucleotide sequence: |
| | | ATGtctgctggagacatgagagctgccaacctttggccaagcccgct |
| | | catgatcaaacgctctaagaagaacagcctggccttgtccctgacgg |
| | | ccgaccagatggtcagtgccttgttggatgctgagccccccatactc |
| | | tattccgagtatgatcctaccagacccttcagtgaagcttcgatgat |
| | | gggcttactgaccaacctggcagacagggagCTGGTTCACATGATCA |
| | | ACTGGGCGAAGAGGGTGCCAGGCTTTGTGGATTTGACCCTCCATGAT |
| | | CAGGTCCACCTTCTAGAATGTGCCTGGATGGAGATCCTGATGATTGG |
| | | TGTGGTCTGGCGCTCCATGGAGCACCCAGTGAAGCTACTGTTTGCTC |
| | | CTAACTTGCTCTTGGACAGGGACCAGGGAAAATGTGTAGAGGGCCTG |
| | | GTGGAGATCTTCGACATGCTGCTGGCTACATCATCTCGGTTCCGCAT |
| | | GATGAATCTGCAGGGAGAGGAGTTTGTGTGCCTCAAATCTATTATTT |
| | | TGCTTAATTCTGGAGTGTACACATTTCTGCCCAGCACCCTGAAGTCT |
| | | CTGGAAGAGAAGGACCATATCCACCGAGTCCTGGACAAGATCACAGA |
| | | CACTTTGATCCACCTGATGGCCAAGGCAGGCCTGACCCTGCAGCAGC |
| | | AGCACCAGCGGCTGGCCCAGCTCCTCCTCATCCTCTCCCACATCAGG |
| | | CACATGAGTAACAAAGGCATGGAGCTGctgtacagcatgaagtgcaa |
| | | gaacgtggtgcccctctatGaCctgctgctggaggcggcggacgccc |
| | | accgcctacatgcgcccactagccgtggaggggcatccgtggaggag |
| | | acggaccaaagccacttggccactgcgggctctacttcatcgcattc |
| | | cttgcaaaagtattacatcacgggggaggcagagggtttccctgcca |
| | | cagctgggggtggaggttcagggggtggaggttcaggtggtggcggt |
| | | agtgtcgatggcttcgatgtcggtgctcttgagagtttgaggggaaa |
| | | tgcagatttggcttacatcctgagcatggagccctgtggccactgcc |
| | | tcattatcaacaatgtgaacttctgccgtgagtccgggctccgcacc |
| | | cgcactggctccaacatcgactgtgagaagttgcggcgtcgcttctc |
| | | ctcgctgcatttcatggtggaggtgaagggcgacctgactgccaaga |
| | | aaatggtgctggctttgctggagctggcgcggcaggaccacggtgct |
| | | ctggactgctgcgtggtggtcattctctctcacggctgtcaggccag |
| | | ccacctgcagttcccaggggctgtctacggcacagatggatgccctg |
| | | tgtcggtcgagaagattgtgaacatcttcaatgggaccagctgcccc |
| | | agcctgggagggaagcccaagctctttttcatccaggcctgtggtgg |
| | | ggagcagaaagaccatgggtttgaggtggcctccacttcccctgaag |
| | | acgagtcccctggcagtaaccccgagccagatgccaccccgttccag |
| | | gaaggtttgaggaccttcgaccagctggacgccatatctagtttgcc |
| | | cacacccagtgacatctttgtgtcctactctactttcccaggttttg |
| | | tttcctggagggaccccaagagtggctcctggtacgttgagaccctg |
| | | gacgacatctttgagcagtgggctcactctgaagacctgcagtccct |
| | | cctgcttagggtcgctaatgctgtttcggtgaaagggatttataaac |
| | | agatgcctggttgctttaatttcctccggaaaaaacttttctttaaa |
| | | acatcaGGCTCCGGCCAGTGCACAAATTATGCACTGCTGAAGCTCGC |
| | | CGGGGATGTCGAGAGTAACCCAGGACCTGGAAGCGGAGAAGGTCGTG |
| | | GTAGTCTACTAACGTGTGGTGATGTAGAAGAAAATCCTGGACCTATG |
| | | tctGCCGGCGATATGAGAGCCGCTAACCTGTGGCCTTCTCCACTGAT |
| | | GATCAAGCGGAGCAAGAAGAACTCTCTGGCCCTGAGCCTGACCGCCG |
| | | ACCAGATGGTTTCTGCTCTGCTGGATGCCGAGCCTCCTATCCTGTAC |
| | | AGCGAGTACGACCCCACCAGACCTTTTAGCGAGGCCAGCATGATGGG |
| | | CCTGCTGACCAATCTGGCCGACAGAGAACTGGTGCACATGATCAACT |
| | | GGGCCAAGCGCGTGCCCGGCTTTGTGGATCTGACACTGCACGACCAA |
| | | GTGCATCTGCTGGAATGCGCCTGGATGGAAATCCTGATGATCGGCGT |
| | | CGTGTGGCGGAGCATGGAACACCCTGTGAAGCTGCTGTTCGCCCCTA |
| | | ACCTGCTGCTGGACAGAGATCAGGGCAAATGCGTGGAAGGACTGGTG |
| | | GAAATCTTCGACATGCTGCTGGCCACCAGCTCCAGATTCCGGATGAT |
| | | GAACCTGCAGGGCGAAGAGTTCGTGTGCCTGAAGTCCATCATCCTGC |
| | | TGAACAGCGGCGTGTACACCTTCCTGCCTAGCACACTGAAGTCCCTG |
| | | GAAGAGAAGGACCACATCCACAGAGTGCTGGATAAGATCACCGACAC |
| | | ACTGATCCACCTGATGGCCAAGGCCGGACTGACACTTCAGCAGCAGC |
| | | ATCAGAGACTGGCCCAGCTGCTGCTCATCCTGAGCCACATCAGACAC |
| | | ATGAGCAACAAAGGCATGGAACTGCTGTACTCCATGAAGTGCAAGAA |
| | | CGTGGTGCCCCTGTACGATCTGCTGCTCGAAGCCGCCGATGCTCACA |
| | | GACTGCATGCCCCTACATCTAGAGGCGGAGCCAGCGTGGAAGAGACA |
| | | GATCAGTCTCATCTGGCCACCGCCGGCAGCACAAGCTCTCATAGCCT |
| | | GCAGAAGTACTACATCACCGGCGAGGCCGAGGGATTTCCTGCTACTg |
| | | ctGGTGGCGGAGGATCTGGCGGAGGTGGAAGCGGCGGAGGCGGATCT |
| | | GTTGATGGCTTTGATGTGGGCGCCCTGGAAAGCCTGAGAGGCAATGC |
| | | CGATCTGGCCTACATCCTGTCCATGGAACCTTGCGGCCACTGCCTGA |
| | | TTATCAACAACGTGAACTTCTGTCGCGAGAGCGGCCTGAGAACCAGA |
| | | ACCGGCAGCAACATCGACTGCGAGAAGCTGCGGAGAAGATTCAGCAG |
| | | CCTGCACTTTATGGTGGAAGTGAAGGGCGATCTGACCGCCAAGAAAA |
| | | TGGTGCTGGCCCTGCTGGAACTGGCCAGACAAGATCATGGCGCTCTG |
| | | GACTGCTGCGTGGTCGTGATTCTGTCTCATGGCTGCCAGGCCAGCCA |
| | | TCTGCAATTCCCTGGTGCCGTGTATGGCACCGATGGCTGTCCTGTGT |
| | | CCGTGGAAAAGATCGTGAACATCTTCAACGGCACAAGCTGCCCTAGC |
| | | CTCGGCGGAAAGCCCAAGCTGTTCTTCATCCAAGCCTGTGGCGGCGA |
| | | GCAGAAGGATCACGGATTTGAGGTGGCCTCCACCTCTCCTGAGGATG |
| | | AGAGCCCTGGAAGCAACCCCGAGCCTGATGCCACACCTTTCCAAGAG |
| | | GGACTGAGAACCTTCGACCAGCTGGACGCTATCAGCTCCCTGCCTAC |
| | | ACCTAGCGACATCTTCGTGTCCTACAGCACATTCCCCGGCTTCGTGT |
| | | CTTGGAGGGATCCCAAGTCTGGCTCTTGGTACGTGGAAACCCTGGAC |
| | | GACATCTTTGAGCAGTGGGCCCATAGCGAGGACCTGCAGTCTCTGCT |
| | | TCTGAGAGTGGCCAATGCCGTGTCCGTGAAGGGAATCTACAAGCAGA |
| | | TGCCCGGCTGCTTCAACTTCCTGCGGAAGAAGCTGTTTTTCAAGACC |
| | | AGCGGCTCCGGCCAGTGCACAAATTATGCACTGCTGAAGCTCGCCGG |
| | | GGATGTCGAGAGTAACCCAGGACCTGGAAGCGGAGAAGGTCGTGGTA |
| | | GTCTACTAACGTGTGGTGATGTAGAAGAAAATCCTGGACCTatgacc |
| | | gagtacaagcccacggtgcgcctcgccacccgcgacgacgtccccag |
| | | ggccgtacgcaccctcgccgccgcgttcgccgactaccccgccacgc |
| | | gccacacegtcgatccggaccgccacatcgagcgggtcaccgagctg |
| | | caagaactcttcctcacgcgcgtcgggctcgacatcggcaaggtgtg |
| | | ggtcgcggacgacggcgccgcggtggcggtctggaccacgccggaga |
| | | gcgtcgaagcgggggcggtgttcgccgagatcggcccgcgcatggcc |
| | | gagttgagcggttcccggctggccgcgcagcaacagatggaaggcct |
| | | cctggcgccgcaccggcccaaggagcccgcgtggttcctggccaccg |
| | | tcggcgtctcgcccgaccaccagggcaagggtctgggcagegccgtc |
| | | gtgctccccggagtggaggcggccgagcgcgccggggtgcccgcctt |
| | | cctggagacctccgcgccccgcaacctccccttctacgagcggctcg |
| | | gcttcaccgtcaccgccgacgtcgaggtgcccgaaggaccgcgcacc |
| | | tggtgcatgacccgcaagcccggtgcc |
| | | (SEQ ID NO: 22) |
| Construct 10 | ERT2*mut41v2- | SAGDMRAANLWPSPLMIKRSKKNSLALSLTADQMVSALLDAEPPILY |
| | Casp9-Opt2A- | SEYDPTRPFSEASMMGLLTNLADREIVHMINWAKRVPGFVDLTLHDQ |
| | ERT2*mut41v2-Casp9- | VHLLECAWLEILMIGVVWRSMEHPVKLLFAPNLVLDRDQGKCVEGLV |
| | Opt2A-PuroR | EIFDMLLATSSRFRMMNLQGEEFVCLKSIILLNSGVYTFLPSTLKSL |
| | | EEKDHIHRVLDKITDTLIHLMAKAGLTLQQQHQRLAQLLLILSHIRH |
| | | ASNKGMELLYSMKCKNVVPLYDLLLEAADAHRLHAPTSRGGASVEET |
| | | DQSHLATAGSTSSHSLQKYYITGEAEGFPATAGGGGSGGGGSGGGGS |
| | | VDGFDVGALESLRGNADLAYILSMEPCGHCLIINNVNFCRESGLRTR |
| | | TGSNIDCEKLRRRFSSLHFMVEVKGDLTAKKMVLALLELARQDHGAL |
| | | DCCVVVILSHGCQASHLQFPGAVYGTDGCPVSVEKIVNIFNGTSCPS |
| | | LGGKPKLFFIQACGGEQKDHGFEVASTSPEDESPGSNPEPDATPFQE |
| | | GLRTFDQLDAISSLPTPSDIFVSYSTFPGFVSWRDPKSGSWYVETLD |
| | | DIFEQWAHSEDLQSLLLRVANAVSVKGIYKQMPGCFNFLRKKLFFKT |
| | | SGSGQCTNYALLKLAGDVESNPGPGSGEGRGSLLTCGDVEENPGPMS |
| | | AGDMRAANLWPSPLMIKRSKKNSLALSLTADQMVSALLDAEPPILYS |
| | | EYDPTRPFSEASMMGLLTNLADREIVHMINWAKRVPGFVDLTLHDQV |
| | | HLLECAWLEILMIGVVWRSMEHPVKLLFAPNLVLDRDQGKCVEGLVE |
| | | IFDMLLATSSRFRMMNLQGEEFVCLKSIILLNSGVYTFLPSTLKSLE |
| | | EKDHIHRVLDKITDTLIHLMAKAGLTLQQQHQRLAQLLLILSHIRHA |
| | | SNKGMELLYSMKCKNVVPLYDLLLEAADAHRLHAPTSRGGASVEETD |
| | | QSHLATAGSTSSHSLQKYYITGEAEGFPATAGGGGSGGGGSGGGGSV |
| | | DGFDVGALESLRGNADLAYILSMEPCGHCLIINNVNFCRESGLRTRT |
| | | GSNIDCEKLRRRFSSLHFMVEVKGDLTAKKMVLALLELARQDHGALD |
| | | CCVVVILSHGCQASHLQFPGAVYGTDGCPVSVEKIVNIFNGTSCPSL |
| | | GGKPKLFFIQACGGEQKDHGFEVASTSPEDESPGSNPEPDATPFQEG |
| | | LRTFDQLDAISSLPTPSDIFVSYSTFPGFVSWRDPKSGSWYVETLDD |
| | | IFEQWAHSEDLQSLLLRVANAVSVKGIYKQMPGCFNFLRKKLFFKTS |
| | | GSGQCTNYALLKLAGDVESNPGPGSGEGRGSLLTCGDVEENPGPMTE |
| | | YKPTVRLATRDDVPRAVRTLAAAFADYPATRHTVDPDRHIERVTELQ |
| | | ELFLTRVGLDIGKVWVADDGAAVAVWTTPESVEAGAVFAEIGPRMAE |
| | | LSGSRLAAQQQMEGLLAPHRPKEPAWFLATVGVSPDHQGKGLGSAVV |
| | | LPGVEAAERAGVPAFLETSAPRNLPFYERLGFTVTADVEVPEGPRTW |
| | | CMTRKPGA |
| | | (SEQ ID NO: 23) |
| | | Bold = ERT2*mut41 |
| | | Italics = linker |
| | | Plain = Casp9 |
| | | GSGQCTNYALLKLAGDVESNPGPGSGEGRGSLLTCGDVEENPGP |
| | | (SEQ ID NO: 61) = Opt2A linker |
| | | MTEYKPTVRLATRDDVPRAVRTLAAAFADYPATRHTVDPDRHIERVT |
| | | ELQELFLTRVGLDIGKVWVADDGAAVAVWTTPESVEAGAVFAEIGPR |
| | | MAELSGSRLAAQQQMEGLLAPHRPKEPAWFLATVGVSPDHQGKGLGS |
| | | AVVLPGVEAAERAGVPAFLETSAPRNLPFYERLGFTVTADVEVPEGP |
| | | RTWCMTRKPGA |
| | | (SEQ ID NO: 62) = PuroR |
| | | Nucleotide sequence: |
| | | ATGtctgctggagacatgagagctgccaacctttggccaagcccgct |
| | | catgatcaaacgctctaagaagaacagcctggccttgtccctgacgg |
| | | ccgaccagatggtcagtgccttgttggatgctgagccccccatactc |
| | | tattccgagtatgatcctaccagacccttcagtgaagcttcgatgat |
| | | gggcttactgaccaacctggcagacagggagATCgttcacatgatca |
| | | actgggcgaagagggtgccaggctttgtggatttgaccctccatgat |
| | | caggtccaccttctagaatgtgcctggCTAgagatcctgatgattgg |
| | | tGTGgtctggcgctccatggagcacccagtgaagctactgtttgctc |
| | | ctaacttgGTGttggacagggacCAGggaaaatgtgtagagggcCTG |
| | | GTGGAGATCTTCGACATGCTGCTGGCTACATCATCTCGGTTCCGCAT |
| | | GATGAATCTGCAGGGAGAGGAGTTTGTGTGCCTCAAATCTATTATTT |
| | | TGCTTAATTCTGGAGTGTACACATTTCTGCCCAGCACCCTGAAGTCT |
| | | CTGGAAGAGAAGGACCATATCCACCGAGTCCTGGACAAGATCACAGA |
| | | CACTTTGATCCACCTGATGGCCAAGGCAGGCCTGACCCTGCAGCAGC |
| | | AGCACCAGCGGCTGGCCCAGCTCCTCCTCATCCTCTCCCACATCAGG |
| | | CACGCCagtaacaaaggcatggagctgctgtacagcatgaagtgcaa |
| | | gaacgtggtgcccctctatGaCctgctgctggaggcggcggacgccc |
| | | accgcctacatgcgcccactagccgtggaggggcatccgtggaggag |
| | | acggaccaaagccacttggccactgcgggctctacttcatcgcattc |
| | | cttgcaaaagtattacatcacgggggaggcagagggtttccctgcca |
| | | cagctgggggtggaggttcagggggtggaggttcaggtggtggcggt |
| | | agtgtcgatggcttcgatgtcggtgctcttgagagtttgaggggaaa |
| | | tgcagatttggcttacatcctgagcatggagccctgtggccactgcc |
| | | tcattatcaacaatgtgaacttctgccgtgagtccgggctccgcacc |
| | | cgcactggctccaacatcgactgtgagaagttgcggcgtcgcttctc |
| | | ctcgctgcatttcatggtggaggtgaagggcgacctgactgccaaga |
| | | aaatggtgctggctttgctggagctggcgcggcaggaccacggtgct |
| | | ctggactgctgcgtggtggtcattctctctcacggctgtcaggccag |
| | | ccacctgcagttcccaggggctgtctacggcacagatggatgccctg |
| | | tgtcggtcgagaagattgtgaacatcttcaatgggaccagctgcccc |
| | | agcctgggagggaagcccaagctctttttcatccaggcctgtggtgg |
| | | ggagcagaaagaccatgggtttgaggtggcctccacttcccctgaag |
| | | acgagtcccctggcagtaaccccgagccagatgccaccccgttccag |
| | | gaaggtttgaggaccttcgaccagctggacgccatatctagtttgcc |
| | | cacacccagtgacatctttgtgtcctactctactttcccaggttttg |
| | | tttcctggagggaccccaagagtggctcctggtacgttgagaccctg |
| | | gacgacatctttgagcagtgggctcactctgaagacctgcagtccct |
| | | cctgcttagggtcgctaatgctgtttcggtgaaagggatttataaac |
| | | agatgcctggttgctttaatttcctccggaaaaaacttttctttaaa |
| | | acatcaGGCTCCGGCCAGTGCACAAATTATGCACTGCTGAAGCTCGC |
| | | CGGGGATGTCGAGAGTAACCCAGGACCTGGAAGCGGAGAAGGTCGTG |
| | | GTAGTCTACTAACGTGTGGTGATGTAGAAGAAAATCCTGGACCTATG |
| | | TCTGCCGGCGATATGAGAGCCGCCAACCTGTGGCCTTCTCCACTGAT |
| | | GATCAAGCGGAGCAAGAAGAACTCTCTGGCCCTGAGCCTGACCGCCG |
| | | ACCAGATGGTTTCTGCTCTGCTGGATGCCGAGCCTCCTATCCTGTAC |
| | | AGCGAGTACGACCCCACCAGACCTTTTAGCGAGGCCAGCATGATGGG |
| | | CCTGCTGACCAATCTGGCCGACCGGGAAATCGTGCACATGATCAACT |
| | | GGGCCAAGCGCGTGCCCGGCTTTGTGGATCTGACACTGCACGACCAA |
| | | GTGCATCTGCTGGAATGCGCCTGGCTGGAAATCCTGATGATCGGCGT |
| | | TGTGTGGCGGAGCATGGAACACCCTGTGAAGCTGCTGTTCGCCCCTA |
| | | ACCTGGTGCTGGACAGAGATCAGGGCAAATGCGTGGAAGGACTGGTG |
| | | GAAATCTTCGACATGCTGCTGGCCACCAGCTCCAGATTCCGGATGAT |
| | | GAACCTGCAGGGCGAAGAGTTCGTGTGCCTGAAGTCCATCATCCTGC |
| | | TGAACAGCGGCGTGTACACCTTCCTGCCTAGCACACTGAAGTCCCTG |
| | | GAAGAGAAGGACCACATCCACAGAGTGCTGGATAAGATCACCGACAC |
| | | ACTGATCCACCTGATGGCCAAGGCCGGACTGACACTCCAGCAGCAGC |
| | | ATCAGAGACTGGCCCAGCTGCTGCTCATCCTGAGCCACATTAGACAC |
| | | GCCAGCAACAAAGGCATGGAACTGCTGTACTCCATGAAGTGCAAGAA |
| | | CGTGGTGCCCCTGTACGACCTGCTGCTCGAAGCTGCCGATGCTCACA |
| | | GACTGCACGCCCCTACATCTAGAGGCGGAGCCTCTGTGGAAGAGACA |
| | | GACCAGTCTCATCTGGCCACAGCCGGCAGCACATCTTCTCACAGCCT |
| | | GCAGAAGTACTACATCACCGGCGAGGCCGAGGGATTTCCTGCTACAG |
| | | CTGGCGGCGGAGGATCTGGCGGAGGTGGAAGCGGAGGCGGTGGAAGT |
| | | GTGGATGGCTTTGATGTGGGCGCCCTGGAAAGCCTGAGAGGCAATGC |
| | | CGATCTGGCCTACATCCTGTCCATGGAACCTTGCGGCCACTGCCTGA |
| | | TTATCAACAACGTGAACTTCTGTCGCGAGAGCGGCCTGAGAACCAGA |
| | | ACCGGCAGCAATATCGACTGCGAGAAGCTGCGGAGAAGATTCAGCAG |
| | | CCTGCACTTTATGGTGGAAGTCAAGGGCGATCTGACCGCCAAGAAAA |
| | | TGGTGCTGGCCCTGCTGGAACTGGCCAGACAAGATCATGGCGCTCTG |
| | | GACTGCTGCGTGGTCGTGATTCTGTCTCATGGCTGCCAGGCCAGCCA |
| | | TCTGCAATTCCCTGGTGCCGTGTATGGCACCGATGGCTGTCCTGTGT |
| | | CCGTGGAAAAGATCGTGAACATCTTCAACGGCACAAGCTGCCCTAGC |
| | | CTCGGCGGAAAGCCCAAGCTGTTCTTCATCCAAGCCTGTGGCGGCGA |
| | | GCAGAAGGATCACGGATTTGAGGTGGCCTCCACCTCTCCTGAGGATG |
| | | AGAGCCCTGGAAGCAACCCCGAGCCTGATGCCACACCTTTCCAAGAG |
| | | GGACTGAGAACCTTCGACCAGCTGGACGCTATCAGCTCCCTGCCTAC |
| | | ACCTAGCGACATCTTCGTGTCCTACAGCACATTCCCCGGCTTCGTGT |
| | | CTTGGAGGGATCCCAAGTCTGGCTCTTGGTACGTGGAAACCCTGGAC |
| | | GACATCTTTGAGCAGTGGGCCCATAGCGAGGACCTGCAGTCTCTGCT |
| | | TCTGAGAGTGGCCAATGCCGTGTCCGTGAAGGGCATCTACAAGCAGA |
| | | TGCCCGGCTGCTTCAACTTCCTGCGGAAGAAGCTGTTTTTCAAGACC |
| | | AGCGGCTCCGGCCAGTGCACAAATTATGCACTGCTGAAGCTCGCCGG |
| | | GGATGTCGAGAGTAACCCAGGACCTGGAAGCGGAGAAGGTCGTGGTA |
| | | GTCTACTAACGTGTGGTGATGTAGAAGAAAATCCTGGACCTatgacc |
| | | gagtacaagcccacggtgcgcctcgccacccgcgacgacgtccccag |
| | | ggccgtacgcaccctcgccgccgcgttcgccgactaccccgccacgc |
| | | gccacaccgtcgatccggaccgccacatcgagcgggtcaccgagctg |
| | | caagaactcttcctcacgcgcgtcgggctcgacatcggcaaggtgtg |
| | | ggtcgcggacgacggcgccgcggtggcggtctggaccacgccggaga |
| | | gcgtcgaagcgggggcggtgttcgccgagatcggcccgcgcatggcc |
| | | gagttgagcggttcccggctggccgcgcagcaacagatggaaggcct |
| | | cctggcgccgcaccggcccaaggagcccgcgtggttcctggccaccg |
| | | teggcgtctegcccgaccaccagggcaagggtctgggcagcgccgtc |
| | | gtgctccccggagtggaggcggccgagcgcgccggggtgcccgcctt |
| | | cctggagacctccgcgccccgcaacctccccttctacgagcggctcg |
| | | gcttcaccgtcaccgccgacgtcgaggtgcccgaaggaccgcgcacc |
| | | tggtgcatgacccgcaagcccggtgcc |
| | | (SEQ ID NO: 24) |
| Construct 11 | ERT2*mut81-Casp9- | SAGDMRAANLWPSPLMIKRSKKNSLALSLTADQMVSALLDAEPPILY |
| | Opt2A_2.0- | SEYDPTRPFSEASMMGLLTNLADRELVHMINWAKRVPGFVDLTLHDQ |
| | ERT2*mut81-Casp9- | VHLLECAWMEILMIGVVWRSMEHPVKLLFAPNLLLDRDQGKCVEGLV |
| | Opt2A-PuroR | EIFDMLLATSSRFRMMNLQGEEFVCLKSIILLNSGVYTFLPSTLKSL |
| | | EEKDHIHRVLDKITDTLIHLMAKAGLTLQQQHQRLAQLLLILSHIRH |
| | | MSNKGMELLYSMKCKNVVPLYDLLLEAADAHRLHAPTSRGGASVEET |
| | | DQSHLATAGSTSSHSLQKYYITGEAEGFPATAGGGGSGGGGSGGGGS |
| | | VDGFDVGALESLRGNADLAYILSMEPCGHCLIINNVNFCRESGLRTR |
| | | TGSNIDCEKLRRRFSSLHFMVEVKGDLTAKKMVLALLELARQDHGAL |
| | | DCCVVVILSHGCQASHLQFPGAVYGTDGCPVSVEKIVNIFNGTSCPS |
| | | LGGKPKLFFIQACGGEQKDHGFEVASTSPEDESPGSNPEPDATPFQE |
| | | GLRTFDQLDAISSLPTPSDIFVSYSTFPGFVSWRDPKSGSWYVETLD |
| | | DIFEQWAHSEDLQSLLLRVANAVSVKGIYKQMPGCFNFLRKKLFFKT |
| | | SGSGATNFSLLKQAGDVEENPGPGSGEGRGSLLTCGDVEENPGPMSA |
| | | GDMRAANLWPSPLMIKRSKKNSLALSLTADQMVSALLDAEPPILYSE |
| | | YDPTRPFSEASMMGLLTNLADRELVHMINWAKRVPGFVDLTLHDQVH |
| | | LLECAWMEILMIGVVWRSMEHPVKLLFAPNLLLDRDQGKCVEGLVEI |
| | | FDMLLATSSRFRMMNLQGEEFVCLKSIILLNSGVYTFLPSTLKSLEE |
| | | KDHIHRVLDKITDTLIHLMAKAGLTLQQQHQRLAQLLLILSHIRHMS |
| | | NKGMELLYSMKCKNVVPLYDLLLEAADAHRLHAPTSRGGASVEETDQ |
| | | SHLATAGSTSSHSLQKYYITGEAEGFPATAGGGGSGGGGSGGGGSVD |
| | | GFDVGALESLRGNADLAYILSMEPCGHCLIINNVNFCRESGLRTRTG |
| | | SNIDCEKLRRRFSSLHFMVEVKGDLTAKKMVLALLELARQDHGALDC |
| | | CVVVILSHGCQASHLQFPGAVYGTDGCPVSVEKIVNIFNGTSCPSLG |
| | | GKPKLFFIQACGGEQKDHGFEVASTSPEDESPGSNPEPDATPFQEGL |
| | | RTFDQLDAISSLPTPSDIFVSYSTFPGFVSWRDPKSGSWYVETLDDI |
| | | FEQWAHSEDLQSLLLRVANAVSVKGIYKQMPGCFNFLRKKLFFKTSG |
| | | SGQCTNYALLKLAGDVESNPGPGSGEGRGSLLTCGDVEENPGPMTEY |
| | | KPTVRLATRDDVPRAVRTLAAAFADYPATRHTVDPDRHIERVTELQE |
| | | LFLTRVGLDIGKVWVADDGAAVAVWTTPESVEAGAVFAEIGPRMAEL |
| | | SGSRLAAQQQMEGLLAPHRPKEPAWFLATVGVSPDHQGKGLGSAVVL |
| | | PGVEAAERAGVPAFLETSAPRNLPFYERLGFTVTADVEVPEGPRTWC |
| | | MTRKPGA |
| | | (SEQ ID NO: 25) |
| | | Bold = ERT2*mut81 |
| | | Italics = linker |
| | | Plain = Casp9 |
| | | GSGATNFSLLKQAGDVEENPGPGSGEGRGSLLTCGDVEENPGP |
| | | (SEQ ID NO: 63) = Opt2A_2.0 linker |
| | | GSGQCTNYALLKLAGDVESNPGPGSGEGRGSLLTCGDVEENPGP |
| | | (SEQ ID NO: 61) = Opt2A linker |
| | | MTEYKPTVRLATRDDVPRAVRTLAAAFADYPATRHTVDPDRHIERVT |
| | | ELQELFLTRVGLDIGKVWVADDGAAVAVWTTPESVEAGAVFAEIGPR |
| | | MAELSGSRLAAQQQMEGLLAPHRPKEPAWFLATVGVSPDHQGKGLGS |
| | | AVVLPGVEAAERAGVPAFLETSAPRNLPFYERLGFTVTADVEVPEGP |
| | | RTWCMTRKPGA |
| | | (SEQ ID NO: 62) = PuroR |
| | | Nucleotide sequence: |
| | | ATGtctgctggagacatgagagctgccaacctttggccaagcccgct |
| | | catgatcaaacgctctaagaagaacagcctggccttgtccctgacgg |
| | | ccgaccagatggtcagtgccttgttggatgctgagccccccatactc |
| | | tattccgagtatgatcctaccagacccttcagtgaagcttcgatgat |
| | | gggcttactgaccaacctggcagacagggagCTGGTTCACATGATCA |
| | | ACTGGGCGAAGAGGGTGCCAGGCTTTGTGGATTTGACCCTCCATGAT |
| | | CAGGTCCACCTTCTAGAATGTGCCTGGATGGAGATCCTGATGATTGG |
| | | TGTGGTCTGGCGCTCCATGGAGCACCCAGTGAAGCTACTGTTTGCTC |
| | | CTAACTTGCTCTTGGACAGGGACCAGGGAAAATGTGTAGAGGGCCTG |
| | | GTGGAGATCTTCGACATGCTGCTGGCTACATCATCTCGGTTCCGCAT |
| | | GATGAATCTGCAGGGAGAGGAGTTTGTGTGCCTCAAATCTATTATTT |
| | | TGCTTAATTCTGGAGTGTACACATTTCTGCCCAGCACCCTGAAGTCT |
| | | CTGGAAGAGAAGGACCATATCCACCGAGTCCTGGACAAGATCACAGA |
| | | CACTTTGATCCACCTGATGGCCAAGGCAGGCCTGACCCTGCAGCAGC |
| | | AGCACCAGCGGCTGGCCCAGCTCCTCCTCATCCTCTCCCACATCAGG |
| | | CACATGAGTAACAAAGGCATGGAGCTGctgtacagcatgaagtgcaa |
| | | gaacgtggtgcccctctatGaCctgctgctggaggcggcggacgccc |
| | | accgcctacatgcgcccactagccgtggaggggcatccgtggaggag |
| | | acggaccaaagccacttggccactgcgggctctacttcatcgcattc |
| | | cttgcaaaagtattacatcacgggggaggcagagggtttccctgcca |
| | | cagctgggggtggaggttcagggggtggaggttcaggtggtggcggt |
| | | agtgtcgatggcttcgatgtcggtgctcttgagagtttgaggggaaa |
| | | tgcagatttggcttacatcctgagcatggagccctgtggccactgcc |
| | | tcattatcaacaatgtgaacttctgccgtgagtccgggctccgcacc |
| | | cgcactggctccaacatcgactgtgagaagttgcggcgtcgcttctc |
| | | ctcgctgcatttcatggtggaggtgaagggcgacctgactgccaaga |
| | | aaatggtgctggctttgctggagctggcgcggcaggaccacggtgct |
| | | ctggactgctgcgtggtggtcattctctctcacggctgtcaggccag |
| | | ccacctgcagttcccaggggctgtctacggcacagatggatgccctg |
| | | tgtcggtcgagaagattgtgaacatcttcaatgggaccagctgcccc |
| | | agcctgggagggaagcccaagctctttttcatccaggcctgtggtgg |
| | | ggagcagaaagaccatgggtttgaggtggcctccacttcccctgaag |
| | | acgagtcccctggcagtaaccccgagccagatgccaccccgttccag |
| | | gaaggtttgaggaccttcgaccagctggacgccatatctagtttgcc |
| | | cacacccagtgacatctttgtgtcctactctactttcccaggttttg |
| | | tttcctggagggaccccaagagtggctcctggtacgttgagaccctg |
| | | gacgacatctttgagcagtgggctcactctgaagacctgcagtccct |
| | | cctgcttagggtcgctaatgctgtttcggtgaaagggatttataaac |
| | | agatgcctggttgctttaatttcctccggaaaaaacttttctttaaa |
| | | acatcaGGCTCCGGCGCGACGAATTTTAGTCTACTGAAACAAGCGGG |
| | | AGACGTGGAGGAAAACCCTGGACCTGGAAGCGGAGAAGGTCGTGGTA |
| | | GTCTACTAACGTGTGGTGATGTAGAAGAAAATCCTGGACCTATGtct |
| | | GCCGGCGATATGAGAGCCGCTAACCTGTGGCCTTCTCCACTGATGAT |
| | | CAAGCGGAGCAAGAAGAACTCTCTGGCCCTGAGCCTGACCGCCGACC |
| | | AGATGGTTTCTGCTCTGCTGGATGCCGAGCCTCCTATCCTGTACAGC |
| | | GAGTACGACCCCACCAGACCTTTTAGCGAGGCCAGCATGATGGGCCT |
| | | GCTGACCAATCTGGCCGACAGAGAACTGGTGCACATGATCAACTGGG |
| | | CCAAGCGCGTGCCCGGCTTTGTGGATCTGACACTGCACGACCAAGTG |
| | | CATCTGCTGGAATGCGCCTGGATGGAAATCCTGATGATCGGCGTCGT |
| | | GTGGCGGAGCATGGAACACCCTGTGAAGCTGCTGTTCGCCCCTAACC |
| | | TGCTGCTGGACAGAGATCAGGGCAAATGCGTGGAAGGACTGGTGGAA |
| | | ATCTTCGACATGCTGCTGGCCACCAGCTCCAGATTCCGGATGATGAA |
| | | CCTGCAGGGCGAAGAGTTCGTGTGCCTGAAGTCCATCATCCTGCTGA |
| | | ACAGCGGCGTGTACACCTTCCTGCCTAGCACACTGAAGTCCCTGGAA |
| | | GAGAAGGACCACATCCACAGAGTGCTGGATAAGATCACCGACACACT |
| | | GATCCACCTGATGGCCAAGGCCGGACTGACACTTCAGCAGCAGCATC |
| | | AGAGACTGGCCCAGCTGCTGCTCATCCTGAGCCACATCAGACACATG |
| | | AGCAACAAAGGCATGGAACTGCTGTACTCCATGAAGTGCAAGAACGT |
| | | GGTGCCCCTGTACGATCTGCTGCTCGAAGCCGCCGATGCTCACAGAC |
| | | TGCATGCCCCTACATCTAGAGGCGGAGCCAGCGTGGAAGAGACAGAT |
| | | CAGTCTCATCTGGCCACCGCCGGCAGCACAAGCTCTCATAGCCTGCA |
| | | GAAGTACTACATCACCGGCGAGGCCGAGGGATTTCCTGCTACTgctG |
| | | GTGGCGGAGGATCTGGCGGAGGTGGAAGCGGCGGAGGCGGATCTGTT |
| | | GATGGCTTTGATGTGGGCGCCCTGGAAAGCCTGAGAGGCAATGCCGA |
| | | TCTGGCCTACATCCTGTCCATGGAACCTTGCGGCCACTGCCTGATTA |
| | | TCAACAACGTGAACTTCTGTCGCGAGAGCGGCCTGAGAACCAGAACC |
| | | GGCAGCAACATCGACTGCGAGAAGCTGCGGAGAAGATTCAGCAGCCT |
| | | GCACTTTATGGTGGAAGTGAAGGGCGATCTGACCGCCAAGAAAATGG |
| | | TGCTGGCCCTGCTGGAACTGGCCAGACAAGATCATGGCGCTCTGGAC |
| | | TGCTGCGTGGTCGTGATTCTGTCTCATGGCTGCCAGGCCAGCCATCT |
| | | GCAATTCCCTGGTGCCGTGTATGGCACCGATGGCTGTCCTGTGTCCG |
| | | TGGAAAAGATCGTGAACATCTTCAACGGCACAAGCTGCCCTAGCCTC |
| | | GGCGGAAAGCCCAAGCTGTTCTTCATCCAAGCCTGTGGCGGCGAGCA |
| | | GAAGGATCACGGATTTGAGGTGGCCTCCACCTCTCCTGAGGATGAGA |
| | | GCCCTGGAAGCAACCCCGAGCCTGATGCCACACCTTTCCAAGAGGGA |
| | | CTGAGAACCTTCGACCAGCTGGACGCTATCAGCTCCCTGCCTACACC |
| | | TAGCGACATCTTCGTGTCCTACAGCACATTCCCCGGCTTCGTGTCTT |
| | | GGAGGGATCCCAAGTCTGGCTCTTGGTACGTGGAAACCCTGGACGAC |
| | | ATCTTTGAGCAGTGGGCCCATAGCGAGGACCTGCAGTCTCTGCTTCT |
| | | GAGAGTGGCCAATGCCGTGTCCGTGAAGGGAATCTACAAGCAGATGC |
| | | CCGGCTGCTTCAACTTCCTGCGGAAGAAGCTGTTTTTCAAGACCAGC |
| | | GGCTCCGGCCAGTGCACAAATTATGCACTGCTGAAGCTCGCCGGGGA |
| | | TGTCGAGAGTAACCCAGGACCTGGAAGCGGAGAAGGTCGTGGTAGTC |
| | | TACTAACGTGTGGTGATGTAGAAGAAAATCCTGGACCTatgaccgag |
| | | tacaagcccacggtgcgcctcgccacccgcgacgacgtccccagggc |
| | | cgtacgcaccctcgccgccgcgttcgccgactaccccgccacgcgcc |
| | | acacegtcgatccggaccgccacatcgagcgggtcaccgagctgcaa |
| | | gaactcttcctcacgcgcgtcgggctcgacatcggcaaggtgtgggt |
| | | cgcggacgacggcgccgcggtggcggtctggaccacgccggagagcg |
| | | tcgaagcgggggcggtgttcgccgagatcggcccgcgcatggccgag |
| | | ttgagcggttcccggctggccgcgcagcaacagatggaaggcctcct |
| | | ggcgccgcaccggcccaaggagcccgcgtggttcctggccaccgtcg |
| | | gcgtctcgcccgaccaccagggcaagggtctgggcagcgccgtcgtg |
| | | ctccccggagtggaggcggccgagcgcgccggggtgcccgccttcct |
| | | ggagacctccgegccccgcaacctccccttctacgagcggctcggct |
| | | tcaccgtcaccgccgacgtcgaggtgcccgaaggaccgcgcacctgg |
| | | tgcatgacccgcaagcccggtgcc |
| | | (SEQ ID NO: 26) |
| Construct 12 | ERT2*mut41v2-Casp9- | SAGDMRAANLWPSPLMIKRSKKNSLALSLTADQMVSALLDAEPPILY |
| | Opt2A_2.0- | SEYDPTRPFSEASMMGLLTNLADREIVHMINWAKRVPGFVDLTLHDQ |
| | ERT2*mut41v2- | VHLLECAWLEILMIGVVWRSMEHPVKLLFAPNLVLDRDQGKCVEGLV |
| | Casp9-Opt2A-PuroR | EIFDMLLATSSRFRMMNLQGEEFVCLKSIILLNSGVYTFLPSTLKSL |
| | | EEKDHIHRVLDKITDTLIHLMAKAGLTLQQQHQRLAQLLLILSHIRH |
| | | ASNKGMELLYSMKCKNVVPLYDLLLEAADAHRLHAPTSRGGASVEET |
| | | DQSHLATAGSTSSHSLQKYYITGEAEGFPATAGGGGSGGGGSGGGGS |
| | | VDGFDVGALESLRGNADLAYILSMEPCGHCLIINNVNFCRESGLRTR |
| | | TGSNIDCEKLRRRFSSLHFMVEVKGDLTAKKMVLALLELARQDHGAL |
| | | DCCVVVILSHGCQASHLQFPGAVYGTDGCPVSVEKIVNIFNGTSCPS |
| | | LGGKPKLFFIQACGGEQKDHGFEVASTSPEDESPGSNPEPDATPFQE |
| | | GLRTFDQLDAISSLPTPSDIFVSYSTFPGFVSWRDPKSGSWYVETLD |
| | | DIFEQWAHSEDLQSLLLRVANAVSVKGIYKQMPGCFNFLRKKLFFKT |
| | | SGSGATNFSLLKQAGDVEENPGPGSGEGRGSLLTCGDVEENPGPMSA |
| | | GDMRAANLWPSPLMIKRSKKNSLALSLTADQMVSALLDAEPPILYSE |
| | | YDPTRPFSEASMMGLLTNLADREIVHMINWAKRVPGFVDLTLHDQVH |
| | | LLECAWLEILMIGVVWRSMEHPVKLLFAPNLVLDRDQGKCVEGLVEI |
| | | FDMLLATSSRFRMMNLQGEEFVCLKSIILLNSGVYTFLPSTLKSLEE |
| | | KDHIHRVLDKITDTLIHLMAKAGLTLQQQHQRLAQLLLILSHIRHAS |
| | | NKGMELLYSMKCKNVVPLYDLLLEAADAHRLHAPTSRGGASVEETDQ |
| | | SHLATAGSTSSHSLQKYYITGEAEGFPATAGGGGSGGGGSGGGGSVD |
| | | GFDVGALESLRGNADLAYILSMEPCGHCLIINNVNFCRESGLRTRTG |
| | | SNIDCEKLRRRFSSLHFMVEVKGDLTAKKMVLALLELARQDHGALDC |
| | | CVVVILSHGCQASHLQFPGAVYGTDGCPVSVEKIVNIFNGTSCPSLG |
| | | GKPKLFFIQACGGEQKDHGFEVASTSPEDESPGSNPEPDATPFQEGL |
| | | RTFDQLDAISSLPTPSDIFVSYSTFPGFVSWRDPKSGSWYVETLDDI |
| | | FEQWAHSEDLQSLLLRVANAVSVKGIYKQMPGCFNFLRKKLFFKTSG |
| | | SGQCTNYALLKLAGDVESNPGPGSGEGRGSLLTCGDVEENPGPMTEY |
| | | KPTVRLATRDDVPRAVRTLAAAFADYPATRHTVDPDRHIERVTELQE |
| | | LFLTRVGLDIGKVWVADDGAAVAVWTTPESVEAGAVFAEIGPRMAEL |
| | | SGSRLAAQQQMEGLLAPHRPKEPAWFLATVGVSPDHQGKGLGSAVVL |
| | | PGVEAAERAGVPAFLETSAPRNLPFYERLGFTVTADVEVPEGPRTWC |
| | | MTRKPGA |
| | | (SEQ ID NO: 27) |
| | | Bold = ERT2*mut41v2 |
| | | Italics = linker |
| | | Plain = Casp9 |
| | | GSGATNFSLLKQAGDVEENPGPGSGEGRGSLLTCGDVEENPGP |
| | | (SEQ ID NO: 63) = Opt2A_2.0 linker |
| | | GSGQCTNYALLKLAGDVESNPGPGSGEGRGSLLTCGDVEENPGP |
| | | (SEQ ID NO: 61) = Opt2A linker |
| | | MTEYKPTVRLATRDDVPRAVRTLAAAFADYPATRHTVDPDRHIERVT |
| | | ELQELFLTRVGLDIGKVWVADDGAAVAVWTTPESVEAGAVFAEIGPR |
| | | MAELSGSRLAAQQQMEGLLAPHRPKEPAWFLATVGVSPDHQGKGLGS |
| | | AVVLPGVEAAERAGVPAFLETSAPRNLPFYERLGFTVTADVEVPEGP |
| | | RTWCMTRKPGA |
| | | (SEQ ID NO: 62) = PuroR |
| | | Nucleotide sequence: |
| | | ATGtctgctggagacatgagagctgccaacctttggccaagcccgct |
| | | catgatcaaacgctctaagaagaacagcctggccttgtccctgacgg |
| | | ccgaccagatggtcagtgccttgttggatgctgagccccccatactc |
| | | tattccgagtatgatcctaccagacccttcagtgaagcttcgatgat |
| | | gggcttactgaccaacctggcagacagggagATCgttcacatgatca |
| | | actgggcgaagagggtgccaggctttgtggatttgaccctccatgat |
| | | caggtccaccttctagaatgtgcctggCTAgagatcctgatgattgg |
| | | tGTGgtctggcgctccatggagcacccagtgaagctactgtttgctc |
| | | ctaacttgGTGttggacagggacCAGggaaaatgtgtagagggcCTG |
| | | GTGGAGATCTTCGACATGCTGCTGGCTACATCATCTCGGTTCCGCAT |
| | | GATGAATCTGCAGGGAGAGGAGTTTGTGTGCCTCAAATCTATTATTT |
| | | TGCTTAATTCTGGAGTGTACACATTTCTGCCCAGCACCCTGAAGTCT |
| | | CTGGAAGAGAAGGACCATATCCACCGAGTCCTGGACAAGATCACAGA |
| | | CACTTTGATCCACCTGATGGCCAAGGCAGGCCTGACCCTGCAGCAGC |
| | | AGCACCAGCGGCTGGCCCAGCTCCTCCTCATCCTCTCCCACATCAGG |
| | | CACGCCagtaacaaaggcatggagctgctgtacagcatgaagtgcaa |
| | | gaacgtggtgcccctctatGaCctgctgctggaggcggcggacgccc |
| | | accgcctacatgcgcccactagccgtggaggggcatccgtggaggag |
| | | acggaccaaagccacttggccactgcgggctctacttcatcgcattc |
| | | cttgcaaaagtattacatcacgggggaggcagagggtttccctgcca |
| | | cagctgggggtggaggttcagggggtggaggttcaggtggtggcggt |
| | | agtgtcgatggcttcgatgtcggtgctcttgagagtttgaggggaaa |
| | | tgcagatttggcttacatcctgagcatggagccctgtggccactgcc |
| | | tcattatcaacaatgtgaacttctgccgtgagtccgggctccgcacc |
| | | cgcactggctccaacatcgactgtgagaagttgcggcgtcgcttctc |
| | | ctcgctgcatttcatggtggaggtgaagggcgacctgactgccaaga |
| | | aaatggtgctggctttgctggagctggcgcggcaggaccacggtgct |
| | | ctggactgctgcgtggtggtcattctctctcacggctgtcaggccag |
| | | ccacctgcagttcccaggggctgtctacggcacagatggatgccctg |
| | | tgtcggtcgagaagattgtgaacatcttcaatgggaccagctgcccc |
| | | agcctgggagggaagcccaagctctttttcatccaggcctgtggtgg |
| | | ggagcagaaagaccatgggtttgaggtggcctccacttcccctgaag |
| | | acgagtcccctggcagtaaccccgagccagatgccaccccgttccag |
| | | gaaggtttgaggaccttcgaccagctggacgccatatctagtttgcc |
| | | cacacccagtgacatctttgtgtcctactctactttcccaggttttg |
| | | tttcctggagggaccccaagagtggctcctggtacgttgagaccctg |
| | | gacgacatctttgagcagtgggctcactctgaagacctgcagtccct |
| | | cctgcttagggtcgctaatgctgtttcggtgaaagggatttataaac |
| | | agatgcctggttgctttaatttcctccggaaaaaacttttctttaaa |
| | | acatcaGGCTCCGGCGCGACGAATTTTAGTCTACTGAAACAAGCGGG |
| | | AGACGTGGAGGAAAACCCTGGACCTGGAAGCGGAGAAGGTCGTGGTA |
| | | GTCTACTAACGTGTGGTGATGTAGAAGAAAATCCTGGACCTATGTCT |
| | | GCCGGCGATATGAGAGCCGCCAACCTGTGGCCTTCTCCACTGATGAT |
| | | CAAGCGGAGCAAGAAGAACTCTCTGGCCCTGAGCCTGACCGCCGACC |
| | | AGATGGTTTCTGCTCTGCTGGATGCCGAGCCTCCTATCCTGTACAGC |
| | | GAGTACGACCCCACCAGACCTTTTAGCGAGGCCAGCATGATGGGCCT |
| | | GCTGACCAATCTGGCCGACCGGGAAATCGTGCACATGATCAACTGGG |
| | | CCAAGCGCGTGCCCGGCTTTGTGGATCTGACACTGCACGACCAAGTG |
| | | CATCTGCTGGAATGCGCCTGGCTGGAAATCCTGATGATCGGCGTTGT |
| | | GTGGCGGAGCATGGAACACCCTGTGAAGCTGCTGTTCGCCCCTAACC |
| | | TGGTGCTGGACAGAGATCAGGGCAAATGCGTGGAAGGACTGGTGGAA |
| | | ATCTTCGACATGCTGCTGGCCACCAGCTCCAGATTCCGGATGATGAA |
| | | CCTGCAGGGCGAAGAGTTCGTGTGCCTGAAGTCCATCATCCTGCTGA |
| | | ACAGCGGCGTGTACACCTTCCTGCCTAGCACACTGAAGTCCCTGGAA |
| | | GAGAAGGACCACATCCACAGAGTGCTGGATAAGATCACCGACACACT |
| | | GATCCACCTGATGGCCAAGGCCGGACTGACACTCCAGCAGCAGCATC |
| | | AGAGACTGGCCCAGCTGCTGCTCATCCTGAGCCACATTAGACACGCC |
| | | AGCAACAAAGGCATGGAACTGCTGTACTCCATGAAGTGCAAGAACGT |
| | | GGTGCCCCTGTACGACCTGCTGCTCGAAGCTGCCGATGCTCACAGAC |
| | | TGCACGCCCCTACATCTAGAGGCGGAGCCTCTGTGGAAGAGACAGAC |
| | | CAGTCTCATCTGGCCACAGCCGGCAGCACATCTTCTCACAGCCTGCA |
| | | GAAGTACTACATCACCGGCGAGGCCGAGGGATTTCCTGCTACAGCTG |
| | | GCGGCGGAGGATCTGGCGGAGGTGGAAGCGGAGGCGGTGGAAGTGTG |
| | | GATGGCTTTGATGTGGGCGCCCTGGAAAGCCTGAGAGGCAATGCCGA |
| | | TCTGGCCTACATCCTGTCCATGGAACCTTGCGGCCACTGCCTGATTA |
| | | TCAACAACGTGAACTTCTGTCGCGAGAGCGGCCTGAGAACCAGAACC |
| | | GGCAGCAATATCGACTGCGAGAAGCTGCGGAGAAGATTCAGCAGCCT |
| | | GCACTTTATGGTGGAAGTCAAGGGCGATCTGACCGCCAAGAAAATGG |
| | | TGCTGGCCCTGCTGGAACTGGCCAGACAAGATCATGGCGCTCTGGAC |
| | | TGCTGCGTGGTCGTGATTCTGTCTCATGGCTGCCAGGCCAGCCATCT |
| | | GCAATTCCCTGGTGCCGTGTATGGCACCGATGGCTGTCCTGTGTCCG |
| | | TGGAAAAGATCGTGAACATCTTCAACGGCACAAGCTGCCCTAGCCTC |
| | | GGCGGAAAGCCCAAGCTGTTCTTCATCCAAGCCTGTGGCGGCGAGCA |
| | | GAAGGATCACGGATTTGAGGTGGCCTCCACCTCTCCTGAGGATGAGA |
| | | GCCCTGGAAGCAACCCCGAGCCTGATGCCACACCTTTCCAAGAGGGA |
| | | CTGAGAACCTTCGACCAGCTGGACGCTATCAGCTCCCTGCCTACACC |
| | | TAGCGACATCTTCGTGTCCTACAGCACATTCCCCGGCTTCGTGTCTT |
| | | GGAGGGATCCCAAGTCTGGCTCTTGGTACGTGGAAACCCTGGACGAC |
| | | ATCTTTGAGCAGTGGGCCCATAGCGAGGACCTGCAGTCTCTGCTTCT |
| | | GAGAGTGGCCAATGCCGTGTCCGTGAAGGGCATCTACAAGCAGATGC |
| | | CCGGCTGCTTCAACTTCCTGCGGAAGAAGCTGTTTTTCAAGACCAGC |
| | | GGCTCCGGCCAGTGCACAAATTATGCACTGCTGAAGCTCGCCGGGGA |
| | | TGTCGAGAGTAACCCAGGACCTGGAAGCGGAGAAGGTCGTGGTAGTC |
| | | TACTAACGTGTGGTGATGTAGAAGAAAATCCTGGACCTatgaccgag |
| | | tacaagcccacggtgcgcctcgccacccgcgacgacgtccccagggc |
| | | cgtacgcaccctcgccgccgcgttcgccgactaccccgccacgcgcc |
| | | acaccgtcgatccggaccgccacatcgagcgggtcaccgagctgcaa |
| | | gaactcttcctcacgcgcgtcgggctcgacatcggcaaggtgtgggt |
| | | cgcggacgacggcgccgcggtggcggtctggaccacgccggagagcg |
| | | tcgaagcgggggcggtgttcgccgagatcggcccgcgcatggccgag |
| | | ttgagcggttcccggctggccgcgcagcaacagatggaaggcctcct |
| | | ggcgccgcaccggcccaaggagcccgcgtggttcctggccaccgtcg |
| | | gcgtctcgcccgaccaccagggcaagggtctgggcagcgccgtcgtg |
| | | ctccccggagtggaggcggccgagcgcgccggggtgcccgccttcct |
| | | ggagacctccgcgccccgcaacctccccttctacgageggctcggct |
| | | tcaccgtcaccgccgacgtcgaggtgcccgaaggaccgcgcacctgg |
| | | tgcatgacccgcaagcccggtgccTGA |
| | | (SEQ ID NO: 28) |

The modified ERT2 peptide-suicide protein fusion polypeptide encoding constructs (Constructs 9-12) are delivered to human iPSCs via Nucleofection with a GAPDH targeting ribonucleoprotein (RNP) complex comprising of Cpf1 endonuclease and a single guide RNA (sgRNA) targeting the coding sequence of the GAPDH gene, as per Example 2. Individual clones are derived from the transfected pools by puromycin selection and apoptotic efficacy is determined in individual clones edited with Constructs 9-12 by screening clones in the presence of a range of 4-OHT and Endoxifen in PSCs and PSC-derived Myeloid Progenitor cells, as per Example 11.

### Example 14

### Assessment Improved Modified ERT2 Peptide-Suicide Protein Constructs, and Evaluation of Apoptotic Efficacy in PSCs and in PSC-Derived Myeloid Progenitor Cells

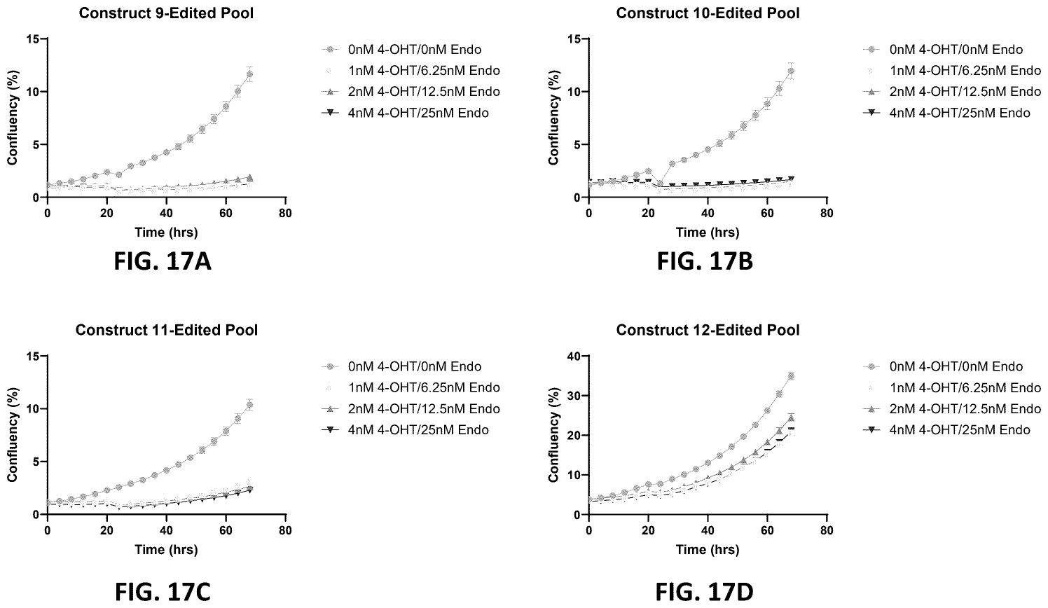

Induced pluripotent stem cells (iPSCs) were edited using Construct 9, 10, 11, or 12 (Table 5). Edited pools of iPSCs were treated with puromycin selection at a concentration of 0.25 μg/mL for 3 days starting at 6 days following Nucleofection and continuing until 9 days following Nucleofection. Then combinations of 4-OHT (from 0 to 4 nM) and endoxifen (from 0 to 25 nM) were added to the edited pools for 3 days. Untreated and treated wells of the edited pools were imaged every 4 hours over the course of the 3 day treatment duration using an Incucyte SX5 (Sartorius) and the confluency in each well was quantified using the onboard Incucyte analysis pipeline. Addition of all tested ranges of combination 4-OHT and endoxifen treatment resulted in cell death, with nearly complete cell death seen for iPSCs edited with Construct 9 (FIG. 17A), 10 (FIG. 17B), and 11 (FIG. 17C). The relative lack of cell death seen for the iPSCs edited with Construct 12 (FIG. 17D) may have been due to incomplete puromycin selection of the edited pool.

While the present disclosure has been particularly shown and described with reference to a preferred embodiment and various alternate embodiments, it will be understood by persons skilled in the relevant art that various changes in form and details can be made therein without departing from the spirit and scope of the present disclosure and appended claims.

All references, issued patents and patent applications cited within the body of the instant specification are hereby incorporated by reference in their entirety, for all purposes.

What is claimed is:

1. A targeting construct comprising:
(a) a first homology arm corresponding to a 5′ target sequence comprising a first region of homology to a target genomic locus;
(b) a nucleotide insert comprising a nucleotide sequence encoding a fusion polypeptide comprising:
(i) a modified estrogen receptor ligand binding domain (ER-LBD); and
(ii) a caspase 9 domain or derivative or functional fragment thereof;
(c) a second homology arm corresponding to a 3′ target sequence comprising second region of homology to the target genomic locus,
wherein the modified ER-LBD comprises an amino acid sequence corresponding to a hormone binding domain of a reference human estrogen receptor sequence (SEQ ID NO: 1), and wherein the modified ER-LBD comprises
a G400V amino acid substitution, an M543A amino acid substitution, an L544A amino acid substitution, and optionally a V595A amino acid substitution, with reference to SEQ ID NO: 1; and
additional amino acid substitutions, wherein the additional amino acid substitutions comprise, with reference to SEQ ID NO: 1:
(i) an L384M substitution, an L391V substitution, a N413D substitution, an M421L substitution, a S463P substitution, and a H524L substitution,
(ii) an L391V substitution, a N413D substitution, a Q414E substitution, a S463P substitution, and a H524F substitution,
(iii) an L354I substitution, a L391V substitution, a N413D substitution, a Q414E substitution, a M421L substitution, a M517A substitution, and a H524F substitution,
(iv) an L354I substitution, a L391V substitution, a L409V substitution, a N413D substitution, a Q414E substitution, and a H524L substitution,
(v) an L391V substitution, a Q414E substitution, an N413D substitution, an S463P substitution, an M421L substitution, an L354I substitution, an L384M substitution, and an H524L substitution,
(vi) an L391V substitution, an N413D substitution, an S463P substitution, an M517A substitution, an M421L substitution, an L354I substitution, and an H524L substitution,
(vii) an N413D substitution, an S463P substitution, an L354I substitution, an L384M substitution, and an H524L substitution, or
(viii) an L391V substitution, an N413D substitution, an S463P substitution, an M517A substitution, an M421L substitution, an L354I substitution, and an H524L substitution,
wherein the targeting construct is configured such that upon its recombination with the target genomic locus, the fusion polypeptide coding sequence is integrated into a STEL gene and becomes operably linked to the STEL gene regulatory element.

2. A targeting construct comprising:
(a) a first homology arm corresponding to a 5′ target sequence comprising a first region of homology to a target genomic locus;
(b) a nucleotide insert comprising a nucleotide sequence encoding a fusion polypeptide comprising:
(i) a first modified estrogen receptor ligand binding domain (ER-LBD);
(ii) a first caspase 9 domain or derivative or functional fragment thereof;
(iii) a second modified estrogen receptor ligand binding domain (ER-LBD); and
(iv) a second caspase 9 domain or derivative or functional fragment thereof;
(c) a second homology arm corresponding to a 3′ target sequence comprising second region of homology to the target genomic locus,
wherein the first modified ER-LBD and the second modified ER-LBD each comprise an amino acid sequence corresponding to a hormone binding domain of a reference human estrogen receptor sequence (SEQ ID NO: 1), and wherein the first modified ER-LBD and the second modified ER-LBD each independently comprise:
a G400V amino acid substitution, an M543A amino acid substitution, an L544A amino acid substitution, and optionally a V595A amino acid substitution, with reference to SEQ ID NO: 1; and
additional amino acid substitutions, wherein the additional amino acid substitutions comprise, with reference to SEQ ID NO: 1:
(i) an L384M substitution, an L391V substitution, a N413D substitution, an M421L substitution, a S463P substitution, and a H524L substitution,
(ii) an L391V substitution, a N413D substitution, a Q414E substitution, a S463P substitution, and a H524F substitution,
(iii) an L354I substitution, a L391V substitution, a N413D substitution, a Q414E substitution, a M421L substitution, a M517A substitution, and a H524F substitution,
(iv) an L354I substitution, a L391V substitution, a L409V substitution, a N413D substitution, a Q414E substitution, and a H524L substitution,
(v) an L391V substitution, a Q414E substitution, an N413D substitution, an S463P substitution, an M421L substitution, an L354I substitution, an L384M substitution, and an H524L substitution,
(vi) an L391V substitution, an N413D substitution, an S463P substitution, an M517A substitution, an M421L substitution, an L354I substitution, and an H524L substitution,
(vii) an N413D substitution, an S463P substitution, an L354I substitution, an L384M substitution, and an H524L substitution, or
(viii) an L391V substitution, an N413D substitution, an S463P substitution, an M517A substitution, an M421L substitution, an L354I substitution, and an H524L substitution,
wherein the targeting construct is configured such that upon its recombination with the target genomic locus, the fusion polypeptide coding sequence is integrated into a STEL gene and becomes operably linked to the STEL gene regulatory element.

3. The targeting construct of claim 1, wherein the additional amino acid substitutions comprise:
(a) an L384M substitution, an L391V substitution, a N413D substitution, an M421L substitution, a S463P substitution, and a H524L substitution, and wherein the modified ER-LBD comprises an amino acid sequence having at least 80%, at least 85%, at least 90%, or at least 95% sequence identity to the amino acid sequence of (SEQ ID NO:2), optionally wherein the modified ER-LBD comprises an amino acid sequence having at least 98% sequence identity to the amino acid sequence of SEQ ID NO:2, optionally wherein the modified ER-LBD comprises an amino acid sequence having at least 99% sequence identity to the amino acid sequence of SEQ ID NO:2, optionally wherein the modified ER-LBD comprises or consists of an amino acid sequence having 100% sequence identity to the amino acid sequence of SEQ ID NO:2;
(b) an L391V substitution, a N413D substitution, a Q414E substitution, a S463P substitution, and a H524F substitution, and wherein the modified ER-LBD comprises an amino acid sequence having at least 80%, at least 85%, at least 90%, or at least 95% sequence identity to the amino acid sequence of SEQ ID NO:3, optionally wherein the modified ER-LBD comprises an amino acid sequence having at least 98% sequence identity to the amino acid sequence of SEQ ID NO:3; optionally wherein the modified ER-LBD comprises an amino acid sequence having at least 99% sequence identity to the amino acid sequence of SEQ ID NO:3, optionally wherein the modified ER-LBD comprises or consists of an amino acid sequence having 100% sequence identity to the amino acid sequence of SEQ ID NO:3;
(c) an L354I substitution, a L391V substitution, a N413D substitution, a Q414E substitution, a M421L substitution, a M517A substitution, and a H524F substitution, and wherein the modified ER-LBD comprises an amino acid sequence having at least 80%, at least 85%, at least 90%, or at least 95% sequence identity to the amino acid sequence of SEQ ID NO:4, optionally wherein the modified ER-LBD comprises an amino acid sequence having at least 98% sequence identity to the amino acid sequence of SEQ ID NO:4, optionally wherein the modified ER-LBD comprises an amino acid sequence having at least 99% sequence identity to the amino acid sequence of SEQ ID NO:4, optionally wherein the modified ER-LBD comprises or consists of an amino acid sequence having 100% sequence identity to the amino acid sequence of SEQ ID NO:4;
(d) an L354I substitution, a L391V substitution, a L409V substitution, a N413D substitution, a Q414E substitution, and a H524L substitution, and wherein the modified ER-LBD comprises an amino acid sequence having at least 80%, at least 85%, at least 90%, or at least 95% sequence identity to the amino acid sequence of SEQ ID NO:5, optionally wherein the modified ER-LBD comprises an amino acid sequence having at least 98% sequence identity to the amino acid sequence of SEQ ID NO:5, optionally wherein the modified ER-LBD comprises an amino acid sequence having at least 99% sequence identity to the amino acid sequence of SEQ ID NO:5, optionally wherein the modified ER-LBD comprises or consists of an amino acid sequence having 100% sequence identity to the amino acid sequence of SEQ ID NO:5;
(e) an L391V substitution, a Q414E substitution, an N413D substitution, an S463P substitution, an M421L substitution, an L354I substitution, an L384M substitution, and an H524L substitution, and the modified ER-LBD comprises an amino acid sequence having at least 80%, at least 85%, at least 90%, or at least 95% sequence identity to the amino acid sequence of SEQ ID NO:40, optionally wherein the modified ER-LBD comprises an amino acid sequence having at least 98% sequence identity to the amino acid sequence of SEQ ID NO:40, optionally wherein the modified ER-LBD comprises an amino acid sequence having at least 99% sequence identity to the amino acid sequence of SEQ ID NO:40, optionally wherein the modified ER-LBD comprises or consists of an amino acid sequence having 100% sequence identity to the amino acid sequence of SEQ ID NO: 40;
(f) an L391V substitution, an N413D substitution, an S463P substitution, an M517A substitution, an M421L substitution, an L354I substitution, and an H524L substitution, and the modified ER-LBD comprises an amino acid sequence having at least 80%, at least 85%, at least 90%, or at least 95% sequence identity to the amino acid sequence of SEQ ID NO:41, optionally wherein the modified ER-LBD comprises an amino acid sequence having at least 98% sequence identity to the amino acid sequence of SEQ ID NO:41, optionally wherein the modified ER-LBD comprises an amino acid sequence having at least 99% sequence identity to the amino acid sequence of SEQ ID NO:41, optionally wherein the modified ER-LBD comprises or consists of an amino acid sequence having 100% sequence identity to the amino acid sequence of SEQ ID NO:41;
(g) an N413D substitution, an S463P substitution, an L354I substitution, an L384M substitution, and an H524L substitution, and the modified ER-LBD comprises an amino acid sequence having at least 80%, at least 85%, at least 90%, or at least 95% sequence identity to the amino acid sequence of SEQ ID NO:42, optionally wherein the modified ER-LBD comprises an amino acid sequence having at least 98% sequence identity to the amino acid sequence of SEQ ID NO:42, optionally wherein the modified ER-LBD comprises an amino acid sequence having at least 99% sequence identity to the amino acid sequence of SEQ ID NO:42, optionally wherein the modified ER-LBD comprises or consists of an amino acid sequence having 100% sequence identity to the amino acid sequence of SEQ ID NO: 42; or
(h) an L391V substitution, an N413D substitution, an S463P substitution, an M517A substitution, an M421L substitution, an L354I substitution, and an H524L substitution, and the modified ER-LBD comprises an amino acid sequence having at least 80%, at least 85%, at least 90%, or at least 95% sequence identity to the amino acid sequence of SEQ ID NO:43, optionally wherein the modified ER-LBD comprises an amino acid sequence having at least 98% sequence identity to the amino acid sequence of SEQ ID NO:43, optionally wherein the modified ER-LBD comprises an amino acid sequence having at least 99% sequence identity to the amino acid sequence of SEQ ID NO:43, optionally wherein the modified ER-LBD comprises or consists of an amino acid sequence having 100% sequence identity to the amino acid sequence of SEQ ID NO:43, optionally wherein the modified ERT2 peptide further comprises an N-terminal serine (S) residue, optionally wherein the modified ER-LBD comprises the V595A amino acid substitution,
optionally wherein the targeting construct comprises an amino acid sequence at least 80%, at least 85%, at least 90%, at least 91%, at least 92%, at least 93%, at least 94%, at least 95%, at least 96%, at least 97%, at least 98%, at least 99%, at least 99.5%, or at least 99.9% identical to an amino acid sequence set forth in SEQ ID NO: 13, 15, 17, 19, 21, 23, 25, 27, 44, 46, 48, or 50, optionally wherein the targeting construct comprises an amino acid sequence set forth in SEQ ID NO: 13, 15, 17, 19, 21, 23, 25, 27, 44, 46, 48, or 50.

4. The targeting construct of claim 1, wherein:
(a) the caspase 9 domain or derivative or functional fragment thereof does not comprise a Caspase Activation and Recruitment Domain (CARD) domain sequence, optionally wherein the caspase 9 domain or derivative or functional fragment thereof comprises (SEQ ID NO:6);
(b) the modified estrogen receptor ligand binding domain (ER-LBD) is N-terminal or C-terminal to the caspase 9 domain or derivative or functional fragment thereof, optionally wherein the fusion polypeptide comprises a linker between the modified estrogen receptor ligand binding domain (ER-LBD) and the caspase 9 domain or derivative or functional fragment thereof.
(c) neither the first nor second caspase 9 domain or derivative or functional fragment thereof comprise a Caspase Activation and Recruitment Domain (CARD) domain sequence, optionally wherein the first caspase 9 domain or derivative or functional fragment thereof and/or the second caspase 9 domain or derivative or functional fragment thereof comprises (SEQ ID NO:6); and/or
(d) the first ER-LBD is N-terminal or C-terminal to the first caspase 9 domain or derivative or functional fragment thereof and/or the second ER-LBD is N-terminal or C-terminal to the second caspase 9 domain or derivative or functional fragment thereof, optionally wherein the fusion polypeptide comprises a linker between the modified estrogen receptor ligand binding domain (ER-LBD) and the caspase 9 domain or derivative or functional fragment thereof.

5. The targeting construct of claim 1, which is configured such that upon its recombination with the target genomic locus, the STEL gene is modified such to incorporate the fusion polypeptide coding sequence 3′ to the STEL protein coding sequence or 5′ to the STEL protein coding sequence.

6. The targeting construct of claim 1, wherein the nucleotide insert further comprises a nucleotide sequence encoding a separator sequence, optionally wherein thee targeting construct is configured such that upon recombination of the targeting construct with the target genomic locus, the separator sequence coding sequence is positioned between the coding sequence of the STEL protein and the fusion polypeptide coding sequence, optionally wherein the separator sequence is (a) an internal ribosome entry site (“IRES”) or (b) a self-cleaving peptide, optionally wherein the self-cleaving peptide is a 2A peptide, optionally wherein the self-cleaving peptide is T2A, P2A, E2A, F2A, PQR, Opt2A, or Opt2A_2.0.

7. The targeting construct of claim 1, wherein the STEL gene encodes a polypeptide involved in one or more of: glycolysis, ribonucleopolypeptide complex formation, focal adhesion, cell-substrate adherens junction, cell-substrate junction, cell anchoring, extracellular exosome, extracellular vesicle, intracellular organelle, anchoring junction, RNA binding, nucleic acid binding (e.g., rRNA or mRNA binding), and polypeptide binding, optionally wherein the STEL gene encodes a ribosomal polypeptide, optionally wherein the STEL gene is RPL13A, RPLP0, RPL10, RPL13, RPSJ8, RPL3, RPLP1, RPL15, RPL41, RPL11, RPL32, RPL18 A, RPL19, RPL28, RPL29, RPL9, RPL8, RPL6, RPL18, RPL7, RPL7A, RPL21, RPL37A, RPL 12, RPL5, RPL34, RPL35A, RPL30, RPL24, RPL39, RPL37, RPL14, RPL27A, RPLP2, RPL23A, RPL26, RPL36, RPL35, RPL23, RPL4, or RPL22, optionally wherein the STEL gene encodes a ribosomal polypeptide small subunit (RPS), optionally wherein the STEL gene is RPS2, RPS19, RPS14, RPS3A, RPS12, RPS3, RPS6, RPS23, RPS27A, RPS8, RPS4X, RPS7, RPS24, RPS27, RPS15A, RPS9, RPS28, RPS13, RPSA, RPS5, RPS 16, RPS25, RPS15, RPS20, or RPS11, optionally wherein the STEL gene encodes a mitochondrial polypeptide, optionally wherein the STEL gene is MT-CO1, MT-CO2, MT-ND4, MT-ND1, or MT-ND2, optionally wherein the STEL gene encodes an actin polypeptide, optionally wherein the STEL gene is ACTG1 or ACTB, optionally wherein the STEL gene encodes a eukaryotic translation factor, optionally wherein the STEL gene is EEF1A1, EEF2, or EIF1m optionally wherein the STEL gene encodes a histone, optionally wherein the STEL gene is H3F3A or H3F3B, optionally wherein the STEL gene is FTL, FTH1, TPT1, IMSB10, GAPDH, PTMA, GNB2L1, NACA, YBX1, NPM1, FAU, UBA52, HSP90AB1, MYL6, SERF2, or SRP14, optionally wherein the STEL gene is GAPDH, optionally wherein the STEL gene is RPL13A, optionally wherein the STEL gene is RPL7, optionally wherein the STEL gene is RPLP0.

8. The targeting construct of claim 1, wherein the nucleotide insert further comprises a transgene, optionally wherein the transgene is linked to the fusion polypeptide coding sequence, optionally wherein the transgene encodes a therapeutic polypeptide, optionally wherein the fusion polypeptide coding sequence and transgene are connected via a nucleotide sequence encoding a separator sequence, optionally wherein the separator sequence is an internal ribosome entry site (“IRES”), optionally wherein the separator sequence is a nucleotide sequence encoding a self-cleaving peptide (the “self-cleaving peptide coding sequence”), optionally wherein the self-cleaving peptide is a 2A peptide, optionally wherein the self-cleaving peptide is T2A, P2A, E2A, F2A, PQR, Opt2A, or Opt2A_2.0.

9. A system comprising:
(a) the targeting construct of claim 1;
(b) a CRISPR-associated endonuclease (“Cas polypeptide”) or a nucleic acid encoding a Cas polypeptide; and
(c) a guide RNA (“gRNA”) comprising a scaffold for binding the Cas polypeptide and a spacer sequence corresponding to the STEL gene, or a nucleic acid encoding the gRNA,
optionally wherein the guide RNA is a single guide RNA (“sgRNA”),
optionally wherein the system comprises the Cas polypeptide and gRNA,
optionally wherein the system is in the form of a ribonucleoprotein particle (“RNP”).

10. A method of producing a gene-edited target cell, comprising:
(a) introducing the system of claim 9 into a target cell; and
(b) culturing the target cell under conditions in which gene editing occurs, thereby producing gene-edited target cell,
optionally wherein the gene-targeted cell is of ectoderm lineage, optionally wherein the gene-edited target cell is a neuron,
optionally wherein the gene-targeted cell if of mesoderm lineage, optionally wherein the gene-edited target cell is a cardiomyocyte.

11. The method of claim 10, wherein the target cell is:
(a) a stem cell or a cell differentiated from a stem cell, optionally wherein the target cell is a stem cell, optionally wherein the stem cell is a human embryonic stem cell,
(b) an induced pluripotent stem cell (“iPSC”) or a cell differentiated therefrom, optionally wherein the target cell is (a) a regulatory T cell, a myeloid cell, a dendritic cell, a macrophage (e.g., an immunosuppressive macrophage), a myeloid progenitor cell, or a precursor or progenitor cell thereof;
(c) a cell in the human nervous system, optionally selected from dopaminergic neuron, a microglial cell, an oligodendrocyte, an astrocyte, a cortical neuron, a spinal or oculomotor neuron, an enteric neuron, a Placode-derived cell, a Schwann cell, and a trigeminal or sensory neuron, or a precursor or progenitor cell thereof;
(d) a cell in the human cardiovascular system, optionally selected from a cardiomyocyte, an endothelial cell, and a nodal cell, or a precursor or progenitor cell thereof;
(e) a cell in the human metabolic system, optionally selected from a hepatocyte, a cholangiocyte, and a pancreatic beta cell, or a precursor or progenitor cell thereof, or
(f) a cell in the human ocular system, optionally selected from a retinal pigment epithelial cell, a photoreceptor cone cell, a photoreceptor rod cell, a bipolar cell, a ganglion cell, or a precursor or progenitor cell thereof.

12. A gene-edited target cell obtained or obtainable by the method of claim 10.

13. A gene-edited target cell comprising a STEL gene that comprises a nucleic acid encoding a fusion polypeptide under the transcriptional control of a STEL gene regulatory element, the fusion polypeptide comprising:
(a) a modified estrogen receptor ligand binding domain (ER-LBD); and
(b) a caspase 9 domain or derivative or functional fragment thereof,
wherein the modified ER-LBD comprises an amino acid sequence corresponding to a hormone binding domain of a reference human estrogen receptor sequence (SEQ ID NO: 1), and wherein the modified ER-LBD comprises
a G400V amino acid substitution, an M543A amino acid substitution, an L544A amino acid substitution, and optionally a V595A amino acid substitution, with reference to SEQ ID NO: 1; and
additional amino acid substitutions, wherein the additional amino acid substitutions comprise, with reference to SEQ ID NO: 1:
(i) an L384M substitution, an L391V substitution, a N413D substitution, an M421L substitution, a S463P substitution, and a H524L substitution,
(ii) an L391V substitution, a N413D substitution, a Q414E substitution, a S463P substitution, and a H524F substitution,
(iii) an L354I substitution, a L391V substitution, a N413D substitution, a Q414E substitution, a M421L substitution, a M517A substitution, and a H524F substitution,
(iv) an L354I substitution, a L391V substitution, a L409V substitution, a N413D substitution, a Q414E substitution, and a H524L substitution,
(v) an L391V substitution, a Q414E substitution, an N413D substitution, an S463P substitution, an M421L substitution, an L354I substitution, an L384M substitution, and an H524L substitution,
(vi) an L391V substitution, an N413D substitution, an S463P substitution, an M517A substitution, an M421L substitution, an L354I substitution, and an H524L substitution,
(vii) an N413D substitution, an S463P substitution, an L354I substitution, an L384M substitution, and an H524L substitution, or
(viii) an L391V substitution, an N413D substitution, an S463P substitution, an M517A substitution, an M421L substitution, an L354I substitution, and an H524L substitution.

14. A pharmaceutical composition comprising the gene-edited target cell of claim 13 and a pharmaceutically acceptable carrier.

15. A method of treating a patient in need thereof, comprising administering to the patient the gene-edited target cell of claim 12 or 13 or a pharmaceutical composition thereof, optionally wherein the method further comprises controlling the gene-edited target cell population in the patient by:
(a) monitoring, optionally, the gene-edited target cell population in the patient; and/or
(b) administering an inducer of the modified ER-LBD if the patient experiences adverse events related to the gene-edited target cell population.

16. A method of mitigating adverse events or a safety risk associated with cell therapy in the form of the gene-edited target cells of claim 13 or pharmaceutical composition thereof, the method comprising administering to a patient who received the gene-edited target cells or pharmaceutical composition an inducer of a modified ER-LBD if the patient experiences adverse events or a safety risk related to the gene-edited target cells or pharmaceutical composition, optionally wherein the inducer of the modified ER-LBD is tamoxifen or a tamoxifen metabolite, optionally wherein the inducer of the modified ER-LBD is tamoxifen, optionally wherein the inducer of the modified ER-LBD is a tamoxifen metabolite, optionally wherein the inducer of the modified ER-LBD is 4-hydroxytamoxifen, N-desmethyltamoxifen, tamoxifen-N-oxide, or endoxifen, optionally wherein a combination of two or more inducers of the modified ER-LBD is administered, optionally wherein the combination comprises 4-OHT and endoxifen, optionally wherein the combination has a synergistic effect and/or utilizes reduced dosing (e.g., reduced dosing amount and/or frequency) than would be required a single inducer of the modified ER-LBD.

17. The method of claim 13, wherein the modified ER-LBD comprises an amino acid sequence corresponding to a hormone binding domain of a reference human estrogen receptor sequence (SEQ ID NO: 1), and wherein the modified ER-LBD comprises
a G400V amino acid substitution, an M543A amino acid substitution, an L544A amino acid substitution, and optionally a V595A amino acid substitution, with reference to SEQ ID NO: 1; and
additional amino acid substitutions, wherein the additional amino acid substitutions comprise, with reference to SEQ ID NO: 1:
(i) an L384M substitution, an L391V substitution, a N413D substitution, an M421L substitution, a S463P substitution, and a H524L substitution,
(ii) an L391V substitution, a N413D substitution, a Q414E substitution, a S463P substitution, and a H524F substitution,
(iii) an L354I substitution, a L391V substitution, a N413D substitution, a Q414E substitution, a M421L substitution, a M517A substitution, and a H524F substitution,
(iv) an L354I substitution, a L391V substitution, a L409V substitution, a N413D substitution, a Q414E substitution, and a H524L substitution,
(v) an L391V substitution, a Q414E substitution, an N413D substitution, an S463P substitution, an M421L substitution, an L354I substitution, an L384M substitution, and an H524L substitution,
(vi) an L391V substitution, an N413D substitution, an S463P substitution, an M517A substitution, an M421L substitution, an L354I substitution, and an H524L substitution,
(vii) an N413D substitution, an S463P substitution, an L354I substitution, an L384M substitution, and an H524L substitution, or
(viii) an L391V substitution, an N413D substitution, an S463P substitution, an M517A substitution, an M421L substitution, an L354I substitution, and an H524L substitution.

18. The method of claim 13, wherein
(a) the caspase 9 domain or derivative or functional fragment thereof does not comprise a Caspase Activation and Recruitment Domain (CARD) domain sequence, optionally wherein the caspase 9 domain or derivative or functional fragment thereof comprises (SEQ ID NO:6)
(b) the modified estrogen receptor ligand binding domain (ER-LBD) is N-terminal or C-terminal to the caspase 9 domain or derivative or functional fragment thereof, optionally wherein the fusion polypeptide comprises a linker between the modified estrogen receptor ligand binding domain (ER-LBD) and the caspase 9 domain or derivative or functional fragment thereof.
(c) neither the first nor second caspase 9 domain or derivative or functional fragment thereof comprise a Caspase Activation and Recruitment Domain (CARD) domain sequence, optionally wherein the first caspase 9 domain or derivative or functional fragment thereof and/or the second caspase 9 domain or derivative or functional fragment thereof comprises (SEQ ID NO:6); and/or
(d) the first ER-LBD is N-terminal or C-terminal to the first caspase 9 domain or derivative or functional fragment thereof and/or the second ER-LBD is N-terminal or C-terminal to the second caspase 9 domain or derivative or functional fragment thereof, optionally wherein the fusion polypeptide comprises a linker between the modified estrogen receptor ligand binding domain (ER-LBD) and the caspase 9 domain or derivative or functional fragment thereof.

19. The method of claim 13, wherein the STEL gene encodes a polypeptide involved in one or more of: glycolysis, ribonucleopolypeptide complex formation, focal adhesion, cell-substrate adherens junction, cell-substrate junction, cell anchoring, extracellular exosome, extracellular vesicle, intracellular organelle, anchoring junction, RNA binding, nucleic acid binding (e.g., rRNA or mRNA binding), and polypeptide binding, optionally wherein the STEL gene encodes a ribosomal polypeptide, optionally wherein the STEL gene is RPL13A, RPLP0, RPL10, RPL13, RPSJ8, RPL3, RPLP1, RPL15, RPL41, RPL11, RPL32, RPL18 A, RPL19, RPL28, RPL29, RPL9, RPL8, RPL6, RPL18, RPL7, RPL7A, RPL21, RPL37A, RPL 12, RPL5, RPL34, RPL35A, RPL30, RPL24, RPL39, RPL37, RPL14, RPL27A, RPLP2, RPL23A, RPL26, RPL36, RPL35, RPL23, RPL4, or RPL22, optionally wherein the STEL gene encodes a ribosomal polypeptide small subunit (RPS), optionally wherein the STEL gene is RPS2, RPS19, RPS14, RPS3A, RPS12, RPS3, RPS6, RPS23, RPS27A, RPS8, RPS4X, RPS7, RPS24, RPS27, RPS15A, RPS9, RPS28, RPS13, RPSA, RPS5, RPS 16, RPS25, RPS15, RPS20, or RPS11, optionally wherein the STEL gene encodes a mitochondrial polypeptide, optionally wherein the STEL gene is MT-CO1, MT-CO2, MT-ND4, MT-ND1, or MT-ND2, optionally wherein the STEL gene encodes an actin polypeptide, optionally wherein the STEL gene is ACTG1 or ACTB, optionally wherein the STEL gene encodes a eukaryotic translation factor, optionally wherein the STEL gene is EEF1A1, EEF2, or EIF1m optionally wherein the STEL gene encodes a histone, optionally wherein the STEL gene is H3F3A or H3F3B, optionally wherein the STEL gene is FTL, FTH1, TPT1, IMSB10, GAPDH, PTMA, GNB2L1, NACA, YBX1, NPM1, FAU, UBA52, HSP90AB1, MYL6, SERF2, or SRP14, optionally wherein the STEL gene is GAPDH, optionally wherein the STEL gene is RPL13A, optionally wherein the STEL gene is RPL7, optionally wherein the STEL gene is RPLP0.

20. The method of claim 13, wherein the target cell is:
(a) a stem cell or a cell differentiated from a stem cell, optionally wherein the target cell is a stem cell, optionally wherein the stem cell is a human embryonic stem cell,
(b) an induced pluripotent stem cell (“iPSC”) or a cell differentiated therefrom, optionally wherein the target cell is (a) a regulatory T cell, a myeloid cell, a dendritic cell, a macrophage (e.g., an immunosuppressive macrophage), a myeloid progenitor cell, or a precursor or progenitor cell thereof;
(c) a cell in the human nervous system, optionally selected from dopaminergic neuron, a microglial cell, an oligodendrocyte, an astrocyte, a cortical neuron, a spinal or oculomotor neuron, an enteric neuron, a Placode-derived cell, a Schwann cell, and a trigeminal or sensory neuron, or a precursor or progenitor cell thereof;
(d) a cell in the human cardiovascular system, optionally selected from a cardiomyocyte, an endothelial cell, and a nodal cell, or a precursor or progenitor cell thereof;
(e) a cell in the human metabolic system, optionally selected from a hepatocyte, a cholangiocyte, and a pancreatic beta cell, or a precursor or progenitor cell thereof, or
(f) a cell in the human ocular system, optionally selected from a retinal pigment epithelial cell, a photoreceptor cone cell, a photoreceptor rod cell, a bipolar cell, a ganglion cell, or a precursor or progenitor cell thereof.


SEQUENCE LISTING (63 sequences)

SEQ ID NO: 1 - protein, 595 aa, synthetic construct [sequence omitted]

SEQ ID NO: 2 - protein, 312 aa, synthetic construct
AGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGVVWRSMEHPVKLLFAPNLLLDRDQGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPAT

SEQ ID NO: 3 - protein, 312 aa, synthetic construct
AGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGVVWRSMEHPVKLLFAPNLLLDRDEGKCVEGMVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMEFLYSMKCKNVVPLYDLLLEAADAHRLHAPTSRGGASVEETDQSHLATAGSTSSHSLQKYYITGEAEGFPAT

SEQ ID NO: 4 - protein, 312 aa, synthetic construct
AGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWLEILMIGVVWRSMEHPVKLLFAPNLLLDRDEGKCVEGLVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHASNKGMEFLYSMKCKNVVPLYDLLLEAADAHRLHAPTSRGGASVEETDQSHLATAGSTSSHSLQKYYITGEAEGFPAT

SEQ ID NO: 5 - protein, 312 aa, synthetic construct
AGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWLEILMIGVVWRSMEHPVKLLFAPNLVLDRDEGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPAT

SEQ ID NO: 6 - protein, 280 aa, synthetic construct
MDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 7 - DNA, 800 bp, synthetic construct
  source 1..800: mol_type = other DNA
ttggtatcgtggaaggactcatggtatgagagctggggaatgggactgaggctcccacctttctcatccaagactggctcctccctgccggggctgcgtgcaaccctggggttgggggttctggggactggctttcccataatttcctttcaaggtggggagggaggtagaggggtgatgtggggagtacgctgcagggcctcactccttttgcagaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgtggctggggccagagactggctcttaaaaagtgcagggtctggcgccctctggtggctggctcagaaaaagggccctgacaactctttacatcttctaggtatgacaacgaatttggctacagcaacagggtggtggacctcatggcccacatggcctccaaggag

SEQ ID NO: 8 - DNA, 800 bp, synthetic construct
  source 1..800: mol_type = other DNA
gacccctggaccaccagccaaagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaaccagttacttgtcctgtcttattctagggtctggggcagaggggagggaagctgggcttgtgtcaaggtgagacattcttgctggggagggacctggtatgttctcctcagactgagggtagggcctccaaacagccttgcttgcttcgagaaccatttgcttcccgctcagacgtcttgagtgctacaggaagctggcaccactacttcagagaacaaggccttttcctctcctcgctccagtcctaggctatctgctgttggccaaacatggaagaagctattctgtgggcagccccagggaggctgacaggtggaggaagtcagggctcgcactgggctctgacgctgactggttagtggagctcagcctggagctgagctgcagcgggcaattccagcttggcctccgcagctgtgaggtcttgagcacgtgctctattgctttctgtgccctcgtgtcttatctgaggacatcgtggccagcccctaaggtcttcaagcaggattcatctaggtaaaccaagtacctaaaaccatgcccaaggcggtaaggactatataatgtttaaaaatcggtaaaaatgcccacctcgcatagt

SEQ ID NO: 9 - DNA, 953 bp, synthetic construct
  source 1..953: mol_type = other DNA
ttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatggccacagaattcatggtgagcaagggcgaggagctgatcaaggagaacatgcacagcaagctgtacctggaaggcagcgtgaacggccaccagttcaagtgcacccacgaaggggagggcaagccctacgagggcacccagaccaacaggatcaaggtggtggagggaggccccctgccgttcgcattcgacatcctggccaccatgtttatgtacgggagcaaggccttcatcaagtaccccaagggcctccccgattattttaagcagtccttccctgagggcttcacatgggagagagtcatggtgttcgaagacgggggcgtgctgaccgccacccaggacaccagcctccaggacggctgcctcatctacaacgtgaagctgagaggggtgaacttcccagccaacggccccgtgatgaagcagacaacactgggctgggagcccagcaccgagaccctgtaccccgctgacggcgccctggaaggcagatgcgacatggccctgaagctcgtgggcgggggccacctgcactgcaacttcaagaccacatacaaatccaagaaacccgctacaaacctcaagatgcccggcgtccactacgtggaccgcagactggaaagaatcaaggaggccgacaacgagacctacgtcgagcagcacgaggtggctgtggccagatactgcgacctccctagcaaactggggcacaaacttaatggcatggacgagctgtacaagtga

SEQ ID NO: 10 - skipped

SEQ ID NO: 11 - protein, 19 aa, synthetic construct
ATNFSLLKQAGDVEENPGP

SEQ ID NO: 12 - protein, 40 aa, synthetic construct
ATNFSLLKQAGDVEENPGPGSGEGRGSLLTCGDVEENPGP

SEQ ID NO: 13 - protein, 855 aa, synthetic construct
SAGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWLEILMIGVVWRSMEHPVKLLFAPNLVLDRDQGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHASNKGMELLYSMKCKNVVPLYDLLLEAADAHRLHAPTSRGGASVEETDQSHLATAGSTSSHSLQKYYITGEAEGFPATAGGGGSGGGGSGGGGSVDGFDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSGSGQCTNYALLKLAGDVESNPGPGSGEGRGSLLTCGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 14 - DNA, 2568 bp, synthetic construct
  source 1..2568: mol_type = other DNA
atgtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagatcgttcacatgatcaactgggcgaagagggtgccaggctttgtggatttgaccctccatgatcaggtccaccttctagaatgtgcctggctagagatcctgatgattggtgtggtctggcgctccatggagcacccagtgaagctactgtttgctcctaacttggtgttggacagggaccagggaaaatgtgtagagggcctggtggagatcttcgacatgctgctggctacatcatctcggttccgcatgatgaatctgcagggagaggagtttgtgtgcctcaaatctattattttgcttaattctggagtgtacacatttctgcccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacgccagtaacaaaggcatggagctgctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagctgggggtggaggttcagggggtggaggttcaggtggtggcggtagtgtcgatggcttcgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcaggctccggccagtgcacaaattatgcactgctgaagctcgccggggatgtcgagagtaacccaggacctggaagcggagaaggtcgtggtagtctactaacgtgtggtgatgtagaagaaaatcctggacctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcc

SEQ ID NO: 15 - protein, 855 aa, synthetic construct
SAGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWMEILMIGVVWRSMEHPVKLLFAPNLLLDRDEGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPATAGGGGSGGGGSGGGGSVDGFDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSGSGQCTNYALLKLAGDVESNPGPGSGEGRGSLLTCGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 16 - DNA, 2568 bp, synthetic construct
  source 1..2568: mol_type = other DNA
atgtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagatcgttcacatgatcaactgggcgaagagggtgccaggctttgtggatttgaccctccatgatcaggtccaccttctagaatgtgcctggatggagatcctgatgattggtgtggtctggcgctccatggagcacccagtgaagctactgtttgctcctaacttgctcttggacagggacgagggaaaatgtgtagagggcctggtggagatcttcgacatgctgctggctacatcatctcggttccgcatgatgaatctgcagggagaggagtttgtgtgcctcaaatctattattttgcttaattctggagtgtacacatttctgcccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagctgctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagctgggggtggaggttcagggggtggaggttcaggtggtggcggtagtgtcgatggcttcgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcaggctccggccagtgcacaaattatgcactgctgaagctcgccggggatgtcgagagtaacccaggacctggaagcggagaaggtcgtggtagtctactaacgtgtggtgatgtagaagaaaatcctggacctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcc

SEQ ID NO: 17 - protein, 855 aa, synthetic construct
SAGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWMEILMIGLVWRSMEHPVKLLFAPNLLLDRDQGKCVEGMVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPATAGGGGSGGGGSGGGGSVDGFDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSGSGQCTNYALLKLAGDVESNPGPGSGEGRGSLLTCGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 18 - DNA, 2568 bp, synthetic construct
  source 1..2568: mol_type = other DNA
atgtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagatcgttcacatgatcaactgggcgaagagggtgccaggctttgtggatttgaccctccatgatcaggtccaccttctagaatgtgcctggatggagatcctgatgattggtctcgtctggcgctccatggagcacccagtgaagctactgtttgctcctaacttgctcttggacagggaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgcccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagctgctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagctgggggtggaggttcagggggtggaggttcaggtggtggcggtagtgtcgatggcttcgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcaggctccggccagtgcacaaattatgcactgctgaagctcgccggggatgtcgagagtaacccaggacctggaagcggagaaggtcgtggtagtctactaacgtgtggtgatgtagaagaaaatcctggacctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcc

SEQ ID NO: 19 - protein, 855 aa, synthetic construct
SAGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWLEILMIGVVWRSMEHPVKLLFAPNLLLDRDQGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHASNKGMELLYSMKCKNVVPLYDLLLEAADAHRLHAPTSRGGASVEETDQSHLATAGSTSSHSLQKYYITGEAEGFPATAGGGGSGGGGSGGGGSVDGFDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSGSGQCTNYALLKLAGDVESNPGPGSGEGRGSLLTCGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 20 - DNA, 2568 bp, synthetic construct
  source 1..2568: mol_type = other DNA
atgtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagatcgttcacatgatcaactgggcgaagagggtgccaggctttgtggatttgaccctccatgatcaggtccaccttctagaatgtgcctggctagagatcctgatgattggtgtggtctggcgctccatggagcacccagtgaagctactgtttgctcctaacttgctcttggacagggaccagggaaaatgtgtagagggcctggtggagatcttcgacatgctgctggctacatcatctcggttccgcatgatgaatctgcagggagaggagtttgtgtgcctcaaatctattattttgcttaattctggagtgtacacatttctgcccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacgccagtaacaaaggcatggagctgctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagctgggggtggaggttcagggggtggaggttcaggtggtggcggtagtgtcgatggcttcgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcaggctccggccagtgcacaaattatgcactgctgaagctcgccggggatgtcgagagtaacccaggacctggaagcggagaaggtcgtggtagtctactaacgtgtggtgatgtagaagaaaatcctggacctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcc

SEQ ID NO: 21 - protein, 1512 aa, synthetic construct
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGVVWRSMEHPVKLLFAPNLLLDRDQGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPATAGGGGSGGGGSGGGGSVDGFDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSGSGQCTNYALLKLAGDVESNPGPGSGEGRGSLLTCGDVEENPGPMSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGVVWRSMEHPVKLLFAPNLLLDRDQGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPATAGGGGSGGGGSGGGGSVDGFDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSGSGQCTNYALLKLAGDVESNPGPGSGEGRGSLLTCGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 22 - DNA, 4539 bp, synthetic construct
  source 1..4539: mol_type = other DNA
atgtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggatggagatcctgatgattggtgtggtctggcgctccatggagcacccagtgaagctactgtttgctcctaacttgctcttggacagggaccagggaaaatgtgtagagggcctggtggagatcttcgacatgctgctggctacatcatctcggttccgcatgatgaatctgcagggagaggagtttgtgtgcctcaaatctattattttgcttaattctggagtgtacacatttctgcccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagctgctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagctgggggtggaggttcagggggtggaggttcaggtggtggcggtagtgtcgatggcttcgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcaggctccggccagtgcacaaattatgcactgctgaagctcgccggggatgtcgagagtaacccaggacctggaagcggagaaggtcgtggtagtctactaacgtgtggtgatgtagaagaaaatcctggacctatgtctgccggcgatatgagagccgctaacctgtggccttctccactgatgatcaagcggagcaagaagaactctctggccctgagcctgaccgccgaccagatggtttctgctctgctggatgccgagcctcctatcctgtacagcgagtacgaccccaccagaccttttagcgaggccagcatgatgggcctgctgaccaatctggccgacagagaactggtgcacatgatcaactgggccaagcgcgtgcccggctttgtggatctgacactgcacgaccaagtgcatctgctggaatgcgcctggatggaaatcctgatgatcggcgtcgtgtggcggagcatggaacaccctgtgaagctgctgttcgcccctaacctgctgctggacagagatcagggcaaatgcgtggaaggactggtggaaatcttcgacatgctgctggccaccagctccagattccggatgatgaacctgcagggcgaagagttcgtgtgcctgaagtccatcatcctgctgaacagcggcgtgtacaccttcctgcctagcacactgaagtccctggaagagaaggaccacatccacagagtgctggataagatcaccgacacactgatccacctgatggccaaggccggactgacacttcagcagcagcatcagagactggcccagctgctgctcatcctgagccacatcagacacatgagcaacaaaggcatggaactgctgtactccatgaagtgcaagaacgtggtgcccctgtacgatctgctgctcgaagccgccgatgctcacagactgcatgcccctacatctagaggcggagccagcgtggaagagacagatcagtctcatctggccaccgccggcagcacaagctctcatagcctgcagaagtactacatcaccggcgaggccgagggatttcctgctactgctggtggcggaggatctggcggaggtggaagcggcggaggcggatctgttgatggctttgatgtgggcgccctggaaagcctgagaggcaatgccgatctggcctacatcctgtccatggaaccttgcggccactgcctgattatcaacaacgtgaacttctgtcgcgagagcggcctgagaaccagaaccggcagcaacatcgactgcgagaagctgcggagaagattcagcagcctgcactttatggtggaagtgaagggcgatctgaccgccaagaaaatggtgctggccctgctggaactggccagacaagatcatggcgctctggactgctgcgtggtcgtgattctgtctcatggctgccaggccagccatctgcaattccctggtgccgtgtatggcaccgatggctgtcctgtgtccgtggaaaagatcgtgaacatcttcaacggcacaagctgccctagcctcggcggaaagcccaagctgttcttcatccaagcctgtggcggcgagcagaaggatcacggatttgaggtggcctccacctctcctgaggatgagagccctggaagcaaccccgagcctgatgccacacctttccaagagggactgagaaccttcgaccagctggacgctatcagctccctgcctacacctagcgacatcttcgtgtcctacagcacattccccggcttcgtgtcttggagggatcccaagtctggctcttggtacgtggaaaccctggacgacatctttgagcagtgggcccatagcgaggacctgcagtctctgcttctgagagtggccaatgccgtgtccgtgaagggaatctacaagcagatgcccggctgcttcaacttcctgcggaagaagctgtttttcaagaccagcggctccggccagtgcacaaattatgcactgctgaagctcgccggggatgtcgagagtaacccaggacctggaagcggagaaggtcgtggtagtctactaacgtgtggtgatgtagaagaaaatcctggacctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcc

SEQ ID NO: 23 - protein, 1512 aa, synthetic construct
SAGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWLEILMIGVVWRSMEHPVKLLFAPNLVLDRDQGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHASNKGMELLYSMKCKNVVPLYDLLLEAADAHRLHAPTSRGGASVEETDQSHLATAGSTSSHSLQKYYITGEAEGFPATAGGGGSGGGGSGGGGSVDGFDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSGSGQCTNYALLKLAGDVESNPGPGSGEGRGSLLTCGDVEENPGPMSAGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWLEILMIGVVWRSMEHPVKLLFAPNLVLDRDQGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHASNKGMELLYSMKCKNVVPLYDLLLEAADAHRLHAPTSRGGASVEETDQSHLATAGSTSSHSLQKYYITGEAEGFPATAGGGGSGGGGSGGGGSVDGFDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSGSGQCTNYALLKLAGDVESNPGPGSGEGRGSLLTCGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 24 - DNA, 4539 bp, synthetic construct
  source 1..4539: mol_type = other DNA
atgtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagatcgttcacatgatcaactgggcgaagagggtgccaggctttgtggatttgaccctccatgatcaggtccaccttctagaatgtgcctggctagagatcctgatgattggtgtggtctggcgctccatggagcacccagtgaagctactgtttgctcctaacttggtgttggacagggaccagggaaaatgtgtagagggcctggtggagatcttcgacatgctgctggctacatcatctcggttccgcatgatgaatctgcagggagaggagtttgtgtgcctcaaatctattattttgcttaattctggagtgtacacatttctgcccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacgccagtaacaaaggcatggagctgctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagctgggggtggaggttcagggggtggaggttcaggtggtggcggtagtgtcgatggcttcgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcaggctccggccagtgcacaaattatgcactgctgaagctcgccggggatgtcgagagtaacccaggacctggaagcggagaaggtcgtggtagtctactaacgtgtggtgatgtagaagaaaatcctggacctatgtctgccggcgatatgagagccgccaacctgtggccttctccactgatgatcaagcggagcaagaagaactctctggccctgagcctgaccgccgaccagatggtttctgctctgctggatgccgagcctcctatcctgtacagcgagtacgaccccaccagaccttttagcgaggccagcatgatgggcctgctgaccaatctggccgaccgggaaatcgtgcacatgatcaactgggccaagcgcgtgcccggctttgtggatctgacactgcacgaccaagtgcatctgctggaatgcgcctggctggaaatcctgatgatcggcgttgtgtggcggagcatggaacaccctgtgaagctgctgttcgcccctaacctggtgctggacagagatcagggcaaatgcgtggaaggactggtggaaatcttcgacatgctgctggccaccagctccagattccggatgatgaacctgcagggcgaagagttcgtgtgcctgaagtccatcatcctgctgaacagcggcgtgtacaccttcctgcctagcacactgaagtccctggaagagaaggaccacatccacagagtgctggataagatcaccgacacactgatccacctgatggccaaggccggactgacactccagcagcagcatcagagactggcccagctgctgctcatcctgagccacattagacacgccagcaacaaaggcatggaactgctgtactccatgaagtgcaagaacgtggtgcccctgtacgacctgctgctcgaagctgccgatgctcacagactgcacgcccctacatctagaggcggagcctctgtggaagagacagaccagtctcatctggccacagccggcagcacatcttctcacagcctgcagaagtactacatcaccggcgaggccgagggatttcctgctacagctggcggcggaggatctggcggaggtggaagcggaggcggtggaagtgtggatggctttgatgtgggcgccctggaaagcctgagaggcaatgccgatctggcctacatcctgtccatggaaccttgcggccactgcctgattatcaacaacgtgaacttctgtcgcgagagcggcctgagaaccagaaccggcagcaatatcgactgcgagaagctgcggagaagattcagcagcctgcactttatggtggaagtcaagggcgatctgaccgccaagaaaatggtgctggccctgctggaactggccagacaagatcatggcgctctggactgctgcgtggtcgtgattctgtctcatggctgccaggccagccatctgcaattccctggtgccgtgtatggcaccgatggctgtcctgtgtccgtggaaaagatcgtgaacatcttcaacggcacaagctgccctagcctcggcggaaagcccaagctgttcttcatccaagcctgtggcggcgagcagaaggatcacggatttgaggtggcctccacctctcctgaggatgagagccctggaagcaaccccgagcctgatgccacacctttccaagagggactgagaaccttcgaccagctggacgctatcagctccctgcctacacctagcgacatcttcgtgtcctacagcacattccccggcttcgtgtcttggagggatcccaagtctggctcttggtacgtggaaaccctggacgacatctttgagcagtgggcccatagcgaggacctgcagtctctgcttctgagagtggccaatgccgtgtccgtgaagggcatctacaagcagatgcccggctgcttcaacttcctgcggaagaagctgtttttcaagaccagcggctccggccagtgcacaaattatgcactgctgaagctcgccggggatgtcgagagtaacccaggacctggaagcggagaaggtcgtggtagtctactaacgtgtggtgatgtagaagaaaatcctggacctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcc

SEQ ID NO: 25 - protein, 1511 aa, synthetic construct
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGVVWRSMEHPVKLLFAPNLLLDRDQGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPATAGGGGSGGGGSGGGGSVDGFDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSGSGATNFSLLKQAGDVEENPGPGSGEGRGSLLTCGDVEENPGPMSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGVVWRSMEHPVKLLFAPNLLLDRDQGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPATAGGGGSGGGGSGGGGSVDGFDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSGSGQCTNYALLKLAGDVESNPGPGSGEGRGSLLTCGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 26 - DNA, 4536 bp, synthetic construct
  source 1..4536: mol_type = other DNA
atgtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggatggagatcctgatgattggtgtggtctggcgctccatggagcacccagtgaagctactgtttgctcctaacttgctcttggacagggaccagggaaaatgtgtagagggcctggtggagatcttcgacatgctgctggctacatcatctcggttccgcatgatgaatctgcagggagaggagtttgtgtgcctcaaatctattattttgcttaattctggagtgtacacatttctgcccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagctgctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagctgggggtggaggttcagggggtggaggttcaggtggtggcggtagtgtcgatggcttcgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcaggctccggcgcgacgaattttagtctactgaaacaagcgggagacgtggaggaaaaccctggacctggaagcggagaaggtcgtggtagtctactaacgtgtggtgatgtagaagaaaatcctggacctatgtctgccggcgatatgagagccgctaacctgtggccttctccactgatgatcaagcggagcaagaagaactctctggccctgagcctgaccgccgaccagatggtttctgctctgctggatgccgagcctcctatcctgtacagcgagtacgaccccaccagaccttttagcgaggccagcatgatgggcctgctgaccaatctggccgacagagaactggtgcacatgatcaactgggccaagcgcgtgcccggctttgtggatctgacactgcacgaccaagtgcatctgctggaatgcgcctggatggaaatcctgatgatcggcgtcgtgtggcggagcatggaacaccctgtgaagctgctgttcgcccctaacctgctgctggacagagatcagggcaaatgcgtggaaggactggtggaaatcttcgacatgctgctggccaccagctccagattccggatgatgaacctgcagggcgaagagttcgtgtgcctgaagtccatcatcctgctgaacagcggcgtgtacaccttcctgcctagcacactgaagtccctggaagagaaggaccacatccacagagtgctggataagatcaccgacacactgatccacctgatggccaaggccggactgacacttcagcagcagcatcagagactggcccagctgctgctcatcctgagccacatcagacacatgagcaacaaaggcatggaactgctgtactccatgaagtgcaagaacgtggtgcccctgtacgatctgctgctcgaagccgccgatgctcacagactgcatgcccctacatctagaggcggagccagcgtggaagagacagatcagtctcatctggccaccgccggcagcacaagctctcatagcctgcagaagtactacatcaccggcgaggccgagggatttcctgctactgctggtggcggaggatctggcggaggtggaagcggcggaggcggatctgttgatggctttgatgtgggcgccctggaaagcctgagaggcaatgccgatctggcctacatcctgtccatggaaccttgcggccactgcctgattatcaacaacgtgaacttctgtcgcgagagcggcctgagaaccagaaccggcagcaacatcgactgcgagaagctgcggagaagattcagcagcctgcactttatggtggaagtgaagggcgatctgaccgccaagaaaatggtgctggccctgctggaactggccagacaagatcatggcgctctggactgctgcgtggtcgtgattctgtctcatggctgccaggccagccatctgcaattccctggtgccgtgtatggcaccgatggctgtcctgtgtccgtggaaaagatcgtgaacatcttcaacggcacaagctgccctagcctcggcggaaagcccaagctgttcttcatccaagcctgtggcggcgagcagaaggatcacggatttgaggtggcctccacctctcctgaggatgagagccctggaagcaaccccgagcctgatgccacacctttccaagagggactgagaaccttcgaccagctggacgctatcagctccctgcctacacctagcgacatcttcgtgtcctacagcacattccccggcttcgtgtcttggagggatcccaagtctggctcttggtacgtggaaaccctggacgacatctttgagcagtgggcccatagcgaggacctgcagtctctgcttctgagagtggccaatgccgtgtccgtgaagggaatctacaagcagatgcccggctgcttcaacttcctgcggaagaagctgtttttcaagaccagcggctccggccagtgcacaaattatgcactgctgaagctcgccggggatgtcgagagtaacccaggacctggaagcggagaaggtcgtggtagtctactaacgtgtggtgatgtagaagaaaatcctggacctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcc

SEQ ID NO: 27 - protein, 1511 aa, synthetic construct
SAGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWLEILMIGVVWRSMEHPVKLLFAPNLVLDRDQGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHASNKGMELLYSMKCKNVVPLYDLLLEAADAHRLHAPTSRGGASVEETDQSHLATAGSTSSHSLQKYYITGEAEGFPATAGGGGSGGGGSGGGGSVDGFDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSGSGATNFSLLKQAGDVEENPGPGSGEGRGSLLTCGDVEENPGPMSAGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWLEILMIGVVWRSMEHPVKLLFAPNLVLDRDQGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHASNKGMELLYSMKCKNVVPLYDLLLEAADAHRLHAPTSRGGASVEETDQSHLATAGSTSSHSLQKYYITGEAEGFPATAGGGGSGGGGSGGGGSVDGFDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSGSGQCTNYALLKLAGDVESNPGPGSGEGRGSLLTCGDVEENPGPMTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 28 - DNA, 4539 bp, synthetic construct
  source 1..4539: mol_type = other DNA
atgtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagatcgttcacatgatcaactgggcgaagagggtgccaggctttgtggatttgaccctccatgatcaggtccaccttctagaatgtgcctggctagagatcctgatgattggtgtggtctggcgctccatggagcacccagtgaagctactgtttgctcctaacttggtgttggacagggaccagggaaaatgtgtagagggcctggtggagatcttcgacatgctgctggctacatcatctcggttccgcatgatgaatctgcagggagaggagtttgtgtgcctcaaatctattattttgcttaattctggagtgtacacatttctgcccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacgccagtaacaaaggcatggagctgctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagctgggggtggaggttcagggggtggaggttcaggtggtggcggtagtgtcgatggcttcgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcaggctccggcgcgacgaattttagtctactgaaacaagcgggagacgtggaggaaaaccctggacctggaagcggagaaggtcgtggtagtctactaacgtgtggtgatgtagaagaaaatcctggacctatgtctgccggcgatatgagagccgccaacctgtggccttctccactgatgatcaagcggagcaagaagaactctctggccctgagcctgaccgccgaccagatggtttctgctctgctggatgccgagcctcctatcctgtacagcgagtacgaccccaccagaccttttagcgaggccagcatgatgggcctgctgaccaatctggccgaccgggaaatcgtgcacatgatcaactgggccaagcgcgtgcccggctttgtggatctgacactgcacgaccaagtgcatctgctggaatgcgcctggctggaaatcctgatgatcggcgttgtgtggcggagcatggaacaccctgtgaagctgctgttcgcccctaacctggtgctggacagagatcagggcaaatgcgtggaaggactggtggaaatcttcgacatgctgctggccaccagctccagattccggatgatgaacctgcagggcgaagagttcgtgtgcctgaagtccatcatcctgctgaacagcggcgtgtacaccttcctgcctagcacactgaagtccctggaagagaaggaccacatccacagagtgctggataagatcaccgacacactgatccacctgatggccaaggccggactgacactccagcagcagcatcagagactggcccagctgctgctcatcctgagccacattagacacgccagcaacaaaggcatggaactgctgtactccatgaagtgcaagaacgtggtgcccctgtacgacctgctgctcgaagctgccgatgctcacagactgcacgcccctacatctagaggcggagcctctgtggaagagacagaccagtctcatctggccacagccggcagcacatcttctcacagcctgcagaagtactacatcaccggcgaggccgagggatttcctgctacagctggcggcggaggatctggcggaggtggaagcggaggcggtggaagtgtggatggctttgatgtgggcgccctggaaagcctgagaggcaatgccgatctggcctacatcctgtccatggaaccttgcggccactgcctgattatcaacaacgtgaacttctgtcgcgagagcggcctgagaaccagaaccggcagcaatatcgactgcgagaagctgcggagaagattcagcagcctgcactttatggtggaagtcaagggcgatctgaccgccaagaaaatggtgctggccctgctggaactggccagacaagatcatggcgctctggactgctgcgtggtcgtgattctgtctcatggctgccaggccagccatctgcaattccctggtgccgtgtatggcaccgatggctgtcctgtgtccgtggaaaagatcgtgaacatcttcaacggcacaagctgccctagcctcggcggaaagcccaagctgttcttcatccaagcctgtggcggcgagcagaaggatcacggatttgaggtggcctccacctctcctgaggatgagagccctggaagcaaccccgagcctgatgccacacctttccaagagggactgagaaccttcgaccagctggacgctatcagctccctgcctacacctagcgacatcttcgtgtcctacagcacattccccggcttcgtgtcttggagggatcccaagtctggctcttggtacgtggaaaccctggacgacatctttgagcagtgggcccatagcgaggacctgcagtctctgcttctgagagtggccaatgccgtgtccgtgaagggcatctacaagcagatgcccggctgcttcaacttcctgcggaagaagctgtttttcaagaccagcggctccggccagtgcacaaattatgcactgctgaagctcgccggggatgtcgagagtaacccaggacctggaagcggagaaggtcgtggtagtctactaacgtgtggtgatgtagaagaaaatcctggacctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 29 - skipped

SEQ ID NO: 30 - protein, 10 aa, synthetic construct
GSGSGSGSGG

SEQ ID NO: 31 - protein, 4 aa, synthetic construct
GGGS

SEQ ID NO: 32 - skipped

SEQ ID NO: 33 - protein, 4 aa, synthetic construct
GGSG

SEQ ID NO: 34 - protein, 4 aa, synthetic construct
GGGS

SEQ ID NO: 35 - protein, 5 aa, synthetic construct
GGGGS

SEQ ID NO: 36 - protein, 9 aa, synthetic construct
GGSGGSGGS

SEQ ID NO: 37 - protein, 10 aa, synthetic construct
GGGGSGGGGS

SEQ ID NO: 38 - protein, 15 aa, synthetic construct
GGGGSGGGGSGGGGS

SEQ ID NO: 39 - protein, 19 aa, synthetic construct
GGGGSGGGGSGGGGSVDGF

SEQ ID NO: 40 - protein, 312 aa, synthetic construct
AGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWMEILMIGVVWRSMEHPVKLLFAPNLLLDRDEGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPAT

SEQ ID NO: 41 - protein, 312 aa, synthetic construct
AGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWLEILMIGVVWRSMEHPVKLLFAPNLLLDRDQGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHASNKGMELLYSMKCKNVVPLYDLLLEAADAHRLHAPTSRGGASVEETDQSHLATAGSTSSHSLQKYYITGEAEGFPAT

SEQ ID NO: 42 - protein, 312 aa, synthetic construct
AGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWMEILMIGLVWRSMEHPVKLLFAPNLLLDRDQGKCVEGMVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPAT

SEQ ID NO: 43 - protein, 312 aa, synthetic construct
AGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWLEILMIGVVWRSMEHPVKLLFAPNLVLDRDQGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHASNKGMELLYSMKCKNVVPLYDLLLEAADAHRLHAPTSRGGASVEETDQSHLATAGSTSSHSLQKYYITGEAEGFPAT

SEQ ID NO: 44 - protein, 611 aa, synthetic construct
AGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWMEILMIGVVWRSMEHPVKLLFAPNLLLDRDQGKCVEGLVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPATGGGGSGGGGSGGGGSVDGFMDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 45 - protein, 1833 aa, synthetic construct
ATGGCTGGAGACATGAGAGCTGCCAACCTTTGGCCAAGCCCGCTCATGATCAAACGCTCTAAGAAGAACAGCCTGGCCTTGTCCCTGACGGCCGACCAGATGGTCAGTGCCTTGTTGGATGCTGAGCCCCCCATACTCTATTCCGAGTATGATCCTACCAGACCCTTCAGTGAAGCTTCGATGATGGGCTTACTGACCAACCTGGCAGACAGGGAGCTGGTTCACATGATCAACTGGGCGAAGAGGGTGCCAGGCTTTGTGGATTTGACCCTCCATGATCAGGTCCACCTTCTAGAATGTGCCTGGATGGAGATCCTGATGATTGGTGTGGTCTGGCGCTCCATGGAGCACCCAGTGAAGCTACTGTTTGCTCCTAACTTGCTCTTGGACAGGGACCAGGGAAAATGTGTAGAGGGCCTGGTGGAGATCTTCGACATGCTGCTGGCTACATCATCTCGGTTCCGCATGATGAATCTGCAGGGAGAGGAGTTTGTGTGCCTCAAATCTATTATTTTGCTTAATTCTGGAGTGTACACATTTCTGCCCAGCACCCTGAAGTCTCTGGAAGAGAAGGACCATATCCACCGAGTCCTGGACAAGATCACAGACACTTTGATCCACCTGATGGCCAAGGCAGGCCTGACCCTGCAGCAGCAGCACCAGCGGCTGGCCCAGCTCCTCCTCATCCTCTCCCACATCAGGCACATGAGTAACAAAGGCATGGAGCTGCTGTACAGCATGAAGTGCAAGAACGTGGTGCCCCTCTATGACCTGCTGCTGGAGGCGGCGGACGCCCACCGCCTACATGCGCCCACTAGCCGTGGAGGGGCATCCGTGGAGGAGACGGACCAAAGCCACTTGGCCACTGCGGGCTCTACTTCATCGCATTCCTTGCAAAAGTATTACATCACGGGGGAGGCAGAGGGTTTCCCTGCCACAGGGGGTGGAGGTTCAGGGGGTGGAGGTTCAGGTGGTGGCGGTAGTGTCGATGGCTTCGATGTCGGTGCTCTTGAGAGTTTGAGGGGAAATGCAGATTTGGCTTACATCCTGAGCATGGAGCCCTGTGGCCACTGCCTCATTATCAACAATGTGAACTTCTGCCGTGAGTCCGGGCTCCGCACCCGCACTGGCTCCAACATCGACTGTGAGAAGTTGCGGCGTCGCTTCTCCTCGCTGCATTTCATGGTGGAGGTGAAGGGCGACCTGACTGCCAAGAAAATGGTGCTGGCTTTGCTGGAGCTGGCGCGGCAGGACCACGGTGCTCTGGACTGCTGCGTGGTGGTCATTCTCTCTCACGGCTGTCAGGCCAGCCACCTGCAGTTCCCAGGGGCTGTCTACGGCACAGATGGATGCCCTGTGTCGGTCGAGAAGATTGTGAACATCTTCAATGGGACCAGCTGCCCCAGCCTGGGAGGGAAGCCCAAGCTCTTTTTCATCCAGGCCTGTGGTGGGGAGCAGAAAGACCATGGGTTTGAGGTGGCCTCCACTTCCCCTGAAGACGAGTCCCCTGGCAGTAACCCCGAGCCAGATGCCACCCCGTTCCAGGAAGGTTTGAGGACCTTCGACCAGCTGGACGCCATATCTAGTTTGCCCACACCCAGTGACATCTTTGTGTCCTACTCTACTTTCCCAGGTTTTGTTTCCTGGAGGGACCCCAAGAGTGGCTCCTGGTACGTTGAGACCCTGGACGACATCTTTGAGCAGTGGGCTCACTCTGAAGACCTGCAGTCCCTCCTGCTTAGGGTCGCTAATGCTGTTTCGGTGAAAGGGATTTATAAACAGATGCCTGGTTGCTTTAATTTCCTCCGGAAAAAACTTTTCTTTAAAACATCA

SEQ ID NO: 46 - protein, 611 aa, synthetic construct
AGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGVVWRSMEHPVKLLFAPNLLLDRDEGKCVEGMVEIFDMLLATSSRFRMMNLQGEEFVCLKSIILLNSGVYTFLPSTLKSLEEKDHIHRVLDKITDTLIHLMAKAGLTLQQQHQRLAQLLLILSHIRHMSNKGMEFLYSMKCKNVVPLYDLLLEAADAHRLHAPTSRGGASVEETDQSHLATAGSTSSHSLQKYYITGEAEGFPATGGGGSGGGGSGGGGSVDGFMDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 47 - DNA, 1833 bp, synthetic construct
  source 1..1833: mol_type = other DNA
atggctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtgtggtctggcgctccatggagcacccagtgaagctactgtttgctcctaacttgctcttggacagggacgagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgcccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagtttctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagggggtggaggttcagggggtggaggttcaggtggtggcggtagtgtcgatggcttcgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatca

SEQ ID NO: 48 - protein, 611 aa, synthetic construct
AGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWLEILMIGVVWRSMEHPVKLLFAPNLLLDRDEGKCVEGLVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHASNKGMEFLYSMKCKNVVPLYDLLLEAADAHRLHAPTSRGGASVEETDQSHLATAGSTSSHSLQKYYITGEAEGFPATGGGGSGGGGSGGGGSVDGFMDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 49 - DNA, 1833 bp, synthetic construct
  source 1..1833: mol_type = other DNA
atggctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagatcgttcacatgatcaactgggcgaagagggtgccaggctttgtggatttgaccctccatgatcaggtccaccttctagaatgtgcctggctagagatcctgatgattggtgtggtctggcgctccatggagcacccagtgaagctactgtttgctcctaacttgctcttggacagggacgagggaaaatgtgtagagggcctggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacgccagtaacaaaggcatggagtttctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagggggtggaggttcagggggtggaggttcaggtggtggcggtagtgtcgatggcttcgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatca

SEQ ID NO: 50 - protein, 611 aa, synthetic construct
AGDMRAANLWPSPLMIKRSKKNSLALSLTADQMVSALLDAEPPILYSEYDPTRPFSEASMMGLLTNLADREIVHMINWAKRVPGFVDLTLHDQVHLLECAWLEILMIGVVWRSMEHPVKLLFAPNLVLDRDEGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMELLYSMKCKNVVPLYDLLLEAADAHRLHAPTSRGGASVEETDQSHLATAGSTSSHSLQKYYITGEAEGFPATGGGGSGGGGSGGGGSVDGFMDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 51 - DNA, 1833 bp, synthetic construct
  source 1..1833: mol_type = other DNA
atggctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagatcgttcacatgatcaactgggcgaagagggtgccaggctttgtggatttgaccctccatgatcaggtccaccttctagaatgtgcctggctagagatcctgatgattggtgtggtctggcgctccatggagcacccagtgaagctactgtttgctcctaacttggtgttggacagggacgagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagctgctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagggggtggaggttcagggggtggaggttcaggtggtggcggtagtgtcgatggcttcgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatca

SEQ ID NO: 52 - DNA, 57 bp, synthetic construct
  source 1..57: mol_type = other DNA
gcgacgaattttagtctactgaaacaagcgggagacgtggaggaaaaccctggacct

SEQ ID NO: 53 - protein, 18 aa, synthetic construct
EGRGSLLTCGDVEENPGP

SEQ ID NO: 54 - DNA, 54 bp, synthetic construct
  source 1..54: mol_type = other DNA
gaagggcgcgggtctctcctcacttgtggagatgttgaggaaaatccaggacca

SEQ ID NO: 55 - protein, 41 aa, synthetic construct
QCTNYALLKLAGDVESNPGPGSGEGRGSLLTCGDVEENPGP

SEQ ID NO: 56 - DNA, 123 bp, synthetic construct
  source 1..123: mol_type = other DNA
cagtgcacaaattatgcactgctgaagctcgccggggatgtcgagagtaacccaggacctggaagcggagaaggtcgtggtagtctactaacgtgtggtgatgtagaagaaaatcctggacct

SEQ ID NO: 57 - DNA, 120 bp, synthetic construct
  source 1..120: mol_type = other DNA
gcgacgaattttagtctactgaaacaagcgggagacgtggaggaaaaccctggacctggaagcggagaaggtcgtggtagtctactaacgtgtggtgatgtagaagaaaatcctggacct

SEQ ID NO: 58 - DNA, 30 bp, synthetic construct
  source 1..30: mol_type = other DNA
tttcatcttctaggtatgacaacgattaca

SEQ ID NO: 59 - DNA, 30 bp, synthetic construct
  source 1..30: mol_type = other DNA
tgtaatcgttgtcatacctagaagatgaaa

SEQ ID NO: 60 - DNA, 21 bp, synthetic construct
  source 1..21: mol_type = other DNA
atcttctaggtatgacaacga

SEQ ID NO: 61 - protein, 44 aa, synthetic construct
GSGQCTNYALLKLAGDVESNPGPGSGEGRGSLLTCGDVEENPGP

SEQ ID NO: 62 - protein, 199 aa, synthetic construct
MTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 63 - protein, 43 aa, synthetic construct
GSGATNFSLLKQAGDVEENPGPGSGEGRGSLLTCGDVEENPGP